(19) United States
(12) Patent Application Publication
(10) Pub. No.: US 2024/0165146 A1
(43) Pub. Date: May 23, 2024

(54) Conjugated Antisense Compounds for Use in Therapy

(71) Applicant: Ionis Pharmaceuticals, Inc., Carlsbad, CA (US)

(72) Inventors: Nicholas J. Viney, Carlsbad, CA (US); Richard S. Geary, Carlsbad, CA (US); Yanfeng Wang, Carlsbad, CA (US); Zhengrong Yu, Carlsbad, CA (US); Rudy Gunawan, Apex, NC (US)

(73) Assignee: Ionis Pharmaceuticals, Inc., Carlsbad, CA (US)

(21) Appl. No.: 18/340,192

(22) Filed: Jun. 23, 2023

Related U.S. Application Data
(63) Continuation of application No. 16/947,310, filed on Jul. 28, 2020, now abandoned.
Application No. 16/947,310 is a continuation of application No. 15/771,598, filed on Apr. 27, 2018, now abandoned.
(60) Provisional application No. 62/252,397, filed on Nov. 6, 2015.

(51) Int. Cl. A61K 31/712; A61K 9/00; A61K 48/00; A61P 3/06; C07H 19/20; C07H 21/00; C07H 21/02; C07H 21/04; C12N 15/113

(52) U.S. Cl. CPC A61K 31/712; A61K 9/0019; A61K 48/0016; A61K 48/005; A61K 48/0083; A61P 3/06; C07H 19/20; C07H 21/00; C07H 21/02; C07H 21/04; C12N 15/113; C12N 2310/341; C12N 2310/3515

(57) ABSTRACT

Provided herein are methods of administering gapmer oligomeric compounds with GalNAc conjugate groups to a human.

### SEQUENCE LISTING

The present application contains a Sequence Listing which has been submitted electronically in XML format. Said XML copy, created on Feb. 8, 2023, is named “CORE0138SEQ.xml” and is 325,051 bytes in size. The information in the electronic format of the sequence listing is incorporated herein by reference in its entirety.

### BACKGROUND OF THE INVENTION

The principle behind antisense technology is that an antisense compound hybridizes to a target nucleic acid and modulates the amount, activity, and/or function of the target nucleic acid. For example in certain instances, antisense compounds result in altered transcription or translation of a target. Such modulation of expression can be achieved by, for example, target mRNA degradation or occupancy-based inhibition. An example of modulation of RNA target function by degradation is RNase H-based degradation of the target RNA upon hybridization with a DNA-like antisense compound. Another example of modulation of gene expression by target degradation is RNA interference (RNAi). RNAi refers to antisense-mediated gene silencing through a mechanism that utilizes the RNA-induced silencing complex (RISC). An additional example of modulation of RNA target function is by an occupancy-based mechanism such as is employed naturally by microRNA. MicroRNAs are small non-coding RNAs that regulate the expression of protein-coding RNAs. The binding of an antisense compound to a microRNA prevents that microRNA from binding to its messenger RNA targets, and thus interferes with the function of the microRNA. MicroRNA mimics can enhance native microRNA function. Certain antisense compounds alter splicing of pre-mRNA. Regardless of the specific mechanism, sequence-specificity makes antisense compounds attractive as tools for target validation and gene functionalization, as well as therapeutics to selectively modulate the expression of genes involved in the pathogenesis of diseases.

Antisense technology is an effective means for modulating the expression of one or more specific gene products and can therefore prove to be uniquely useful in a number of therapeutic, diagnostic, and research applications. Chemically modified nucleosides may be incorporated into antisense compounds to enhance one or more properties, such as nuclease resistance, pharmacokinetics or affinity for a target nucleic acid. In 1998, the antisense compound, Vitravene® (fomivirsen; developed by Isis Pharmaceuticals Inc., Carlsbad, CA) was the first antisense drug to achieve marketing clearance from the U.S. Food and Drug Administration (FDA), and is currently a treatment of cytomegalovirus (CMV)-induced retinitis in AIDS patients.

New chemical modifications have improved the potency and efficacy of antisense compounds, uncovering the potential enhancing subcutaneous administration, decreasing potential for side effects, and leading to improvements in patient convenience. Chemical modifications increasing potency of antisense compounds allow administration of lower doses, which reduces the potential for toxicity, as well as decreasing overall cost of therapy. Modifications increasing the resistance to degradation result in slower clearance from the body, allowing for less frequent dosing. Different types of chemical modifications can be combined in one compound to further optimize the compound's efficacy.

One chemical modification used to improve the activity of RNAse H dependent (gapmer) antisense compounds in vivo is conjugation to a conjugate group, such as a GalNAc cluster. Conjugation to a conjugate group has been shown to improve potency in vivo in non-human subjects, for example including the use of RNAse H dependent (gapmer) antisense compounds conjugated to GalNAc clusters as disclosed in WO 2014/179620. Prior to the present invention, no RNAse H dependent (gapmer) antisense compounds conjugated to GalNAc clusters had been tested in humans to achieve target reduction.

### SUMMARY OF THE INVENTION

The present disclosure provides gapmer oligomeric compounds comprising a conjugate group, wherein the conjugate group comprises a GalNAc cluster, for use in a method of treating a disease or condition in a human, wherein the method comprises administering not more than 1500 mg of the oligomeric compound to the human during a dosing period.

While it was known that oligomeric gapmer compounds comprising a GalNAc cluster had improved in vivo potency from work in non-human subjects (e.g. WO 2014/179620), the inventors were the first to test this class of compounds in humans. It was discovered that the oligomeric gapmer compounds comprising a GalNAc cluster are particularly effective when administered to a human subject. The improvement provided in humans was unexpectedly greater than the improvement seen in the non-human subjects. Amongst the improvements observed included increased potency relative to that expected from the earlier work using non-human subjects. A further improvement observed included increased half-life relative to that expected from the work using non-human subjects.

Following this discovery, one aspect of the invention is oligomeric gapmer compounds comprising a GalNAc cluster for use a method of treating a disease or condition in a human by using lower than expected doses, and yet still providing excellent reduction of a given target nucleic acid. In addition, following the first testing of these oligomeric gapmer compounds in humans, a further aspect of the invention is that the oligomeric gapmer compounds comprising a GalNAc cluster may be administered to a human subject only once a week, only once a month, or only once every three months, and yet still provide excellent reduction of a given target nucleic acid. See, e.g., Viney, et al. Lancet, 2016, September 2016: 388: 2239-53.

The disclosure also provides unit dosage forms with low amounts of the oligomeric gapmer compound useful in these methods thanks to their relatively low drug amounts.

The present disclosure provides the following non-limiting numbered embodiments:
Embodiment 1: An oligomeric compound comprising a modified oligonucleotide consisting of 12-22 linked nucleosides comprising a region having a gapmer motif, and a conjugate group comprising a GalNAc cluster,
   for use in treating or preventing a disease or condition in a human, wherein the treatment comprises administering not more than 1500 mg of the oligomeric compound to the human during a dosing period.
Embodiment 2: The oligomeric compound for use according to embodiment 1, wherein the gapmer motif is a sugar motif.
Embodiment 3: The oligomeric compound for use according to any of embodiments 1-2, wherein the modified oligonucleotide is a gapmer.
Embodiment 4: The oligomeric compound for use according to any of embodiments 1-3, wherein the modified oligonucleotide has a gapmer motif comprising:
   a 5′-region consisting of 1-5 linked 5′-region nucleosides;
   a central region consisting of 6-10 linked central region nucleosides; and
   a 3′-region consisting of 1-5 linked 3′-region nucleosides; wherein each of the 5′-region nucleosides and each of the 3′-region nucleosides comprises a modified sugar moiety and each of the central region nucleosides comprises an unmodified DNA sugar moiety.
Embodiment 5: The oligomeric compound for use according to any of embodiments 1-4, wherein the 5′-region consists of 2 linked 5′-region nucleosides.
Embodiment 6: The oligomeric compound for use according to any of embodiments 1-4, wherein the 5′-region consists of 3 linked 5′-region nucleosides.
Embodiment 7: The oligomeric compound for use according to any of embodiments 1-4, wherein the 5′-region consists of 4 linked 5′-region nucleosides.
Embodiment 8: The oligomeric compound for use according to any of embodiments 1-4, wherein the 5′-region consists of 5 linked 5′-region nucleosides.
Embodiment 9: The oligomeric compound for use according to any of embodiments 1 to 8, wherein the 3′-region consists of 2 linked 3′-region nucleosides.
Embodiment 10: The oligomeric compound for use according to any of embodiments 1 to 8, wherein the 3′-region consists of 3 linked 3′-region nucleosides.
Embodiment 11: The oligomeric compound for use according to any of embodiments 1 to 8, wherein the 3′-region consists of 4 linked 3′-region nucleosides.
Embodiment 12: The oligomeric compound for use according to any of embodiments 1 to 8, wherein the 3′-region consists of 5 linked 3′-region nucleosides.
Embodiment 13: The oligomeric compound for use according to any of embodiments 1 to 12, wherein the central region consists of 6 linked central region nucleosides.
Embodiment 14: The oligomeric compound for use according to any of embodiments 1 to 12, wherein the central region consists of 7 linked central region nucleosides.
Embodiment 15: The oligomeric compound for use according to any of embodiments 1 to 12, wherein the central region consists of 8 linked central region nucleosides.
Embodiment 16: The oligomeric compound for use according to any of embodiments 1 to 12, wherein the central region consists of 9 linked central region nucleosides.
Embodiment 17: The oligomeric compound for use according to any of embodiments 1 to 12, wherein the central region consists of 10 linked central region nucleosides.
Embodiment 18: The oligomeric compound for use according to any of embodiments 1-4, wherein the 5′-region consists of 2 linked 5′-region nucleosides, the 3′-region consists of 2 linked 3′-region nucleosides, and the central region consists of 8 linked central region nucleosides.
Embodiment 19: The oligomeric compound for use according to any of embodiments 1-4, wherein the 5′-region consists of 2 linked 5′-region nucleosides, the 3′-region consists of 2 linked 3′-region nucleosides, and the central region consists of 9 linked central region nucleosides.
Embodiment 20: The oligomeric compound for use according to any of embodiments 1-4, wherein the 5′-region consists of 2 linked 5′-region nucleosides, the 3′-region consists of 2 linked 3′-region nucleosides, and the central region consists of 10 linked central region nucleosides.
Embodiment 21: The oligomeric compound for use according to any of embodiments 1-4, wherein the 5′-region consists of 2 linked 5′-region nucleosides, the 3′-region consists of 2 linked 3′-region nucleosides, the central region consists of 10 linked central region nucleosides: and wherein each 5′-region nucleoside and each 3′-region nucleoside are 2′MOE nucleosides.
Embodiment 22: The oligomeric compound for use according to any of embodiments 1-4, wherein the 5′-region consists of 5 linked 5′-region nucleosides, the 3′-region consists of 5 linked 3′-region nucleosides, and the central region consists of 10 linked central region nucleosides.
Embodiment 23: The oligomeric compound for use according to any of embodiments 1-4, wherein the 5′-region consists of 5 linked 5′-region nucleosides, the 3′-region consists of 5 linked 3′-region nucleosides, and the central region consists of 8 linked central region nucleosides.
Embodiment 24: The oligomeric compound for use according to any of embodiments 1-4, wherein the 5′-region consists of 4 linked 5′-region nucleosides, the 3′-region consists of 4 linked 3′-region nucleosides, and the central region consists of 8 linked central region nucleosides.
Embodiment 25: The oligomeric compound for use according to any of embodiments 1 to 24, wherein at least one 5′-region nucleoside is a 2′-modified nucleoside.
Embodiment 26: The oligomeric compound for use according to any of embodiments 1 to 24, wherein each 5′-region nucleoside is a 2′-modified nucleoside.
Embodiment 27: The oligomeric compound for use according to embodiment 25 or 26, wherein the 2′-modified nucleoside is selected from among 2′-F, 2′-OCH₃, and 2′-MOE.
Embodiment 28: The oligomeric compound for use according to embodiment 25 or 26, wherein the 2′-modified nucleoside is 2′-MOE.
Embodiment 29: The oligomeric compound for use according to embodiment 25 or 26, wherein the 2′-modified nucleoside is 2′-OCH₃.
Embodiment 30: The oligomeric compound for use according to embodiment 25 or 26, wherein the 2′-modified nucleoside is 2′-F.
Embodiment 31: The oligomeric compound for use according to any of embodiments 1 to 20, 22 to 25, or 27 to 29, wherein at least one 5′-region nucleoside is a bicyclic nucleoside.
Embodiment 32: The oligomeric compound for use according to any of embodiments 1 to 20 or 22 to 26, wherein each 5′-region nucleoside is a bicyclic nucleoside.
Embodiment 33: The oligomeric compound for use according to any of embodiments 31 or 32, wherein the bicyclic nucleoside is selected from among cEt or LNA.
Embodiment 34: The oligomeric compound for use according to embodiment 33, wherein the bicyclic nucleoside is cEt.
Embodiment 35: The oligomeric compound for use according to embodiment 33, wherein the bicyclic nucleoside is LNA.
Embodiment 36: The oligomeric compound for use according to any of embodiments 1 to 35, wherein at least one 3′-region nucleoside is a 2′-modified nucleoside.
Embodiment 37: The oligomeric compound for use according to any of embodiments 1 to 35, wherein each 3′-region nucleoside is a 2′-modified nucleoside.
Embodiment 38: The oligomeric compound for use according to embodiment 36 or 37, wherein the 2′-modified nucleoside is selected from among 2′-F, 2′-OCH₃, or 2′-MOE.
Embodiment 39: The oligomeric compound for use according to embodiment 38, wherein the 2′-modified nucleoside is 2′-MOE.
Embodiment 40: The oligomeric compound for use according to embodiment 38, wherein the 2′-modified nucleoside is 2′-OCH₃.
Embodiment 41: The oligomeric compound for use according to embodiment 38, wherein the 2′-modified nucleoside is 2′-F.
Embodiment 42: The oligomeric compound for use according to any of embodiments 1 to 20, 22 to 36, or 38 to 42, wherein at least one 3′-region nucleoside is a bicyclic nucleoside.
Embodiment 43: The oligomeric compound for use according to any of embodiments 1 to 35, wherein each 3′-region nucleoside is a bicyclic nucleoside.
Embodiment 44: The oligomeric compound for use according to any of embodiments 42 or 43, wherein the bicyclic nucleoside is selected from among cEt or LNA.
Embodiment 45: The oligomeric compound for use according to embodiment 44, wherein the bicyclic nucleoside is cEt.
Embodiment 46: The oligomeric compound for use according to embodiment 44, wherein the bicyclic nucleoside is LNA.
Embodiment 47: The oligomeric compound for use according to any of embodiments 1 to 46, wherein the GalNAc cluster comprises 1-3 GalNAc ligands.
Embodiment 48: The oligomeric compound for use according to any of embodiments 1 to 47, wherein the GalNAc cluster comprises a cell-targeting moiety having the formula:

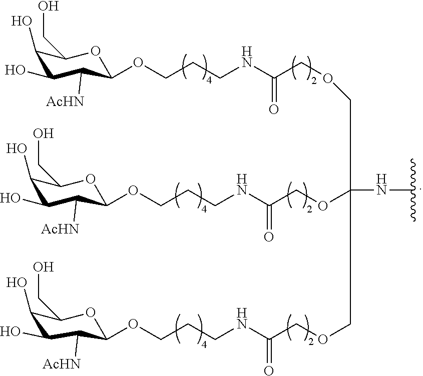

Embodiment 49: The oligomeric compound for use according to any of embodiments 1 to 47, wherein the GalNAc cluster comprises a cell-targeting moiety having the formula:

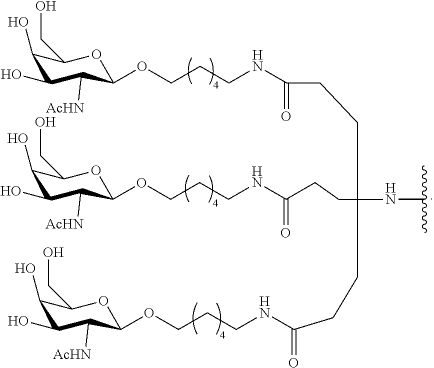

Embodiment 50: The oligomeric compound for use according to any of embodiments 1 to 47, wherein the GalNAc cluster comprises a cell-targeting moiety having the formula:

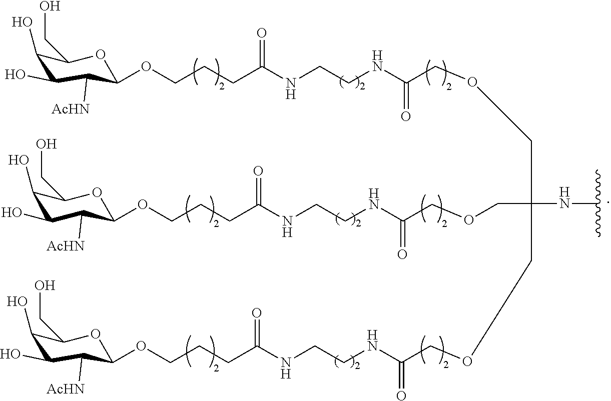

Embodiment 51: The oligomeric compound for use according to any of embodiments 1 to 47, wherein the GalNAc cluster comprises a cell-targeting moiety having the formula:

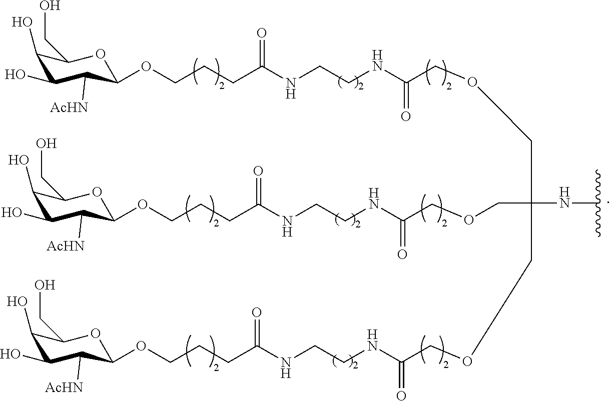

Embodiment 52: The oligomeric compound for use according to any of embodiments 1 to 47, wherein the GalNAc cluster comprises a cell-targeting moiety having the formula:

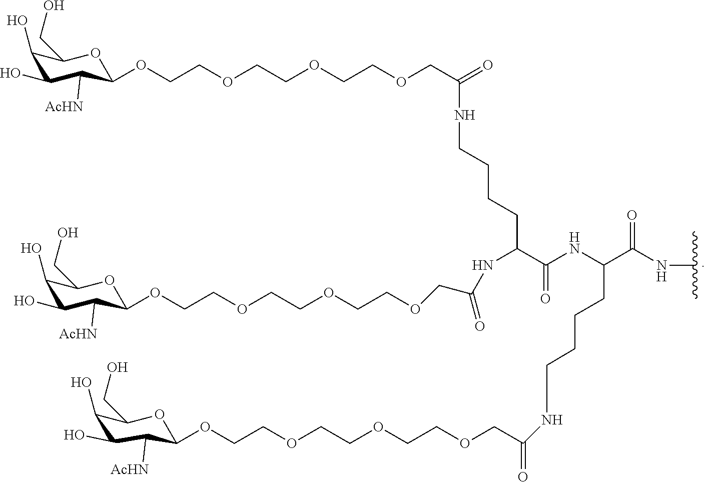

Embodiment 53: The oligomeric compound for use according to any of embodiments 1 to 47, wherein the GalNAc cluster comprises a cell-targeting moiety having the formula:

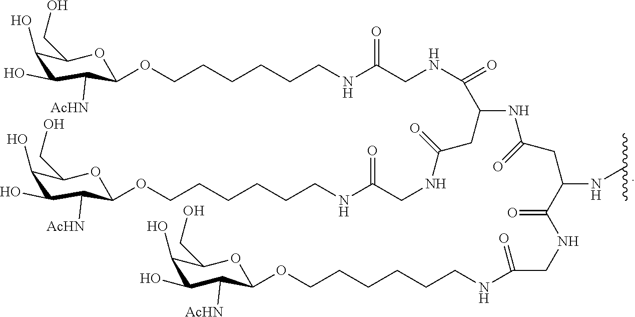

Embodiment 54: The oligomeric compound for use according to any of embodiments 1 to 53, wherein the modified oligonucleotide comprises at least one modified internucleoside linkage.
Embodiment 55: The oligomeric compound for use according to any of embodiments 1 to 54, wherein each internucleoside linkage of the modified oligonucleotide is a modified internucleoside linkage.
Embodiment 56: The oligomeric compound for use according to any of embodiments 1 to 54, wherein at least one internucleoside linkage is a phosphorothioate internucleoside linkage.
Embodiment 57: The oligomeric compound for use according to any of embodiments 54 or 56 wherein the modified oligonucleotide comprises at least one unmodified phosphodiester (or phosphate) internucleoside linkage.
Embodiment 58: The oligomeric compound for use according to any of embodiments 54 to 57, wherein each internucleoside linkage is either an unmodified phosphodiester internucleoside linkage or a phosphorothioate internucleoside linkage.
Embodiment 59: The oligomeric compound for use according to any of embodiments 1 to 58, wherein the modified oligonucleotide comprises at least one modified nucleobase.
Embodiment 60: The oligomeric compound for use according to embodiment 59, wherein the modified nucleobase is a 5-Me cytosine.
Embodiment 61: The oligomeric compound for use according to any of embodiments 1 to 60, wherein the modified oligonucleotide consists of 12-20 linked nucleosides.
Embodiment 62: The oligomeric compound for use according to any of embodiments 1 to 60, wherein the modified oligonucleotide consists of 14-20 linked nucleosides.
Embodiment 63: The oligomeric compound for use according to any of embodiments 1 to 60, wherein the modified oligonucleotide consists of 16-20 linked nucleosides.
Embodiment 64: The oligomeric compound for use according to any of embodiments 1 to 60, wherein the modified oligonucleotide consists of 18-20 linked nucleosides.
Embodiment 65: The oligomeric compound for use according to any of embodiments 1 to 60, wherein the modified oligonucleotide consists of 20 linked nucleosides.
Embodiment 66: The oligomeric compound for use according to any preceding embodiment, wherein the oligomeric compound: (i) consists of 20 linked nucleosides; (ii) the 5′-region consists of 5 linked 5′-region nucleosides and each 5′-region nucleoside is 2′-MOE; (iii) the central region consists of 10 linked central region nucleosides; (iv) the 3′-region consists of 5 linked 3′-region nucleosides and each 3′-region nucleoside is 2′-MOE; (v) the modified oligonucleotide comprises at least one modified internucleoside linkage; and (vi) the GalNAc cluster comprises a cell-targeting moiety according to any of embodiments 39-44.
Embodiment 67: The oligomeric compound for use according to any preceding embodiment, wherein the oligomeric compound: (i) consists of 20 linked nucleosides; (ii) the 5′-region consists of 5 linked 5′-region nucleosides and each 5′-region nucleoside is selected from cEt and LNA; (iii) the central region consists of 10 linked central region nucleosides; (iv) the 3′-region consists of 5 linked 3′-region nucleosides and each 3′-region nucleoside is selected from cEt and LNA; (v) the modified oligonucleotide comprises at least one modified internucleoside linkage; and (vi) the GalNAc cluster comprises a cell-targeting moiety according to any of embodiments 39-44.
Embodiment 68: The oligomeric compound for use according to any preceding embodiment, wherein the treatment comprises administering not more than 1000 mg of the oligomeric compound to the human during the dosing period.
Embodiment 69: The oligomeric compound for use according to any preceding embodiment, wherein the treatment comprises administering not more than 500 mg of the oligomeric compound to the human during the dosing period.
Embodiment 70: The oligomeric compound for use according to any preceding embodiment, wherein the treatment comprises administering not more than 250 mg of the oligomeric compound to the human during the dosing period.
Embodiment 71: The oligomeric compound for use according to any preceding embodiment, wherein the treatment comprises administering not more than 100 mg of the oligomeric compound to the human during the dosing period.
Embodiment 72: The oligomeric compound for use according to any of embodiments 1-71, wherein the dosing period is three months.
Embodiment 73: The oligomeric compound for use according to any of embodiments 1-71, wherein the dosing period is two months.
Embodiment 74: The oligomeric compound for use according to any of embodiments 1-71, wherein the dosing period is one month.
Embodiment 75: The oligomeric compound for use according to any of embodiments 1-71, wherein the dosing period is four weeks.
Embodiment 76: The oligomeric compound for use according to any of embodiments 1-71, wherein the dosing period is three weeks.
Embodiment 77: The oligomeric compound for use according to any of embodiments 1-71, wherein the dosing period is two weeks.
Embodiment 78: The oligomeric compound for use according to any of embodiments 1-71, wherein the dosing period is one week.
Embodiment 79: The oligomeric compound for use according to any preceding embodiment, wherein the treatment comprises administering a unit dose comprising not more than 250 mg of the oligomeric compound.
Embodiment 80: The oligomeric compound for use according to any preceding embodiment, wherein the treatment comprises administering a unit dose comprising not more than 100 mg of the oligomeric compound.
Embodiment 81: The oligomeric compound for use according to any preceding embodiment, wherein the treatment comprises administering a unit dose comprising not more than 75 mg of the oligomeric compound.
Embodiment 82: The oligomeric compound for use according to any preceding embodiment, wherein the treatment comprises administering a unit dose comprising not more than 50 mg of the oligomeric compound.
Embodiment 83: The oligomeric compound for use according to any preceding embodiment, wherein the treatment comprises administering a unit dose comprising not more than 40 mg of the oligomeric compound.
Embodiment 84: The oligomeric compound for use according to any preceding embodiment, wherein the treatment comprises administering a unit dose comprising not more than 30 mg of the oligomeric compound.
Embodiment 85: The oligomeric compound for use according to any preceding embodiment, wherein the treatment comprises administering a unit dose comprising not more than 25 mg of the oligomeric compound.
Embodiment 86: The oligomeric compound for use according to any preceding embodiment, wherein the treatment comprises administering a unit dose comprising not more than 20 mg of the oligomeric compound.
Embodiment 87: The oligomeric compound for use according to any preceding embodiment, wherein the treatment comprises administering a unit dose comprising not more than 15 mg of the oligomeric compound.
Embodiment 88: The oligomeric compound for use according to any of embodiments 1 to 78, wherein the treatment comprises administering a unit dose of from 75 mg to 85 mg, optionally 80 mg.
Embodiment 89: The oligomeric compound for use according to any of embodiments 1 to 78, wherein the treatment comprises administering a unit dose of from 55 mg to 65 mg, optionally 60 mg.
Embodiment 90: The oligomeric compound for use according to any of embodiments 1 to 78, wherein the treatment comprises administering a unit dose of from 45 mg to 55 mg, optionally 50 mg.
Embodiment 91: The oligomeric compound for use according to any of embodiments 1 to 78, wherein the treatment comprises administering a unit dose of from 35 mg to 45 mg, optionally 40 mg.
Embodiment 92: The oligomeric compound for use according to any of embodiments 1 to 78, wherein the treatment comprises administering a unit dose of from 25 mg to 35 mg, optionally 30 mg.
Embodiment 93: The oligomeric compound for use according to any of embodiments 1 to 78, wherein the treatment comprises administering a unit dose of from 15 mg to 25 mg, optionally 20 mg.
Embodiment 94: The oligomeric compound for use according to any of embodiments 1 to 78, wherein the treatment comprises administering a unit dose of from 5 mg to 15 mg, optionally 10 mg.
Embodiment 95: The oligomeric compound for use according to any of embodiments 1 to 78, wherein the treatment comprises administering a unit dose comprising not less than 1 mg of the oligomeric compound.
Embodiment 96: The oligomeric compound for use according to embodiment 1 to 78, wherein the treatment comprises administering a unit dose comprising not less than 2.5 mg of the oligomeric compound
Embodiment 97: The oligomeric compound for use according to embodiment 1 to 78, wherein the treatment comprises administering a unit dose comprising not less than 5 mg of the oligomeric compound
Embodiment 98: The oligomeric compound for use according to any of embodiments 79-94, wherein the treatment comprises administering not more than 1 unit dose to the human during the dosing period.
Embodiment 99: The oligomeric compound for use according to any of embodiments 79-94, wherein the treatment comprises administering not more than 2 unit doses to the human during the dosing period.
Embodiment 100: The oligomeric compound for use according to any of embodiments 79-94, wherein the treatment comprises administering not more than 3 unit doses to the human during the dosing period.
Embodiment 101: The oligomeric compound for use according to any of embodiments 79-94, wherein the treatment comprises administering not more than 4 unit doses to the human during the dosing period.
Embodiment 102: The oligomeric compound for use according to any of embodiments 79-94, wherein the treatment comprises administering not more than 5 unit doses to the human during the dosing period.
Embodiment 103: The oligomeric compound for use according to any of embodiments 79-94, wherein the treatment comprises administering not more than 6 unit doses to the human during the dosing period.
Embodiment 104: The oligomeric compound for use according to any of embodiments 79-94, wherein the treatment comprises administering a loading dose.
Embodiment 105: The oligomeric compound for use according to any of embodiments 79-94, wherein the treatment comprises administering a maintenance dose.
Embodiment 106: The oligomeric compound for use according to embodiment 104 or 105, wherein the loading dose is given prior to the maintenance dose.
Embodiment 107: The oligomeric compound for use according to any of embodiments 104-106, wherein the loading dose consists of 3 unit doses administered in the loading dose period.
Embodiment 108: The oligomeric compound for use according to any of embodiments 104-106, wherein the loading dose consists of 4 unit doses administered in the loading dose period.
Embodiment 109: The oligomeric compound for use according to any of embodiments 104-106, wherein the loading dose consists of 5 unit doses administered in the loading dose period.
Embodiment 110: The oligomeric compound for use according to any of embodiments 104-106, wherein the loading dose consists of 6 unit doses administered in the loading dose period.
Embodiment 111: The oligomeric compound for use according to any of embodiments 104-110, wherein the loading dose is given over a period of 4 weeks.
Embodiment 112: The oligomeric compound for use according to embodiment 110 or 111, wherein the initial loading dose is given at day 1, and subsequent loading doses are given at days 3, 5, 8, 15, and 22.
Embodiment 113: The oligomeric compound for use according to any of embodiments 104-112, wherein the maintenance dose is given once every week.
Embodiment 114: The oligomeric compound for use according to any of embodiments 104-112, wherein the maintenance dose is given every two weeks.
Embodiment 115: The oligomeric compound for use according to any of embodiments 104-112, wherein the maintenance dose is given three weeks.
Embodiment 116: The oligomeric compound for use according to any of embodiments 104-112, wherein the maintenance dose is given every four weeks.
Embodiment 117: The oligomeric compound for use according to any of embodiments 104-112, wherein the maintenance dose is given every month.
Embodiment 118: The oligomeric compound for use according to any of embodiments 104-112, wherein the maintenance dose is given every two months.
Embodiment 119: The oligomeric compound for use according to any of embodiments 104-112, wherein the maintenance dose is given every three months.
Embodiment 120: The oligomeric compound for use according to any preceding embodiment, wherein the oligomeric compound is administered by injection.
Embodiment 121: The oligomeric compound for use according to embodiment 120, wherein the oligomeric compound is administered by subcutaneous injection, optionally by subcutaneous injection into the abdomen, thigh, or upper arm.
Embodiment 122: The oligomeric compound for use according to embodiment 120 or embodiment 121, wherein the oligomeric compound is formulated in a sterile liquid and optionally wherein each unit dose of the oligomeric compound is not more than 1 mL of the sterile liquid.
Embodiment 123: The oligomeric compound for use according to embodiment 122, wherein each unit dose of the oligomeric compound is not more than 0.8 mL of the sterile liquid.
Embodiment 124: The oligomeric compound for use according to embodiment 122, wherein each unit dose of the oligomeric compound is not more than 0.5 mL of the sterile liquid.
Embodiment 125: The oligomeric compound for use according to embodiment 122, wherein each unit dose of the oligomeric compound is not more than 0.25 mL of the sterile liquid.
Embodiment 126: The oligomeric compound for use according to any of embodiments 122 to 125, wherein the sterile liquid is selected from among: sterile saline and water.
Embodiment 127: The oligomeric compound for use according to embodiment 126, wherein the sterile liquid further comprises a buffer.
Embodiment 128: The oligomeric compound for use according to embodiment 126 or 127, wherein the sterile liquid further comprises sodium chloride.
Embodiment 129: The oligomeric compound for use according to any preceding embodiment, wherein the oligomeric compound is formulated as a sodium salt.
Embodiment 130: The oligomeric compound for use according to any preceding embodiment, wherein the oligomeric compound is targeted to a nucleic acid molecule encoding human Apolipoprotein CIII (ApoCIII).
Embodiment 131: The oligomeric compound for use according to embodiment 130, wherein the treatment reduces the fasting plasma triglyceride concentration in the human by at least 30%, when the fasting plasma triglyceride concentration in the human is measured at the start and end of the dosing period.
Embodiment 132: The oligomeric compound for use according to any preceding embodiment, wherein the oligomeric compound is targeted to a nucleic acid molecule encoding human Angiopoietin-like 3 (ANGPTL3).
Embodiment 133: The oligomeric compound for use according to embodiment 132, wherein the treatment reduces the fasting plasma ANGPTL3 concentration in the human by at least 30%, when the fasting plasma ANGPTL3 concentration in the human is measured at the start and end of the dosing period.
Embodiment 134: A pharmaceutical composition, comprising:
   (i) an oligomeric compound comprising a modified oligonucleotide consisting of 12-22 linked nucleosides in a gapmer motif, and a conjugate group comprising a GalNAc cluster, wherein the oligomeric compound is an oligomeric compound as defined in any one of embodiments 1-133, and
   (ii) one or more pharmaceutically acceptable carriers or diluents, wherein the pharmaceutical composition is formulated for use in a treatment as set forth in any one of embodiments 1-131.
Embodiment 135: A sterile sealed container which contains the pharmaceutical composition of claim 134.
Embodiment 136: A sterile container according to any of claim 134 or 135, wherein the container is a vial.
Embodiment 137: The sterile container according to any of claim 134 or 135, wherein the container is a syringe.
Embodiment 138: The sterile container according to claim 136 or 137, wherein the container is for single use.
Embodiment 139: A packaged pharmaceutical product comprising: (a) multiple unit dosage forms each comprising a sealed sterile container according to any of claims 135-137; and (b) printed instructions describing the administration of the unit dosage forms for a treatment as set forth in any of claims 1-133.
Embodiment 140: A method comprising administering a unit dose of an oligomeric compound to a human subject in need thereof, wherein the oligomeric compound comprises a modified oligonucleotide and a conjugate group comprising a GalNAc cluster, and wherein the modified oligonucleotide consists of 12-22 linked nucleosides and comprises a region having a gapmer motif.
Embodiment 141: The method of claim 140, wherein the modified oligonucleotide has a gapmer motif.
Embodiment 142: The method of claim 141, wherein the gapmer motif is a sugar motif.
Embodiment 143: The method of any of embodiments 140-142, wherein the unit dose is 120 mg.
Embodiment 144: The method of any of embodiments 140-142, wherein the unit dose is 100 mg.
Embodiment 145: The method of any of embodiments 140-142, wherein the unit dose is 80 mg.
Embodiment 146: The method of any of embodiments 140-142, wherein the unit dose is 60 mg.
Embodiment 147: The method of any of embodiments 140-142, wherein the unit dose is 40 mg.
Embodiment 148: The method of any of embodiments 140-142, wherein the unit dose is 30 mg.
Embodiment 149: The method of any of embodiments 140-142, wherein the unit dose is 20 mg.
Embodiment 150: The method of any of embodiments 140-142, wherein the unit dose is 15 mg.
Embodiment 151: The method of any of embodiments 140-142, wherein the unit dose is 10 mg.
Embodiment 152: The method of any of embodiments 140 to 151, wherein the unit dose is administered once every week.
Embodiment 153: The method of any of embodiments 140 to 151, wherein the unit dose is administered once every 2 weeks.
Embodiment 154: The method of any of embodiments 140 to 151, wherein the unit dose is administered once every 3 weeks.
Embodiment 155: The method of any of embodiments 140 to 151, wherein the unit dose is administered once every 4 weeks.
Embodiment 156: The method of any of embodiments 140 to 151, wherein the unit dose is administered once every month.
Embodiment 157: The method of any of embodiments 140 to 151, wherein the unit dose is administered once every 2 months.
Embodiment 158: The method of any of embodiments 140 to 151, wherein the unit dose is administered once every 3 months.
Embodiment 159: The method of any of embodiments 140 to 151, wherein the unit dose is administered on day 1, 3, 5, 8, 15, 22, and once per week thereafter.
Embodiment 160: The method of any of embodiments 140 to 159, wherein the subject has one or more symptoms of a cardiovascular disease or disorder.
Embodiment 161: The method of embodiment 160, wherein one or more symptoms of the cardiovascular disease or disorder are ameliorated.

In certain embodiments, the present disclosure provides an oligomeric compound for use in treating or preventing a disease or condition in a human, wherein the treatment comprises administering one or more doses of the oligomeric compound to the human in (a) a loading or induction phase, and (b) a maintenance phase. In certain embodiments, a dose of the oligomeric compound is administered to the human during the maintenance phase once per week, once every two weeks, once per month, once every two months or once quarterly, for as long as needed, effective, and/or tolerated.

In some embodiments, the treatment comprises administering not more than not more than 450 mg, not more than 400 mg, not more than 350 mg, not more than 300 mg, not more then 250 mg, not more than 200 mg, not more than 150 mg, not more than 100 mg, not more than 75 mg, not more than 50 mg, not more than 40 mg, not more than 30 mg, not more than 25 mg, not more than 20 mg, or not more than 15 mg, of the oligomeric compound to the human during the dosing period.

### BRIEF DESCRIPTION OF THE FIGURES

FIGS. 1A-C illustrate the predicted Lp(a) levels as a result of different weekly dosing regimens. Doses of 20 mg (FIG. 1A), 30 mg (FIG. 1B) or 40 mg (FIG. 1C) shows a steady state reduction of Lp (a) of ≥80%.

FIGS. 2A-B illustrate the predicted Lp(a) levels as a result of different monthly dosing regimens. Doses of 60 mg (FIG. 2A) and 80 mg (FIG. 2B) Lp(a) show a steady state reduction of Lp (a) of about 80%.

FIG. 3 illustrates the predicted Lp (a) levels as a result of a 2-month dosing regimen (e.g. one dose every two months). An 80 mg dose every 2-months shows a steady state reduction of Lp (a) of about 80%.

FIG. 4 illustrates the predicted Lp (a) levels as a result of a quarterly dosing regimen. An 80 mg dose every quarter shows a steady state reduction of Lp (a) of 80% and maximum reduction of Lp (a) of >90%.

FIGS. 5A-D illustrate the predicted Lp(a) levels as a result of different monthly dosing regimens. Figures are shown modeling the effect on Lp(a) by monthly administration of ISIS 681257 at doses of 20 mg (FIG. 5A), 40 mg (FIG. 5B), 60 mg (FIG. 5C), and 80 mg (FIG. 5D). The dark middle line represents the predicted dose, while the uppermost and lowermost lines represent the 90% Confidence Interval.

FIGS. 6A-D illustrate the predicted Lp(a) levels as a result of different weekly dosing regimens. FIGS. 6A-D show modeling of the effect on Lp(a) by weekly administration of ISIS 681257 at doses of 5 mg (FIG. 6A), 10 mg (FIG. 6B), 20 mg (FIG. 6C), and 30 mg (FIG. 6D). The dark middle line represents the predicted dose, while the uppermost and lowermost lines represent the 90% Confidence Interval.

### DETAILED DESCRIPTION

It is to be understood that both the foregoing general description and the following detailed description are exemplary and explanatory only and are not restrictive of the disclosure. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting. Also, terms such as “element” or “component” encompass both elements and components comprising one unit and elements and components that comprise more than one subunit. unless specifically stated otherwise.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated by reference in their entirety for any purpose.

### A. Definitions

Unless specific definitions are provided. the nomenclature used in connection with, and the procedures and techniques of, analytical chemistry, synthetic organic chemistry, and medicinal and pharmaceutical chemistry described herein are those well known and commonly used in the art. Standard techniques may be used for chemical synthesis, and chemical analysis. Certain such techniques and procedures may be found for example in “Carbohydrate Modifications in Antisense Research” Edited by Sangvi and Cook, American Chemical Society, Washington D.C., 1994; “Remington's Pharmaceutical Sciences,” Mack Publishing Co., Easton, Pa., 21^{st} edition, 2005; and “Antisense Drug Technology, Principles, Strategies, and Applications” Edited by Stanley T. Crooke, CRC Press, Boca Raton, Florida; and Sambrook et al., “Molecular Cloning, A laboratory Manual,” 2^{nd} Edition, Cold Spring Harbor Laboratory Press, 1989, which are hereby incorporated by reference for any purpose. Where permitted, all patents, applications, published applications and other publications and other data referred to throughout in the disclosure are incorporated by reference herein in their entirety.

Unless otherwise indicated, the following terms have the following meanings:

As used herein. “2′-deoxynucleoside” means a nucleoside comprising 2′-H(H) furanosyl sugar moiety, as found in naturally occurring deoxyribonucleic acids (DNA). In certain embodiments, a 2′-deoxynucleoside may comprise a modified nucleobase or may comprise an RNA nucleobase (uracil).

As used herein, “2′-substituted nucleoside” or “2-modified nucleoside” means a nucleoside comprising a 2′-substituted or 2′-modified sugar moiety. As used herein, “2′-substituted” or “2-modified” in reference to a sugar moiety means a sugar moiety comprising at least one 2′-substituent group other than H or OH.

As used herein, “antisense activity” means any detectable and/or measurable change attributable to the hybridization of an antisense compound to its target nucleic acid. In certain embodiments, antisense activity is a decrease in the amount or expression of a target nucleic acid or protein encoded by such target nucleic acid compared to target nucleic acid levels or target protein levels in the absence of the antisense compound.

As used herein, “antisense compound” means a compound comprising an antisense oligonucleotide and optionally one or more additional features, such as a conjugate group or terminal group.

As used herein, “antisense oligonucleotide” means an oligonucleotide having a nucleobase sequence that is at least partially complementary to a target nucleic acid.

As used herein, “ameliorate” in reference to a treatment means improvement in at least one symptom relative to the same symptom in the absence of the treatment. In certain embodiments, amelioration is the reduction in the severity or frequency of a symptom or the delayed onset or slowing of progression in the severity or frequency of a symptom.

As used herein, “bicyclic nucleoside” or “BNA” means a nucleoside comprising a bicyclic sugar moiety. As used herein, “bicyclic sugar” or “bicyclic sugar moiety” means a modified sugar moiety comprising two rings, wherein the second ring is formed via a bridge connecting two of the atoms in the first ring thereby forming a bicyclic structure. In certain embodiments, the first ring of the bicyclic sugar moiety is a furanosyl moiety. In certain embodiments, the bicyclic sugar moiety does not comprise a furanosyl moiety.

As used herein, “branching group” means a group of atoms having at least 3 positions that are capable of forming covalent linkages to at least 3 groups. In certain embodiments, a branching group provides a plurality of reactive sites for connecting tethered ligands to an oligonucleotide via a conjugate linker and/or a cleavable moiety.

As used herein, “cell-targeting moiety” means a conjugate group or portion of a conjugate group that is capable of binding to a particular cell type or particular cell types.

As used herein, “cleavable moiety” means a bond or group of atoms that is cleaved under physiological conditions, for example, inside a cell, an animal, or a human.

As used herein, “complementary” in reference to an oligonucleotide means that at least 70% of the nucleobases of such oligonucleotide or one or more regions thereof and the nucleobases of another nucleic acid or one or more regions thereof are capable of hydrogen bonding with one another when the nucleobase sequence of the oligonucleotide and the other nucleic acid are aligned in opposing directions. Complementary nucleobases means nucleobases that are capable of forming hydrogen bonds with one another. Complementary nucleobase pairs include adenine (A) and thymine (T), adenine (A) and uracil (U), cytosine (C) and guanine (G), 5-methyl cytosine (^{m}C) and guanine (G). Complementary oligonucleotides and/or nucleic acids need not have nucleobase complementarity at each nucleoside. Rather, some mismatches are tolerated. As used herein, “fully complementary” or “100% complementary” in reference to oligonucleotides means that such oligonucleotides are complementary to another oligonucleotide or nucleic acid at each nucleoside of the oligonucleotide.

As used herein, “conjugate group” means a group of atoms that is directly or indirectly attached to an oligonucleotide. Conjugate groups include a conjugate moiety and a conjugate linker that attaches the conjugate moiety to the oligonucleotide.

As used herein, “conjugate linker” means a group of atoms comprising at least one bond that connects a conjugate moiety to an oligonucleotide.

As used herein, “conjugate moiety” means a group of atoms that is attached to an oligonucleotide via a conjugate linker.

As used herein, “contiguous” in the context of an oligonucleotide refers to nucleosides, nucleobases, sugar moieties, or internucleoside linkages that are immediately adjacent to each other. For example, “contiguous nucleobases” means nucleobases that are immediately adjacent to each other in a sequence.

As used herein, “double-stranded antisense compound” means an antisense compound comprising two oligomeric compounds that are complementary to each other and form a duplex, and wherein one of the two said oligomeric compounds comprises an antisense oligonucleotide.

As used herein, “fully modified” in reference to a modified oligonucleotide means a modified oligonucleotide in which each sugar moiety is modified. “Uniformly modified” in reference to a modified oligonucleotide means a fully modified oligonucleotide in which each sugar moiety is the same. For example, the nucleosides of a uniformly modified oligonucleotide can each have a 2′-MOE modification but different nucleobase modifications, and the internucleoside linkages may be different.

As used herein, “gapmer” means an antisense oligonucleotide comprising an internal region having a plurality of nucleosides that support RNase H cleavage positioned between external regions having one or more nucleosides, wherein the nucleosides comprising the internal region are chemically distinct from the nucleoside or nucleosides comprising the external regions. The internal region may be referred to as the “gap” and the external regions may be referred to as the “wings.”

As used herein, “hybridization” means the pairing or annealing of complementary oligonucleotides and/or nucleic acids. While not limited to a particular mechanism, the most common mechanism of hybridization involves hydrogen bonding, which may be Watson-Crick, Hoogsteen or reversed Hoogsteen hydrogen bonding. between complementary nucleobases.

As used herein, “inhibiting the expression or activity” refers to a reduction or blockade of the expression or activity relative to the expression of activity in an untreated or control sample and does not necessarily indicate a total elimination of expression or activity.

As used herein, the terms “internucleoside linkage” means a group or bond that forms a covalent linkage between adjacent nucleosides in an oligonucleotide. As used herein “modified internucleoside linkage” means any internucleoside linkage other than a naturally occurring, phosphate internucleoside linkage. Non-phosphate linkages are referred to herein as modified internucleoside linkages. “Phosphorothioate linkage” means a modified phosphate linkage in which one of the non-bridging oxygen atoms is replaced with a sulfur atom. A phosphorothioate internucleoside linkage is a modified internucleoside linkage. Modified internucleoside linkages include linkages that comprise abasic nucleosides. As used herein, “abasic nucleoside” means a sugar moiety in an oligonucleotide or oligomeric compound that is not directly connected to a nucleobase. In certain embodiments, an abasic nucleoside is adjacent to one or two nucleosides in an oligonucleotide.

As used herein, “linker-nucleoside” means a nucleoside that links, either directly or indirectly, an oligonucleotide to a conjugate moiety. Linker-nucleosides are located within the conjugate linker of an oligomeric compound. Linker-nucleosides are not considered part of the oligonucleotide portion of an oligomeric compound even if they are contiguous with the oligonucleotide.

As used herein, “non-bicyclic modified sugar” or “non-bicyclic modified sugar moiety” means a modified sugar moiety that comprises a modification, such as a substitutent, that does not form a bridge between two atoms of the sugar to form a second ring.

As used herein, “linked nucleosides” are nucleosides that are connected in a continuous sequence (i.e. no additional nucleosides are present between those that are linked).

As used herein, “mismatch” or “non-complementary” means a nucleobase of a first oligonucleotide that is not complementary with the corresponding nucleobase of a second oligonucleotide or target nucleic acid when the first and second oligomeric compound are aligned.

As used herein, “MOE” means methoxyethyl. “2′-MOE” means a —OCH₂CH₂OCH₃ group at the 2′ position of a furanosyl ring.

As used herein, “motif” means the pattern of unmodified and/or modified sugar moieties, nucleobases, and/or internucleoside linkages, in an oligonucleotide.

As used herein, “naturally occurring” means found in nature.

As used herein, “nucleobase” means a naturally occurring nucleobase or a modified nucleobase. As used herein a “naturally occurring nucleobase” is adenine (A), thymine (T), cytosine (C), uracil (U), and guanine (G). As used herein, a modified nucleobase is a group of atoms capable of pairing with at least one naturally occurring nucleobase. A universal base is a nucleobase that can pair with any one of the five unmodified nucleobases. As used herein, “nucleobase sequence” means the order of contiguous nucleobases in a nucleic acid or oligonucleotide independent of any sugar or internucleoside linkage modification.

As used herein, “nucleoside” means a compound comprising a nucleobase and a sugar moiety. The nucleobase and sugar moiety are each, independently, unmodified or modified. As used herein, “modified nucleoside” means a nucleoside comprising a modified nucleobase and/or a modified sugar moiety.

As used herein, “oligomeric compound” means a compound consisting of an oligonucleotide and optionally one or more additional features, such as a conjugate group or terminal group.

As used herein, “oligonucleotide” means a strand of linked nucleosides connected via internucleoside linkages, wherein each nucleoside and internucleoside linkage may be modified or unmodified. Unless otherwise indicated, oligonucleotides consist of 8-50 linked nucleosides. As used herein, “modified oligonucleotide” means an oligonucleotide, wherein at least one nucleoside or internucleoside linkage is modified. As used herein, “unmodified oligonucleotide” means an oligonucleotide that does not comprise any nucleoside modifications or internucleoside modifications.

As used herein, “pharmaceutically acceptable carrier or diluent” means any substance suitable for use in administering to an animal. Certain such carriers enable pharmaceutical compositions to be formulated as, for example, tablets, pills, dragees, capsules, liquids, gels, syrups, slurries, suspension and lozenges for the oral ingestion by a subject. In certain embodiments, a pharmaceutically acceptable carrier or diluent is sterile water; sterile saline; or sterile buffer solution.

As used herein “pharmaceutically acceptable salts” means physiologically and pharmaceutically acceptable salts of compounds, such as oligomeric compounds, i.e., salts that retain the desired biological activity of the parent compound and do not impart undesired toxicological effects thereto. As used herein “pharmaceutical composition” means a mixture of substances suitable for administering to a subject. For example, a pharmaceutical composition may comprise an antisense compound and a sterile aqueous solution. In certain embodiments, a pharmaceutical composition shows activity in free uptake assay in certain cell lines.

As used herein, “phosphorus moiety” means a group of atoms comprising a phosphorus atom. In certain embodiments, a phosphorus moiety comprises a mono-, di-, or tri-phosphate, or phosphorothioate.

As used herein “prodrug” means a therapeutic agent in a form outside the body that is converted to a different form within the body or cells thereof, and wherein the converted form is the active form. Typically conversion of a prodrug within the body is facilitated by the action of an enzymes (e.g., endogenous or viral enzyme) or chemicals present in cells or tissues and/or by physiologic conditions.

As used herein, “RNAi compound” means an antisense compound that acts, at least in part, through RISC or Ago2 to modulate a target nucleic acid and/or protein encoded by a target nucleic acid. RNAi compounds include, but are not limited to double-stranded siRNA, single-stranded RNA (ssRNA), and microRNA, including microRNA mimics. In certain embodiments, an RNAi compound modulates the amount, activity, and/or splicing of a target nucleic acid. The term RNAi compound excludes antisense oligonucleotides that act through RNase H.

As used herein, the term “single-stranded” in reference to an antisense compound means such a compound consisting of one oligomeric compound that is not paired with a second oligomeric compound to form a duplex. “Self-complementary” in reference to an oligonucleotide means an oligonucleotide that at least partially hybridizes to itself. A compound consisting of one oligomeric compound, wherein the oligonucleotide of the oligomeric compound is self-complementary, is a single-stranded compound. A single-stranded antisense or oligomeric compound may be capable of binding to a complementary oligomeric compound to form a duplex.

As used herein, “standard cell assay” means the assay described in Example X and reasonable variations thereof.

As used herein, “standard in vivo experiment” means the procedure described in Example X and reasonable variations thereof.

As used herein, “sugar moiety” means an unmodified sugar moiety or a modified sugar moiety. As used herein, “unmodified sugar moiety” means a 2′-OH(H) furanosyl moiety, as found in RNA (an “unmodified RNA sugar moiety”), or a 2′-H(H) moiety, as found in DNA (an “unmodified DNA sugar moiety”). Unmodified sugar moieties have one hydrogen at each of the 1′, 3′, and 4′ positions, an oxygen at the 3′ position, and two hydrogens at the 5′ position. As used herein, “modified sugar moiety” or “modified sugar” means a modified furanosyl sugar moiety or a sugar surrogate. As used herein, modified furanosyl sugar moiety means a furanosyl sugar comprising a non-hydrogen substituent in place of at least one hydrogen of an unmodified sugar moiety. In certain embodiments, a modified furanosyl sugar moiety is a 2′-substituted sugar moiety. Such modified furanosyl sugar moieties include bicyclic sugars and non-bicyclic sugars. As used herein, “sugar surrogate” means a modified sugar moiety having other than a furanosyl moiety that can link a nucleobase to another group, such as an internucleoside linkage, conjugate group, or terminal group in an oligonucleotide. Modified nucleosides comprising sugar surrogates can be incorporated into one or more positions within an oligonucleotide and such oligonucleotides are capable of hybridizing to complementary oligomeric compounds or nucleic acids.

As used herein, “target nucleic acid,” “target RNA,” “target RNA transcript” and “nucleic acid target” mean a nucleic acid that an antisense compound is designed to affect.

As used herein, “target region” means a portion of a target nucleic acid to which an antisense compound is designed to hybridize.

As used herein, “terminal group” means a chemical group or group of atoms that is covalently linked to a terminus of an oligonucleotide.

As used herein, “loading dose” means one or more doses given during the loading dose period.

As used herein, “loading dose period” means a period of time prior to the start of the maintenance dose period when one or more doses are administered to a human at a more frequent interval than during the maintenance dose period. For example, in certain embodiments, patients may receive up to 6 doses in an initial 4 week period of time, and then a subsequent maintenance dose each week after receiving the last loading dose. E.g. a patient may receive an initial dose on day 1, and subsequent doses on days 3, 5, 8, 15, and 22; and then doses every 7 days after day 22. Thus, the first six doses represent the loading dose period, and each subsequent dose administered at 7 day intervals represents the maintenance dose.

In some embodiments, the oligomeric compound is administered to the human during a loading dose period and a maintenance dose period, wherein: (i) the loading dose period precedes the maintenance dose period, (ii) the loading dose period comprises administering multiple loading doses; (iii) the maintenance dose period comprises administering multiple maintenance doses; (iv) each dose administered during the loading dose period comprises the same (mg) amount of the oligomeric compound as each dose administered during the maintenance dose period; and (v) the doses are administered less frequently during the maintenance dose period than during the loading dose period.

The loading dose period may be at least three weeks, at least four weeks, at least five weeks, at least six weeks, at least seven weeks or at least eight weeks, or the loading dose period may be at least one month, at least two months, at least three months, at least four months, at least five months or at least six months. Alternatively, the loading dose period may be up to three weeks, up to four weeks, up to five weeks, up to six weeks, up to seven weeks or up to eight weeks, or the loading dose period may be up to one month, up to two months, up to three months, up to four months, up to five months or up to six months.

The maintenance dose period may be at least three weeks, at least four weeks, at least five weeks, at least six weeks, at least seven weeks or at least eight weeks, or the maintenance dose period may be at least one month, at least two months, at least three months, at least four months, at least five months or at least six months.

As used herein, “dosing period” means the period of time between when a human subject receives the first dose and when the human subject receives a final dose. It is envisaged that dosing of the patient may continue after the end of the dosing period, such that a first dosing period is followed by one or more further dosing periods during which the same of a different dosing regimen is used. For example, a human subject may receive 6 doses in a first dosing period where the first and last dose are given 4 weeks apart. Subsequently, the human subject may then start a second dosing period where the human subject receives doses at regular intervals (e.g. one unit dose per week, one unit dose per month, or one unit dose per quarter).

As used herein, the term “unit dose” refers to the specific amount of the oligomeric compound administered to the human at a particular time point (e.g. the specific amount of the oligomeric compound administered to the human in a single subcutaneous injection). Each unit dose forms part of a multi-dose regimen, as described herein.

As used herein, the term “unit dosage form” denotes the physical form in which each unit dose is presented for administration.

As used herein, the term “sterile liquid” means and liquid suitable for administration to a human subject. In certain embodiments, sterile liquids comprise liquids that are substantially free from viable microorganisms or bacteria. In certain embodiments, sterile liquids comprise USP grade water or USP grade saline.

As used herein, the term “GalNac cluster” means a cell-targeting moiety having 1-4 GalNAc ligands.

### I. Certain Oligonucleotides

In certain embodiments. the invention provides oligonucleotides, which consist of linked nucleosides. Oligonucleotides may be unmodified oligonucleotides (RNA or DNA) or may be modified oligonucleotides. Modified oligonucleotides comprise at least one modification relative to unmodified RNA or DNA (i.e., comprise at least one modified nucleoside (comprising a modified sugar moiety and/or a modified nucleobase) and/or at least one modified internucleoside linkage).

### A. Certain Modified Nucleosides

Modified nucleosides comprise a modified sugar moiety or a modified nucleobase or both a modified sugar moiety and a modified nucleobase.

### 1. Certain Sugar Moieties

In certain embodiments. modified sugar moieties are non-bicyclic modified sugar moieties. In certain embodiments, modified sugar moieties are bicyclic or tricyclic sugar moieties. In certain embodiments, modified sugar moieties are sugar surrogates. Such sugar surrogates may comprise one or more substitutions corresponding to those of other types of modified sugar moieties.

In certain embodiments, modified sugar moieties are non-bicyclic modified sugar moieties comprising a furanosyl ring with one or more acyclic substituent, including but not limited to substituents at the 2′, 4′, and/or 5′ positions. In certain embodiments one or more acyclic substituent of non-bicyclic modified sugar moieties is branched. Examples of 2′-substituent groups suitable for non-bicyclic modified sugar moieties include but are not limited to: 2′-F, 2′-OCH₃ (“OMe” or “O-methyl”), and 2′-O(CH₂)₂OCH₃ (“MOE”). In certain embodiments, 2′-substituent groups are selected from among: halo, allyl, amino, azido, SH, CN, OCN, CF₃, OCF₃, O—C₁-C₁₀ alkoxy, O—C₁-C₁₀ substituted alkoxy, O—C₁-C₁₀ alkyl, O—C₁-C₁₀ substituted alkyl, S-alkyl, N(Rₘ)-alkyl, O-alkenyl, S-alkenyl, N(Rₘ)-alkenyl, O-alkynyl, S-alkynyl, N(Rₘ)-alkynyl, O-alkylenyl-O-alkyl, alkynyl, alkaryl, aralkyl, O-alkaryl, O-aralkyl, O(CH₂)₂SCH₃, O(CH₂)₂ON(Rₘ)(Rₙ) or OCH₂C(═O)—N(Rₘ)(Rₙ), where each Rₘ and Rₙ is, independently, H, an amino protecting group, or substituted or unsubstituted C₁-C₁₀ alkyl, and the 2′-substituent groups described in Cook et al., U.S. Pat. No. 6,531,584; Cook et al., U.S. Pat. No. 5,859,221; and Cook et al., U.S. Pat. No. 6,005,087. Certain embodiments of these 2′-substituent groups can be further substituted with one or more substituent groups independently selected from among: hydroxyl, amino, alkoxy, carboxy, benzyl, phenyl, nitro (NO₂), thiol, thioalkoxy, thioalkyl, halogen, alkyl, aryl, alkenyl and alkynyl. Examples of 4′-substituent groups suitable for non-bicyclic modified sugar moieties include but are not limited to alkoxy (e.g., methoxy), alkyl, and those described in Manoharan et al., WO 2015/106128. Examples of 5′-substituent groups suitable for non-bicyclic modified sugar moieties include but are not limited to: 5′-methyl (R or S), 5′-vinyl, and 5′-methoxy. In certain embodiments, non-bicyclic modified sugars comprise more than one non-bridging sugar substituent, for example, 2′-F-5′-methyl sugar moieties and the modified sugar moieties and modified nucleosides described in Migawa et al., WO 2008/101157 and Rajeev et al., US2013/0203836.).

In certain embodiments, a 2′-substituted nucleoside or 2′-non-bicyclic modified nucleoside comprises a sugar moiety comprising a non-bridging 2′-substituent group selected from: F, NH₂, N₃, OCF₃, OCH₃, O(CH₂)₃NH₂, CH₂CH═CH₂, OCH₂CH═CH₂, OCH₂CH₂OCH₃, O(CH₂)₂SCH₃, O(CH₂)₂ON(Rₘ)(Rₙ), O(CH₂)₂O(CH₂)₂N(CH₃)₂, and N-substituted acetamide (OCH₂C(═O)—N(Rₘ)(Rₙ)), where each Rₘ and Rₙ is, independently, H, an amino protecting group, or substituted or unsubstituted C₁-C₁₀ alkyl.

In certain embodiments, a 2′-substituted nucleoside or 2′-non-bicyclic modified nucleoside comprises a sugar moiety comprising a non-bridging 2′-substituent group selected from: F, OCF₃, OCH₃, OCH₂CH₂OCH₃, O(CH₂)₂SCH₃, O(CH₂)₂ON(CH₃)₂, O(CH₂)₂O(CH₂)₂N(CH₃)₂, and OCH₂C(═O)—N(H)CH₃ (“NMA”).

In certain embodiments, a 2′-substituted nucleoside or 2′-non-bicyclic modified nucleoside comprises a sugar moiety comprising a non-bridging 2′-substituent group selected from: F, OCH₃, and OCH₂CH₂OCH₃.

Nucleosides comprising modified sugar moieties, such as non-bicyclic modified sugar moieties, may be referred to by the position(s) of the substitution(s) on the sugar moiety of the nucleoside. For example, nucleosides comprising 2′-substituted or 2-modified sugar moieties are referred to as 2′-substituted nucleosides or 2-modified nucleosides.

Certain modified sugar moieties comprise a bridging sugar substituent that forms a second ring resulting in a bicyclic sugar moiety. In certain such embodiments, the bicyclic sugar moiety comprises a bridge between the 4′ and the 2′ furanose ring atoms. Examples of such 4′ to 2′ bridging sugar substituents include but are not limited to: 4′-CH₂-2′, 4′-(CH₂)₂-2′, 4′-(CH₂)₃-2′, 4′-CH₂—O-2′ (“LNA”), 4′-CH₂—S-2′, 4′-(CH₂)₂—O-2′ (“ENA”), 4′-CH(CH₃)—O-2′ (referred to as “constrained ethyl” or “cEt” when in the S configuration), 4′-CH₂—O—CH₂-2′, 4′-CH₂—N(R)-2′, 4′-CH(CH₂OCH₃)—O-2′ (“constrained MOE” or “cMOE”) and analogs thereof (see, e.g., Seth et al., U.S. Pat. No. 7,399,845, Bhat et al., U.S. Pat. No. 7,569,686, Swayze et al., U.S. Pat. No. 7,741,457, and Swayze et al., U.S. Pat. No. 8,022,193), 4′-C(CH₃)(CH₃)—O-2′ and analogs thereof (see, e.g., Seth et al., U.S. Pat. No. 8,278,283), 4′-CH₂—N(OCH₃)-2′ and analogs thereof (see, e.g., Prakash et al., U.S. Pat. No. 8,278,425), 4′-CH₂—O—N(CH₃)-2′ (see, e.g., Allerson et al., U.S. Pat. No. 7,696,345 and Allerson et al., U.S. Pat. No. 8,124,745), 4′-CH₂—C(H)(CH₃)-2′ (see, e.g., Zhou, et al., J. Org. Chem., 2009, 74, 118-134), 4′-CH₂—C(═CH₂)-2′ and analogs thereof (see e.g., Seth et al., U.S. Pat. No. 8,278,426), 4′-C(RₐR_{b})—N(R)—O-2′, 4′-C(RₐR_{b})—O—N(R)-2′, 4′-CH₂—O—N(R)-2′, and 4′-CH₂—N(R)—O-2′, wherein each R, Rₐ, and R_{b} is, independently, H, a protecting group, or C₁-C₁₂ alkyl (see, e.g. Imanishi et al., U.S. Pat. No. 7,427,672).

In certain embodiments, such 4′ to 2′ bridges independently comprise from 1 to 4 linked groups independently selected from: —[C(Rₐ)(R_{b})]ₙ—, —[C(Rₐ)(R_{b})]ₙ—O—, —C(Rₐ)═C(R_{b})—, —C(Rₐ)═N—, —C(═NRₐ)—, —C(═O)—, —C(═S)—, —O—, —Si(Rₐ)₂—, —S(═O)ₓ—, and —N(Rₐ)—;
wherein:
x is 0, 1, or 2;
n is 1, 2, 3, or 4;
each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and
each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl, or a protecting group.

Additional bicyclic sugar moieties are known in the art, see, for example: Freier et al., Nucleic Acids Research, 1997, 25(22), 4429-4443, Albaek et al., J. Org. Chem., 2006, 71, 7731-7740, Singh et al., Chem. Commun., 1998, 4, 455-456; Koshkin et al., Tetrahedron, 1998, 54, 3607-3630; Wahlestedt et al., Proc. Natl. Acad. Sci. U.S.A., 2000, 97, 5633-5638; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 20017, 129, 8362-8379; Elayadi et al., Curr. Opinion Invens. Drugs. 2001, 2, 558-561; Braasch et al., Chem. Biol., 2001, 8, 1-7; Orum et al., Curr. Opinion Mol. Ther., 2001, 3, 239-243; Wengel et al., U.S. Pat. No. 7,053,207; Imanishi et al., U.S. Pat. No. 6,268,490; Imanishi et al. U.S. Pat. No. 6,770,748; Imanishi et al., U.S. RE44,779; Wengel et al., U.S. Pat. No. 6,794,499; Wengel et al., U.S. Pat. No. 6,670,461; Wengel et al., U.S. Pat. No. 7,034,133; Wengel et al., U.S. Pat. No. 8,080,644; Wengel et al., U.S. Pat. No. 8,034,909; Wengel et al., U.S. Pat. No. 8,153,365; Wengel et al., U.S. Pat. No. 7,572,582; and Ramasamy et al., U.S. Pat. No. 6,525,191; Torsten et al., WO 2004/106356; Wengel et al., WO 1999/014226; Seth et al., WO 2007/134181; Seth et al., U.S. Pat. No. 7,547,684; Seth et al., U.S. Pat. No. 7,666,854; Seth et al., U.S. Pat. No. 8,088,746; Seth et al., U.S. Pat. No. 7,750,131; Seth et al., U.S. Pat. No. 8,030,467; Seth et al., U.S. Pat. No. 8,268,980; Seth et al., U.S. Pat. No. 8,546,556; Seth et al., U.S. Pat. No. 8,530,640; Migawa et al., U.S. Pat. No. 9,012,421; Seth et al., U.S. Pat. No. 8,501,805; and U.S. Patent Publication Nos. Allerson et al., US2008/0039618 and Migawa et al., US2015/0191727.

In certain embodiments, bicyclic sugar moieties and nucleosides incorporating such bicyclic sugar moieties are further defined by isomeric configuration. For example, an LNA nucleoside (described herein) may be in the α-L configuration or in the β-D configuration.

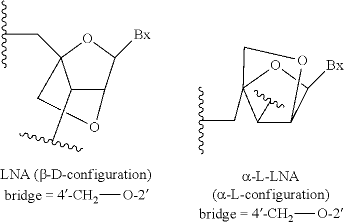

α-L-methyleneoxy (4′-CH₂—O-2′) or α-L-LNA bicyclic nucleosides have been incorporated into antisense oligonucleotides that showed antisense activity (Frieden et al., Nucleic Acids Research, 2003, 21, 6365-6372). Herein, general descriptions of bicyclic nucleosides include both isomeric configurations. When the positions of specific bicyclic nucleosides (e.g., LNA or cEt) are identified in exemplified embodiments herein, they are in the β-D configuration, unless otherwise specified.

In certain embodiments, modified sugar moieties comprise one or more non-bridging sugar substituent and one or more bridging sugar substituent (e.g., 5′-substituted and 4′-2′ bridged sugars).

In certain embodiments, modified sugar moieties are sugar surrogates. In certain such embodiments, the oxygen atom of the sugar moiety is replaced, e.g., with a sulfur, carbon or nitrogen atom. In certain such embodiments, such modified sugar moieties also comprise bridging and/or non-bridging substituents as described herein. For example, certain sugar surrogates comprise a 4′-sulfur atom and a substitution at the 2′-position (see, e.g., Bhat et al., U.S. Pat. No. 7,875,733 and Bhat et al., U.S. Pat. No. 7,939,677) and/or the 5′ position.

In certain embodiments, sugar surrogates comprise rings having other than 5 atoms. For example, in certain embodiments, a sugar surrogate comprises a six-membered tetrahydropyran (“THP”). Such tetrahydropyrans may be further modified or substituted. Nucleosides comprising such modified tetrahydropyrans include but are not limited to hexitol nucleic acid (“HNA”), anitol nucleic acid (“ANA”), manitol nucleic acid (“MNA”) (see, e.g., Leumann, C J. Bioorg. & Med. Chem. 2002, 10, 841-854), fluoro HNA:

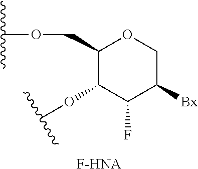

(“F-HNA”, see e.g. Swayze et al., U.S. Pat. No. 8,088,904; Swayze et al., U.S. Pat. No. 8,440,803; Swayze et al., U.S. Pat. No. 8,796,437; and Swayze et al., U.S. Pat. No. 9,005,906; F-HNA can also be referred to as a F-THP or 3′-fluoro tetrahydropyran), and nucleosides comprising additional modified THP compounds having the formula:

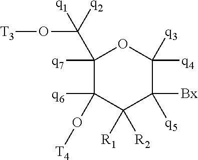

wherein, independently, for each of said modified THP nucleoside:
Bx is a nucleobase moiety;
T₃ and T₄ are each, independently, an internucleoside linking group linking the modified THP nucleoside to the remainder of an oligonucleotide or one of T₃ and T₄ is an internucleoside linking group linking the modified THP nucleoside to the remainder of an oligonucleotide and the other of T₃ and T₄ is H, a hydroxyl protecting group, a linked conjugate group, or a 5′ or 3′-terminal group;
q₁, q₂, q₃, q₄, q₅, q₆ and q₇ are each, independently, H, C₁-C₆ alkyl, substituted C₁-C₆ alkyl, C₂-C₆ alkenyl, substituted C₂-C₆ alkenyl, C₂-C₆ alkynyl, or substituted C₂-C₆ alkynyl; and
each of R₁ and R₂ is independently selected from among: hydrogen, halogen, substituted or unsubstituted alkoxy, NJ₁J₂, SJ₁, N₃, OC(═X)J₁, OC(═X)NJ₁J₂, NJ₃C(═X)NJ₁J₂, and CN, wherein X is O, S or NJ₁, and each J₁, J₂, and J₃ is, independently, H or C₁-C₆ alkyl.

In certain embodiments, modified THP nucleosides are provided wherein q₁, q₂, q₃, q₄, q₅, q₆ and q₇ are each H. In certain embodiments, at least one of q₁, q₂, q₃, q₄, q₅, q₆ and q₇ is other than H. In certain embodiments, at least one of q₁, q₂, q₃, q₄, q₅, q₆ and q₇ is methyl. In certain embodiments, modified THP nucleosides are provided wherein one of R₁ and R₂ is F. In certain embodiments, R₁ is F and R₂ is H, in certain embodiments, R₁ is methoxy and R₂ is H, and in certain embodiments, R₁ is methoxyethoxy and R₂ is H.

In certain embodiments, sugar surrogates comprise rings having more than 5 atoms and more than one heteroatom. For example, nucleosides comprising morpholino sugar moieties and their use in oligonucleotides have been reported (see, e.g., Braasch et al., Biochemistry, 2002, 41, 4503-4510 and Summerton et al., U.S. Pat. No. 5,698,685; Summerton et al., U.S. Pat. No. 5,166,315; Summerton et al., U.S. Pat. No. 5,185,444; and Summerton et al., U.S. Pat. No. 5,034,506). As used here, the term “morpholino” means a sugar surrogate having the following structure:

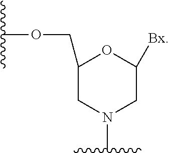

In certain embodiments, morpholinos may be modified, for example by adding or altering various substituent groups from the above morpholino structure. Such sugar surrogates are referred to herein as “modified morpholinos.”

In certain embodiments, sugar surrogates comprise acyclic moieties. Examples of nucleosides and oligonucleotides comprising such acyclic sugar surrogates include but are not limited to: peptide nucleic acid (“PNA”), acyclic butyl nucleic acid (see, e.g., Kumar et al., Org. Biomol. Chem., 2013, 11, 5853-5865), and nucleosides and oligonucleotides described in Manoharan et al., WO2011/133876.

Many other bicyclic and tricyclic sugar and sugar surrogate ring systems are known in the art that can be used in modified nucleosides).

### 2. Certain Modified Nucleobases

In certain embodiments, modified oligonucleotides comprise one or more nucleoside comprising an unmodified nucleobase. In certain embodiments, modified oligonucleotides comprise one or more nucleoside comprising a modified nucleobase.

In certain embodiments, modified nucleobases are selected from: 5-substituted pyrimidines, 6-azapyrimidines, alkyl or alkynyl substituted pyrimidines, alkyl substituted purines, and N-2, N-6 and O-6 substituted purines. In certain embodiments, modified nucleobases are selected from: 2-aminopropyladenine, 5-hydroxymethyl cytosine, xanthine, hypoxanthine, 2-aminoadenine, 6-N-methylguanine, 6-N-methyladenine, 2-propyladenine , 2-thiouracil, 2-thiothymine and 2-thiocytosine, 5-propynyl (—C═C—CH₃) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one and 9-(2-aminoethoxy)-1,3-diazaphenoxazine-2-one (G-clamp). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in Merigan et al., U.S. Pat. No. 3,687,808, those disclosed in The Concise Encyclopedia Of Polymer Science And Engineering, Kroschwitz, J. I., Ed., John Wiley & Sons, 1990, 858-859; Englisch et al., Angewandte Chemie, International Edition, 1991, 30, 613; Sanghvi, Y. S., Chapter 15. Antisense Research and Applications, Crooke, S. T. and Lebleu, B., Eds., CRC Press, 1993, 273-288; and those disclosed in Chapters 6 and 15, Antisense Drug Technology, Crooke S. T., Ed., CRC Press, 2008, 163-166 and 442-443.

Publications that teach the preparation of certain of the above noted modified nucleobases as well as other modified nucleobases include without limitation, Manohara et al., US2003/0158403: Manoharan et al., US2003/0175906; Dinh et al., U.S. Pat. No. 4,845,205; Spielvogel et al., U.S. Pat. No. 5,130,302; Rogers et al., U.S. Pat. No. 5,134,066; Bischofberger et al., U.S. Pat. No. 5,175,273; Urdea et al., U.S. Pat. No. 5,367,066; Benner et al., U.S. Pat. No. 5,432,272; Matteucci et al., U.S. Pat. No. 5,434,257; Gmeiner et al., U.S. Pat. No. 5,457,187; Cook et al., U.S. Pat. No. 5,459,255; Frochler et al., U.S. Pat. No. 5,484,908; Matteucci et al., U.S. Pat. No. 5,502,177; Hawkins et al., U.S. Pat. No. 5,525,711; Haralambidis et al., U.S. Pat. No. 5,552,540; Cook et al., U.S. Pat. No. 5,587,469; Frochler et al., U.S. Pat. No. 5,594,121; Switzer et al., U.S. Pat. No. 5,596,091; Cook et al., U.S. Pat. No. 5,614,617; Frochler et al., U.S. Pat. No. 5,645,985; Cook et al., U.S. Pat. No. 5,681,941; Cook et al., U.S. Pat. No. 5,811,534; Cook et al., U.S. Pat. No. 5,750,692; Cook et al., U.S. Pat. No. 5,948,903; Cook et al., U.S. Pat. No. 5,587,470; Cook et al., U.S. Pat. No. 5,457,191; Matteucci et al., U.S. Pat. No. 5,763,588; Frochler et al., U.S. Pat. No. 5,830,653; Cook et al., U.S. Pat. No. 5,808,027; Cook et al., U.S. Pat. No. 6,166,199; and Matteucci et al., U.S. Pat. No. 6,005,096.

### B. Certain Modified Internucleoside Linkages

In certain embodiments, nucleosides of modified oligonucleotides may be linked together using any internucleoside linkage. The two main classes of internucleoside linking groups are defined by the presence or absence of a phosphorus atom. Representative phosphorus-containing internucleoside linkages include but are not limited to phosphates, which contain a phosphodiester bond (“P═O”) (also referred to as unmodified or naturally occurring linkages), phosphotriesters, methylphosphonates, phosphoramidates, and phosphorothioates (“P═S”), and phosphorodithioates (“HS—P═S”). Representative non-phosphorus containing internucleoside linking groups include but are not limited to methylenemethylimino (—CH₂—N(CH₃)—O—CH₂—), thiodiester, thionocarbamate (—O—C(═O)(NH)—S—); siloxane (—O—SiH₂—O—); and N,N′-dimethylhydrazine (—CH₂—N(CH₃)—N(CH₃)—). Modified internucleoside linkages, compared to naturally occurring phosphate linkages, can be used to alter, typically increase, nuclease resistance of the oligonucleotide. In certain embodiments, internucleoside linkages having a chiral atom can be prepared as a racemic mixture, or as separate enantiomers. Representative chiral internucleoside linkages include but are not limited to alkylphosphonates and phosphorothioates. Methods of preparation of phosphorous-containing and non-phosphorous-containing internucleoside linkages are well known to those skilled in the art.

Neutral internucleoside linkages include, without limitation, phosphotriesters, methylphosphonates, MMI (3′-CH₂—N(CH₃)—O-5′), amide-3 (3′-CH₂—C(═O)—N(H)-5′), amide-4 (3′-CH₂—N(H)—C(═O)-5′), formacetal (3′-O—CH₂—O-5′), methoxypropyl, and thioformacetal (3′-S—CH₂—O-5′). Further neutral internucleoside linkages include nonionic linkages comprising siloxane (dialkylsiloxane), carboxylate ester, carboxamide, sulfide, sulfonate ester and amides (See for example: Carbohydrate Modifications in Antisense Research; Y. S. Sanghvi and P. D. Cook, Eds., ACS Symposium Series 580; Chapters 3 and 4, 40-65). Further neutral internucleoside linkages include nonionic linkages comprising mixed N, O, S and CH₂ component parts.

### C. Certain Motifs

In certain embodiments, modified oligonucleotides comprise one or more modified nucleoside comprising a modified sugar. In certain embodiments, modified oligonucleotides comprise one or more modified nucleosides comprising a modified nucleobase. In certain embodiments, modified oligonucleotides comprise one or more modified internucleoside linkage. In such embodiments, the modified, unmodified, and differently modified sugar moieties, nucleobases, and/or internucleoside linkages of a modified oligonucleotide define a pattern or motif. In certain embodiments, the patterns of sugar moieties, nucleobases, and internucleoside linkages are each independent of one another. Thus, a modified oligonucleotide may be described by its sugar motif, nucleobase motif and/or internucleoside linkage motif (as used herein, nucleobase motif describes the modifications to the nucleobases independent of the sequence of nucleobases).

### 1. Certain Sugar Motifs

In certain embodiments. oligonucleotides comprise one or more type of modified sugar and/or unmodified sugar moiety arranged along the oligonucleotide or region thereof in a defined pattern or sugar motif. In certain instances, such sugar motifs include but are not limited to any of the sugar modifications discussed herein.

In certain embodiments, modified oligonucleotides comprise or consist of a region having a gapmer motif, which comprises two external regions or “wings” and a central or internal region or “gap.” The three regions of a gapmer motif (the 5′-wing, the gap, and the 3′-wing) form a contiguous sequence of nucleosides wherein at least some of the sugar moieties of the nucleosides of each of the wings differ from at least some of the sugar moieties of the nucleosides of the gap. Specifically, at least the sugar moieties of the nucleosides of each wing that are closest to the gap (the 3′-most nucleoside of the 5′-wing and the 5′-most nucleoside of the 3′-wing) differ from the sugar moiety of the neighboring gap nucleosides, thus defining the boundary between the wings and the gap (i.e., the wing/gap junction). In certain embodiments, the sugar moieties within the gap are the same as one another. In certain embodiments, the gap includes one or more nucleoside having a sugar moiety that differs from the sugar moiety of one or more other nucleosides of the gap. In certain embodiments, the sugar motifs of the two wings are the same as one another (symmetric gapmer). In certain embodiments, the sugar motif of the 5′-wing differs from the sugar motif of the 3′-wing (asymmetric gapmer).

In certain embodiments, the wings of a gapmer comprise 1-5 nucleosides. In certain embodiments, the wings of a gapmer comprise 2-5 nucleosides. In certain embodiments, the wings of a gapmer comprise 3-5 nucleosides. In certain embodiments, the nucleosides of a gapmer are all modified nucleosides.

In certain embodiments, the gap of a gapmer comprises 7-12 nucleosides. In certain embodiments, the gap of a gapmer comprises 7-10 nucleosides. In certain embodiments, the gap of a gapmer comprises 8-10 nucleosides. In certain embodiments, the gap of a gapmer comprises 10 nucleosides. In certain embodiment, each nucleoside of the gap of a gapmer is an unmodified 2′-deoxy nucleoside.

In certain embodiments, the gapmer is a deoxy gapmer. In such embodiments, the nucleosides on the gap side of each wing/gap junction are unmodified 2′-deoxy nucleosides and the nucleosides on the wing sides of each wing/gap junction are modified nucleosides. In certain such embodiments, each nucleoside of the gap is an unmodified 2′-deoxy nucleoside. In certain such embodiments, each nucleoside of each wing is a modified nucleoside.

In certain embodiments, modified oligonucleotides comprise or consist of a region having a fully modified sugar motif. In such embodiments, each nucleoside of the fully modified region of the modified oligonucleotide comprises a modified sugar moiety. In certain such embodiments, each nucleoside to the entire modified oligonucleotide comprises a modified sugar moiety. In certain embodiments, modified oligonucleotides comprise or consist of a region having a fully modified sugar motif, wherein each nucleoside within the fully modified region comprises the same modified sugar moiety, referred to herein as a uniformly modified sugar motif. In certain embodiments, a fully modified oligonucleotide is a uniformly modified oligonucleotide. In certain embodiments, each nucleoside of a uniformly modified comprises the same 2′-modification.

### 2. Certain Nucleobase Motifs

In certain embodiments, oligonucleotides comprise modified and/or unmodified nucleobases arranged along the oligonucleotide or region thereof in a defined pattern or motif. In certain embodiments, each nucleobase is modified. In certain embodiments, none of the nucleobases are modified. In certain embodiments, each purine or each pyrimidine is modified. In certain embodiments, each adenine is modified. In certain embodiments, each guanine is modified. In certain embodiments, each thymine is modified. In certain embodiments, each uracil is modified. In certain embodiments, each cytosine is modified. In certain embodiments, some or all of the cytosine nucleobases in a modified oligonucleotide are 5-methylcytosines.

In certain embodiments, modified oligonucleotides comprise a block of modified nucleobases. In certain such embodiments, the block is at the 3′-end of the oligonucleotide. In certain embodiments the block is within 3 nucleosides of the 3′-end of the oligonucleotide. In certain embodiments, the block is at the 5′-end of the oligonucleotide. In certain embodiments the block is within 3 nucleosides of the 5′-end of the oligonucleotide.

In certain embodiments, oligonucleotides having a gapmer motif comprise a nucleoside comprising a modified nucleobase. In certain such embodiments, one nucleoside comprising a modified nucleobase is in the central gap of an oligonucleotide having a gapmer motif. In certain such embodiments, the sugar moiety of said nucleoside is a 2′-deoxyribosyl moiety. In certain embodiments, the modified nucleobase is selected from: a 2-thiopyrimidine and a 5-propynepyrimidine.

### 3. Certain Internucleoside Linkage Motifs

In certain embodiments, oligonucleotides comprise modified and/or unmodified internucleoside linkages arranged along the oligonucleotide or region thereof in a defined pattern or motif. In certain embodiments, essentially each internucleoside linking group is a phosphate internucleoside linkage (P═O). In certain embodiments, each internucleoside linking group of a modified oligonucleotide is a phosphorothioate (P═S). In certain embodiments, each internucleoside linking group of a modified oligonucleotide is independently selected from a phosphorothioate and phosphate internucleoside linkage. In certain embodiments, the sugar motif of a modified oligonucleotide is a gapmer and the internucleoside linkages within the gap are all modified. In certain such embodiments, some or all of the internucleoside linkages in the wings are unmodified phosphate linkages. In certain embodiments, the terminal internucleoside linkages are modified.

### D. Certain Lengths

In certain embodiments, oligonucleotides (including modified oligonucleotides) can have any of a variety of ranges of lengths. In certain embodiments, oligonucleotides consist of X to Y linked nucleosides, where X represents the fewest number of nucleosides in the range and Y represents the largest number nucleosides in the range. In certain such embodiments, X and Y are each independently selected from 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50; provided that X≤Y. For example, in certain embodiments, oligonucleotides consist of 12 to 13, 12 to 14, 12 to 15, 12 to 16, 12 to 17, 12 to 18, 12 to 19, 12 to 20, 12 to 21, 12 to 22, 12 to 23, 12 to 24, 12 to 25, 12 to 26, 12 to 27, 12 to 28, 12 to 29, 12 to 30, 13 to 14, 13 to 15, 13 to 16, 13 to 17, 13 to 18, 13 to 19, 13 to 20, 13 to 21, 13 to 22, 13 to 23, 13 to 24, 13 to 25, 13 to 26, 13 to 27, 13 to 28, 13 to 29, 13 to 30, 14 to 15, 14 to 16, 14 to 17, 14 to 18, 14 to 19, 14 to 20, 14 to 21, 14 to 22, 14 to 23, 14 to 24, 14 to 25, 14 to 26, 14 to 27, 14 to 28, 14 to 29, 14 to 30, 15 to 16, 15 to 17, 15 to 18, 15 to 19, 15 to 20, 15 to 21, 15 to 22, 15 to 23, 15 to 24, 15 to 25, 15 to 26, 15 to 27, 15 to 28, 15 to 29, 15 to 30, 16 to 17, 16 to 18, 16 to 19, 16 to 20, 16 to 21, 16 to 22, 16 to 23, 16 to 24, 16 to 25, 16 to 26, 16 to 27, 16 to 28, 16 to 29, 16 to 30, 17 to 18, 17 to 19, 17 to 20, 17 to 21, 17 to 22, 17 to 23, 17 to 24, 17 to 25, 17 to 26, 17 to 27, 17 to 28, 17 to 29, 17 to 30, 18 to 19, 18 to 20, 18 to 21, 18 to 22, 18 to 23, 18 to 24, 18 to 25, 18 to 26, 18 to 27, 18 to 28, 18 to 29, 18 to 30, 19 to 20, 19 to 21, 19 to 22, 19 to 23, 19 to 24, 19 to 25, 19 to 26, 19 to 29, 19 to 28, 19 to 29, 19 to 30, 20 to 21, 20 to 22, 20 to 23, 20 to 24, 20 to 25, 20 to 26, 20 to 27, 20 to 28, 20 to 29, 20 to 30, 21 to 22, 21 to 23, 21 to 24, 21 to 25, 21 to 26, 21 to 27, 21 to 28, 21 to 29, 21 to 30, 22 to 23, 22 to 24, 22 to 25, 22 to 26, 22 to 27, 22 to 28, 22 to 29, 22 to 30, 23 to 24, 23 to 25, 23 to 26, 23 to 27, 23 to 28, 23 to 29, 23 to 30, 24 to 25, 24 to 26, 24 to 27, 24 to 28, 24 to 29, 24 to 30, 25 to 26, 25 to 27, 25 to 28, 25 to 29, 25 to 30, 26 to 27, 26 to 28, 26 to 29, 26 to 30, 27 to 28, 27 to 29, 27 to 30, 28 to 29, 28 to 30, or 29 to 30 linked nucleosides

### E. Certain Modified Oligonucleotides

In certain embodiments, the above modifications (sugar, nucleobase, internucleoside linkage) are incorporated into a modified oligonucleotide. In certain embodiments, modified oligonucleotides are characterized by their modification motifs and overall lengths. In certain embodiments, such parameters are each independent of one another. Thus, unless otherwise indicated, each internucleoside linkage of an oligonucleotide having a gapmer sugar motif may be modified or unmodified and may or may not follow the gapmer modification pattern of the sugar modifications. For example, the internucleoside linkages within the wing regions of a sugar gapmer may be the same or different from one another and may be the same or different from the internucleoside linkages of the gap region of the sugar motif. Likewise, such sugar gapmer oligonucleotides may comprise one or more modified nucleobase independent of the gapmer pattern of the sugar modifications. Furthermore, in certain instances, an oligonucleotide is described by an overall length or range and by lengths or length ranges of two or more regions (e.g., a regions of nucleosides having specified sugar modifications), in such circumstances it may be possible to select numbers for each range that result in an oligonucleotide having an overall length falling outside the specified range. In such circumstances, both elements must be satisfied. For example, in certain embodiments, a modified oligonucleotide consists if of 15-20 linked nucleosides and has a sugar motif consisting of three regions, A, B, and C, wherein region A consists of 2-6 linked nucleosides having a specified sugar motif, region B consists of 6-10 linked nucleosides having a specified sugar motif, and region C consists of 2-6 linked nucleosides having a specified sugar motif. Such embodiments do not include modified oligonucleotides where A and C each consist of 6 linked nucleosides and B consists of 10 linked nucleosides (even though those numbers of nucleosides are permitted within the requirements for A, B, and C) because the overall length of such oligonucleotide is 22, which exceeds the upper limit of the overall length of the modified oligonucleotide (20). Herein, if a description of an oligonucleotide is silent with respect to one or more parameter, such parameter is not limited. Thus, a modified oligonucleotide described only as having a gapmer sugar motif without further description may have any length, internucleoside linkage motif, and nucleobase motif. Unless otherwise indicated, all modifications are independent of nucleobase sequence.

### F. Nucleobase Sequence

In certain embodiments, oligonucleotides (unmodified or modified oligonucleotides) are further described by their nucleobase sequence. In certain embodiments oligonucleotides have a nucleobase sequence that is complementary to a second oligonucleotide or an identified reference nucleic acid, such as a target nucleic acid. In certain such embodiments, a region of an oligonucleotide has a nucleobase sequence that is complementary to a second oligonucleotide or an identified reference nucleic acid, such as a target nucleic acid. In certain embodiments, the nucleobase sequence of a region or entire length of an oligonucleotide is at least 50%, at least 60%, at least 70%, at least 80%, at least 90%, at least 95%, or 100% complementary to the second oligonucleotide or nucleic acid, such as a target nucleic acid.

### II. Certain Oligomeric Compounds

In certain embodiments, the invention provides oligomeric compounds, which consist of an oligonucleotide (modified or unmodified) and optionally one or more conjugate groups and/or terminal groups. Conjugate groups consist of one or more conjugate moiety and a conjugate linker which links the conjugate moiety to the oligonucleotide. Conjugate groups may be attached to either or both ends of an oligonucleotide and/or at any internal position. In certain embodiments, conjugate groups are attached to the 2′-position of a nucleoside of a modified oligonucleotide. In certain embodiments, conjugate groups that are attached to either or both ends of an oligonucleotide are terminal groups. In certain such embodiments, conjugate groups or terminal groups are attached at the 3′ and/or 5′-end of oligonucleotides. In certain such embodiments, conjugate groups (or terminal groups) are attached at the 3′-end of oligonucleotides. In certain embodiments, conjugate groups are attached near the 3′-end of oligonucleotides. In certain embodiments, conjugate groups (or terminal groups) are attached at the 5′-end of oligonucleotides. In certain embodiments, conjugate groups are attached near the 5′-end of oligonucleotides.

Examples of terminal groups include but are not limited to conjugate groups, capping groups, phosphate moieties, protecting groups, abasic nucleosides, modified or unmodified nucleosides, and two or more nucleosides that are independently modified or unmodified.

### A. Certain Conjugate Groups

In certain embodiments, oligonucleotides are covalently attached to one or more conjugate groups. In certain embodiments, conjugate groups modify one or more properties of the attached oligonucleotide, including but not limited to pharmacodynamics, pharmacokinetics, stability, binding, absorption, tissue distribution, cellular distribution, cellular uptake, charge and clearance. In certain embodiments, conjugate groups impart a new property on the attached oligonucleotide, e.g., fluorophores or reporter groups that enable detection of the oligonucleotide. Certain conjugate groups and conjugate moieties have been described previously, for example: cholesterol moiety (Letsinger et al., Proc. Natl. Acad. Sci. USA, 1989, 86, 6553-6556), cholic acid (Manoharan et al., Bioorg. Med. Chem. Lett., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Lett., 1993, 3, 2765-2770), a thiocholesterol (Oberhauser et al., Nucl. Acids Res., 1992, 20, 533-538), an aliphatic chain, e.g., do-decan-diol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid a palmityl moiety (Mishra et al., Biochim. Biophys. Acta, 1995, 1264, 229-237), an octadecylamine or hexylamino-carbonyl-oxycholesterol moiety (Crooke et al., J. Pharmacol. Exp. Ther., 1996, 277, 923-937), a tocopherol group (Nishina et al., Molecular Therapy Nucleic Acids, 2015, 4, e220; and Nishina et al., Molecular Therapy, 2008, 16, 734-740), or a GalNAc cluster (e.g., WO2014/179620).

### 1. Conjugate Moieties

Conjugate moieties include, without limitation, intercalators, reporter molecules, polyamines, polyamides, peptides, carbohydrates (e.g., GalNAc), vitamin moieties, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins, fluorophores, and dyes.

In certain embodiments, a conjugate moiety comprises an active drug substance, for example, aspirin, warfarin, phenylbutazone, ibuprofen, suprofen, fen-bufen, ketoprofen, (S)-(+)-pranoprofen, carprofen, dansylsarcosine, 2,3,5-triiodobenzoic acid, fingolimod, flufenamic acid, folinic acid, a benzothiadiazide, chlorothiazide, a diazepine, indo-methicin, a barbiturate, a cephalosporin, a sulfa drug, an antidiabetic, an antibacterial or an antibiotic.

### 2. Conjugate Linkers

Conjugate moieties are attached to oligonucleotides through conjugate linkers. In certain oligomeric compounds, the conjugate linker is a single chemical bond (i.e., the conjugate moiety is attached directly to an oligonucleotide through a single bond). In certain oligomeric compounds, a conjugate moiety is attached to an oligonucleotide via a more complex conjugate linker comprising one or more conjugate linker moieities, which are sub-units making up a conjugate linker. In certain embodiments, the conjugate linker comprises a chain structure, such as a hydrocarbyl chain, or an oligomer of repeating units such as ethylene glycol, nucleosides, or amino acid units.

In certain embodiments, a conjugate linker comprises one or more groups selected from alkyl, amino, oxo, amide, disulfide, polyethylene glycol, ether, thioether, and hydroxylamino. In certain such embodiments, the conjugate linker comprises groups selected from alkyl, amino, oxo, amide and ether groups. In certain embodiments, the conjugate linker comprises groups selected from alkyl and amide groups. In certain embodiments, the conjugate linker comprises groups selected from alkyl and ether groups. In certain embodiments, the conjugate linker comprises at least one phosphorus moiety. In certain embodiments, the conjugate linker comprises at least one phosphate group. In certain embodiments, the conjugate linker includes at least one neutral linking group.

In certain embodiments, conjugate linkers, including the conjugate linkers described above, are bifunctional linking moieties, e.g., those known in the art to be useful for attaching conjugate groups to parent compounds, such as the oligonucleotides provided herein. In general, a bifunctional linking moiety comprises at least two functional groups. One of the functional groups is selected to bind to a particular site on a parent compound and the other is selected to bind to a conjugate group. Examples of functional groups used in a bifunctional linking moiety include but are not limited to electrophiles for reacting with nucleophilic groups and nucleophiles for reacting with electrophilic groups. In certain embodiments, bifunctional linking moieties comprise one or more groups selected from amino, hydroxyl, carboxylic acid, thiol, alkyl, alkenyl, and alkynyl. Examples of conjugate linkers include but are not limited to pyrrolidine, 8-amino-3,6-dioxaoctanoic acid (ADO), succinimidyl 4-(N-maleimidomethyl) cyclohexane-1-carboxylate (SMCC) and 6-aminohexanoic acid (AHEX or AHA). Other conjugate linkers include but are not limited to substituted or unsubstituted C₁-C₁₀ alkyl, substituted or unsubstituted C₂-C₁₀ alkenyl or substituted or unsubstituted C₂-C₁₀ alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

In certain embodiments, conjugate linkers comprise 1-10 linker-nucleosides In certain embodiments, such linker-nucleosides are modified nucleosides. In certain embodiments such linker-nucleosides comprise a modified sugar moiety. In certain embodiments, linker-nucleosides are unmodified. In certain embodiments, linker-nucleosides comprise an optionally protected heterocyclic base selected from a purine, substituted purine, pyrimidine or substituted pyrimidine. In certain embodiments, a cleavable moiety is a nucleoside selected from uracil, thymine, cytosine, 4-N-benzoylcytosine, 5-methylcytosine, 4-N-benzoyl-5-methylcytosine, adenine, 6-N-benzoyladenine, guanine and 2-N-isobutyrylguanine. It is typically desirable for linker-nucleosides to be cleaved from the oligomeric compound after it reaches a target tissue. Accordingly, linker-nucleosides are typically linked to one another and to the remainder of the oligomeric compound through cleavable bonds. In certain embodiments, such cleavable bonds are phosphodiester bonds.

Herein, linker-nucleosides are not considered to be part of the oligonucleotide. Accordingly, in embodiments in which an oligomeric compound comprises an oligonucleotide consisting of a specified number or range of linked nucleosides and/or a specified percent complementarity to a reference nucleic acid and the oligomeric compound also comprises a conjugate group comprising a conjugate linker comprising linker-nucleosides, those linker-nucleosides are not counted toward the length of the oligonucleotide and are not used in determining the percent complementarity of the oligonucleotide for the reference nucleic acid. For example, an oligomeric compound may comprise (1) a modified oligonucleotide consisting of 8-30 nucleosides and (2) a conjugate group comprising 1-10 linker-nucleosides that are contiguous with the nucleosides of the modified oligonucleotide. The total number of contiguous linked nucleosides in such an oligomeric compound is more than 30. Alternatively, an oligomeric compound may comprise a modified oligonucleotide consisting of 8-30 nucleosides and no conjugate group. The total number of contiguous linked nucleosides in such an oligomeric compound is no more than 30. Unless otherwise indicated conjugate linkers comprise no more than 10 linker-nucleosides. In certain embodiments, conjugate linkers comprise no more than 5 linker-nucleosides. In certain embodiments, conjugate linkers comprise no more than 3 linker-nucleosides. In certain embodiments, conjugate linkers comprise no more than 2 linker-nucleosides. In certain embodiments, conjugate linkers comprise no more than 1 linker-nucleoside.

In certain embodiments, it is desirable for a conjugate group to be cleaved from the oligonucleotide. For example, in certain circumstances oligomeric compounds comprising a particular conjugate moiety are better taken up by a particular cell type, but once the oligomeric compound has been taken up, it is desirable that the conjugate group be cleaved to release the unconjugated or parent oligonucleotide. Thus, certain conjugate linkers may comprise one or more cleavable moieties. In certain embodiments, a cleavable moiety is a cleavable bond. In certain embodiments, a cleavable moiety is a group of atoms comprising at least one cleavable bond. In certain embodiments, a cleavable moiety comprises a group of atoms having one, two, three, four, or more than four cleavable bonds. In certain embodiments, a cleavable moiety is selectively cleaved inside a cell or subcellular compartment, such as a lysosome. In certain embodiments, a cleavable moiety is selectively cleaved by endogenous enzymes, such as nucleases.

In certain embodiments, a cleavable bond is selected from among: an amide, an ester, an ether, one or both esters of a phosphodiester, a phosphate ester, a carbamate, or a disulfide. In certain embodiments, a cleavable bond is one or both of the esters of a phosphodiester. In certain embodiments, a cleavable moiety comprises a phosphate or phosphodiester. In certain embodiments, the cleavable moiety is a phosphate linkage between an oligonucleotide and a conjugate moiety or conjugate group.

In certain embodiments, a cleavable moiety comprises or consists of one or more linker-nucleosides. In certain such embodiments, the one or more linker-nucleosides are linked to one another and/or to the remainder of the oligomeric compound through cleavable bonds. In certain embodiments, such cleavable bonds are unmodified phosphodiester bonds. In certain embodiments, a cleavable moiety is 2′-deoxy nucleoside that is attached to either the 3′ or 5′-terminal nucleoside of an oligonucleotide by a phosphate internucleoside linkage and covalently attached to the remainder of the conjugate linker or conjugate moiety by a phosphate or phosphorothioate linkage. In certain such embodiments, the cleavable moiety is 2′-deoxyadenosine.

### 3. Certain Cell-Targeting Conjugate Moieties

In certain embodiments, a conjugate group comprises a cell-targeting conjugate moiety. In certain embodiments, a conjugate group has the general formula:

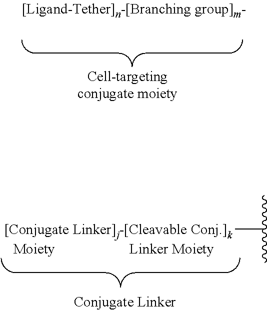

wherein n is from 1 to about 3, m is 0 when n is 1, m is 1 when n is 2 or greater, j is 1 or 0, and k is 1 or 0.

In certain embodiments, n is 1, j is 1 and k is 0. In certain embodiments, n is 1, j is 0 and k is 1. In certain embodiments, n is 1, j is 1 and k is 1. In certain embodiments, n is 2, j is 1 and k is 0. In certain embodiments, n is 2, j is 0 and k is 1. In certain embodiments, n is 2, j is 1 and k is 1. In certain embodiments, n is 3, j is 1 and k is 0. In certain embodiments, n is 3, j is 0 and k is 1. In certain embodiments, n is 3, j is 1 and k is 1.

In certain embodiments, conjugate groups comprise cell-targeting moieties that have at least one tethered ligand. In certain embodiments, cell-targeting moieties comprise two tethered ligands covalently attached to a branching group. In certain embodiments, cell-targeting moieties comprise three tethered ligands covalently attached to a branching group.

In certain embodiments, the cell-targeting moiety comprises a branching group comprising one or more groups selected from alkyl, amino, oxo, amide, disulfide, polyethylene glycol, ether, thioether and hydroxylamino groups. In certain embodiments, the branching group comprises a branched aliphatic group comprising groups selected from alkyl, amino, oxo, amide, disulfide, polyethylene glycol, ether, thioether and hydroxylamino groups. In certain such embodiments, the branched aliphatic group comprises groups selected from alkyl, amino, oxo, amide and ether groups. In certain such embodiments, the branched aliphatic group comprises groups selected from alkyl, amino and ether groups. In certain such embodiments, the branched aliphatic group comprises groups selected from alkyl and ether groups. In certain embodiments, the branching group comprises a mono or polycyclic ring system.

In certain embodiments, each tether of a cell-targeting moiety comprises one or more groups selected from alkyl, substituted alkyl, ether, thioether, disulfide, amino, oxo, amide, phosphodiester, and polyethylene glycol, in any combination. In certain embodiments, each tether is a linear aliphatic group comprising one or more groups selected from alkyl, ether, thioether, disulfide, amino, oxo, amide, and polyethylene glycol, in any combination. In certain embodiments, each tether is a linear aliphatic group comprising one or more groups selected from alkyl, phosphodiester, ether, amino, oxo, and amide, in any combination. In certain embodiments, each tether is a linear aliphatic group comprising one or more groups selected from alkyl, ether, amino, oxo, and amide, in any combination. In certain embodiments, each tether is a linear aliphatic group comprising one or more groups selected from alkyl, amino, and oxo, in any combination. In certain embodiments. each tether is a linear aliphatic group comprising one or more groups selected from alkyl and oxo, in any combination. In certain embodiments, each tether is a linear aliphatic group comprising one or more groups selected from alkyl and phosphodiester, in any combination. In certain embodiments, each tether comprises at least one phosphorus linking group or neutral linking group. In certain embodiments, each tether comprises a chain from about 6 to about 20 atoms in length. In certain embodiments, each tether comprises a chain from about 10 to about 18 atoms in length. In certain embodiments, each tether comprises about 10 atoms in chain length.

In certain embodiments, each ligand of a cell-targeting moiety has an affinity for at least one type of receptor on a target cell. In certain embodiments, each ligand has an affinity for at least one type of receptor on the surface of a mammalian liver cell. In certain embodiments, each ligand has an affinity for the hepatic asialoglycoprotein receptor (ASGP-R). In certain embodiments, each ligand is a carbohydrate. In certain embodiments, each ligand is, independently selected from galactose, N-acetyl galactoseamine (GalNAc), mannose, glucose, glucosamine and fucose. In certain embodiments, each ligand is N-acetyl galactoseamine (GalNAc). In certain embodiments, the cell-targeting moiety comprises 3 GalNAc ligands. In certain embodiments, the cell-targeting moiety comprises 2 GalNAc ligands. In certain embodiments, the cell-targeting moiety comprises 1 GalNAc ligand.

In certain embodiments, each ligand of a cell-targeting moiety is a carbohydrate, carbohydrate derivative, modified carbohydrate, polysaccharide, modified polysaccharide, or polysaccharide derivative. In certain such embodiments, the conjugate group comprises a carbohydrate cluster (see, e.g., Maier et al., “Synthesis of Antisense Oligonucleotides Conjugated to a Multivalent Carbohydrate Cluster for Cellular Targeting,” Bioconjugate Chemistry, 2003, 14, 18-29 or Rensen et al., “Design and Synthesis of Novel N-Acetylgalactosamine-Terminated Glycolipids for Targeting of Lipoproteins to the Hepatic Asiaglycoprotein Receptor,” J. Med. Chem. 2004, 47, 5798-5808). In certain such embodiments, each ligand is an amino sugar or a thio sugar. For example, amino sugars may be selected from any number of compounds known in the art, such as sialic acid, α-D-galactosamine, β-muramic acid, 2-deoxy-2-methylamino-L-glucopyranose, 4,6-dideoxy-4-formamido-2,3-di-O-methyl-D-mannopyranose, 2-deoxy-2-sulfoamino-D-glucopyranose and N-sulfo-D-glucosamine, and N-glycoloyl-α-neuraminic acid. For example, thio sugars may be selected from 5-Thio-β-D-glucopyranose, methyl 2,3,4-tri-O-acetyl-1-thio-6-O-trityl-α-D-glucopyranoside, 4-thio-β-D-galactopyranose, and ethyl 3,4,6,7-tetra-O-acetyl-2-deoxy-1,5-dithio-α-D-gluco-heptopyranoside.

In certain embodiments, conjugate groups comprise a cell-targeting moiety having the formula:

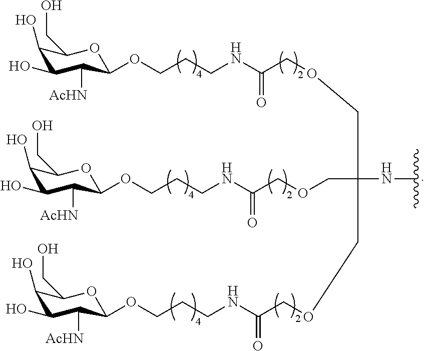

In certain embodiments, conjugate groups comprise a cell-targeting moiety having the formula:

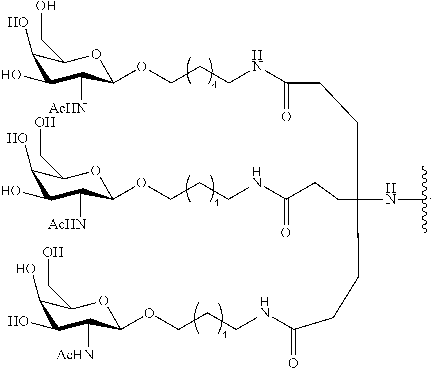

In certain embodiments, conjugate groups comprise a cell-targeting moiety having the formula:

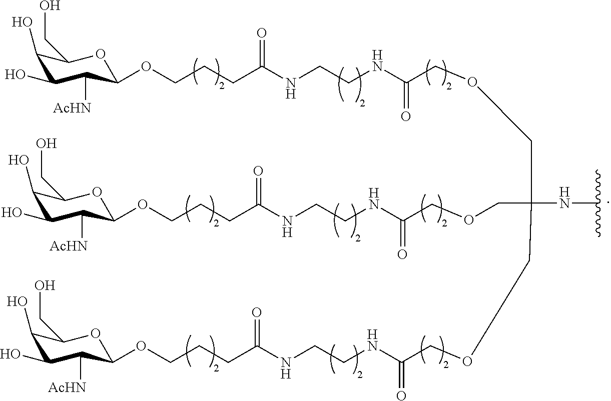

In certain embodiments, conjugate groups comprise a cell-targeting moiety having the formula:

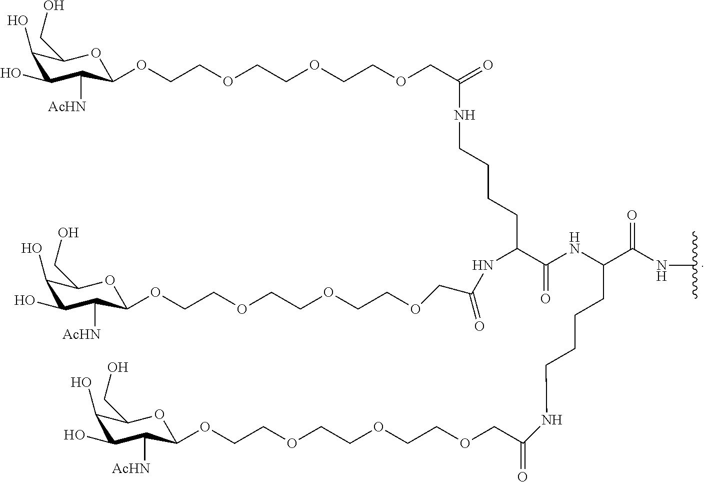

In certain embodiments, conjugate groups comprise a cell-targeting moiety having the formula:

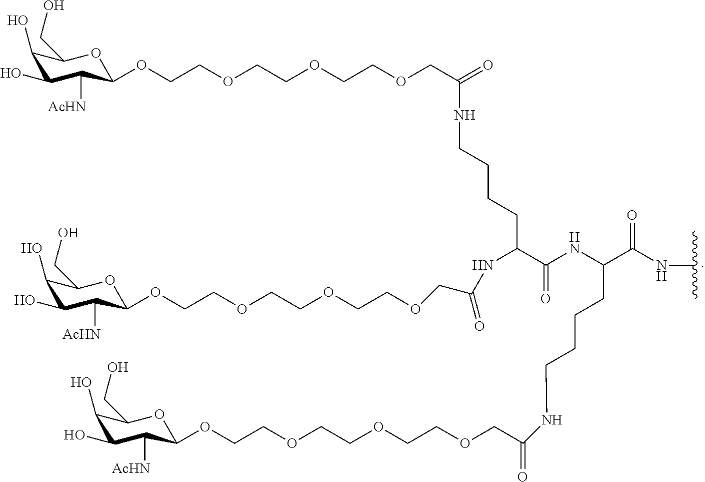

In certain embodiments, oligomeric compounds comprise a conjugate group described herein as “LICA-1”. LICA-1 has the formula:

In certain embodiments, oligomeric compounds comprising LICA-1 have the formula:

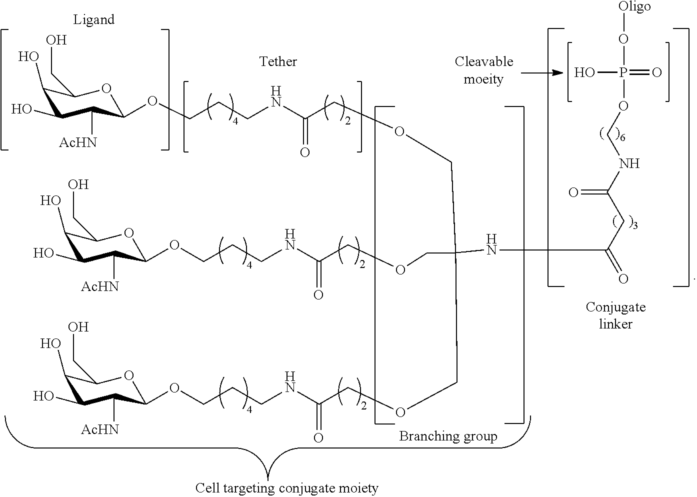

wherein oligo is an oligonucleotide.

Representative United States patents, United States patent application publications, international patent application publications, and other publications that teach the preparation of certain of the above noted conjugate groups, oligomeric compounds comprising conjugate groups, tethers, conjugate linkers, branching groups, ligands, cleavable moieties as well as other modifications include without limitation, U.S. Pat. Nos. 5,994,517, 6,300,319, 6,660,720, 6,906,182, 7,262,177, 7,491,805, 8,106,022, 7,723,509, US 2006/0148740, US 2011/0123520, WO 2013/033230 and WO 2012/037254, Biessen et al., J. Med. Chem. 1995, 38, 1846-1852, Lee et al., Bioorganic & Medicinal Chemistry 2011,19, 2494-2500, Rensen et al., J. Biol. Chem. 2001, 276, 37577-37584, Rensen et al., J. Med. Chem. 2004, 47, 5798-5808, Sliedregt et al., J. Med. Chem. 1999, 42, 609-618, and Valentijn et al., Tetrahedron, 1997, 53, 759-770.

In certain embodiments, oligomeric compounds comprise modified oligonucleotides comprising a gapmer or fully modified sugar motif and a conjugate group comprising at least one, two, or three GalNAc ligands. In certain embodiments antisense compounds and oligomeric compounds comprise a conjugate group found in any of the following references: Lee, Carbohydr Res, 1978, 67, 509-514; Connolly et al., J Biol Chem, 1982, 257, 939-945; Pavia et al., Int J Pep Protein Res, 1983, 22, 539-548; Lee et al., Biochem, 1984, 23, 4255-4261; Lee et al., Glycoconjugate J, 1987, 4, 317-328: Toyokuni et al., Tetrahedron Lett, 1990, 31, 2673-2676; Biessen et al., J Med Chem, 1995, 38, 1538-1546; Valentijn et al., Tetrahedron, 1997, 53, 759-770; Kim et al., Tetrahedron Lett, 1997, 38, 3487-3490; Lee et al., Bioconjug Chem, 1997, 8, 762-765; Kato et al., Glycobiol, 2001, 11, 821-829; Rensen et al., J Biol Chem, 2001, 276, 37577-37584: Lee et al., Methods Enzymol, 2003, 362, 38-43; Westerlind et al., Glycoconj J, 2004, 21, 227-241; Lee et al., Bioorg Med Chem Lett, 2006, 16(19), 5132-5135; Maierhofer et al., Bioorg Med Chem, 2007, 15, 7661-7676; Khorev et al., Bioorg Med Chem, 2008, 16, 5216-5231; Lee et al., Bioorg Med Chem, 2011, 19, 2494-2500; Kornilova et al., Analyt Biochem, 2012, 425, 43-46; Pujol et al., Angew Chemie Int Ed Engl, 2012, 51, 7445-7448; Biessen et al., J Med Chem, 1995, 38, 1846-1852; Sliedregt et al., J Med Chem, 1999, 42, 609-618; Rensen et al., J Med Chem, 2004, 47, 5798-5808; Rensen et al., Arterioscler Thromb Vasc Biol, 2006, 26, 169-175; van Rossenberg et al., Gene Ther, 2004, 11, 457-464; Sato et al., J Am Chem Soc, 2004, 126, 14013-14022; Lee et al., J Org Chem, 2012, 77, 7564-7571; Biessen et al., FASEB J, 2000, 14, 1784-1792; Rajur et al., Bioconjug Chem, 1997, 8, 935-940; Duff et al., Methods Enzymol, 2000, 313, 297-321; Maier et al., Bioconjug Chem, 2003, 14, 18-29; Jayaprakash et al., Org Lett, 2010, 12, 5410-5413; Manoharan, Antisense Nucleic Acid Drug Dev, 2002, 12, 103-128; Merwin et al., Bioconjug Chem, 1994, 5, 612-620; Tomiya et al., Bioorg Med Chem, 2013, 21, 5275-5281; International applications WO1998/013381; WO2011/038356; WO1997/046098; WO2008/098788; WO2004/101619; WO2012/037254; WO2011/120053; WO2011/100131; WO2011/163121; WO2012/177947; WO2013/033230; WO2013/075035; WO2012/083185; WO2012/083046; WO2009/082607; WO2009/134487; WO2010/144740; WO2010/148013; WO1997/020563; WO2010/088537; WO2002/043771; WO2010/129709; WO2012/068187; WO2009/126933; WO2004/024757; WO2010/054406; WO2012/089352; WO2012/089602; WO2013/166121; WO2013/165816; U.S. Pat. Nos. 4,751,219; 8,552,163; 6,908,903; 7,262,177; 5,994,517; 6,300,319; 8,106,022; 7,491,805; 7,491,805; 7,582,744; 8,137,695; 6,383,812; 6,525,031; 6,660,720; 7,723,509; 8,541,548; 8,344,125; 8,313,772; 8,349,308; 8,450,467; 8,501,930; 8,158,601; 7,262,177; 6,906,182; 6,620,916; 8,435,491; 8,404,862; 7,851,615; Published U.S. Patent Application Publications US2011/0097264; US2011/0097265; US2013/0004427; US2005/0164235; US2006/0148740; US2008/0281044; US2010/0240730; US2003/0119724; US2006/0183886; US2008/0206869; US2011/0269814; US2009/0286973; US2011/0207799; US2012/0136042; US2012/0165393; US2008/0281041; US2009/0203135; US2012/0035115; US2012/0095075; US2012/0101148; US2012/0128760; US2012/0157509; US2012/0230938; US2013/0109817; US2013/0121954; US2013/0178512; US2013/0236968; US2011/0123520; US2003/0077829; US2008/0108801; and US2009/0203132.

In certain embodiments, compounds of the invention are single-stranded. In certain embodiments, oligomeric compounds are paired with a second oligonucleotide or oligomeric compound to form a duplex, which is double-stranded.

### III. Certain Antisense Compounds

In certain embodiments, the present invention provides antisense compounds, which comprise or consist of an oligomeric compound comprising an antisense oligonucleotide, having a nucleobase sequences complementary to that of a target nucleic acid. In certain embodiments, antisense compounds are single-stranded. Such single-stranded antisense compounds typically comprise or consist of an oligomeric compound that comprises or consists of a modified oligonucleotide and optionally a conjugate group. In certain embodiments, antisense compounds are double-stranded. Such double-stranded antisense compounds comprise a first oligomeric compound having a region complementary to a target nucleic acid and a second oligomeric compound having a region complementary to the first oligomeric compound. The first oligomeric compound of such double stranded antisense compounds typically comprises or consists of a modified oligonucleotide and optionally a conjugate group. The oligonucleotide of the second oligomeric compound of such double-stranded antisense compound may be modified or unmodified. Either or both oligomeric compounds of a double-stranded antisense compound may comprise a conjugate group. The oligomeric compounds of double-stranded antisense compounds may include non-complementary overhanging nucleosides.

In certain embodiments, oligomeric compounds of antisense compounds are capable of hybridizing to a target nucleic acid, resulting in at least one antisense activity. In certain embodiments, antisense compounds selectively affect one or more target nucleic acid. Such selective antisense compounds comprises a nucleobase sequence that hybridizes to one or more target nucleic acid, resulting in one or more desired antisense activity and does not hybridize to one or more non-target nucleic acid or does not hybridize to one or more non-target nucleic acid in such a way that results in significant undesired antisense activity.

In certain antisense activities, hybridization of an antisense compound to a target nucleic acid results in recruitment of a protein that cleaves the target nucleic acid. For example, certain antisense compounds result in RNase H mediated cleavage of the target nucleic acid. RNase H is a cellular endonuclease that cleaves the RNA strand of an RNA:DNA duplex. The DNA in such an RNA:DNA duplex need not be unmodified DNA. In certain embodiments, the invention provides antisense compounds that are sufficiently “DNA-like” to elicit RNase H activity. Further, in certain embodiments, one or more non-DNA-like nucleoside in the gap of a gapmer is tolerated.

In certain antisense activities, an antisense compound or a portion of an antisense compound is loaded into an RNA-induced silencing complex (RISC), ultimately resulting in cleavage of the target nucleic acid. For example, certain antisense compounds result in cleavage of the target nucleic acid by Argonaute. Antisense compounds that are loaded into RISC are RNAi compounds. RNAi compounds may be double-stranded (siRNA) or single-stranded (ssRNA).

In certain embodiments, hybridization of an antisense compound to a target nucleic acid does not result in recruitment of a protein that cleaves that target nucleic acid. In certain such embodiments, hybridization of the antisense compound to the target nucleic acid results in alteration of splicing of the target nucleic acid. In certain embodiments, hybridization of an antisense compound to a target nucleic acid results in inhibition of a binding interaction between the target nucleic acid and a protein or other nucleic acid. In certain such embodiments, hybridization of an antisense compound to a target nucleic acid results in alteration of translation of the target nucleic acid.

Antisense activities may be observed directly or indirectly. In certain embodiments, observation or detection of an antisense activity involves observation or detection of a change in an amount of a target nucleic acid or protein encoded by such target nucleic acid, a change in the ratio of splice variants of a nucleic acid or protein, and/or a phenotypic change in a cell or animal.

### IV. Certain Target Nucleic Acids

In certain embodiments, antisense compounds comprise or consist of an oligonucleotide comprising a region that is complementary to a target nucleic acid. In certain embodiments, the target nucleic acid is an endogenous RNA molecule. In certain embodiments, the target nucleic acid encodes a protein. In certain such embodiments, the target nucleic acid is selected from: an mRNA and a pre-mRNA, including intronic, exonic and untranslated regions. In certain embodiments, the target RNA is an mRNA. In certain embodiments, the target nucleic acid is a pre-mRNA. In certain such embodiments, the target region is entirely within an intron. In certain embodiments, the target region spans an intron/exon junction. In certain embodiments, the target region is at least 50% within an intron.

In certain embodiments, the target nucleic acid is a non-coding RNA. In certain such embodiments, the target non-coding RNA is selected from: a long-non-coding RNA, a short non-coding RNA, an intronic RNA molecule, a snoRNA, a scaRNA, a microRNA (including pre-microRNA and mature microRNA), a ribosomal RNA, and promoter directed RNA. In certain embodiments, the target nucleic acid is a nucleic acid other than a mature mRNA. In certain embodiments, the target nucleic acid is a nucleic acid other than a mature mRNA or a microRNA. In certain embodiments, the target nucleic acid is a non-coding RNA other than a microRNA. In certain embodiments, the target nucleic acid is a non-coding RNA other than a microRNA or an intronic region of a pre-mRNA. In certain embodiments, the target nucleic acid is a long non-coding RNA. In certain embodiments, the target nucleic acid is a non-coding RNA associated with splicing of other pre-mRNAs. In certain embodiments, the target nucleic acid is a nuclear-retained non-coding RNA.

In certain embodiments, antisense compounds described herein are complementary to a target nucleic acid comprising a single-nucleotide polymorphism (SNP). In certain such embodiments, the antisense compound is capable of modulating expression of one allele of the SNP-containing target nucleic acid to a greater or lesser extent than it modulates another allele. In certain embodiments, an antisense compound hybridizes to a (SNP)-containing target nucleic acid at the single-nucleotide polymorphism site.

In certain embodiments, antisense compounds are at least partially complementary to more than one target nucleic acid. For example, antisense compounds of the present invention may mimic microRNAs, which typically bind to multiple targets.

### A. Complementarity/Mismatches to the Target Nucleic Acid

In certain embodiments, antisense compounds comprise antisense oligonucleotides that are complementary to the target nucleic acid over the entire length of the oligonucleotide. In certain embodiments, such oligonucleotides are 99% complementary to the target nucleic acid. In certain embodiments, such oligonucleotides are 95% complementary to the target nucleic acid. In certain embodiments, such oligonucleotides are 90% complementary to the target nucleic acid. In certain embodiments, such oligonucleotides are 85% complementary to the target nucleic acid. In certain embodiments, such oligonucleotides are 80% complementary to the target nucleic acid. In certain embodiments, antisense oligonucleotides are at least 80% complementary to the target nucleic acid over the entire length of the oligonucleotide and comprise a region that is 100% or fully complementary to a target nucleic acid. In certain such embodiments, the region of full complementarity is from 6 to 20 nucleobases in length. In certain such embodiments, the region of full complementarity is from 10 to 18 nucleobases in length. In certain such embodiments, the region of full complementarity is from 18 to 20 nucleobases in length.

In certain embodiments, the oligomeric compounds of antisense compounds comprise one or more mismatched nucleobases relative to the target nucleic acid. In certain such embodiments, antisense activity against the target is reduced by such mismatch, but activity against a non-target is reduced by a greater amount. Thus, in certain such embodiments selectivity of the antisense compound is improved. In certain embodiments, the mismatch is specifically positioned within an oligonucleotide having a gapmer motif. In certain such embodiments, the mismatch is at position 1, 2, 3, 4, 5, 6, 7, or 8 from the 5′-end of the gap region. In certain such embodiments, the mismatch is at position 9, 8, 7, 6, 5, 4, 3, 2, 1 from the 3′-end of the gap region. In certain such embodiments, the mismatch is at position 1, 2, 3, or 4 from the 5′-end of the wing region. In certain such embodiments, the mismatch is at position 4, 3, 2, or 1 from the 3′-end of the wing region.

### B. Apolipoprotein (a) (Apo(a))

In certain embodiments, conjugated antisense compounds target any apo(a) nucleic acid. In certain embodiments, the target nucleic acid encodes an apo(a) target protein that is clinically relevant. In such embodiments, modulation of the target nucleic acid results in clinical benefit.

The targeting process usually includes determination of at least one target region, segment, or site within the target nucleic acid for the antisense interaction to occur such that the desired effect will result.

In certain embodiments, a target region is a structurally defined region of the nucleic acid. For example, in certain such embodiments, a target region may encompass a 3′ UTR, a 5′ UTR, an exon, an intron, a coding region, a translation initiation region, translation termination region, or other defined nucleic acid region or target segment.

In certain embodiments, a target segment is at least about an 8-nucleobase portion of a target region to which a conjugated antisense compound is targeted. Target segments can include DNA or RNA sequences that comprise at least 8 consecutive nucleobases from the 5′-terminus of one of the target segments (the remaining nucleobases being a consecutive stretch of the same DNA or RNA beginning immediately upstream of the 5′-terminus of the target segment and continuing until the DNA or RNA comprises about 8 to about 30 nucleobases). Target segments are also represented by DNA or RNA sequences that comprise at least 8 consecutive nucleobases from the 3′-terminus of one of the target segments (the remaining nucleobases being a consecutive stretch of the same DNA or RNA beginning immediately downstream of the 3′-terminus of the target segment and continuing until the DNA or RNA comprises about 8 to about 30 nucleobases). Target segments can also be represented by DNA or RNA sequences that comprise at least 8 consecutive nucleobases from an internal portion of the sequence of a target segment, and may extend in either or both directions until the conjugated antisense compound comprises about 8 to about 30 nucleobases.

In certain embodiments, antisense compounds targeted to an apo(a) nucleic acid can be modified as described herein. In certain embodiments, the antisense compounds can have a modified sugar moiety, an unmodified sugar moiety or a mixture of modified and unmodified sugar moieties as described herein. In certain embodiments, the antisense compounds can have a modified internucleoside linkage, an unmodified internucleoside linkage or a mixture of modified and unmodified internucleoside linkages as described herein. In certain embodiments, the antisense compounds can have a modified nucleobase, an unmodified nucleobase or a mixture of modified and unmodified nucleobases as described herein. In certain embodiments, the antisense compounds can have a motif as described herein.

In certain embodiments, antisense compounds targeted to apo(a) nucleic acids can be conjugated as described herein.

One apo(a) protein is linked via a disulfide bond to a single apolipoprotein B (apoB) protein to form a lipoprotein(a) (Lp(a)) particle. The apo(a) protein shares a high degree of homology with plasminogen particularly within the kringle IV type 2 repetitive domain. It is thought that the kringle repeat domain in apo(a) may be responsible for its pro-thrombotic and anti-fibrinolytic properties, potentially enhancing atherosclerotic progression. Apo(a) is transcriptionally regulated by IL-6 and in studies in rheumatoid arthritis patients treated with an IL-6 inhibitor (tocilizumab), plasma levels were reduced by 30% after 3 month treatment. Apo(a) has been shown to preferentially bind oxidized phospholipids and potentiate vascular inflammation. Further, studies suggest that the Lp(a) particle may also stimulate endothelial permeability, induce plasminogen activator inhibitor type-1 expression and activate macrophage interleukin-8 secretion. Importantly, recent genetic association studies revealed that Lp(a) was an independent risk factor for myocardial infarction, stroke, peripheral vascular disease and abdominal aortic aneurysm. Further, in the Precocious Coronary Artery Disease (PROCARDIS) study, Clarke et al. described robust and independent associations between coronary heart disease and plasma Lp(a) concentrations. Additionally, Solfrizzi et al., suggested that increased serum Lp(a) may be linked to an increased risk for Alzheimer's Disease (AD). Antisense compounds targeting apo(a) have been previously disclosed in WO2005/000201 and US2010-0331390, herein incorporated by reference in its entirety. An antisense oligonucleobase targeting Apo(a), ISIS-APOA_{Rx}, was assessed in a Phase I clinical trial to study it's safety profile.

### Certain Conjugated Antisense Compounds Targeted to an Apo(a) Nucleic Acid

In certain embodiments, conjugated antisense compounds are targeted to an Apo(a) nucleic acid having the sequence of GENBANK® Accession No. NM_005577.2, incorporated herein as SEQ ID NO: 1; GENBANK Accession No. NT_007422.12 truncated from nucleotides 3230000 to 3380000, incorporated herein as SEQ ID NO: 2; GENBANK Accession No. NT_025741.15 truncated from nucleotides 65120000 to 65258000, designated herein as SEQ ID NO: 3; and GENBANK Accession No. NM_005577.1, incorporated herein as SEQ ID NO: 4. In certain such embodiments, a conjugated antisense compound is at least 90%, at least 95%, or 100% complementary to any of the nucleobase sequences of SEQ ID NOs: 1-4.

TABLE A
Antisense Compounds targeted to
Apo(a) SEQ ID NO: 1

| | Target | | | SEQ |
|---|---|---|---|---|
| ISIS | Start | Sequence | | ID |
| No | Site | (5′-3′) | Motif | NO |
| 494372 | 3901 | TGCTCCGTTG | eeeeeddddd | 7 |
| | | GTGCTTGTTC | dddddeeeee | |
| 494283 | 584 | TCTTCCTGTG | eeeeeddddd | 8 |
| | 926 | ACAGTGGTGG | dddddeeeee | |
| | 1610 | | | |
| | 1952 | | | |
| | 2294 | | | |
| | 3320 | | | |
| 494284 | 585 | TTCTTCCTGT | eeeeeddddd | 9 |
| | 927 | GACAGTGGTG | dddddeeeee | |
| | 1611 | | | |
| | 1953 | | | |
| | 2295 | | | |
| | 3321 | | | |
| 494286 | 587 | GGTTCTTCCT | eeeeeddddd | 10 |
| | 929 | GTGACAGTGG | dddddeeeee | |
| | 1613 | | | |
| | 1955 | | | |
| | 2297 | | | |
| 494301 | 628 | CGACTATGCG | eeeeeddddd | 11 |
| | 970 | AGTGTGGTGT | dddddeeeee | |
| | 1312 | | | |
| | 1654 | | | |
| | 1996 | | | |
| | 2338 | | | |
| | 2680 | | | |
| | 3022 | | | |
| 494302 | 629 | CCGACTATGC | eeeeeddddd | 12 |
| | 971 | GAGTGTGGTG | dddddeeeee | |
| | 1313 | | | |
| | 1655 | | | |
| | 1997 | | | |
| | 2339 | | | |
| | 2681 | | | |
| | 3023 | | | |

In certain embodiments, the present disclosure provides conjugated antisense compounds represented by the following structure. In certain embodiments, the antisense compound comprises the conjugated modified oligonucleotide ISIS 681257. In certain embodiments, the antisense compound comprises the conjugated modified oligonucleotide ISIS 681257 or a salt thereof. In certain embodiments, the antisense compound consists of the conjugated modified oligonucleotide ISIS 681257.

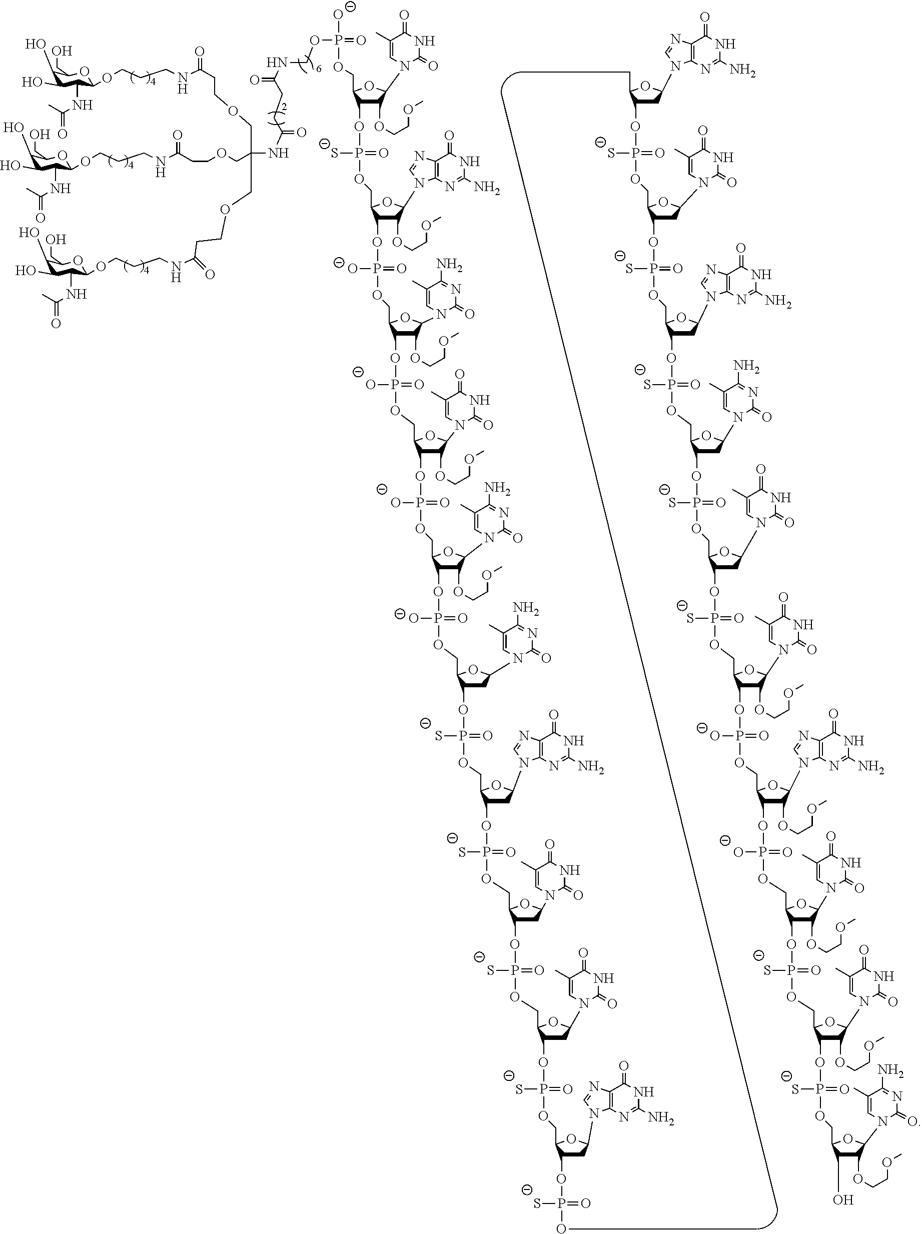

### Apo(a) Therapeutic Indications

In certain embodiments, the invention provides methods for using a conjugated antisense compound targeted to an apo(a) nucleic acid for modulating the expression of apo(a) in a subject. In certain embodiments, the expression of apo(a) is reduced.

In certain embodiments, provided herein are methods of treating a subject comprising administering one or more pharmaceutical compositions as described herein. In certain embodiments, the invention provides methods for using a conjugated antisense compound targeted to an apo(a) nucleic acid in a pharmaceutical composition for treating a subject. In certain embodiments, the individual has an apo(a) related disease. In certain embodiments, the individual has an Lp(a) related disease. In certain embodiments, the individual has an inflammatory, cardiovascular and/or a metabolic disease, disorder or condition.

In certain embodiments, the subject has an inflammatory, cardiovascular and/or metabolic disease, disorder or condition.

In certain embodiments, the cardiovascular diseases, disorders or conditions (CVD) include, but are not limited to, elevated Lp(a) associated CVD risk, recurrent cardiovascular events with elevated Lp(a), aortic stenosis (e.g., calcific aortic stenosis associated with elevated Lp(a)), aneurysm (e.g., abdominal aortic aneurysm), angina, arrhythmia, atherosclerosis, cerebrovascular disease, coronary artery disease, coronary heart disease, dyslipidemia, hypercholesterolemia, hyperlipidemia, hypertension, hypertriglyceridemia, myocardial infarction, peripheral vascular disease (e.g., peripheral artery disease), stroke and the like.

In certain embodiments, the compounds targeted to apo(a) described herein modulate physiological markers or phenotypes of the cardiovascular disease, disorder or condition. For example, administration of the compounds to animals can decrease Lp(a), LDL and cholesterol levels in those animals compared to untreated animals. In certain embodiments, the modulation of the physiological markers or phenotypes can be associated with inhibition of apo(a) by the compounds.

In certain embodiments, the physiological markers of the cardiovascular disease, disorder or condition can be quantifiable. For example, Lp(a), LDL or cholesterol levels can be measured and quantified by, for example, standard lipid tests. For such markers, in certain embodiments, the marker can be decreased by about 5, 10, 15, 20, 25, 30, 35, 40, 45, 50, 55, 60, 65, 70, 75, 80, 85, 90, 95 or 99%, or a range defined by any two of these values.

Also, provided herein are methods for preventing, treating or ameliorating a symptom associated with the cardiovascular disease, disorder or condition in a subject in need thereof. In certain embodiments, provided is a method for reducing the rate of onset of a symptom associated with the cardiovascular disease, disorder or condition. In certain embodiments, provided is a method for reducing the severity of a symptom associated with the cardiovascular disease, disorder or condition. In such embodiments, the methods comprise administering a therapeutically effective amount of a compound targeted to an apo(a) nucleic acid to an individual in need thereof.

The cardiovascular disease, disorder or condition can be characterized by numerous physical symptoms. Any symptom known to one of skill in the art to be associated with the cardiovascular disease, disorder or condition can be prevented, treated, ameliorated or otherwise modulated with the compounds and methods described herein. In certain embodiments, the symptom can be any of, but not limited to, angina, chest pain, shortness of breath, palpitations, weakness, dizziness, nausea, sweating, tachycardia, bradycardia, arrhythmia, atrial fibrillation, swelling in the lower extremities, cyanosis, fatigue, fainting, numbness of the face, numbness of the limbs, claudication or cramping of muscles, bloating of the abdomen or fever.

In certain embodiments, the metabolic diseases, disorders or conditions include, but are not limited to, hyperglycemia, prediabetes, diabetes (type I and type II), obesity, insulin resistance, metabolic syndrome and diabetic dyslipidemia.

In certain embodiments, compounds targeted to apo(a) as described herein modulate physiological markers or phenotypes of the metabolic disease, disorder or condition. For example, administrion of the compounds to animals can decrease glucose and insulin resistance levels in those animals compared to untreated animals. In certain embodiments, the modulation of the physiological markers or phenotypes can be associated with inhibition of apo(a) by the compounds.

In certain embodiments, physiological markers of the metabolic disease, disorder or condition can be quantifiable. For example, glucose levels or insulin resistance can be measured and quantified by standard tests known in the art. For such markers, in certain embodiments, the marker can be decreased by about 5, 10, 15, 20, 25, 30, 35, 40, 45, 50, 55, 60, 65, 70, 75, 80, 85, 90, 95 or 99%, or a range defined by any two of these values. In another example, insulin sensitivity can be measured and quantified by standard tests known in the art. For such markers, in certain embodiments, the marker can be increase by about 5, 10, 15, 20, 25, 30, 35, 40, 45, 50, 55, 60, 65, 70, 75, 80, 85, 90, 95 or 99%, or a range defined by any two of these values.

Also, provided herein are methods for preventing, treating or ameliorating a symptom associated with the metabolic disease, disorder or condition in a subject in need thereof. In certain embodiments, provided is a method for reducing the rate of onset of a symptom associated with the metabolic disease, disorder or condition. In certain embodiments, provided is a method for reducing the severity of a symptom associated with the metabolic disease, disorder or condition. In such embodiments, the methods comprise administering a therapeutically effective amount of a compound targeted to an apo(a) nucleic acid to an individual in need thereof.

The metabolic disease, disorder or condition can be characterized by numerous physical symptoms. Any symptom known to one of skill in the art to be associated with the metabolic disease, disorder or condition can be prevented, treated, ameliorated or otherwise modulated with the compounds and methods described herein. In certain embodiments, the symptom can be any of, but not limited to, excessive urine production (polyuria), excessive thirst and increased fluid intake (polydipsia), blurred vision, unexplained weight loss and lethargy.

In certain embodiments, the inflammatory diseases, disorders or conditions include, but are not limited to, elevated Lp(a) associated CVD risk, recurrent cardiovascular events with elevated Lp(a), aortic stenosis (e.g., calcific aortic valve stenosis associated with high Lp(a)), coronary artey disease (CAD), Alzheimer's Disease and thromboembolic diseases, disorder or conditions. Certain thromboembolic diseases, disorders or conditions include, but are not limited to, stroke, thrombosis, myocardial infarction and peripheral vascular disease.

In certain embodiments, the compounds targeted to apo(a) described herein modulate physiological markers or phenotypes of the inflammatory disease. disorder or condition. For example, administration of the compounds to animals can decrease inflammatory cytokine or other inflammatory markers levels in those animals compared to untreated animals. In certain embodiments, the modulation of the physiological markers or phenotypes can be associated with inhibition of apo(a) by the compounds.

In certain embodiments, the physiological markers of the inflammatory disease, disorder or condition can be quantifiable. For example, cytokine levels can be measured and quantified by standard tests known in the art. For such markers, in certain embodiments, the marker can be decreased by at least about 5%, 10%, 15%, 20%, 25%, 30%, 35%, 40%, 45%, 50%, 55%, 60%, 65%, 70%, 75%, 80%, 85%, 90%, 95% or 99%, or a range defined by any two of these values.

Also, provided herein are methods for preventing, treating or ameliorating a symptom associated with the inflammatory disease, disorder or condition in a subject in need thereof. In certain embodiments, provided is a method for reducing the rate of onset of a symptom associated with the inflammatory disease, disorder or condition. In certain embodiments, provided is a method for reducing the severity of a symptom associated with the inflammatory disease, disorder or condition. In such embodiments, the methods comprise administering a therapeutically effective amount of a compound targeted to an apo(a) nucleic acid to an individual in need thereof.

In certain embodiments, provided are methods of treating an individual with an apo(a) related disease, disorder or condition comprising administering a therapeutically effective amount of one or more pharmaceutical compositions as described herein. In certain embodiments, the individual has elevated apo(a) levels. In certain embodiments, provided are methods of treating an individual with an Lp(a) related disease, disorder or condition comprising administering a therapeutically effective amount of one or more pharmaceutical compositions as described herein. In certain embodiments, the individual has elevated Lp(a) levels. In certain embodiments, the individual has an inflammatory, cardiovascular and/or metabolic disease, disorder or condition. In certain embodiments, administration of a therapeutically effective amount of an antisense compound targeted to an apo(a) nucleic acid is accompanied by monitoring of apo(a) or Lp(a) levels. In certain embodiments, administration of a therapeutically effective amount of an antisense compound targeted to an apo(a) nucleic acid is accompanied by monitoring of markers of inflammatory, cardiovascular and/or metabolic disease, or other disease process associated with the expression of apo(a), to determine an individual's response to the antisense compound. An individual's response to administration of the antisense compound targeting apo(a) can be used by a physician to determine the amount and duration of therapeutic intervention with the compound.

In certain embodiments, administration of an antisense compound targeted to an apo(a) nucleic acid results in reduction of apo(a) expression by at least about 15%, 20%, 25%, 30%, 35%, 40%, 45%, 50%, 55%, 60%, 65%, 70%, 75%, 80%, 85%, 90%, 95% or 99%, or a range defined by any two of these values. In certain embodiments, apo(a) expression is reduced to at least ≤100 mg/dl, ≤90 mg/dL, ≤80 mg/dL, ≤70 mg/dL, ≤60 mg/dL, ≤50 mg/dL, ≤40 mg/dL, ≤30 mg/dL, ≤20 mg/dL or ≤10 mg/dL.

In certain embodiments, administration of an antisense compound targeted to an apo(a) nucleic acid results in reduction of Lp(a) expression by at least about 15%, 20%, 25%, 30%, 35%, 40%, 45%, 50%, 55%, 60%, 65%, 70%, 75%, 80%, 85%, 90%, 95% or 99%, or a range defined by any two of these values. In certain embodiments, Lp(a) expression is reduced to at least ≤200 mg/dL, ≤190 mg/dL, ≤180 mg/dL, ≤175 mg/dL, ≤170 mg/dL, ≤160 mg/dL, ≤150 mg/dL, ≤140 mg/dL, ≤130 mg/dL, ≤120 mg/dL, ≤110 mg/dL, ≤100 mg/dL, ≤90 mg/dL, ≤80 mg/dL, ≤70 mg/dL, ≤60 mg/dL, ≤55 mg/dL, ≤50 mg/dL, ≤45 mg/dL, ≤40 mg/dL, ≤35 mg/dL, ≤30 mg/dL, ≤25 mg/dL, ≤20 mg/dL, ≤15 mg/dL, or ≤10 mg/dL.

In certain embodiments, the invention provides methods for using a conjugated antisense compound targeted to an apo(a) nucleic acid in the preparation of a medicament. In certain embodiments, pharmaceutical compositions comprising a conjugated antisense compound targeted to apo(a) are used for the preparation of a medicament for treating a patient suffering or susceptible to an inflammatory, cardiovascular and/or a metabolic disease, disorder or condition.

### Apo(a) Treatment Populations

Certain subjects with high Lp(a) levels are at a significant risk of various diseases (Lippi et al., Clinica Chimica Acta, 2011, 412:797-801; Solfrizz et al.). In many subjects with high Lp(a) levels, current treatments cannot reduce their Lp(a) levels to safe levels. Apo(a) plays an important role in the formation of Lp(a), hence reducing apo(a) can reduce Lp(a) and prevent, treat or ameliorate a disease associated with Lp(a).

In certain embodiments, treatment with the compounds and methods disclosed herein is indicated for a human animal with elevated apo(a) levels and/or Lp(a) levels. In certain embodiments, the human has apo(a) levels ≥10 mg/dL, ≥20 mg/dL, ≥30 mg/dL, ≥40 mg/dL, ≥50 mg/dL, ≥60 mg/dL, ≥70 mg/dL, ≥80 mg/dL, ≥90 mg/dL or ≥100 mg/dL. In certain embodiments, the human has Lp(a) levels ≥10 mg/dL, ≥15 mg/dL, ≥20 mg/dL, ≥25 mg/dL, ≥30 mg/dL, ≥35 mg/dL, ≥40 mg/dL, ≥50 mg/dL, ≥60 mg/dL, ≥70 mg/dL, ≥80 mg/dL, ≥90 mg/dL, ≥100 mg/dL, ≥110 mg/dL, ≥120 mg/dL, ≥130 mg/dL, ≥140 mg/dL, ≥150 mg/dL, ≥160 mg/dL, ≥170 mg/dL, ≥175 mg/dL, ≥180 mg/dL, ≥190 mg/dL, ≥200 mg/dL.

### C. Apolipoprotein C-III (ApoCIII)

ApoCIII is a constituent of HDL and of triglyceride (TG)-rich lipoproteins. Elevated ApoCIII levels are associated with elevated TG levels and diseases such as cardiovascular disease, metabolic syndrome, obesity and diabetes. Elevated TG levels are associated with pancreatitis. ApoCIII slows clearance of TG-rich lipoproteins by inhibiting lipolysis through inhibition of lipoprotein lipase (LPL) and through interfering with lipoprotein binding to cell-surface glycosaminoglycan matrix. Antisense compounds targeting ApoCIII have been previously disclosed in WO2004/093783 and WO2012/149495, each herein incorporated by reference in its entirety. Currently, an antisense oligonucleotide targeting ApoCIII, ISIS-APOCIII_{Rx}, is in Phase II clinical trials to assess its effectiveness in the treatment of diabetes or hypertriglyceridemia. However, there is still a need to provide patients with additional and more potent treatment options.

a. Certain Conjugated Antisense Compounds Targeted to an ApoCIII Nucleic Acid

In certain embodiments, conjugated antisense compounds are targeted to an ApoCIII nucleic acid having the sequence of GENBANK® Accession No. NT_033899.8 truncated from nucleobases 20262640 to 20266603, incorporated herein as SEQ ID NO: 6. In certain such embodiments, a conjugated antisense compound is at least 90%, at least 95%, or 100% complementary to SEQ ID NO: 6. In certain embodiments, such conjugated antisense compounds comprise a conjugate comprising 1-3 GalNAc ligands. In certain embodiments, such antisense compounds comprise a conjugate disclosed herein.

In certain embodiments, conjugated antisense compounds are targeted to an ApoCIII nucleic acid having the sequence of GENBANK® Accession No. NM_000040.1, incorporated herein as SEQ ID NO: 5. In certain such embodiments, a conjugated antisense compound is at least 90%, at least 95%, or 100% complementary to SEQ ID NO: 5. In certain embodiments, such conjugated antisense compounds comprise a conjugate comprising 1-3 GalNAc ligands. In certain embodiments, such antisense compounds comprise a conjugate disclosed herein.

In certain embodiments, a conjugated antisense compound targeted to SEQ ID NO: 5 comprises an at least 8 consecutive nucleobase sequence of SEQ ID NO: 13. In certain embodiments, a conjugated antisense compound targeted to SEQ ID NO: 5 comprises a nucleobase sequence of SEQ ID NO: 13. In certain embodiments, such conjugated antisense compounds comprise a conjugate comprising 1-3 GalNAc ligands. In certain embodiments, such antisense compounds comprise a conjugate disclosed herein.

TABLE 5
Antisense Compounds targeted to
ApoCIII SEQ ID NO: 5

| | Target | | | SEQ |
|---|---|---|---|---|
| ISIS | Start | Sequence | | ID |
| No | Site | (5′-3′) | Motif | NO |
| 304801 | 508 | AGCTTCTTGT | eeeeeddddd | 13 |
| | | CCAGCTTTAT | dddddeeeee | |
| 647535 | 508 | AGCTTCTTGT | eeeeeddddd | 13 |
| | | CCAGCTTTAT | dddddeeeee | |
| | | | od | |
| 616468 | 508 | AGCTTCTTGT | eeeeeddddd | 13 |
| | | CCAGCTTTAT | dddddeeeee | |
| 647536 | 508 | AGCTTCTTGT | eeoeoeoeod | 13 |
| | | CCAGCTTTAT | ddddddddde | |
| | | | oeoeeeod | |

In certain embodiments, the present disclosure provides conjugated antisense compounds represented by the following structure. In certain embodiments, the antisense compound comprises the conjugated modified oligonucleotide ISIS 678354. In certain embodiments, the antisense compound comprises the conjugated modified oligonucleotide ISIS 678354 or a salt thereof. In certain embodiments, the antisense compound consists of the conjugated modified oligonucleotide ISIS 678354.

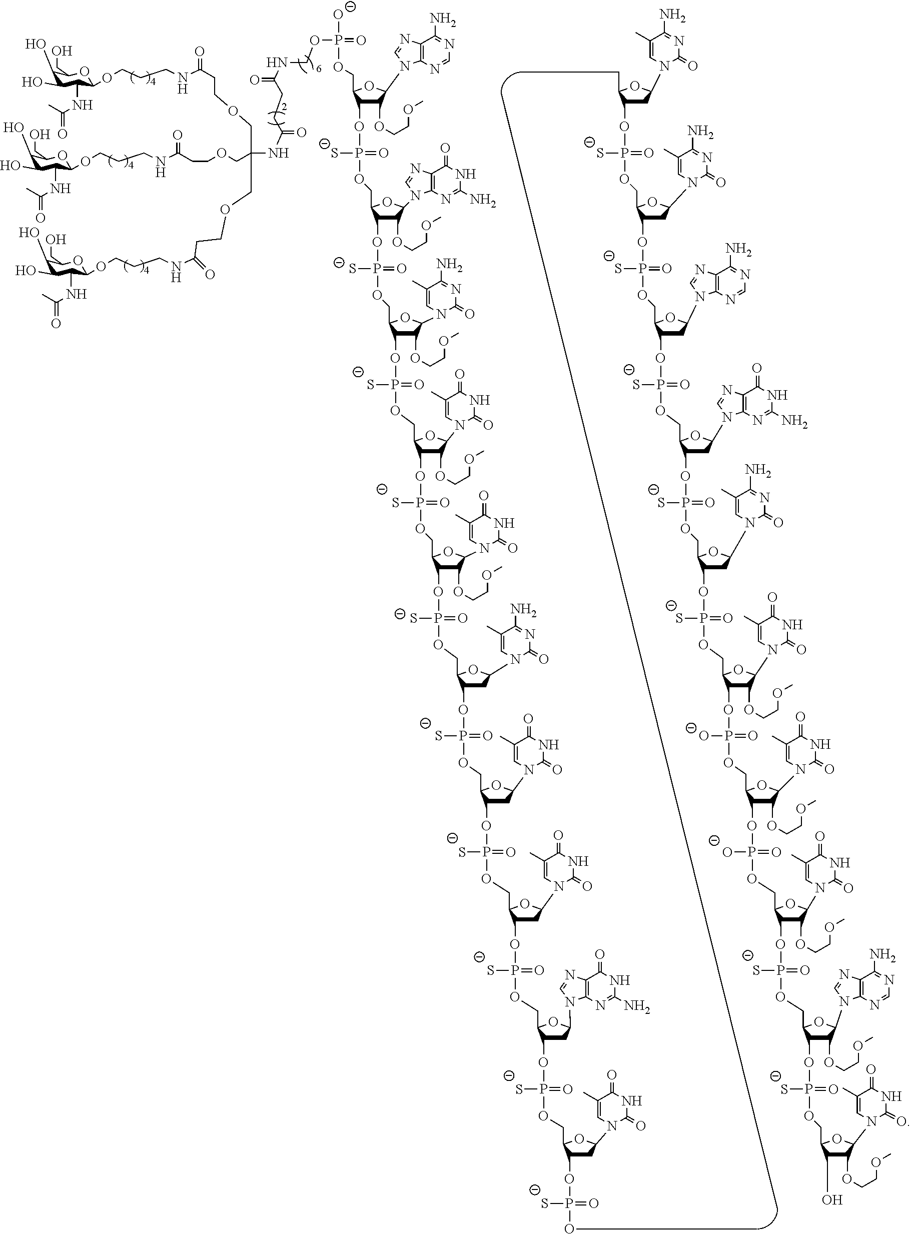

b. ApoCIII Therapeutic Indications

In certain embodiments, the invention provides methods for using a conjugated antisense compound targeted to an ApoCIII nucleic acid for modulating the expression of ApoCIII in a subject. In certain embodiments, the expression of ApoCIII is reduced.

In certain embodiments, the invention provides methods for using a conjugated antisense compound targeted to an ApoCIII nucleic acid in a pharmaceutical composition for treating a subject. In certain embodiments, the subject has a cardiovascular and/or metabolic disease, disorder or condition. In certain embodiments, the subject has hypertriglyceridemia, non-familial hypertriglyceridemia, familial hypertriglyceridemia, heterozygous familial hypertriglyceridemia, homozygous familial hypertriglyceridemia, mixed dyslipidemia, atherosclerosis, a risk of developing atherosclerosis, coronary heart disease, a history of coronary heart disease, early onset coronary heart disease, one or more risk factors for coronary heart disease, type II diabetes, type II diabetes with dyslipidemia, dyslipidemia (e.g., lipodystrophy), hyperlipidemia, hypercholesterolemia, hyperfattyacidemia, hepatic steatosis, non-alcoholic steatohepatitis, pancreatitis and/or non-alcoholic fatty liver disease.

In certain embodiments, the invention provides methods for using a conjugated antisense compound targeted to an ApoCIII nucleic acid in the preparation of a medicament.

a. Certain ApoCIII Dosing Regimens

In certain embodiments, ISIS 678354 is administered to a subject in need thereof. In certain embodiments, 20 mg of ISIS 678354 is administered to a human subject. In certain embodiments, 40 mg of ISIS 678354 is administered to a human subject. In certain embodiments, 80 mg of ISIS 678354 is administered to a human subject. In certain embodiments, 120 mg of ISIS 678354 is administered to a human subject.

In certain embodiments, ISIS 678354 is administered to a subject in need thereof. In certain embodiments, 20 mg of ISIS 678354 is administered to a human subject during a dosing period. In certain embodiments, 40 mg of ISIS 678354 is administered to a human subject during a dosing period. In certain embodiments, 80 mg of ISIS 678354 is administered to a human subject during a dosing period. In certain embodiments, 120 mg of ISIS 678354 is administered to a human subject during a dosing period. In certain embodiments, the dosing period is one week. In certain embodiments, only one dose is given during the dosing period. In certain embodiments, the dosing period is one week.

In certain embodiments, 20 mg of ISIS 678354 is administered to a human subject each week. In certain embodiments, 40 mg of ISIS 678354 is administered to a human subject each week. In certain embodiments, 80 mg of ISIS 678354 is administered to a human subject each week. In certain embodiments, 120 mg of ISIS 678354 is administered to a human subject each week.

### D. Angiopoietin-Like 3

The angiopoietins are a family of secreted growth factors. Together with their respective endothelium-specific receptors, the angiopoietins play important roles in angiogenesis. One family member, angiopoietin-like 3 (also known as angiopoietin-like protein 3, ANGPT5, ANGPTL3, or angiopoietin 5), is predominantly expressed in the liver, and is thought to play a role in regulating lipid metabolism (Kaplan et al., J. Lipid Res., 2003, 44, 136-143). Genome-wide association scans (GWAS) surveying the genome for common variants associated with plasma concentrations of HDL, LDL and triglyceride found an association between triglycerides and single-nucleotide polymorphisms (SNPs) near ANGPTL3 (Willer et al., Nature Genetics, 2008, 40(2):161-169). Individuals with homozygous ANGPTL3 loss-of-function mutations present with low levels of all atherogenic plasma lipids and lipoproteins, such as total cholesterol (TC) and TG, low density lipoprotein cholesterol (LDL-C), apoliprotein B (apoB), non-HDL-C, as well as HDL-C (Romeo et al. 2009, J Clin Invest, 119(1):70-79; Musunuru et al. 2010 N Engl J Med, 363:2220-2227; Martin-Campos et al. 2012, Clin Chim Acta, 413:552-555; Minicocci et al. 2012, J Clin Endocrinol Metab, 97:e1266-1275; Noto et al. 2012, Arterioscler Thromb Vasc Biol, 32:805-809; Pisciotta et al. 2012, Circulation Cardiovasc Genet, 5:42-50). This clinical phenotype has been termed familial combined hypolipidemia (FHBL2). Despite reduced secretion of VLDL, subjects with FHBL2 do not have increased hepatic fat content. They also appear to have lower plasma glucose and insulin levels, and importantly, both diabetes and cardiovascular disease appear to be absent from these subjects. No adverse clinical phenotypes have been reported to date (Minicocci et al. 2013, J of Lipid Research, 54:3481-3490). Reduction of ANGPTL3 has been shown to lead to a decrease in TG, cholesterol and LDL levels in animal models (U.S. Ser. No. 13/520,997; PCT Publication WO 2011/085271). Mice deficient in ANGPTL3 have very low plasma triglyceride (TG) and cholesterol levels, while overexpression produces the opposite effects (Koishi et al. 2002; Koster 2005; Fujimoto 2006). Accordingly, the potential role of ANGPTL3 in lipid metabolism makes it an attractive target for therapeutic intervention.

In certain embodiments, the present disclosure provides conjugated antisense compounds represented by the following structure. In certain embodiments, the antisense compound comprises the conjugated modified oligonucleotide ISIS 703802. In certain embodiments, the antisense compound comprises the conjugated modified oligonucleotide ISIS 703802 or a salt thereof. In certain embodiments, the antisense compound consists of the conjugated modified oligonucleotide ISIS 703802.

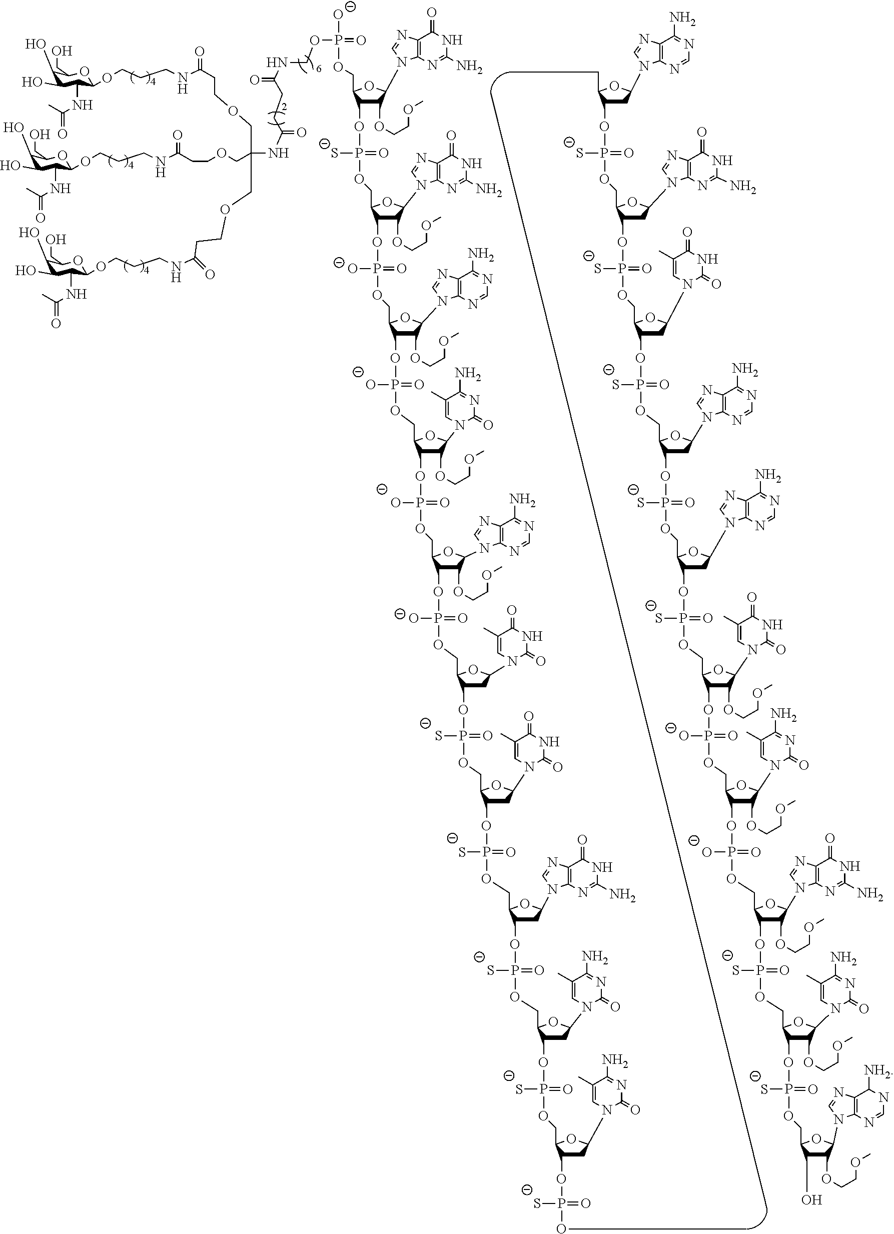

b. ANGPTL3 Therapeutic Indications

In certain embodiments, the invention provides methods for using a conjugated antisense compound targeted to an ANGPTL3 nucleic acid for modulating the expression of ANGPTL3 in a subject. In certain embodiments, the expression of ANGPTL3 is reduced.

In certain embodiments, the invention provides methods for using a conjugated antisense compound targeted to an ANGPTL3 nucleic acid in a pharmaceutical composition for treating a subject. In certain embodiments, the subject has a metabolic disease and/or cardiovascular disease. In certain embodiments, the subject has combined hyperlipidemia (e.g., familial or non-familial), hypercholesterolemia (e.g., familial homozygous hypercholesterolemia (HoFH), familial heterozygous hypercholesterolemia (HeFH)), dyslipidemia, lipodystrophy, hypertriglyceridemia (e.g., heterozygous LPL deficiency, homozygous LPL deficiency), coronary artery disease (CAD), familial chylomicronemia syndrome (FCS), hyperlipoproteinemia Type IV), metabolic syndrome, non-alcoholic fatty liver disease (NAFLD), nonalcoholic steatohepatitis (NASH), diabetes (e.g., Type 2 diabetes, Type 2 diabetes with dyslipidemia), insulin resistance (e.g., insulin resistance with dyslipidemia), vascular wall thickening, high blood pressure (e.g., pulmonary arterial hypertension), sclerosis (e.g., atherosclerosis, systemic sclerosis, progressive skin sclerosis and proliferative obliterative vasculopathy such as digital ulcers and pulmonary vascular involvement), or a combination thereof.

In certain embodiments, the compounds targeted to ANGPTL3 described herein modulate lipid and/or energy metabolism in a subject. In certain embodiments, the compounds targeted to ANGPTL3 described herein modulate physiological markers or phenotypes of hypercholesterolemia, dyslipidemia, lipodystrophy, hypertriglyceridemia, metabolic syndrome, NAFLD, NASH and/or diabetes. For example, administration of the compounds to a subject can modulate one or more of VLDL, non-esterified fatty acids (NEFA), LDL, cholesterol, triglyceride, glucose, insulin or ANGPTL3 levels. In certain embodiments, the modulation of the physiological markers or phenotypes can be associated with inhibition of ANGPTL3 by the compounds.

In certain embodiments, administration of an antisense compound targeted to an ANGPTL3 nucleic acid results in reduction of ANGPTL3 expression by about at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99%, or a range defined by any two of these values.

c. Certain ANGPTL3 Dosing Regimens

In certain embodiments, ISIS 703802 is administered to a subject in need thereof. In certain embodiments, 20 mg of ISIS 703802 is administered to a human subject. In certain embodiments, 40 mg of ISIS 703802 is administered to a human subject. In certain embodiments, 80 mg of ISIS 703802 is administered to a human subject. In certain embodiments, 120 mg of ISIS 703802 is administered to a human subject.

In certain embodiments, ISIS 703802 is administered to a subject in need thereof. In certain embodiments, 20 mg of ISIS 703802 is administered to a human subject during a dosing period. In certain embodiments, 40 mg of ISIS 703802 is administered to a human subject during a dosing period. In certain embodiments, 80 mg of ISIS 703802 is administered to a human subject during a dosing period. In certain embodiments, 120 mg of ISIS 703802 is administered to a human subject during a dosing period. In certain embodiments, the dosing period is one week. In certain embodiments, only one dose is given during the dosing period. In certain embodiments, the dosing period is one week.

In certain embodiments, 20 mg of ISIS 703802 is administered to a human subject each week. In certain embodiments, 40 mg of ISIS 703802 is administered to a human subject each week. In certain embodiments, 80 mg of ISIS 703802 is administered to a human subject each week. In certain embodiments, 120 mg of ISIS 703802 is administered to a human subject each week.

### V. Certain Pharmaceutical Compositions

In certain embodiments, the present invention provides pharmaceutical compositions comprising one or more antisense compound or a salt thereof. In certain such embodiments. the pharmaceutical composition comprises a suitable pharmaceutically acceptable diluent or carrier. In certain embodiments, a pharmaceutical composition comprises a sterile saline solution and one or more antisense compound. In certain embodiments, such pharmaceutical composition consists of a sterile saline solution and one or more antisense compound. In certain embodiments, the sterile saline is pharmaceutical grade saline. In certain embodiments, a pharmaceutical composition comprises one or more antisense compound and sterile water. In certain embodiments, a pharmaceutical composition consists of one antisense compound and sterile water. In certain embodiments, the sterile water is pharmaceutical grade water. In certain embodiments, a pharmaceutical composition comprises one or more antisense compound and phosphate-buffered saline (PBS). In certain embodiments, a pharmaceutical composition consists of one or more antisense compound and sterile PBS. In certain embodiments, the sterile PBS is pharmaceutical grade PBS.

In certain embodiments, pharmaceutical compositions comprise one or more or antisense compound and one or more excipients. In certain such embodiments, excipients are selected from water, salt solutions, alcohol, polyethylene glycols, gelatin, lactose, amylase, magnesium stearate, talc, silicic acid, viscous paraffin, hydroxymethylcellulose and polyvinylpyrrolidone.

In certain embodiments, antisense compounds may be admixed with pharmaceutically acceptable active and/or inert substances for the preparation of pharmaceutical compositions or formulations. Compositions and methods for the formulation of pharmaceutical compositions depend on a number of criteria, including, but not limited to, route of administration, extent of disease, or dose to be administered.

In certain embodiments, pharmaceutical compositions comprising an antisense compound encompass any pharmaceutically acceptable salts of the antisense compound, esters of the antisense compound, or salts of such esters. In certain embodiments, pharmaceutical compositions comprising antisense compounds comprising one or more antisense oligonucleotide, upon administration to an animal, including a human, are capable of providing (directly or indirectly) the biologically active metabolite or residue thereof. Accordingly, for example, the disclosure is also drawn to pharmaceutically acceptable salts of antisense compounds, prodrugs, pharmaceutically acceptable salts of such prodrugs, and other bioequivalents. Suitable pharmaceutically acceptable salts include, but are not limited to, sodium and potassium salts. In certain embodiments, prodrugs comprise one or more conjugate group attached to an oligonucleotide, wherein the conjugate group is cleaved by endogenous nucleases within the body.

Lipid moieties have been used in nucleic acid therapies in a variety of methods. In certain such methods, the nucleic acid, such as an antisense compound, is introduced into preformed liposomes or lipoplexes made of mixtures of cationic lipids and neutral lipids. In certain methods, DNA complexes with mono- or poly-cationic lipids are formed without the presence of a neutral lipid. In certain embodiments, a lipid moiety is selected to increase distribution of a pharmaceutical agent to a particular cell or tissue. In certain embodiments, a lipid moiety is selected to increase distribution of a pharmaceutical agent to fat tissue. In certain embodiments, a lipid moiety is selected to increase distribution of a pharmaceutical agent to muscle tissue.

In certain embodiments, pharmaceutical compositions comprise a delivery system. Examples of delivery systems include, but are not limited to, liposomes and emulsions. Certain delivery systems are useful for preparing certain pharmaceutical compositions including those comprising hydrophobic compounds. In certain embodiments, certain organic solvents such as dimethylsulfoxide are used.

In certain embodiments, pharmaceutical compositions comprise one or more tissue-specific delivery molecules designed to deliver the one or more pharmaceutical agents of the present invention to specific tissues or cell types. For example, in certain embodiments. pharmaceutical compositions include liposomes coated with a tissue-specific antibody. In certain embodiments, pharmaceutical compositions comprise a co-solvent system. Certain of such co-solvent systems comprise, for example, benzyl alcohol, a nonpolar surfactant, a water-miscible organic polymer, and an aqueous phase. In certain embodiments, such co-solvent systems are used for hydrophobic compounds. A non-limiting example of such a co-solvent system is the VPD co-solvent system, which is a solution of absolute ethanol comprising 3% w/v benzyl alcohol, 8% w/v of the nonpolar surfactant Polysorbate 80™ and 65% w/v polyethylene glycol 300. The proportions of such co-solvent systems may be varied considerably without significantly altering their solubility and toxicity characteristics. Furthermore, the identity of co-solvent components may be varied: for example, other surfactants may be used instead of Polysorbate 80™; the fraction size of polyethylene glycol may be varied; other biocompatible polymers may replace polyethylene glycol, e.g., polyvinyl pyrrolidone; and other sugars or polysaccharides may substitute for dextrose.

In certain embodiments, pharmaceutical compositions are prepared for oral administration. In certain embodiments, pharmaceutical compositions are prepared for buccal administration. In certain embodiments, a pharmaceutical composition is prepared for administration by injection (e.g., intravenous, subcutaneous, intramuscular, etc.). In certain of such embodiments, a pharmaceutical composition comprises a carrier and is formulated in aqueous solution, such as water or physiologically compatible buffers such as Hanks's solution, Ringer's solution, or physiological saline buffer. In certain embodiments, other ingredients are included (e.g., ingredients that aid in solubility or serve as preservatives). In certain embodiments, injectable suspensions are prepared using appropriate liquid carriers, suspending agents and the like. Certain pharmaceutical compositions for injection are presented in unit dosage form, e.g., in ampoules or in multi-dose containers. Certain pharmaceutical compositions for injection are suspensions, solutions or emulsions in oily or aqueous vehicles, and may contain formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes. Aqueous injection suspensions may contain.

### VI. Certain Routes of Administration (Application Specific)

In certain embodiments, the present disclosure provides methods of administering a pharmaceutical composition comprising an oligonucleotide of the present disclosure to a human. Suitable administration routes include, but are not limited to, oral, rectal, transmucosal, intestinal, enteral, topical, suppository, through inhalation, intrathecal, intracerebroventricular, intraperitoneal, intranasal, intraocular, intratumoral, and parenteral (e.g., intravenous, intramuscular, intramedullary, and subcutaneous). In certain embodiments, pharmaceutical intrathecals are administered to achieve local rather than systemic exposures. For example, pharmaceutical compositions may be injected directly in the area of desired effect (e.g., into the liver).

### NONLIMITING DISCLOSURE AND INCORPORATION BY REFERENCE

Each of the literature and patent publications listed herein is incorporated by reference in its entirety.

While certain compounds, compositions and methods described herein have been described with specificity in accordance with certain embodiments, the following examples serve only to illustrate the compounds described herein and are not intended to limit the same. Each of the references, GenBank accession numbers, and the like recited in the present application is incorporated herein by reference in its entirety.

Although the sequence listing accompanying this filing identifies each sequence as either “RNA” or “DNA” as required, in reality, those sequences may be modified with any combination of chemical modifications. One of skill in the art will readily appreciate that such designation as “RNA” or “DNA” to describe modified oligonucleotides is, in certain instances, arbitrary. For example, an oligonucleotide comprising a nucleoside comprising a 2′-OH sugar moiety and a thymine base could be described as a DNA having a modified sugar (2′-OH in place of one 2′-H of DNA) or as an RNA having a modified base (thymine (methylated uracil) in place of a uracil of RNA). Accordingly, nucleic acid sequences provided herein, including, but not limited to those in the sequence listing, are intended to encompass nucleic acids containing any combination of natural or modified RNA and/or DNA, including, but not limited to such nucleic acids having modified nucleobases. By way of further example and without limitation, an oligomeric compound having the nucleobase sequence “ATCGATCG” encompasses any oligomeric compounds having such nucleobase sequence, whether modified or unmodified, including, but not limited to, such compounds comprising RNA bases, such as those having sequence “AUCGAUCG” and those having some DNA bases and some RNA bases such as “AUCGATCG” and oligomeric compounds having other modified nucleobases, such as “AT^{m}CGAUCG,” wherein ^{m}C indicates a cytosine base comprising a methyl group at the 5-position.

The compounds described herein include variations in which one or more atoms are replaced with a non-radioactive isotope or radioactive isotope of the indicated element. For example, compounds herein that comprise hydrogen atoms encompass all possible deuterium substitutions for each of the ¹H hydrogen atoms. Isotopic substitutions encompassed by the compounds herein include but are not limited to: ²H or ³H in place of ¹H, ¹³C or ¹⁴C in place of ¹²C, ¹⁵N in place of ¹⁴N, ¹⁷O or ¹⁸O in place of ¹⁶O, and ³³S, ³⁴S, ³⁵S, or ³⁶S in place of ³²S. In certain embodiments, non-radioactive isotopic substitutions may impart new properties on the oligomeric compound that are beneficial for use as a therapeutic or research tool. In certain embodiments, radioactive isotopic substitutions may make the compound suitable for research or diagnostic purposes such as imaging.

### EXAMPLES

The following examples illustrate certain embodiments of the present disclosure and are not limiting. Moreover, where specific embodiments are provided, the inventors have contemplated generic application of those specific embodiments. For example, disclosure of an oligonucleotide having a particular motif provides reasonable support for additional oligonucleotides having the same or similar motif. And, for example, where a particular high-affinity modification appears at a particular position, other high-affinity modifications at the same position are considered suitable, unless otherwise indicated.

### Example 1: ISIS 681257 Clinical Trial

As described herein, a double-blinded, placebo-controlled, dose-escalation Phase 1 study was performed on healthy volunteers with elevated Lp(a) to assess safety, tolerability, pharmacokinetics (PK) and pharmacodynamics (PD) after administration of single and multiple doses of ISIS 681257. ISIS 681257 was previously disclosed in WO 2014/179625 and is also described hereinabove. ISIS 681257 has been shown to be potent in inhibiting Lp(a) and tolerable when administered to non-human subjects. This subsequent study revealed unexpectedly improved properties of ISIS 681257 when administered to human subjects.

### Screening

Up to 28 days prior to treatment, subjects were screened for eligibility to participate in the study. Admission criteria for the study include the following:
1. Healthy males or females aged 18-65 inclusive and weighing≥50 kg at the time of informed consent
2. BMI<35.0 kg/m2
3. Subjects must have Lp(a)≥75 nanomoles/liter (nmol/L) (≥30 mg/dL) at Screening. The Lp(a) value obtained via the Lp(a) pre-screening protocol may also be used to meet this criterion if measured within 6 months of dosing.

### Study Drug

Solutions of the Study Drug ISIS 681257 (100 mg/mL, 0.8 mL) contained in stoppered glass vials was used. Vials were for single use only. Doses of ISIS 681257 solution and placebo (0.9% sterile saline) were prepared by an unblinded pharmacist (or qualified delegate). A trained professional administered the ISIS 681257 or placebo blindly as a subcutaneous (sc) injection(s) in the abdomen, thigh, or outer area of the upper arm on each dosing day.

### Treatment and Post-Treatment Evaluation

Subjects enrolled in the study were split into 2 treatment arms: Single Ascending Dose (SAD) or Multiple Ascending Dose (MAD).

### Example 1A: Single Ascending Dose (SAD)

Approximately 28 subjects were enrolled in the SAD arm of this study, grouped into cohorts of 4 or 8 subjects randomized 3:1, ISIS 681257 to placebo. The subjects were administered placebo or ISIS 681257 at the doses listed in Table 1.

TABLE 1
Single Ascending Doses

| Cohort (n) | Dose |
|---|---|
| A (4) | 10 mg |
| B (4) | 20 mg |
| C (4) | 40 mg |
| D (8) | 80 mg |
| E (8) | 120 mg |

After treatment with a single dose of ISIS 681257 or placebo, the subjects were followed for up to 90 days to monitor the safety, tolerability, PK and PD of the drug. During the follow-up period, subjects return to the Study Center for visits on Study Days 2, 3, 8, 15 and 30 post-treatment (and Days 50, 70 and 90 post-treatment for Cohorts C, D and E) for safety and laboratory evaluations (blood draws), monitoring, concomitant medication usage recording, and adverse event (AE) collection. Collection of urine and feces was also performed on certain days. All visits by the subject for post-treatment assessment had a visit window of up to ±1 days.

Analysis of serum samples showed dose dependent reductions in Lp(a) levels after a single dose of ISIS 681257 as measured 2 days, 4 days, 8 days, 15 days and 30 days post-treatment (Cohorts C, D and E were also assessed about 50 days, 70 days and 90 days post-treatment). Results, presented as a mean percent change in Lp(a) from baseline, are shown in Table 2.

TABLE 2
Dose-dependent Change in Lp(a) after
a Single Dose of ISIS 681257

| | % Change from Baseline | | | | | | | |
|---|---|---|---|---|---|---|---|---|
| | Day | Day | Day | Day | Day | Day | Day | Day |
| Cohort | 2 | 4 | 8 | 15 | 30 | 50 | 70 | 90 |
| Placebo | −1.7 | −2.4 | −9.6 | 0.3 | 6.8 | −14 | −9 | 3.9 |
| A | 4 | −5 | −16 | −20 | −26 | — | — | — |
| B | 7 | 8 | 2 | −22 | −33 | — | — | — |
| C | −2 | −13 | −33 | −41 | −43 | −35 | −26 | −26 |
| D | −21 | −35 | −50 | −70 | −79 | −71 | −52 | −46 |
| E | −11 | −25 | −50 | −76 | −85 | −75 | −61 | −44 |

Additionally, analysis of apo(a) isoforms, lipoprotein-associated phospholipase A2 (Lp-PLA2), secretory phospholipase A2 (sPLA2), oxidized phospholipid associated with apolipoprotein B (OxPL-apoB), and oxidized phospholipid associated with apolipoprotein(a) (OxPL-apo(a)) were performed.

During scheduled visits to the Study Center, the safety and tolerability of ISIS 681257 was clinically assessed in the subjects. Clinical staff assessed safety and tolerability by collecting and/or measuring one or more of the following: adverse events (AEs), quality of life assessments, concomitant medication/procedure information, vital signs, physical examination results (e.g., injection site reactions (ISRs) or flu-like symptoms (FLSs)), waist circumference, skinfold measurements, DEXA scans, electrocardiograms (ECGs), liver MRIs and echocardiograms.

Laboratory measurements such as serum chemistry (e.g., ALT, AST, bilirubin, creatinine, BUN), urinalysis, coagulation (e.g., aPTT (sec), PT (sec), INR, plasminogen), complement (e.g., C5a, Bb), hematology (e.g., hematocrit, white blood cells, platelets), immune function, thyroid function, inflammation (hsCRP), lipid panel (e.g., total cholesterol, HDL, LDL, TG, apoB, VLDL), ISIS 681257 plasma trough concentrations, and/or immunogenicity testing were performed on subject samples to assess the health and safety of each subject and the PD of the drug.

Laboratory measurements of subject samples were also used for PK profiling of the drug. For example, samples were used for measuring the amount and stability of ISIS 681257 and/or metabolites thereof, assessing drug binding proteins, and/or assessing other actions of ISIS 681257 with plasma constituents.

Both single dose treatment and multiple dose treatment with ISIS 681257 did not result in any safety or tolerability issues, at any of the clinically revelant doses tested. No ISRs were observed and no side effects were noted in any laboratory tests and the liver enzymes ALT and AST were not elevated.

The above results were surprising, because earlier experiments involving both the unconjugated compound (ISIS 494372) and the GalNAc conjugated compound (ISIS 681257) had suggested that the GalNAc conjugated compound would have significantly lower potency and/or a shorter duration of action in humans than was observed following the first dosing of humans reported herein (e.g. see Examples 89, 100 and 108 of WO 2014/179625 and Tsimikas et al., Lancet, 2015 Oct. 10; 386:1472-83). In light of these surprising results, when treating humans, the GalNAc conjugated compound (ISIS 681257, or a salt thereof) can be administered at lower doses and/or less frequently than expected based on the earlier in vivo testing of the GalNAc conjugated compound. This can provide one or more very significant improvements in treating humans, e.g. reduced cost of treatment, improved patient compliance, reduced volume of administered medicinal product and/or potentially reduced risk of potential adverse events via lower dose administration regimens.

### Example 1B: Multiple Ascending Dose (MAD)

Thirty subjects were enrolled in the MAD arm of this study, grouped into cohorts of 10 randomized 4:1, ISIS 681257 to placebo. The subjects were administered a placebo or ISIS 681257 at the doses listed in Table 3. A total of 6 doses of drug or placebo was administered to each subject: loading doses were administered in the first week on Study Days 1 (first dose), 3, 5 and 8; maintenance doses were then administered weekly on Study Days 15 and 22.

TABLE 3
Multiple Ascending Doses

| Cohort | ISIS 681257 | # of | Total |
|---|---|---|---|
| (n) | Dose | Doses | Drug Dose |
| AA (10) | 10 mg | 6 | 60 mg |
| BB (10) | 20 mg | 6 | 120 mg |
| CC (10) | 40 mg | 6 | 240 mg |

During treatment with ISIS 681257 or placebo and for up to 13 weeks after treatment, the subjects were monitored for safety, tolerability, PK and PD of the drug. During the treatment period and the follow-up period, subjects return to the Study Center for visits on Study Days 5, 8, 15, 22, 29, 36, 50, 64, 85 and 113 for safety and clinical laboratory evaluations (blood draws), monitoring, concomitant medication usage recording, and AE event collection. Collection of urine and feces was also performed on certain days. All visits by the subject for post-treatment assessment had a visit window of up to ±1 days.

Analysis of serum samples showed reductions in Lp(a) levels after multiple doses of ISIS 681257 as measured 5 days, 8 days, 15 days, 22 days, 29 days and 36 days after start of treatment. Results, presented as a mean percent change in Lp(a) from baseline, are shown in Table 4. Surprisingly, after a single dose of ISIS 681257, levels of Lp(a) continue to fall, reaching a nadir at about day 50 for the AA cohort. This demonstrates that the effective half life of ISIS 681257 appears to be much longer than anticipated. Additionally, cohorts BB and CC demonstrate continued reduction in Lp(a) through 36 days after administration of ISIS 681257.

TABLE 4
Dose-dependent Reductions in Lp(a) after a Multiple Doses of ISIS-681257

| | % Change from Baseline | | | | | | | | | |
|---|---|---|---|---|---|---|---|---|---|---|
| | Day | Day | Day | Day | Day | Day | Day | Day | Day | Day |
| Cohort | 5 | 8 | 15 | 22 | 29 | 36 | 50 | 64 | 85 | 113 |
| Placebo | −2.6 | −11 | −11 | −4 | −3 | −10 | −1 | 18 | −1 | 10 |
| AA | −9 | −23 | −43 | −50 | −61 | −72 | −68 | −66 | −52 | −39 |
| BB | −10 | −20 | −52 | −68 | −75 | −80 | −80 | −77 | −64 | −52 |
| CC | −19 | −44 | −71 | −84 | −90 | −94 | −90 | −85 | −73 | −58 |

TABLE 5
ED50 Values in Human

| | ISIS | ISIS |
|---|---|---|
| Regimen | 494372 | 681257 |
| Weekly Dose | 145 mg | 4.5 mg |
| ED50 | (1.5 ml) | (0.05 ml) |

In human subjects, ISIS 681257 displayed dose-dependent, durable, statistically significant reductions in Lp(a) and an ED50 of 4.5 mg. ISIS 681257 was unexpectedly found to be ≥30-fold more potent than ISIS 494372 (an unconjugated antisense compound of the same nucleobase sequence and length; previously described in WO 2013/177468). Earlier experiments involving both ISIS 494372 and ISIS 681257 (reported in WO 2014/179625) had indicated that the GalNAc conjugated compound benefits from higher in vivo potency in mice, but these earlier experiments did not reveal or predict the unexpected ≥30-fold improvement in humans. Additionally, for the 10 mg multi-dose cohort, data points past Day 36 indicate that the nadir of Lp(a) levels for 6 of the 8 patients was not achieved until about Day 50, indicating that in humans ISIS 681257 showed an unexpected long half-life (T½) compared to ISIS 494372 (Tsimikas et al., Lancet, 2015 Oct. 10; 386:1472-83) and this was likewise not revealed or predicted by earlier experiments in mice involving both ISIS 494372 and ISIS 681257.

Additionally, analysis of apo(a) isoforms, lipoprotein-associated phospholipase A2 (Lp-PLA2), secretory phospholipase A2 (sPLA2), oxidized phospholipid associated with apolipoprotein B (OxPL-apoB), and oxidized phospholipid associated with apolipoprotein(a) (OxPL-apo(a)) were performed. The results showed a significant reduction in LDL cholesterol, apolipoprotein B (apoB), and oxidised phospholipids (OxPL) associated with apoB and apo(a). Therefore, the reductions in Lp(a) occurred alongside significant reductions in proinflammatory OxPL, as well as reductions in LDL-C and apoB-100, which is consistent with a salutary effect on several causal pathways that mediate cardiovascular disease and calcific aortic valve stenosis. See Viney, et al. Lancet, 2016, September 2016; 388: 2239-53.

During scheduled visits to the Study Center, the safety and tolerability of ISIS 681257 was clinically assessed in the subjects. Clinical staff assessed safety and tolerability by collecting and/or measuring one or more of the following: adverse events (AEs), quality of life assessments, concomitant medication/procedure information, vital signs, physical examination results (e.g., injection site reactions (ISRs) or flu-like symptoms (FLSs)), waist circumference, skinfold measurements, DEXA scans, electrocardiograms (ECGs), liver MRIs and echocardiograms.

Laboratory measurements such as serum chemistry (e.g., ALT, AST, bilirubin, creatinine, BUN), urinalysis, coagulation (e.g., aPTT (sec), PT (sec), INR, plasminogen), complement (e.g., C5a, Bb), hematology (e.g., hematocrit, white blood cells, platelets), immune function, thyroid function, inflammation (hsCRP), lipid panel (e.g., total cholesterol, HDL, LDL, TG, apoB, VLDL), ISIS 681257 plasma trough concentrations. and/or immunogenicity testing were performed on subject samples to assess the health and safety of each subject and the PD of the drug.

Laboratory measurements of subject samples were also used for PK profiling of the drug. For example, samples were used for measuring the amount and stability of ISIS 681257 and/or metabolites thereof, assessing drug binding proteins, and/or assessing other actions of ISIS 681257 with plasma constituents.

Multiple dose treatments with ISIS 681257 did not result in any safety or tolerability issues. No ISR or FLS were observed. Liver enzymes ALT and AST were not elevated.

The ≥30-fold improvement in potency in humans was significantly greater than that expected. The above results were surprising, because earlier experiments involving both the unconjugated compound (ISIS 494372) and the GalNAc conjugated compound (ISIS 681257) had suggested that the GalNAc conjugated compound would have significantly lower potency and/or a shorter duration of action in humans than was observed following the first dosing of humans reported herein (e.g. see Examples 89, 100 and 108 of WO 2014/179625 and Tsimikas et al., Lancet, 2015 Oct. 10; 386:1472-83). In light of these surprising results, when treating humans, the GalNAc conjugated compound (ISIS 681257, or a salt thereof) can be administered at lower doses and/or less frequently than expected based on the earlier in vivo testing of the GalNAc conjugated compound. This can provide one or more very significant improvements in treating humans, e.g. reduced cost of treatment, improved patient compliance, reduced volume of administered medicinal product and/or potentially reduced risk of potential adverse events via lower dose administration regimens.

### Example 2: Dose Regimens

Modeling based on the Phase I clinical trial results was performed to assess optimal clinical dose regimens for ISIS 681257.

### Weekly Dosing

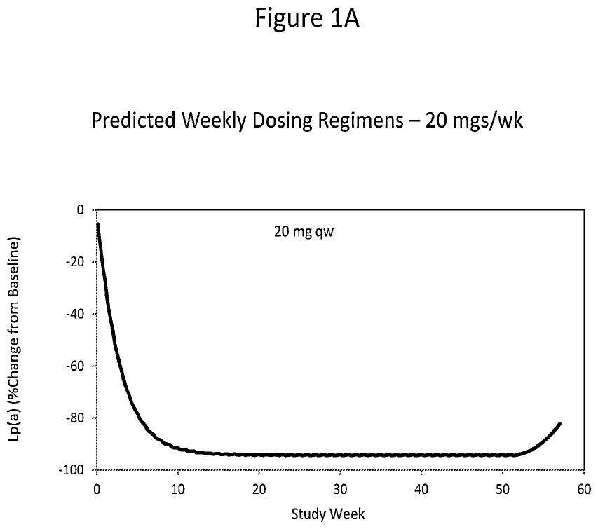

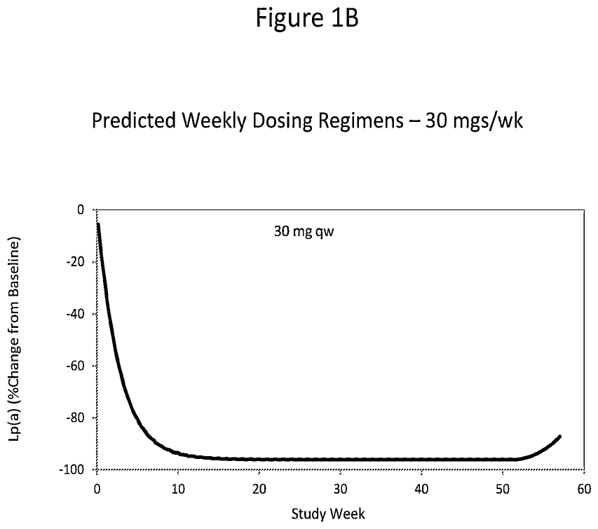

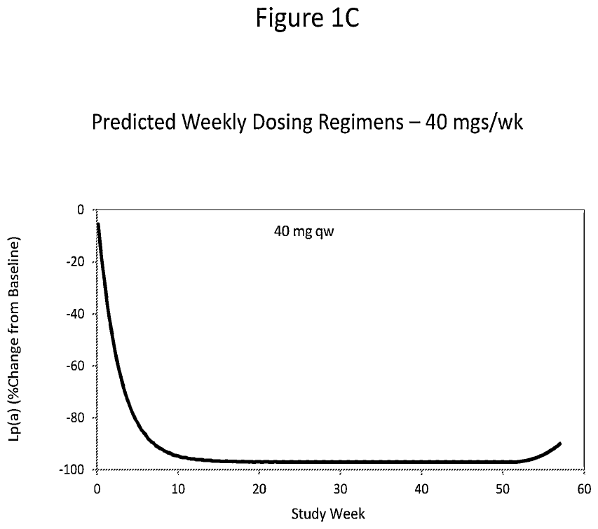

FIGS. 1A-C. Predicted Weekly Dosing Regimens. Charts are shown modeling the effect on Lp(a) by weekly administration of ISIS 681257 at doses of 20 mg (FIG. 1A), 30 mg (FIG. 1B) or 40 mg (FIG. 1C). Lp(a) shows a steady state reduction of ≥80%.

### Monthly Dosing

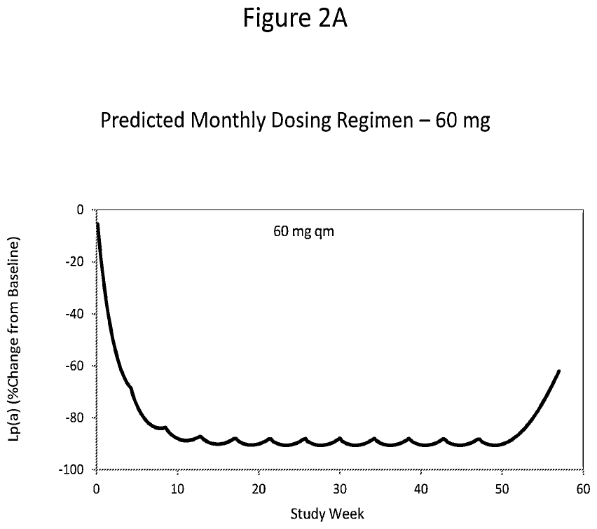

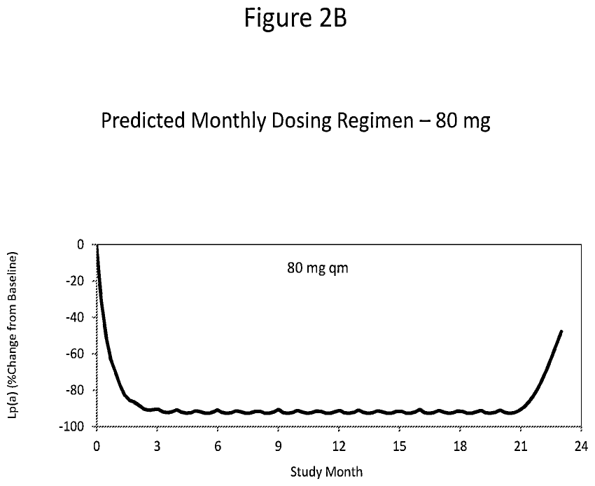

FIGS. 2A-B. Predicted Monthly Dosing Regimens. Chart are shown modeling the effect on Lp(a) by monthly administration of ISIS 681257 at dose of 60 mg (FIG. 2A) and 80 mg (FIG. 2B). Lp(a) shows a steady state reduction of about 80%.

### Two Months Dosing

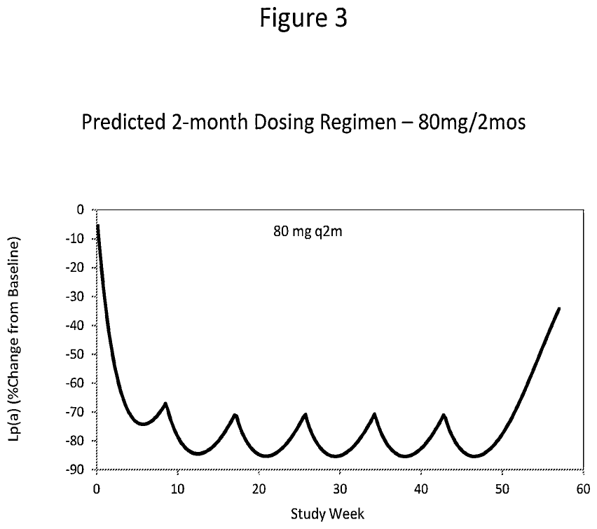

FIG. 3. Predicted 2-month Dosing Regimen. A chart is shown modeling the effect on Lp(a) by administration of ISIS 681257 at an 80 mg dose every 2-months. Lp(a) shows a steady state reduction of about 80%.

### Quarterly Dosing

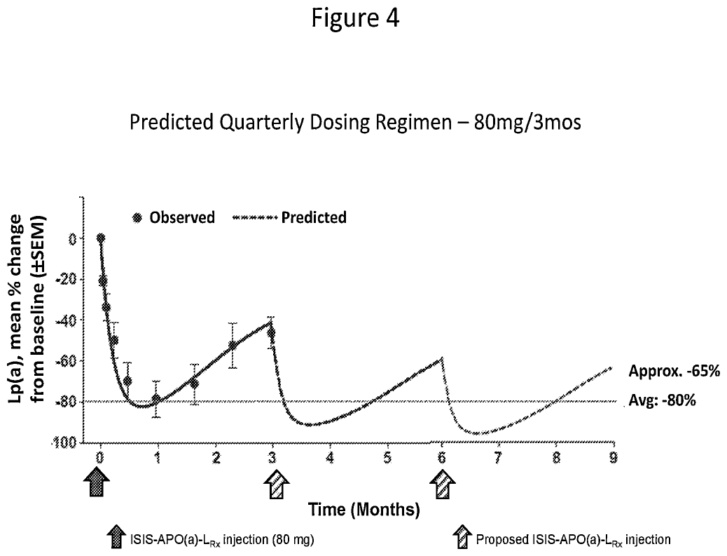

FIG. 4. Predicted Quarterly Dosing Regimen. A chart is shown modeling the effect on Lp(a) by quarterly administration of ISIS 681257 at an 80 mg dose. Lp(a) shows a steady state reduction of 80% and maximum reduction of >90%.

### Example 3: Dose Regimens

After completion of the phase 1 study described above, further modeling was performed to assess optimal clinical dose regimens for ISIS 681257.

### Weekly Dosing

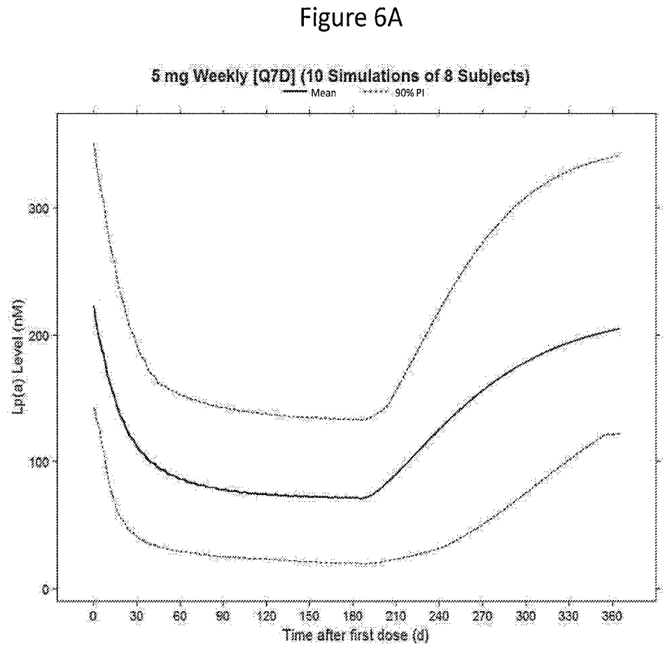

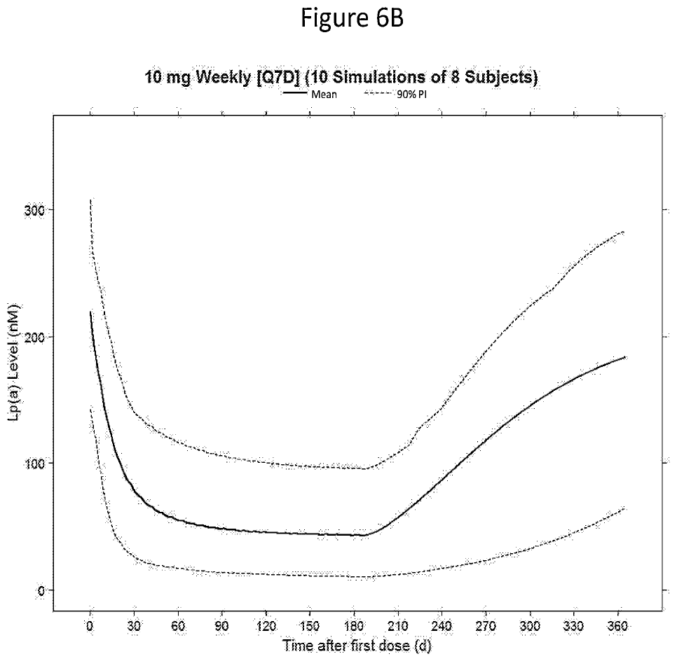

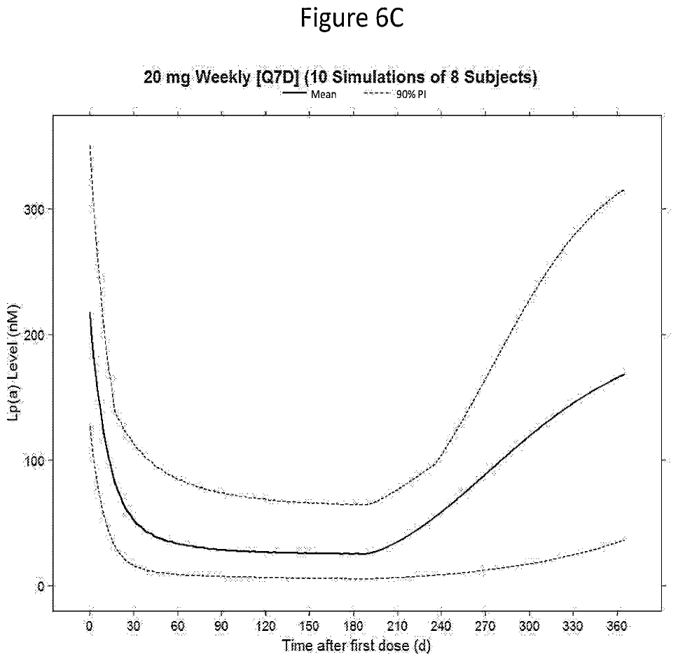

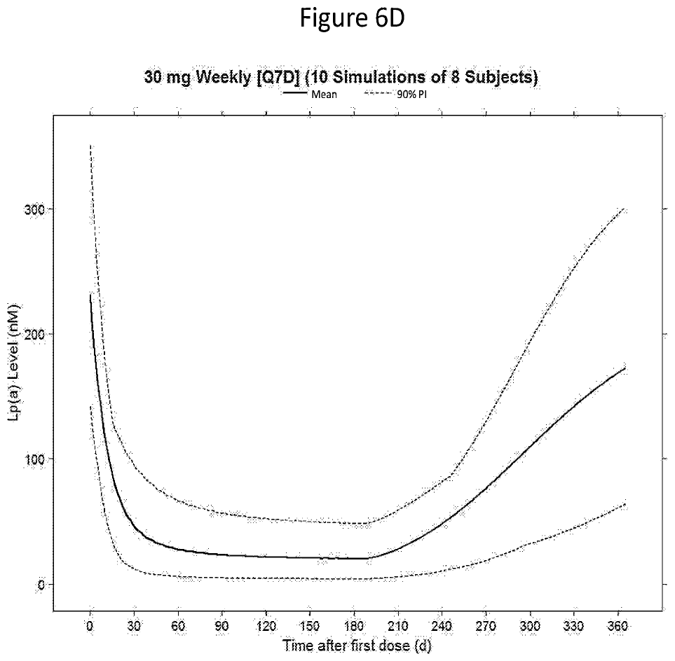

FIGS. 6A-D. Predicted Weekly Dosing Regimens. Charts are shown modeling the effect on Lp(a) by weekly administration of ISIS 681257 at doses of 5 mg (FIG. 6A), 10 mg (FIG. 6B), 20 mg (FIG. 6C), and 30 mg (FIG. 6D). The dark middle line represents the predicted dose, while the uppermost and lowermost lines represent the 90% Confidence Interval.

### Monthly Dosing

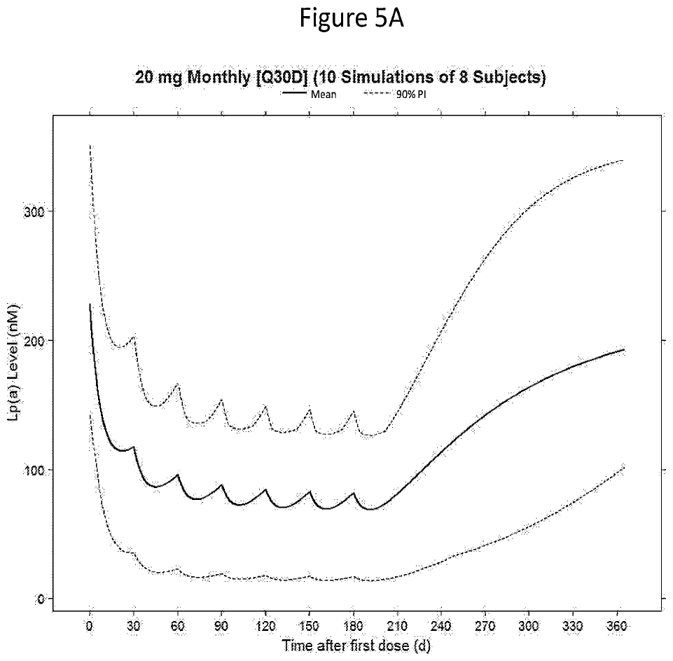

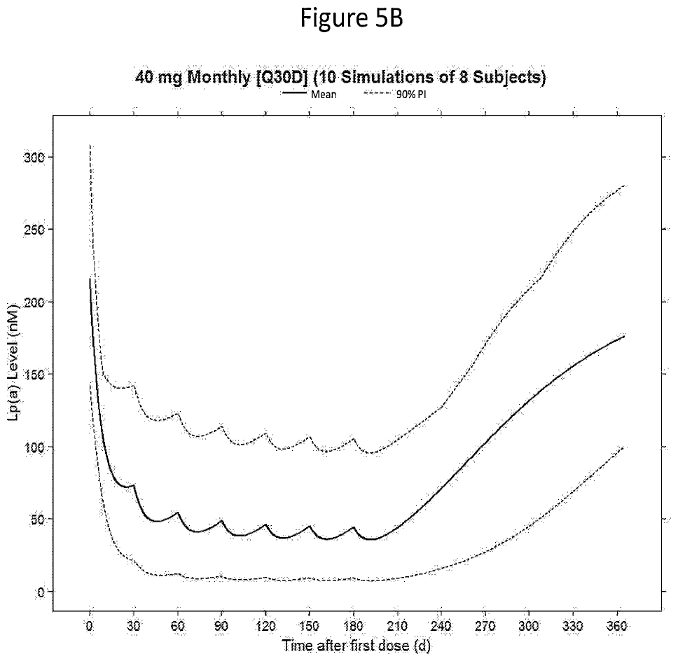

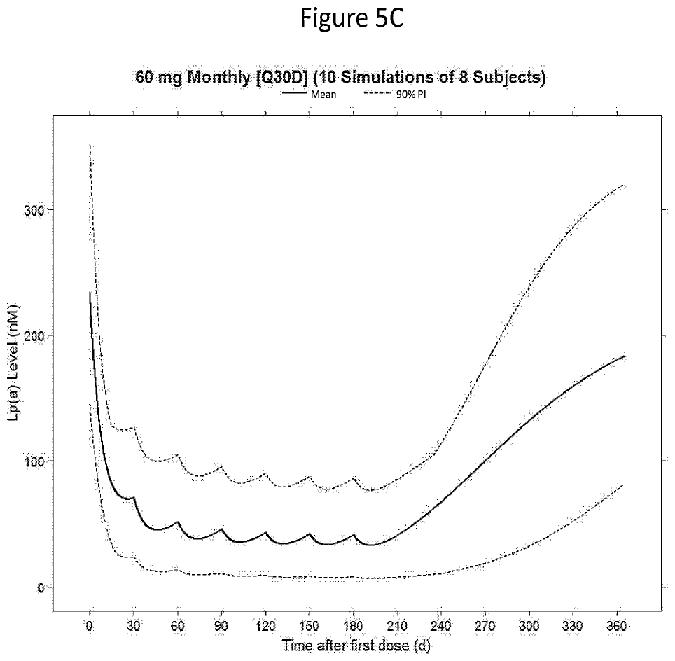

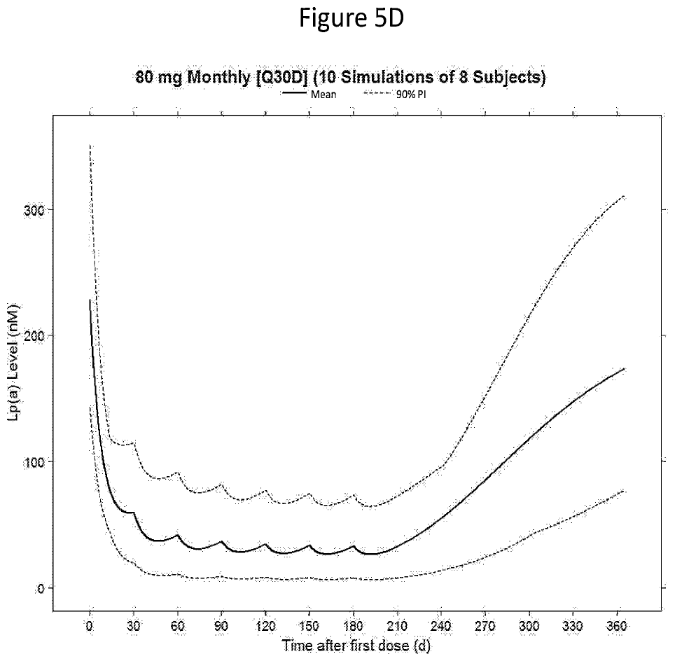

FIGS. 5A-D. Predicted Monthly Dosing Regimens. Charts are shown modeling the effect on Lp(a) by monthly administration of ISIS 681257 at doses of 20 mg (FIG. 5A), 40 mg (FIG. 5B), 60 mg (FIG. 5C), and 80 mg (FIG. 5D). The dark middle line represents the predicted dose, while the uppermost and lowermost lines represent the 90% Confidence Interval.

### Example 4: A Randomized, Double-Blind, Placebo-Controlled, Dose-Ranging Phase 2 Study of ISIS 681257 Administered Subcutaneously to Patients with Hyperlipoproteinemia(a) and Established Cardiovascular Disease (CVD)

The study described herein is to evaluate the safety, including tolerability, of ISIS 681257 and to assess the efficacy of different doses and dosing regimens of ISIS 681257 for reduction of plasma Lp(a) levels in patients with hyperlipoproteinemia(a) and established cardiovascular disease (CVD). CVD is defined as documented coronary artery disease, stroke, or peripheral artery disease. Patients must also have Lp(a) plasma level of ≥60 mg/dL. ISIS 681257 may provide therapeutic benefits to patients that have hyperlipoproteinemia(a) and established CVD.

Patient doses may be either 10 mg or 20 mg of ISIS 681257 administered once per week via subcutaneous injection for 52 weeks. Additional patient doses may be either 20 mg, 40 mg, or 60 mg administered once every 4 weeks via subcutaneous injection for up to 13 administrations. The primary endpoint is the percent change in plasma Lp(a) from baseline at the primary analysis time point for ISIS 681257 treatment groups compared to placebo. The primary analysis time point is at Week 25 for patients who received every 4-week dosing and at Week 27 for patients who received weekly dosing. Secondary empoints may comprise the effect of ISIS 681257 as compared to placebo at the primary analysis time point on any one of the following:
Percent change from baseline in LDL-C;
Proportion of patients who achieve plasma Lp(a)≤50 mg/dL;
Proportion of patients who achieve plasma Lp(a)≤30 mg/dL;
Percent change from baseline in apoB;
Percent change from baseline in OxPL-apo(a); and/or
Percent change from baseline in OxPL-apoB.
   This study may reveal unexpectedly improved properties of ISIS 681257 when administered to human subjects with hyperlipoproteinemia(a) and established cardiovascular disease (CVD). Treatment with ISIS 681257 may produce reduction in Lp(a) in patients with hyperlipoproteinemia(a) and established cardiovascular disease (CVD).

### Example 5: ISIS 678354 Clinical Trial

The study described herein is to evaluate safety and tolerability of single and multiple doses (both weekly and every 4 weeks dosing regimens) of ISIS 678354 administered subcutaneously (SC) to healthy subjects with elevated triglycerides (TG). ISIS 678354 may provide therapeutic benefits to patients that have elevated triglycerides.

The study described herein has both a single ascending dose arm and a multiple ascending dose arm. In the single dose arm, patient doses may be either 20 mg, 40 mg, 80 mg, or 120 mg of ISIS 678354 administered via subcutaneous injection. In the multiple dose arm, patient doses may be either 20 mg, 40 mg, or 80 mg administered once every week via subcutaneous injection for up to 6 administrations. The pharmacodynamics of ISIS 678354 will then be measured for each patient to assess: Absolute and percent change from baseline in fasting TG, apoC-III, LDL-C, HDL-C, VLDL-C, TC, non-HDL-C, apolipoprotein A-1 (apoA-I), apoB, LDL:HDL ratio, TC:HDL ratio, and lipoprotein (a) (Lp(a)). As used herein, “LDL-C” means low-density lipoprotein cholesterol. As used herein, “HDL-C” means high-density lipoprotein cholesterol. As used herein, “VLDL-C” means very low-density lipoprotein cholesterol.

This study may reveal unexpectedly improved properties of ISIS 678354 when administered to human subjects with elevated triglycerides. Treatment with ISIS 678354 may produce reduction in triglycerides. Treatment with ISIS 678354 may produce reduction in LDL-C. Treatment with ISIS 678354 may produce reduction in VLDL-C. Treatment with ISIS 678354 may produce increase in HDL-C.

### Example 6: ISIS 703802 Clinical Trial

The study described herein is to evaluate the safety and tolerability of single and multiple doses of ISIS 703802 administered subcutaneously (SC) to healthy subjects with elevated triglycerides (TG) and subjects with familial hypercholesterolemia. ISIS 703802 may provide therapeutic benefits to patients that have elevated triglycerides and/or familial hypercholesterolemia.

The study described herein has both a single ascending dose arm and a multiple ascending dose arm. In the single dose arm, patient doses may be either 20 mg, 40 mg, 80 mg, or 120 mg of ISIS 703802 administered via subcutaneous injection. In the multiple dose arm, patient doses may be either 20 mg, 40 mg, 80 mg, or 120 mg administered once every week via subcutaneous injection for up to 6 administrations. The pharmacodynamics of ISIS 703802 will then be measured for each patient to assess plasma angiopoietin-like 3 (ANGPTL3), total cholesterol (TC), low density lipoprotein cholesterol (LDL-C), high density lipoprotein cholesterol (HDL-C), non-high density lipoprotein cholesterol (non-HDL-C), very low density lipoprotein cholesterol (VLDL-C), and TG.

This study may reveal unexpectedly improved properties of ISIS 703802 when administered to human subjects with elevated triglycerides. Treatment with ISIS 703802 may produce reduction in triglycerides. Treatment with ISIS 703802 may produce reduction in LDL-C. Treatment with ISIS 703802 may produce reduction or amelioration of one or more symptoms associated with familial hypercholesterolemia.

1.-163. (canceled)

164. A method of treating or preventing a disease or condition in a human comprising administering an oligomeric compound to the human, wherein the oligomeric compound comprises a modified oligonucleotide consisting of 12-22 linked nucleosides comprising a region having a gapmer motif and a conjugate group comprising a GalNAc cluster, wherein the GalNAc cluster comprises a cell-targeting moiety having the formula:

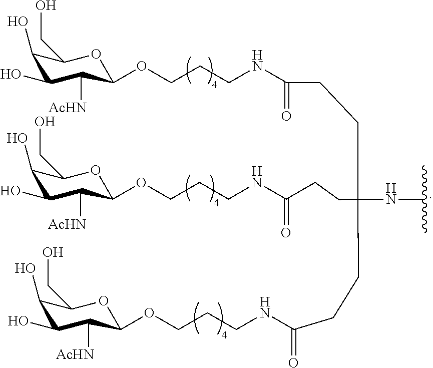

165. A method of treating or preventing a disease or condition in a human comprising administering an oligomeric compound to the human, wherein the oligomeric compound comprises a modified oligonucleotide consisting of 12-22 linked nucleosides comprising a region having a gapmer motif and a conjugate group comprising a GalNAc cluster, wherein the GalNAc cluster comprises a cell-targeting moiety having the formula:

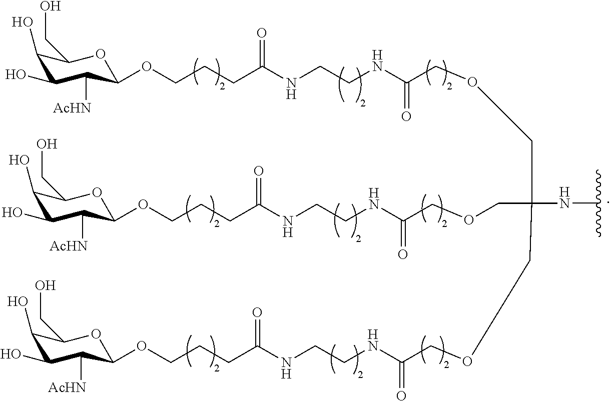

166. A method of treating or preventing a disease or condition in a human comprising administering an oligomeric compound to the human, wherein the oligomeric compound comprises a modified oligonucleotide consisting of 12-22 linked nucleosides comprising a region having a gapmer motif and a conjugate group comprising a GalNAc cluster, wherein the GalNAc cluster comprises a cell-targeting moiety having the formula:

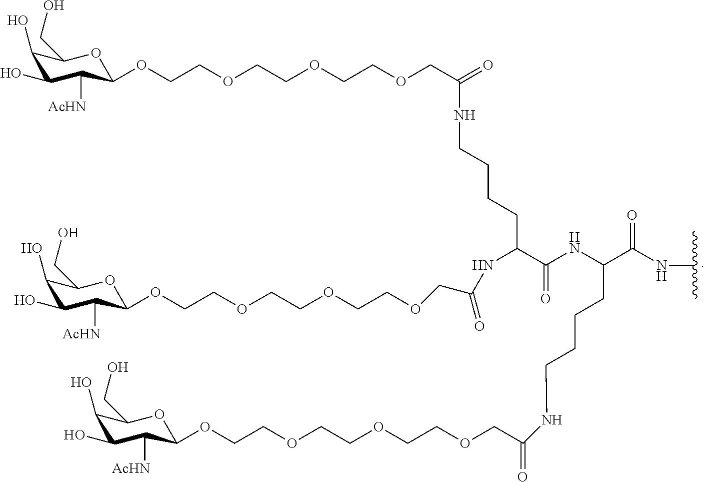

167. A method of treating or preventing a disease or condition in a human comprising administering an oligomeric compound to the human, wherein the oligomeric compound comprises a modified oligonucleotide consisting of 12-22 linked nucleosides comprising a region having a gapmer motif and a conjugate group comprising a GalNAc cluster, wherein the GalNAc cluster comprises a cell-targeting moiety having the formula:

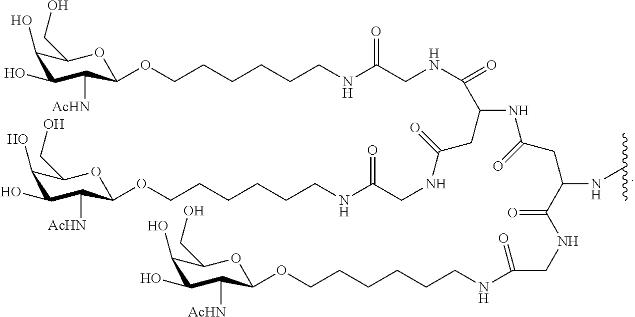

168. The method according to claim 164, wherein the gapmer motif is a sugar motif.

169. The method according to claim 164, wherein the modified oligonucleotide has a gapmer motif comprising:
a 5′-region consisting of 1-5 linked 5′-region nucleosides;
a central region consisting of 6-10 linked central region nucleosides; and
a 3′-region consisting of 1-5 linked 3′-region nucleosides; wherein
each of the 5′-region nucleosides and each of the 3′-region nucleosides comprises a modified sugar moiety and each of the central region nucleosides comprises an unmodified DNA sugar moiety.

170. The method according to claim 169, wherein at least one 5′-region nucleoside is a 2′-modified nucleoside.

171. The method according to claim 169, wherein each 5′-region nucleoside is a 2′-modified nucleoside.

172. The method according to claim 170, wherein the 2′-modified nucleoside is selected from 2′-F, 2′-OCH₃, and 2′-MOE.

173. The method according to claim 169, wherein at least one 3′-region nucleoside is a 2′-modified nucleoside.

174. The method according to claim 169, wherein each 3′-region nucleoside is a 2′-modified nucleoside.

175. The method according to claim 173, wherein the 2′-modified nucleoside is selected from 2′-F, 2′-OCH₃, and 2′-MOE.

176. The method according to claim 164, wherein at least one internucleoside linkage is a phosphorothioate internucleoside linkage.

177. The method according to claim 164, wherein the modified oligonucleotide comprises at least one unmodified phosphodiester internucleoside linkage.

178. The method according to claim 164, wherein each internucleoside linkage is either an unmodified phosphodiester internucleoside linkage or a phosphorothioate internucleoside linkage.

179. The method according to claim 164, wherein the modified oligonucleotide comprises at least one modified nucleobase.

180. The method according to claim 179, wherein the modified nucleobase is a 5-Me cytosine.

181. The method according to claim 164, wherein the modified oligonucleotide consists of 16-20 linked nucleosides.

182. The method according to claim 164, wherein the oligomeric compound is administered at a dose of not more than 120 mg during a dosing period, wherein the dosing period is one week, two weeks, three weeks, four weeks, one month, two months, or three months.


SEQUENCE LISTING (13 sequences)

SEQ ID NO: 1 - RNA, 6489 nt, Homo sapiens
  source 1..6489: mol_type = mRNA
aggtacctttggggctggctttctcaaggaagcccagctccctgtgattgagaatgaagtgtgcaatcgctatgactgggattgggacacactttctgggcactgctggccagtcccaaaatggaacataaggaagtggttcttctacttcttttatttctgaaatcagcagcacctgagcaaagccatgtggtccaggattgctaccatggtgatggacagagttatcgaggcacgtactccaccactgtcacaggaaggacctgccaagcttggtcatctatgacaccacatcaacataataggaccacagaaaactacccaaatgctggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaagcaccgactgagcaaaggcctggggtgcaggagtgctaccatggtaatggacagagttatcgaggcacatactccaccactgtcacaggaagaacctgccaagcttggtcatctatgacaccacactcgcatagtcggaccccagaatactacccaaatgctggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaagcaccgactgagcaaaggcctggggtgcaggagtgctaccatggtaatggacagagttatcgaggcacatactccaccactgtcacaggaagaacctgccaagcttggtcatctatgacaccacactcgcatagtcggaccccagaatactacccaaatgctggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaagcaccgactgagcagaggcctggggtgcaggagtgctaccacggtaatggacagagttatcgaggcacatactccaccactgtcactggaagaacctgccaagcttggtcatctatgacaccacactcgcatagtcggaccccagaatactacccaaatgctggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaagcaccgactgagcaaaggcctggggtgcaggagtgctaccatggtaatggacagagttatcgaggcacatactccaccactgtcacaggaagaacctgccaagcttggtcatctatgacaccacactcgcatagtcggaccccagaatactacccaaatgctggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaagcaccgactgagcaaaggcctggggtgcaggagtgctaccatggtaatggacagagttatcgaggcacatactccaccactgtcacaggaagaacctgccaagcttggtcatctatgacaccacactcgcatagtcggaccccagaatactacccaaatgctggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaagcaccgactgagcaaaggcctggggtgcaggagtgctaccatggtaatggacagagttatcgaggcacatactccaccactgtcacaggaagaacctgccaagcttggtcatctatgacaccacactcgcatagtcggaccccagaatactacccaaatgctggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaagcaccgactgagcagaggcctggggtgcaggagtgctaccacggtaatggacagagttatcgaggcacatactccaccactgtcactggaagaacctgccaagcttggtcatctatgacaccacactcgcatagtcggaccccagaatactacccaaatgctggcttgatcatgaactactgcaggaatccagatcctgtggcagccccttattgttatacgagggatcccagtgtcaggtgggagtactgcaacctgacacaatgctcagacgcagaagggactgccgtcgcgcctccaactattaccccgattccaagcctagaggctccttctgaacaagcaccaactgagcaaaggcctggggtgcaggagtgctaccacggaaatggacagagttatcaaggcacatacttcattactgtcacaggaagaacctgccaagcttggtcatctatgacaccacactcgcatagtcggaccccagcatactacccaaatgctggcttgatcaagaactactgccgaaatccagatcctgtggcagccccttggtgttatacaacagatcccagtgtcaggtgggagtactgcaacctgacacgatgctcagatgcagaatggactgccttcgtccctccgaatgttattctggctccaagcctagaggctttttttgaacaagcactgactgaggaaacccccggggtacaggactgctactaccattatggacagagttaccgaggcacatactccaccactgtcacaggaagaacttgccaagcttggtcatctatgacaccacaccagcatagtcggaccccagaaaactacccaaatgctggcctgaccaggaactactgcaggaatccagatgctgagattcgcccttggtgttacaccatggatcccagtgtcaggtgggagtactgcaacctgacacaatgcctggtgacagaatcaagtgtccttgcaactctcacggtggtcccagatccaagcacagaggcttcttctgaagaagcaccaacggagcaaagccccggggtccaggattgctaccatggtgatggacagagttatcgaggctcattctctaccactgtcacaggaaggacatgtcagtcttggtcctctatgacaccacactggcatcagaggacaacagaatattatccaaatggtggcctgaccaggaactactgcaggaatccagatgctgagattagtccttggtgttataccatggatcccaatgtcagatgggagtactgcaacctgacacaatgtccagtgacagaatcaagtgtccttgcgacgtccacggctgtttctgaacaagcaccaacggagcaaagccccacagtccaggactgctaccatggtgatggacagagttatcgaggctcattctccaccactgttacaggaaggacatgtcagtcttggtcctctatgacaccacactggcatcagagaaccacagaatactacccaaatggtggcctgaccaggaactactgcaggaatccagatgctgagattcgcccttggtgttataccatggatcccagtgtcagatgggagtactgcaacctgacgcaatgtccagtgatggaatcaactctcctcacaactcccacggtggtcccagttccaagcacagagcttccttctgaagaagcaccaactgaaaacagcactggggtccaggactgctaccgaggtgatggacagagttatcgaggcacactctccaccactatcacaggaagaacatgtcagtcttggtcgtctatgacaccacattggcatcggaggatcccattatactatccaaatgctggcctgaccaggaactactgcaggaatccagatgctgagattcgcccttggtgttacaccatggatcccagtgtcaggtgggagtactgcaacctgacacgatgtccagtgacagaatcgagtgtcctcacaactcccacagtggccccggttccaagcacagaggctccttctgaacaagcaccacctgagaaaagccctgtggtccaggattgctaccatggtgatggacggagttatcgaggcatatcctccaccactgtcacaggaaggacctgtcaatcttggtcatctatgataccacactggcatcagaggaccccagaaaactacccaaatgctggcctgaccgagaactactgcaggaatccagattctgggaaacaaccctggtgttacacaaccgatccgtgtgtgaggtgggagtactgcaatctgacacaatgctcagaaacagaatcaggtgtcctagagactcccactgttgttccagttccaagcatggaggctcattctgaagcagcaccaactgagcaaacccctgtggtccggcagtgctaccatggtaatggccagagttatcgaggcacattctccaccactgtcacaggaaggacatgtcaatcttggtcatccatgacaccacaccggcatcagaggaccccagaaaactacccaaatgatggcctgacaatgaactactgcaggaatccagatgccgatacaggcccttggtgttttaccatggaccccagcatcaggtgggagtactgcaacctgacgcgatgctcagacacagaagggactgtggtcgctcctccgactgtcatccaggttccaagcctagggcctccttctgaacaagactgtatgtttgggaatgggaaaggataccggggcaagaaggcaaccactgttactgggacgccatgccaggaatgggctgcccaggagccccatagacacagcacgttcattccagggacaaataaatgggcaggtctggaaaaaaattactgccgtaaccctgatggtgacatcaatggtccctggtgctacacaatgaatccaagaaaactttttgactactgtgatatccctctctgtgcatcctcttcatttgattgtgggaagcctcaagtggagccgaagaaatgtcctggaagcattgtaggggggtgtgtggcccacccacattcctggccctggcaagtcagtctcagaacaaggtttggaaagcacttctgtggaggcaccttaatatccccagagtgggtgctgactgctgctcactgcttgaagaagtcctcaaggccttcatcctacaaggtcatcctgggtgcacaccaagaagtgaacctcgaatctcatgttcaggaaatagaagtgtctaggctgttcttggagcccacacaagcagatattgccttgctaaagctaagcaggcctgccgtcatcactgacaaagtaatgccagcttgtctgccatccccagactacatggtcaccgccaggactgaatgttacatcactggctggggagaaacccaaggtacctttgggactggccttctcaaggaagcccagctccttgttattgagaatgaagtgtgcaatcactataagtatatttgtgctgagcatttggccagaggcactgacagttgccagggtgacagtggagggcctctggtttgcttcgagaaggacaaatacattttacaaggagtcacttcttggggtcttggctgtgcacgccccaataagcctggtgtctatgctcgtgtttcaaggtttgttacttggattgagggaatgatgagaaataattaattggacgggagacagagtgaagcatcaacctacttagaagctgaaacgtgggtaaggatttagcatgctggaaataatagacagcaatcaaacgaagacactgttcccagctaccagctatgccaaaccttggcatttttggtatttttgtgtataagcttttaaggtctgactgacaaattctgtattaaggtgtcatagctatgacatttgttaaaaataaactctgcacttattttgatttga

SEQ ID NO: 2 - DNA, 150001 bp, Homo sapiens
  source 1..150001: mol_type = genomic DNA
atctttcagcctctatattattttattgtgatttttaatttccttgaattggattttgccattgtgctaaatcttgatgatcttcatttgtatccgtagtctgaattatatttctgtcatttgagttagctcagtcttgttaagaacccttgttggaaaactggtgcagttgtttggaggacatatgaccttctggccatttgatttattggagttcttacgttggttctttctcatgtctctgtgtgggtgtttctttaactgcagtgtagattgagtacagccaatagacttcttctttggaggttttcacagggccaaggccttgtacagggtctttatttgtagctgacttcttgtctttggtttcatagtggggcatgttagcaaaatagttttgctgttgaagttttggggtgtgatccattttttattttaatgattgtgtatttcctttatacctaaaacaagcagaaaaccagtaaaggtctttgagtctctgaattcataactccagcattcatattgcttcctcaggtaagtggggttttcacccagcccttaagggtgttagattattttttatgtgaaattagccagattgtatttctaaacatgatgtaaaacaataatgacaaaagttataataaactagccttcttaccaaatccacatgtctaatgtgtgtgggagggtgttaggcaggggacctgcagctaagggagaggcagacaggccccatggccccaaatctaggatagtatttggtattggttgatgggtgagagaaagagagggaacatctgtgcaggatgtggtatcagcacctggactacatcttagggattccttcttcatttttcagtatgccctgacaataattatatctatcagacttacccccttgaccactggaacactaagactgttttgggatctctgcctgactttctcagaggtgctggtgaggacattatgagtctggaacctagaaaagcgttctgactctgctgactttctcagaggtgctggtgaggacattatgagtctggagccctagaaaagcgttctgactctgccactagccagacagacctggactaggcacgttaactctttgtatgacttgactccaacccctcatttgtaaaaccagcattttcaagtggtgttttccacatcagccttttgcataagctgtcatttgaagaaaggtttttgtttgtttgttttttgtttaacaaaaaggttaaaaaccactggtctagataattgcaaagtttgctttcctttttctgtgctttttctactatttttaaaatgtcatcctccttggtttcttgatccccctttctgcactcctgagtctgggaacactgaggccaactaaaaggaaacttggcaaaagaggaacacctttgggtgtgccaggctgctcccagtgttttgcacttataaaaatttaaatgctgcaaacctctaagacttagatattattgttcctattttacaagtgaggaacctgaggctcagagaaggtgcaggatggcacagggagacctgaattggaaccctggttcccacttactggctgtcgggacttagaaaagtcatgaactctcattgattgttttcttatatgaaatgggggctgcagggttgtcgggggagaaacaataagaatgtgcatcaagtgtcgagcacgtgctacgcactccatcatggcagctcctactaatatacagaatagagttgtatctaacatgactctttcttgcaagtgacagaaaatccaacttaagatggattaagcaaaaaaggggaattcttgttgagctgaaaagtctttaggctcacatgatggccccagggcccaggccctgtccagccatgcagtaggcatcatccttgggcacaaaggtgagattcttgtggtggcagatgctgtggcagctcttgctttgccaggaaagactgaggaaggccactgtccccattaagtgaacaatagttggccaggtctgagaggttgaacttgggtcacaggcctgtccctgaacccatcactgattggctccaacctgcatcagctattacatgctagaggtggaggcaggaccccactcatacccagaagggcaaagggtggatccctcaacaggattatgggatgtagggtgatagactgctgggcagccagaaagcaaacagatcctctccaatacctcaactgatgaaagcaccaagctaaaatcataaggatctgggtgtgaattctggctctaccatcttccatgtgacattgggcagttatttaatctcttttagccttggctttcttacctgtactaacatataaggtgattgtgatgagcatcatcatcgtcaacatcatcatcaccatccacattgccaccaccactcccattatcatcttcatcaacatcatcaccaccgccaccatcaccattatcattaccaccaccgctatcactattatcatcaccctcaacatcatcaccatcatcactatcatcaccaccaccatcatcgttactaccactaccaccaccatcatcaccacagccaccaccaccatcaccatcattactactcagcaccaccatcatcattccaccaccatcaccatcattccaccatcaccattatcattaccaccaccactgtcactattatcatcaccctcaacatcatcaccaccaccatcatcattactaccaccaccaccaccatcaccatcatcatcattctaccaccatcaccattatcatcaccatcaccatcaccaccgctatcatcatgataatcattatcattaccaccaccattagcattatcattaccaccaccatcactatcactatcaccatcaccacgaccactaccaccatcaccaacaccatcattactacccaccaccaccatcatcatcattccaccaccatcacaattattaccaccaccaccatcaccaccaccaccaccatcactatcatcatcagtagacatcatataaccagtttgtagctggcccagagcctacttgctgtttcttctgccccacaaccatccacacatttctaaccaccatcccccactaggcttctgcctcgcctggtctcacctgcaggtccactgagaaaatgattctcagaacactaactagaccatgaggtgccacaaaacataactcaggcctgttcatcaattttctacatgtcaataatgacatcaggtcaattggcgttctcagcctctgagagggaggtcaaagttttcctgctctccccttcatgtttccaggtgttccctgacttggatcaaatgcagagtttggaggtgttgaggccaaggggattttccaggtcagtcgtcatccacaatcaatggactgatcctgccgctggacttaccctgctgccctctccccaaggccccatcagggagggcttcaatcctcttgtcacctgtggcctacctgccctcagagatgacatctctatgtcggccactggatggcagcacctactcgcagaccacatcaactttcctggcaacttgcggtaggttttcaccattatcaggatgtttgccttgctcaaatagcagattctagagaacggtgctccctcacacaactatgtagtccaggtgatgcaccctctgcccgatgcttggtagtcagaaacttccatcatgcagctctgcccagattgagctgagctggcctctggagtgaggtgctgggacaaacatcttccatgctgctcatgtcaactccagatgcagtcaggtttctgaaccaaagtcaatgatctaagtgcagtcaaaggctctgggggaagaaagagagagtgcctcatctcttgcctgtgccatgctcgcaaagcaaggatttttgcaaaattctaatgaaagctgggcttgcaaaattagaaaactggattatttgtgagaacactgaaacatccctgggtgtgtccatctggaaaaacagcatttcctctggcaattttgcaaccgttctatttgaatttggcaaagaaaataaagcagtttttcacaaaagaataaacacaaccaggagaatcttcactctcccaaattgtcaaagaagtataaattagaaaatgaatcaggacaatttcaacctgttagattagctaatatttaaaaattgaacactcatacaagtgtggtgaagtgattgttttctagtgacattttacactgtcataaccttctagaaaataaattggcagtgttattgggagacagaaatatgtctatataatttatgggaacttaggctcagaaaatattaaggaataagaatgaactttatgaacaaagatgtggagggttggaagcaagaggggggccaacgcgcacggggaggaagcatttgggcagtgactccgcagacccaggctcaggttgaactagacaacctccttacacctcagtttccttaactgtagagcaggagtgatggaactgcctgtttcataggactgttgtgaggatgaagtgagatacaccacattataagcttgtgcctggaaaggataatgcttagtaaatgatgactattcttttttattgcaataaaatgtacacagcgtaagagttactattttaaccatttttgcagggtaccaccaagtggcatttagtacattcacagtggtgtgcaaccatcatcatatttccagaatattttcctcatccccaaaggaaacctcatgctcattaatcagtagctctcctttaaaatattagttatgaagatcatagcactatacaaaactcattatgtaatgttgagtgaaaaaatcagggtgtgaaattttgtgatatgatgtaattagtgaaagaagcatacaaaaagtctgaaaatataaaaacaatagcaattgcatttctcagactctacatttaaacattattctttatggttttaaaagcaaagaaaaaggtaaagaaacaacaaccaaccgcaaagcaccatgacaaagctcagattgttaaatccaggtttttggaacatagactcttatatgacgtttacactctccagggttcagagagtctggcagcattgggagctgccttgtgttctacagcctcacggacagacaggaggtccatcaccactgctctgttcttctggagtttccttgtgaacatgttgtggacgtagttaccatttctttcatctttttaaacacaggtacctttggggctggctttctcaaggaagcccagctccctgtgattgagaatgaagtgtgcaatcgctatgagtttctgaatggaagagtcaaatccactgagctctgtgctgggcatttggctggaggcattgacagttgcaaggtaagaaaagatcaagagaccaaagttagtcttgtgctctcctgtctcagtctcagtcccttagacttgagtcccaaagtagcgaattcaagtaggatttaatcaatggaagaccccagtctaagtgttgctcagaaactccctagatctgtcccaaatgtatattcagatcatccaaggggacttcttggggcttgagttccagatcagcagcaagggagccataagtgccataactacctcagaccactcaccctcctggggtgtcccggtggccagggactaaagtggtgatttttctggtagggaaggaggtagagggtacaggacagagactaactgcacacaatatctgagactggagctcagatattgctgatgatcagagttggcgtgtctccccaattgatttacaactggggcttggatactgttttaaacgggaggagcctcctaaccatcttgacacaaccactgacgtgactacactagagatagactctttccacttaattctaccactcttgctttacttcatgagaacgaaaatgtaagattgcaccatgaattcatttgcggaaagattgatactatgcttttattttattttattttattttattttattttattttattttattgagactctcaccccggttgaagtgcactgacgtgattttggctcactgcaacttccacctcctgggttcaagtgaatactccagcctccctagtagctgggattacaggtgcccaccaccacgcctggctaatttttgtatttttagtagagatggggtttcaccacattggcctggctggtctcaaactcctgaccttgtgatccacctgtcttggcctcccaaagtgctgggattacagagttgagccaccgcactcgaccctatgttttatttttaaaaatatttatttatttatttaagccacaactactagaataggaaggattgatattttattaattttatttggtatttattatttttttttctttcctgagacattcttgctctgtcacccaggctggagtgcagtggcacattcttggctcactgcaacctccatctcctgtgttcaagcaattctagtgcctcagcctacttagtagctgggatgactggcatgtgcctccacacccagctaatttttgtattttttgtagagacagggttttggcatgttgcccaggcttgtctcaaactcctggcctcaggtgatccatctgccgtggcctcccaaaatgctgggattataggcatgagccaccaccccctcctggaaggattgatatcttataacataatttataattacagaaaacatgtgagttcactaggaataaataaattttgaagataataaaagattttcacttatgttgtcatttcggcacagtttggtataggatgtggagatgttaacatttatacctagcttgctcgtaaactaagacctgaaagggttgtgtctatcagctgcacccctgggtagcgacacaacctcgggaaggcctcagccccctcctcgtacagcactgcctgttggaaagcttgagggaggctatggatgtgcagcacttggcagagggtctggtcatggaagttaccagcaaatatgagctacttttatgattttattttatccaaaagaaagagaatgaaagaagaggggaggaaacaagactaatcaggaaagatgaaggtctaggggtgagggaaggagtaaggagacataaaggcaatgtggagcagctgaggggggaaatggctttcaccacttcccagcatctattgacattgcactctcaaatattttataagactctatattcaaggtaatgtttgaaccctgctgagccagtggcatgggtctctgagagaatcattaacttaatttgactatctggtttgtgggtgcgtttactctcatgtaagtcaacaatgtcctgggattgggacacactttctgggcactgctggccagtcccaaaatggaacataaggaagtggttcttctacttcttttatttctgaaatcaggtaagacatagtttttttaaattataagaattattttttctcccacaatgtagtaaaaatacatatgccatggctttatgtgcaattcatttaatttttgattcatgaaattcccagttcaaaatcttgtatatgattgaaaaattcttaaaaaaataagtttaatttccccgtgaagactgtcacggtgctggaatgaatgggcagaaaaaataatggttgatttttctaatctaaaagagtgtgcctacatgatggccagtctggctgaaaaataaatagccattgtagctaactatgcaaaggatggctaagctcttcgcttggttctcagtttcattaatttatatcatctctgttcaggtgccatgctcccctcactagcaagttgaaacaatgaaataactctttgaatatgtttggttccttgacctgttcatggagtgggactcagcatttctctctttgttatggcctgagtaaggctttccatcggtatacatttgcttcttatccctggagaaattatacacatccatttgccagatgatatacgcatataatgattcaacaaatactcagggtatttgttgagtgggttaggtccccacatttttatacatacatacacacatacacaccgtgtgtgattgtgaatgtaagtgtgtgtcctttacaaatactagcttatttagctcatggtataggtagggtagcatagtcatccccattttataaacaaagaaatctagacttaggaaaatcatgttatttgtctcgtgaccaaattcccaaatcaaggaaataaagaaacctggatttaagccagatttccaagaaaaaatctagggctcttctcactttttcatctttgttccaacatttgaaaaaataaatctaaacacattccaatgtaactgaagagcaggttaattgtttgccacttgcagaatccaattaagaagagagaagtctggtataaagaaagtgatttgcttccaaagctagcttaggggaagaaatgcagcagtcctgccgtactacttcactttaggagcagaaagtggcacttttaaaaggcaacagaggaggcgagcaaggattcaggggtccatgctagcttgggcaccttatccaccaggtagttgagcagttgcctgctggtgcctttgtgagcagggtgttgtcccttgaggcaaatctctggagggtgagagttttgtagtgggcatgctttggtttataaatcacctgtgaactcaggagttccatcttgaagcacatacatagttagatgaacttgccctgcagggagagtctgatgaaagggaggtagatgcttgcaatttaatctataaattaccagataaaattttacaagttgactttaaagtcaaacacatttgaatttagtggaagccattcaagaaaatatcaaagaaaatacagagcaggagaagattaagcaaagagttttttggggaaattggtgtctatgtctgtgtgtgtagggagtgcaggggatatgaatattctatttcagcccatggaaactaggatgtagatcactgtgaacttattcagcaggctacacccaaaggctagaacaaacttctctgccacaggattaacatatgttttaatcgacctggggggcacattctctgataagctcttttggaaagccaggctttctgtggacgtgttatctttccaatgtgtgctggaatgcccggggagaggaaaaagtttcttttacagccatgctcagtgagaagcggagaaacatcttctattcacaaattgctaagtcttttacacatgcaaatatgcatacacattcacacaccacagtgaggaagaaattctcacaccattaataaaatacatttacttcagtagcaatatacatctacattttgcctataatataaaagtatttttcctattaaaagatttgtttaatgtttcttcaccaacaaataaaccctattaaatccccattgccatatgagccctggaggtgaatcagagaaacaaaaggattgtggaaaaatcatcaggttaaaaaaagaaaaattgattctgttttgggatatttcctagcaacatgagctggggaggggatctcagcagtgatgctctatgaagcataataaaatgacacagttacaggtaacttagttaaagggggaaataaatggaagtttcctctttttgaatatcaattgtagcctgctctgctacatttcaaaaacactcttcaaaatgtttaactgaactcactgtaggaagcaccttattaatttattgtgtgttttgaagtcacactgtgagctatagaatttacccaagcacaactcttcctggaaaagagagttcaaatgagaaacagtgcggggtgaagacatggatatgggcctaaaatatctatttctcaatgatattttgatatatctatcaagtgctttttagtggattaggttcagaatgcatcagccaatgcctgttcaataatccagttttccagcatagagcatattaaattgaggaaggacaaagtcacagaggtggggagcaggtggactgtggccaaggactttgcatgaaacagtgagcgtgcatcctcctccttgccctgccctcatggtctgtgtactctcaggaggtcaggacaggcctttctgagaatgagaatctgttcatctgcctttctactggatacttgtcatcggcatacaaacacatgttctctgcagtgtgtcatctttcagaacctcccctgaccctgtattccctagaagtctcgctgctttcagagccaggcttctctcctgctgccacccccactgctcttctagtcactctttaacccactccatctgcatgtggcccccaccacacccctcaaagtggtcaaggttgtcctgttgcttaattccatggaagcttggctatcttcattttattagcctcttttggcctctcaccctgtgaaaatcactacattttgtgccagagatggagctggcatctccaggcttggaagagggctgctgaagctcagccaggtgtcctaaggagcctcaggacaggggatgctcagtagccttgcaatgggaacacagctgagccccacttggccaccctttgccacaaccaggcagaaagcagcttttgaacagatttgttgcctcagatttgatctcaaagaaaaatcgtgggcagtattggtcccaggttctgcttttttacaatttcctctgaaatctggatgcctatcaacaccttggaaaaactgaattctccccaactaatagtggtgtgtcactgtagtaagcctagtacaaaaatggccttctttgtggaggagcttcatatcctccattttttttttgcttaatttttgcccaagatgagaacataatttagttcactttttatttattcccaacatcatccatgcaccaacatttttgtaactaaaggagggaccattcagaagatgcttatcaactgtcaaagtgacagtgttacaaccaatgcacatattgtaagaaatcaaacaatggcctccaaggttcatttctacacagggattagcagatcaacatcaatcttggcaacacagttgccactgatggtgtcttattttttttatcatgacatggcaatcaagagcaaacatgatttattcttatttaagattttatggttagactaggcagatagctagatatgagcaggaggtggaagcccctgagagaatggaggtctggagaatctgaaaccccagagattacccaagtcctgcatgctagacatgagtggaggagggggaatacctaggtagaaaagaatgccccttaagatgcccagcagtcgctcactgtgcagttaacttttcagaatgctgctagatacatgctgatagggagggaagagggcaaaggagaaattcctaagagatacacggttgcagttagtatacatctgagtgctatacaaccttctttgggtggtggcaagaagcaatgcagccattacgtagaattcatatcaaacacctgtatcacaggtgttaaagaaacaagaaacattgtacttcttgtattcttaataatgatttgcaatattgtctttagtatcactgcaaacctctataaatatgatttttaaaaagtatttctttaggttggaattacttctacgcattgacttatcttcctgggtttcattagccgtacccgttgtactttcttccttaccactgtttatctcaaactcttgagattaaagtatgggctcaggagggagcgaggagcttcaggactctcacggacctccagcacagtgtagctgccttatggaaaagtggccacactgttttctgcactggtccctgcccctactattcctcactgggcagagcacagccaccctggccctgcctgaacattttagtcagtgttggctctgtgcttctctggggaggaaatccaagagacaacccacagcccctctgccatttcagctgcagcagtaccaccgttaatgcccttgggcttgagaaagaagggacctggccacttccctgacacctccagcacacagcagggaaagaattccagtttctctttcttgtgagctttcacctgctactcttcaccaggcaaggctcctggcttgggcccacagtgcaggcacctcgaactcagttgaacatttccactggctgcactctgtgtttttgtggggtgaagctcccagaggtgactgaaagtccttctgccactaacactgcagtcatactgcccttgctgtacttggactagggaaggaaaaaagatcctgagtgctttactcacaccccagtgtgccccagccaccctatggaaaagaggccagtgtgtcatccctgcaagcaccctgaggcccctgcccctgctgcccccaagctgtagagccagaatataaagctggcagaaaaatgtaaaaaggctagactggcttagcctcccagcctacatctttctcctgtgctggatccttcctgctcttgaacatcggactccaagttcttcagctgtgggacttggactgtcttccttgctcctcagattgcaggtggcctattatgggaccttgtaatcttgtgagttaataccacttaataagctcccctttgtgtgagtatatctatatctatagatagatataggtatactcactatatatacacatatatacatatactctctctctctctctctcatatatatatatatataatctcctattagttctgtccctctagagaaccccgactaatacagattttcataccagaagtggttcttgaggaacagaatattaaggatggaattctttcattggttttgggacttctggtgttggctgattaatatgattagaccaaaaaatgctaaggactctacttctaatagtatggagaacactgatagtacttggcctgaattgtttagagagttatgcaaaataaatgcatttgacactactgattcatcacttatgagaggcaaggagtttagtgactctatacataatacctttgactatatgtggagaaccaaggaacataatgaagttggttgattgctcctaagttctctggagaaagagatgaaagaaaatgatgatctcaggggatctgtctcccaccttcagaagcagatactgagccacaaatctgctaagattgccctgaatgagagttttaactcctgtagagaaagagttgaaattgtgaaaaaacagagacaagctgttatcatgcgagtagctgatctgcaacaagaggtgcatgcacagccttgccaggtgtttactgttaaagtgagggcattgactggaaaaaaatgggaccctggaacttggagtggggatgtgtgggagaaccctgatgaagctgaggacactgagtttgtgaactctgatgaaacttttttgccagaagaaacagtttccccatccccagtagtggtaacatcccctccctgacccgtgctgccattagcctttccacctttgtctgaggatgtaaaccctgcactgcttgaggcaacagtgatggccttccctgaggcagctgccaggcaagataatgttgattctcctcaagaggcacccctaatgcccctgaatgcttctagacctataactaggctaaattccttgcgggccccagaggtgaggttcagagtgtgacccatgaggaggtgcattatactctaaaagaactgcttaagctttctaatttatattggcagaaatctggagaacaggcatgggaatggatattaagggtaagggataatggtggaagggacatagagttggatcaagctgaatttattggtttggccctactaagtagggattctgcatttaatgttgcagctcggggacttagaaaaggttctgatagggccgggagcagtggctcacgcctgtaatcccagcaccttgggaggcgggggcgggcagatcacgagatcaggagattgagacaattctggctaaaatggtgaaaccccatctctgctaaaaatacaaaaattagctgggcatggtgatgcgtaactgtaatctcatctacttgggaggctgaggcaagagaactgcttgaacctgtgaggcagagattgcagtgagccaagatcgccccactgcattccagcctggtaacagagcaagactccatttccaaaaaaaaaaaaaaaaaagttataatagtttatttgcttggttagctgaaatatggattaaaagatggtccaatgttagtgagctggaaatgccttggtttaatgtagaggaagtgatccaaaggcttagggagattaggatggtggagtggattagtcactttagacctactcatcccagctgggagggtccagaagatacacccttggccgaagctttgtgaaatagatttgtgagagcagcacctgtatttttgaagagcccgtaattgctcttctctgtatgtcagatctaacagtaggaaccacagtcactcaactacaaaatttaaatacaatgggaataattggatcctgaggtggcaggggccaagtgttggcactgaaccatcaaaggcaaggtgggcataactaccataatagacagcagaggcaaagcagccatcagaatagtctgactcatgtagagctctggcattggctaattaatcatggtgttcctagaagtgaaattgatgggaaacctactgtattcctacttgatttatataaacaaaaaactgccaggtagaatggactaaagactaatctgaattataaaaacagagaatcatgggccctcaatcaatttccagactcgaacctgttacagttccagaacccactgaatgaaggggaggctggatccccttgaggaaggacaccactaggctactgacaacttatgctgttactctttctcccatccttccctaaggagacctctggccttttaccagggtaactgtgtgtactggagaaagggaagtaatgagacatttcagaaagtactggacactggctctgagctgacgttgattccagggtacccaaaacgttattgtggttccccagttaaagtaggggcttatggaggttaggtaattaatggagttttagctcatttctgacttacagtggttccagtgggtccctggacttatcctctggtcattttcccagtgccaaaatgcataatttgtatagacatacttattagctggcagaaatgccacattggctccctgactggtaggatgagggctattatggtgggaaaggccaaacagaagccattagagctgtctctacctagaaaaataaaaaaatcaaaaacaatatcccatccctggagggactgaagtgattagtgtcaccatcaaggacttgaaagacgcaggggtggtgattcccaccacatccctgttcaactctcccatttgacctgtgcagaggacagatggatcttggaaaatgatggtggattattttaagcttaaccaagtggtgactccaattgcagctgctctaccagttgtggttttgttgcttgagcaaattaacacatctcctggtgcctggtatgcagccattggcttggcaagtggctttttctccattcctgtccataagacccaccagaagcaatttgccttcagctgacaaggccagcattatacctttaccaccctacctcaggggtgtatcaactctccagctttgtgtcataatcttatttggagagaccttgctcgcttttcacttccacgagatataacactggtccattacattcatgacattatgatgattggatacagtgagcaagaagtagcaaacacactgaacttattggtgagacatttgtatgccagaggatgggaaataaatccagctaaaatttagggactttctacctcggtaaaatttctagggttccagtggcatgagacctatggagatattccttctaaggtgaagcataacttgctgcgtttggcccctcttacaaccaagaaagaggcacaatgcctggtgggcctatttggattttggaggcaacacattcctcgtttgggtgtgttactctggcccatttatcgagtgacctgaaaggctgccagatttaagtgcagtctagaacaaaagaaggctctgaaacaggtccaggctgctgtgaaagctgctctgccatttgggccacatgaccccgcagatccaatggtgcttgaggtgtcagtggcagatagggatgctgtttggagcctttggcaggcccccataggtgaatcacagtggagacctctaggattttggagcaaggccctgccacttctgcagataactactctccttttgagagacagctattggtctgttattgggctttggtggtaactgaacgtttgactgtgggtcataaagtcaccatgctacctgaacctgcctatcatgaactggttgctttctgacccatctagccatgaagtgggtcagcacagcggcatttcatcatcaaattgaagtggtgtgtatgtgatcgggcttgagcaggtcctgaaggcacaagtaagttacataaggaagtggctcaaatgcccatgttctccactcatgccaccctgccttccctcccccagcctgcaccaatggcctcatggggagttccctatgatcagttgacagaggaagggaagactaaggactggttcatagatggttctgcacgatatgcaggcaccacccgaaagtggacagctgcagcactatatccactttctaaatgcatgtgtacacttgtgctaagaaaatatctttattttatttcctttatttttcctttatcatgtgaccttagatttatggacttcacatcagcatttaagcatttaagtgttgttcatatcagcatttaaatattgttaaccttatgtaataacttttggtttggggattggtgcgtttctggttgtatgaggatagttgtattatattaggcataattatgaccttattattgtctttatttgaagattatgtatgatttcaggatgtgtgtatgggttcaagttgacaaggagttggacttgtgatggttaatactgtcaacttgattggattgaaagatgcaaagtattaatctcggttatgtctgtgagggtgtggcaaaaggagattaacatttgagtcagtgggctgggaaggcagacccacccttaatctgggtacacaccatctaatcaagttccagtgtggccagattgtaaagcagggagaaaaatgtgaaaagactagactgaattagcttcccagcctacatctttctcctgtgccaaatgcttcctgctcttgaacatcggactccaagttcttcagcgttgggagttggactggctttcttgctcctcagcttgcagagggcctgttgtggaaccttgtgatccgctgagttaatactacttaataagatcccctttatatacatataatatattatattatatataatatatataatatatattatatataatatatataatatattatatattatatataatatatattatatattatatataatatatattatatataatatatattatatattatatattatatataatatatattatatataatatatataaaatatatatatatcctattagttctgtccctctagagaaccctgactaatacaatttatgtcattaatctcatttattgatttgtatacattgaaccaaccttatatcccaggaataaaacctacttgattgtggtggattagctttttgatgtactcttggattcaattgctggtattttattgagaatttttgcatctgtgttcatcaaggatattggcttgaagttttctttttttgttgttccatatcagaatgatgacgacctcatagaatgagttagtctgtcctcttttatcttttggaattgtttcaggaggcttgatatcagctcttctttatatgactggtatactttggctaggaatctctctggtccaggggtttttctggtgtaggtttttaattactgattcaacttcagaactcattactcattattgagttctaaaactcactttcatgtactcttcaaaagactgtcttcttctgttgttgagcggggtgttctctcaaggtcgtttaggtgaaggtggttgctggtgttcttctgtatccttactgcttgtctttctctttttttattgactactgaggattaatggtgatgtgtccaactttaactctagattagtctatttctcttttagattgtaactctgttttatatattttgaagctctgttgttaggcatgtgtatttggattgttaggtcttcttgatgatgacctttatcattatgtaatgtttcttcttatctctggaagtattcgttgttctgaagtctatttgtgctgatatgaatacagccttcacagctctattttcactagtatttgtatatctttttctcagcttttaaattgagatgttcagaccatttgcattaaagtagttgttaataggattaaatttaaatctaccattaagttggttatttctctttgtcccatttaaactttgttccttttttcatatttttctgccttcatttatattgagtttatctccacgacttacttattaaattaatttttaatggttttagtattttccacaatgtttataatatatactttgattttttcacattccaccttcaaatgacagaattatactggatatatagaaatcttacatcattgcacttctccttcctccctctcaaaatgttgtgctattgctctttgtaatagaggcttacttctattatgttatagctctcataatacattgacactatttttaccctgaataatcagttgttttttaaagtgattatgactacaaatattttgaataatttctttattttaccatttctggtgctccttatcttttacagtagatcccaatttccatctggagtcacattctttctgtgaaaaacaacctttagcatttcttatagcacgggactgctgttgctgttgtctttcagcttttctttgtctgaagaagtctttattttgccttcagtttttaaaagtgattttgctgagtatagatactgggttgagagtttcattccttgtatcattttaacaatgatgttccattatattccgttttgaatagtttctgactagaaatctgatctttgtttctttgtattcaatagttcctttttctctgactgcctttaagatattctcatctttgtttttcaacagtttgactataatttgtttattattaactttttgtatttattctgcttgaggtttcctgagctccttggatttgcagattgttgatttttattgtttttgtaaaattcatagccattatctattctactgttttgttttttttttcacttctctctctctgtattcttctttttggactgtaagtattcaaatgttagatcattcatattgcttcataaaccttatatgcttcttctgcttttttttttttgtcaggaactctttttttgtatctgtgttggtttggataagttctagtagactatgttcaagtttatggattattttgttagttgtgtctaattgactcctcagtgcattcagagaattcttcatctctgatattataaatctcttcctagcattttcatgttactcttttctatagtttccatctctttgctgaaattctccccctatccatggatattgtccacctttaccacaagattctttaacatattaacataggtatcatacaaacccaaactgatagtttccagatggtgtcttttctgagtctgtctgtcttgattgctttattatttaacagtgacttatcttccctcttcagcttttggtgtgtcttgtaattgtttaatcaaacactgggtatcataaatggaggaacagtagagattgcagtaaatattatttatgctttgaaatgggcacccatcttctgttgaaaatatgttttgtggtcaattgagtcaacctagtaactggttgaactgaatttggcatttgtgcttgttgcttttatcttaaatgcaccacaggtttaaattcctccagtgatgggttgctgctatcttttgcttagagtggggcctggggtgtggaagaattttctcagtgttcctatctattattagattttagcagtcactgcatgcctgcactacagaggggatatcttcatacacataatctaaccccattgaaactgctgtttcttcttaatgaatgctcaatctttggtggaaataaacaaatgctgtatctcctggagccacttcagtcttagtcaggttctgcagggctttgaagggaatgcattctcagtattcttgtgccttatttggatggaacttgaacctgtggtgggtttggagagaaagagtagcagacgtctgctatgttgcaatgcaggatgctgggcacaagaaaatttccagtctctcctccaaggaaataagatttgatcatctacctatccctgagaagtgaagggctttgcctgcggtgctagatgcaaaaccatttttctccccccattgcccagaaacttaaggctttggcttttctgagcagtggtctagggaattgtgcaaggttttcatatttgaccctgacagcccatcaccacctacagcttgcagtgccaaatgtatctccctctgatctctcctgtcctgtggtcctcatgaacattaagaagagatttctaaaaaagagcttgcacatgagcatagtttctggtgagaagaattctgatatgttaacttcctctaaacttttaaataaaatatttctaagaattaaataaagttctagaatgatatgaatctattcctttggttttttgcacgtctgtctgcctgctaatcaagagaagagaatggtcgtaattctcagagactttttcctgtttgtgtcataaatgacttcacatttttttctgttctaagaactattcagcttgatttcttctgttttaattttagcagcacctgagcaaagccatgtggtccaggattgctaccatggtgatggacagagttatcgaggcacgtactccaccactgtcacaggaaggacctgccaagcttggtcatctatgacaccacatcaacataataggaccacagaaaactacccaaatgcgtatgtcattaatcttacagtaagcaaaacaaggtccaagtaaaatttgtcttagaaaaggtgtgcgtcaagctaacttcttatgattaaatttttctcacacatagaatgcatggcaaaatgtctgagaaacattactttgagcaaagagtatgatagaagagaaatgttaagctggctctctttcctgagagtttgataaaatcaggagaatatctggcggtggtgaggccacaataatggaaaatcagaatgtttagacagagtcagcttcaacaacactcactaaaggtcaatgtgatctttaccccttgaaattctataattctaatctccaattcctgaagtgaaggttgtgttggccttttctgtcttggctcacaagtaaatgatatgtgcatatctatggaaaggcgaatctatctttttctatatctatgtctattccaacgggtagaaacaccctgggtcctgagcaccagtggtctgaaggaatacgggttgccaggaagagagaagcaaaggcaggaaggcagatgaaagtaagaaatgagacagatgctaaacaataaaaagtgcgggaagatagacagaagctggggtctgaccacaccatggccagtctttcacacataagtgactaccaaagacaagaaaaaatgatttccgcttgttggacaatagatggtagaggaccaagggaattgcgagagagagaacaatgagatcaactcaacagatgcactggttttcttcctggagacccttcctgcactgaagggcaggagatggagcccaaaaaaaactgtagccatcttgctgaacagaggagggacattggagtttgggattattcaggtggctaggattttctaggcctgctaacaatgagaacagatttgtggaggaaaggagttctagaaatatgcatagaaatctcctcgagtcattggctaaacatgaagctgcatgtacacagaaaatagatccacaagaaagtagggcaaagaacatctacggaagagcagcaactacaatggaacagtgagctcaataaacatgacagagctcaaatagcactaagggatattggagtttggaccacacagaggagagagacttcactgaacatcttgggcattcagtagagacccaggaaaagccatactttaggagtagaattagtatattcttagaataaaggcagctccacacaaacaatagcaaaactgaaaaggaagtctccaagcatcagaatgatgtccaagtcaatgaactgcctctgagaggaaaactcaaccatctttagaggtaaacatcaaagtcaagtggctcagctatgcagtatccacagtgtgaggcctaaatataaaacttgactacacatagaaaccttttagtgtgacccacaagcaggaggaaaatcagccaatacaaacagacccagaagagacagaaatgattagaatggcataaaaatttgacatatcactatataataattgagttctaggatttaagaaaacatgaatatagaatgcaacagacaccttatccagagacagtaagagtataaagagccaaatcgaagaactactaagagatatgtcttaaatgaaaaaattactagatggcctccccatctagttagacatttcagaagaaaataccaaatgaaaaataattgcatagaacctacagaaccagatacacacatacaaaacacacgcatgcatacacacacactcaaacatgtataagcttacaaacacacacacacatccacaaatgctgaaaaatgaaatcaaccgagccacacagacataaaggaaaacataaaaagatttcctacatgtgggaagcaagtcacagaaagggggaaggagattggaacagaaatatatactgaaagcaaggatggctgaaaattttccaaatataaagaagattaaaaaatcacggactcaagaagctcaatggatcagaaaaataatttctaaaatgacaattataggatgccactgggtacatagcagttcaactgtcagagggcaaagacataatacacagaaaaatctcgtaaggaacgggaaaaacaaaaagctgtgtcttgctagaggaacagtgatacaagtgactaatgtgttcccatcagaaacactgcaacctggacacaaaagaataacattaaagtaataaacgtaagaaagaagagctcaactgagaaggctacatccagcaataaaatgccttgaagttcatccatgttggaggaatgcacattgtgcactcccctaaacaaagaaaccggaaactgtaagactttggaatcagcaggcttatgtaacaaaagaggtgaccctaaggaattaaggagaagaagaatagaacaagaagggaactttctgcagcctatataatgaagaacctagcaattggcaaatgtagatgaaaatgctacatgttttcttgatcaaacgtttatatctttttaaatgagagttgacgagttgaagcaaaatgataccaatatatttaactttaccatatgtagaagtaaaaatttgaacatgtagcataaatcatgtagggattaattggaagtgtaccactgtaagtttcttacctcatgcacgatagtatgtaatactaataaaaggttaatgtgtgggttcaaagggatattgcaaatcctagagcaatcacaaagtttttaactctgaggtttgttgtataataacaatattttatgtattcaaaagagggaagccaaggaagaaaaaaaagtctttaaagagctctggctcttagtacatccagttgctcattgaatgagcttcctggaatggagggtctgggactgagactaggccacatgtgtagagccactagagacacaatgttggatccccatggcccataatacatttcccattttctcaggcagccacaggtcatgaatgtgaggatactgagaggttggagcaacgttcttgggaggcataaggaagagcgaatgcttcaagatccccgcagcccaaactcctcagctgctttgcctcctaattcattgttttttgctcctccatagctgtccgacctcttcagatctcttagtcttcctgccatcttcctttatgccatgggacccactgttctttcaactcatcccccagttctggagtggctgtggacagcagaggatagactgagagcaggagagaaggtcctgcccaggaacccattctagagatactgcattctgcctgggagcaagttttccagggcagctttgagaagtcttgcagaaacaaacctacttgaccgacatgatatgggaatgacagacagtaatactatttgcacaatgcttttccatgggaaaggtagagccttttcactaggttttgagtacatggagtgtgagagttgacctggaaaggttatcctccttgatgccatgttttctctgaagaactacatgttcgttgcaactcccacattagaatatgaagtcctaccgagagagatacggagactagacagatacagatgcatttgcatgtgaatacacaatcccacaatacagacgtcaaaacccataccagttattccagagagatggattgggcagaaggcagaaggagaatactctgatcgtttttcggccacgtgtgtgtgttatctcagtgtttctaagaagcgtttgctactttagattttttatttaaaaaaaatagtaataatctattaagtatgagagatgtgcagagaggattagtgatcgagagccatttttgctggtggcaatcatatggtacttttaatgggaatattagaaaggcaccggtaatgaccttgttgcagcacaaaggagagagtgtggggtgcccctgcatgttgtcccacctcttgtgacgtgtatcgttttggaatttccagtggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaaggtaaggagtctgtggccagacatctacacgcttcgatgctgggatgaaaagccatggaaattcccactgatgcagccgccttcaatggtaaacggatgctcgagtgttgcctgagttctaccatgtaggaggaagcctccgtgcactctctgggggagccagcggagtgatttctggtgcaacgtggttgggctttgtctttaggatgggcacaaaccctccagggggatcgacttcaaaattcaccttgttgtaaaacgggctacctcagtgtcccagccaaaatttttattgtaacatgctgtcaggtgtgtcactctttccaagccagtaagcttttccggggatttcttcaagtagccagcattcagagcaatcttcagcattgcagattctgagaaatgtggctctggagcctgtcaccctcgagaaacctaagagggctgcattgattccatgtggccctgggtctatggagcagtacatgagctcccagtgctctaaggctcttcagccctaggctttgaagggagtgatttctcagtattcttaaacctctttctgatgacacttgtacctgtgaggggtctagagagaaagagtagtagactcctactttactacaattcaggatgcagggcatgagaggattccctctctcctccaagggaagaagcttttggcgtgcacacatccctgagaagcaaagtgtctttgtcttcagtcagatacataggaccgttttctgccccatggcccggaagccaaaggccttggctttcatgatcaacggtctagggaaacatgcaaaatttccatgtctgtcccaaactctgcccccgacagccaattaccacctgcagcccgcattgccaaatgcggtgccgtttgcatgaagattcagtagagtttcctagaaaggtgctacctcgtgagctcactttccaatgaggaatctgatctgttgtgtttctctaaggtgtcaggtgaaatatttccaagaacttactacagttctagaatgggaggaatctgttgctttggtgtttgtttgttggtcggttttctcacatccatctgcctatggataaggaaaagagaacggtcgtaattctcatagactcctttctggttgtgtcacaaatggcttcacatgtttctctatgctcagagatactcagcttgatttcccgtgttttcatttcagcaccgactgagcaaaggcctggggtgcaggagtgctaccatggtaatggacagagttatcgaggcacatactccaccactgtcacaggaagaacctgccaagcttggtcatctatgacaccacactcgcatagtcggaccccagaatactacccaaatgcgtatgtctttgttctttaccataagagaagaaagggccaagtgaagtttctgttacaagagatgtgtctcaagctgagttctccgaactcaacttgtgacagatgcagatggcgtagcaaaatgtctcaggatgattgccttggagctaagggtctgagagaagggaaatgttaagctccctctccttcctcctagttctattgagcagaagggaaatctggaggtgaggagatcacattatgaagaaagtcagaatgacaaaggaccagacacttagattacccttccacaacaccaactaaacgtcaatggagactttccagttggaattccgttattctggcttccacttcctgaagggaaggttgcgtttgccttttctctctgggttcaagaggaaagaataggtgcttatttatggacaggtgaattgatctgtttctatatctacgtatattccgattgtcagaaaaacactcgttcctaagtaccagtggcctgaagggatacaggttcccagcaagagaagatccaaggaaggaaggcagatgagagtcagcacagagagggatgctgaaaagtaaaagggatgggtggatggagagaagcccgggtctgaccacccaatggccaatattttggccacaagcgactaccagagacatggaaaaatggtttctacatgtgggacaacagatggtagaggacctagagaattgagagaggggcaatgatgggctccactccgcagatgccttggctttcttcctggatacccttcctgcactgaatagcaaggagatggagcccaagcagactgtagccatcttgctgaatggaggagagggattggagtttgggatgactgtggtagctgaaatttttctaggtctgctagaaataagaactggtttgtgtggaggaaaagagctctacaaatacgcatagaagtctcctccagtcgttggcctgacatgacgctgcctgtgcacaggaaatggttccacgagaaagtgtggcaaagaacatttactgagaaacagcaagtacaagagcacaggaagctcaataaagaagagagagatcacatagcactctgggatactggagttcttcccagctagaccagagagtcctcacggagcacattgccaattcagtggagaccccagaacagccgtaatttaaaggtacacttagtatattactagaataaagtcagctgcagacaaccccttgcacagctggaaagcaagtgtccaagcatcaaatcggtttccaatcaatgaagtgcctgtgagaggaaatctcaactctctttagaagtaaacaacaaagtcgattgcctcagctatgcggtatccgcagagtgagtcctaaatttaaaatctgactacatgtagaaaagcgtttcgtgtgacccatgaccaggaaataaatcgggtaatacaaacaggctcaggaatgagagaaatgattagaattgcgtgaaaatttgacatatcagtatgataactgatttcaaatatttaaaaaaacaacatgcaagaaagcagatatcatatcaagagaaattaacagtacagaatagccaaattaaattaaagaggtagtataaaaaaagtatgtcttaattgaaaaaaattactgtatggccggctgatcaatttagacgtttcagaggaaaacattacccaacacacaattctagagaacctacagaatgagctacacacacacacacacacacacacacacacactgaaaacacacccatactcacacacacgcagaaactcacaagttctaacacacacagacacgcgcacccctgaagaaacagtgaaatataaaattaagcgagcctcacagacatgtaggaaaatatgaaaagatttcctgcatgtgggaagcaagtcacagtaaagagcaagggagtttataatagaaacaaataccagaatcaaggatggctgataacttttcaattacgaagaacattaaaaaaaatcacagaatcgtgaaactcaagggatcatatagggaatttcggaaaaaaaacccaacctgtatgatgtacttttgtacatcacagttcgaaggtaacaaggcaaagatgtaataagaagaaacctgtcacgagaaactggaggaaaaagagctgtgtcttcctacaagtacactgatacaaattgccaatgtgttcacctcagaaacactggaagccagataccagggaatattgttaaaatgataatcaggaacaaaaagagatcaaccgggaatgctgaatccagcaataaaatgccttgaaggtcatccatgtcggataaatgcatattgtgcactgccccaaagaaagaaaccggaaactgtaagaattggaaatcagcaggcttatgtaacaagagaggtgacccgaaggaattaggtagaagaagaattgaacaagaaaggaactttctgcagcccacgtaatgaagaatccagcaattggcaaatgtagatagatgtaaatgcaaaatattttcttgatcaaatttctatatctttgtaaatgagagttgactacttgaaacaaaatgatagcaagatatttaacttcagcatatgtagaggtaagaatttgaaatggtagcataaatcacgaagggattaattcgaagtgtaccgttgtaagtttctttacctcatgcacgatggtgtgtcatattaataaaagggtactgtgcgggttcgaagggatattgcaaatcctagagcaatcacaaaggtttgaactctgaggtttttggtataataagaatagtccatgcattcaaaagagggaagccaaggaagaactagaagtctttcaagagctcaggctcttatacatccagttgctcattgaaccagcttcctggaatggagggtctggggttgagactaggccacaagtctagagtctctagagagacagtgttggaaccccatggcccataatacatttcccattttctcaggcagccagaggtcatgaatgtgaggatactgggaggttggagcaacgttcttgggaggcataaggaagagcgaatgcttcaagatccccgcagcccaaactactcgcctgctttgccccctaatgcatttttctctgctgctccgtagctgtccgacctcttcagatctcttagtccaccctgccgtcttcctttatgccatgggtcccactgttctttcaactcatccccctttccctcagtcccggagtagctgcggccagcagagggtagactgagagcaggagagaaggacctgcctaggaaccccttctagagatactgcatcctgcctgggagcaagttttccagggcagctttgagaagtcttggagaaacaaacctactaaacctgacagacagtaatactatttgcacaatgcttttctgtgggaaaggtagagccttttcactacgtattgagtacatagagtgtgagggttgacctggaacggctatcctcctggatgacgtgtgttttctgaagaactacatgttcgttgcaactcccacattagaatatgaagtcctaccgagagagatacggagactagacagatacagatgcatttgcatgtgaatacacaatcccacaatacagacgtcaaaacccataccagttattccagagagatggattgggcagaaggcagaaggagaatactctgatcgtttttcggccacgtgtgtgtgttatctcagtgtttctaagaagcgtttgctactttagattttttatttaaaaaaatagtaataatctattaagtatgagagatgtgcagagaggattagtgatcgagagccatttttgctggtggcaatcatatggtacttttaatgggaatattagaaaggcaccggtaatgaccttgttgcagcacaaaggagagagtgtggggtgcccctgcatgttgtcccacctcttgtgacgtgtatcgttttggaatttccagtggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaaggtaaggagtctgtggccagacatctacacgcttcgatgctgggatgaaaagccatggaaattcccactgatgcagccgccttcaatggtaaacggatgctcgagtgttgcctgagttctaccatgtaggaggaagcctccgtgcactctctgggggagccagcggagtgatttctggtgcaacgtggttgggctttgtctttaggatgggcacaaaccctccagggggatcgacttcaaaattcaccttgttgtaaaacgggctacctcagtgtcccagccaaaatttttattgtaacatgctgtcaggtgtgtcactctttccaagccagtaagcttttccggggatttcttcaagtagccagcattcagagcaatcttcagcattgcagattctgagaaatgtggctctggagcctgtcaccctcgagaaacctaagagggctgcattgattccatgtggccctgggtctatggagcagtacatgagctcccagtgctctaaggctcttcagccctaggctttgaagggagtgatttctcagtattcttaaacctctttctgatgacacttgtacctgtgaggggtctagagagaaagagtagtagactcctactttactacaattcaggatgcagggcatgagaggattccctctctcctccaagggaagaagcttttggcgtgcacacatccctgagaagcaaagtgtctttgtcttcagtcagatacataggaccgttttctgccccatggcccggaagccaaaggccttggctttcatgatcaacggtctagggaaacatgcaaaatttccatgtctgtcccaaactctgcccccgacagccaattaccacctgcagcccgcattgccaaatgcggtgccgtttgcatgaagattcagtagagtttcctagaaaggtgctacctcgtgagctcactttccaatgaggaatctgatctgttgtgtttctctaaggtgtcaggtgaaatatttccaagaacttactacagttctagaatgggaggaatctgttgctttggtgtttgtttgttggtcggttttctcacatccatctgcctatggataaggaaaagagaacggtcgtaattctcatagactcctttctggttgtgtcacaaatggcttcacatgtttctctatgctcagagatactcagcttgatttcccgtgttttcatttcagcaccgactgagcaaaggcctggggtgcaggagtgctaccatggtaatggacagagttatcgaggcacatactccaccactgtcacaggaagaacctgccaagcttggtcatctatgacaccacactcgcatagtcggaccccagaatactacccaaatgcgtatgtctttgttctttaccataagagaagaaagggccaagtgaagtttctgttacaagagatgtgtctcaagctgagttctccgaactcaacttgtgacagatgcagatggcgtagcaaaatgtctcaggatgattgccttggagctaagggtctgagagaagggaaatgttaagctccctctccttcctcctagttctattgagcagaagggaaatctggaggtgaggagatcacattatgaagaaagtcagaatgacaaaggaccagacacttagattacccttccacaacaccaactaaacgtcaatggagactttccagttggaattccgttattctggcttccacttcctgaagggaaggttgcgtttgccttttctctctgggttcaagaggaaagaataggtgcttatttatggacaggtgaattgatctgtttctatatctacgtatattccgattgtcagaaaaacactcgttcctaagtaccagtggcctgaagggatacaggttcccagcaagagaagatccaaggaaggaaggcagatgagagtcagcacagagagggatgctgaaaagtaaaagggatgggtggatggagagaagcccgggtctgaccacccaatggccaatattttggccacaagcgactaccagagacatggaaaaatggtttctacatgtgggacaacagatggtagaggacctagagaattgagagaggggcaatgatgggctccactccgcagatgccttggctttcttcctggatacccttcctgcactgaatagcaaggagatggagcccaagcagactgtagccatcttgctgaatggaggagagggattggagtttgggatgactgtggtagctgaaatttttctaggtctgctagaaataagaactggtttgtggaggaaaagagctctacaaatacgcatagaagtctcctccagtcgttggcctgacatgacgctgcctgtgcacaggaaatggttccacgagaaagtgtggcaaagaacatttactgagaaacagcaagtacaagagcacaggaagctcaataaagaagagagagatcacatagcactctgggatactggagttcttcccagctagaccagagagtcctcacggagcacattgccaattcagtggagaccccagaacagccgtaatttaaaggtacacttagtatattactagaataaagtcagctgcagacaaccccttgcacagctggaaagcaagtgtccaagcatcaaatcggtttccaatcaatgaagtgcctgtgagaggaaatctcaactctctttagaagtaaacaacaaagtcgattgcctcagctatgcggtatccgcagagtgagtcctaaatttaaaatctgactacatgtagaaaagcgtttcgtgtgacccatgaccaggaaataaatcgggtaatacaaacaggctcaggaatgagagaaatgattagaattgcgtgaaaatttgacatatcagtatgataactgatttcaaatatttaaaaaaacaacatgcaagaaagcagatatcatatcaagagaaattaacagtacagaatagccaaattaaattaaagagctagtataaaaaaagtatgtcttaattgaaaaaaattactgtatggccggctgatcaatttagacgtttcagaggaaaacattacccaacacacaattctagagaacctacagaatgagctacacacacacacacacacacacacacaaactgaaaacacacccatactcacacacacgcagaaactcacaagttctaacacacacagacacgcgcacccctgaagaaacagtgaaatataaaattaagcgagcctcacagacatgtaggaaaatatgaaaagatttcctgcatgtgggaagcaagtcacagtaaagagcaagggagtttggaatagaaacaaataccggaatcaaggatggctgataacttttcaattacgaagaacattaaaaaaaatcacagaatcgtgaaactcaagggatcacatagggaatttcggaaaaaaaacccaacctgtatgatgtacttttgtacatcacagttcgaaggtaacaaggcaaagatataataagaagaaacctgtcacgagaaactggaggaaaaagagctgtgtcttcctacaagtacactgatacaaattgccaatgtgttcacctcagaaacactggaagccagataccagggaatattgttaaaatgataatcaggaacaaaaagagatcaaccgggaatgctgaatccagcaataaaatgccttgaagatcatccatgtcggataaatgcatattgtgcactgccccaaagaaagaaaccggaaactgtaagaattggaaatcagcaggcttatgtaacaagagaggtgacccgaaggaattaggtagaagaagaattgaacaagaaaggaactttctgcagcccacgtaatgaagaatccagcaattggcaaatgtagatagatgtaaatgcaaaatattttcttgatcaaatttctatatctttgtaaatgagagttgactacttgaaacaaaatgatagcaagatatttaacttcagcatatgtagaggtaagaatttgaaatggtagcataaatcacgaagggattaattcgaagtgtaccgttgtaagtttctttacctcatgcacgatggtgtgtcatattaataaaagggtactgtgcgggttcgaagggatattgcaaatcctagagcaatcacaaaggtttgaactctgaggtttttggtataataagaatagtccatgcattcaaaagagggaagccaaggaagaactagaagtctttcaagagctcaggctcttatacatccagttgctcattgaaccagcttcctggaatggagggtctggggttgagactaggccacaagtctagagtctctagagagacagtgttggaaccccatggcccataatacatttcccattttctcaggcagccagaggtcatgaatgtgaggatactgggaggttggagcaacgttcttgggaggcataaggaagagcgaatgcttcaagatccccgcagcccaaactactcgcctgctttgccccctaatgcatttttctctgctgctccgtagctgtccgacctcttcagatctcttagtccaccctgccgtcttcctttatgccatgggtcccactgttctttcaactcatccccctttccctcagtcccggagtagctgcggccagcagagggtagactgagagcaggagagaaggacctgcctaggaaccccttctagagatactgcatcctgcctgggagcaagttttccagggcagctttgagaagtcttggagaaacaaacctactaaacctgacagacagtaatactatttgcacaatgcttttctgtgggaaaggtagagccttttcactacgtattgagtacatagagtgtgagggttgacctggaacggctatcctcctggatgacgtgtgttttctgaagaactacatgttcgttgcaactcccacattagaatatgaagtcctaccgagagagatacggagactagacagatacagatgcatttgcatgtgaatacacaatcccacaatacagacgtcaaaacccataccagttattccagagagatggattgggtaggaggcagaaggagaatactctgatcgtttttcggccacgtgtgtgtgttatctcagtgtttctaagaagcgtttgctactttagattttttatttaaaaaaaatagtaataatctattaagtatgagagatgtgcagagaggattagtgatcgagagccatttttgctggtggcaatcatatggtacttttaatgggaatattagaaaggcaccggtaatgaccttgttgcagcacaaaggagagagtgtggggtgcccctgcatgttgtcccacctcttgtgacgtgtatcgttttggaatttccagtggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaaggtaaggagtctgtggccagacatctacacgcttcgatgctgggatgaaaagccatggaaattcccactgatgcagccgccttcaatggtaaacggatgctcgagtgttgccggagttctgccatgttgggggaagcctccgtgtactctctgggggagccagcggagtgatttctggtgcaacttgggtgggctttgtctttagaatgggcacaaaccttccagggtgatgggcttcacaactcacctccttctaaaatgggctatctcagtgtcttagccaaaatttttattgtaacgtgctgtcaggtgtgtgattctttctgtcgcagtaagcttttctggggatttcttcaagtagccagcagtcagtgcaatcttcagcattgcagatttcaaaaaatgtggctctggagcctgtcatcctcgagaaacctaacagggctgcattaattccatatggtcctgggtctatggagcagtatatgagctcccaatgctctaaggctcttcagtcctaggctttgaagggagtgatttctcagtgttcttaaacctctttctgatggcacttgtacctgtgaggggtctagagagaaaggttagtagacttctcctttactgcaattcaggatgcagggcatgagaagattccctccctcctccaagggaagaaggttttggcgtgcacacatccttgagaagcaaagtgtctttgccttcagtcagatatataggatcgttttctgccccatggcctggaagccagaggccttggctttcatgatcaacgatctagggaaacatgcaaaatttccatgtctttcccctcctctgccctcgacagccaattaccacctgcatcctgcattgccaaatgcagtgccctttgtatgaacattcagtagagtttcatagaaaggtgctacttcgtgagcgcactttgcagtgagaaggagtctgttctgttctgtttttctaaggatttcaggtgaaatatttcctagaacttactacagttctagattggtaggaatctgtaggtttgctgtatgttttttggttggttttctcccatccatctgcctacaggtaagggaaagataacgttcgtaattctcatagactcctttctggttgtgtcataaatggcttcacatatttcgttattctcagagatactcagtttatttcttgtgttttcatttcagcaccgactgagcagaggcctggggtgcaggagtgctaccacggtaatggacagagttatcgaggcacatactccaccactgtcactggaagaacctgccaagcttggtcatctatgacaccacactcgcatagtcggaccccagaatactacccaaatgcgtatgtctttgttctttaccataagagaagaaagggccaagtgaagtttctgttacaagagatgtgtctcaagctgagttctccgaactcaacttgtgacagatgcagatggcgtagcaaaatgtctcaggatgattgccttggagctaagggtctgagagaagggaaatgttaagctccctctccttcctcctagttctattgagcagaagggaaatctggaggtgaggagatcacattatgaagaaagtcagaatgacaaaggaccagacacttagattacccttccacaacaccaactaaacgtcaatggagactttccagttggaattccgttattctggcttccacttcctgaagggaaggttgcgtttgccttttctctctgggttcaagaggaaagaataggtgcttatttatggacaggtgaattgatctgtttctatatctacgtatattccgattgtcagaaaaacactcgttcctaagtaccagtggcctgaagggatacaggttcccagcaagagaagatccaaggaaggaaggcagatgagagccagcacagagagggatgctgaaaagtaaaagggatgggtggatggagagaagcccgggtctgaccacccaatggccaatattttggccacaagcgactaccagagacatggaaaaatggtttctacatgtgggacaacagatggtagaggacctagagaattgagagaggggcaatgatgggctccactccgcagatgccttggctttcttcctggatacccttcctgcactgaatagcaaggagatggagcccaagcagactgtagccatcttgctgaatggaggagagggattggagtttgggatgactgtggtagctgaaatttttctaggtctgctagaaataagaactggtttgtggaggaaaagagctctacaaatacgcatagaagtctcctccagtcgttggcctgacatgacgctgcctgtgcacaggaaatggttccacgagaaagtgtggcaaagaacatttactgagaaacagcaagtacaagagcacaggaagctcaataaagaagagagagatcacatagcactctgggatactggagttcttcccagctagaccagagagtcctcacggagcacattgccaattcagtggagaccccagaacagccgtaatttaaaggtacacttagtatattactagaataaagtcagctgcagacaaccccttgcacagctggaaagcaagtgtccaagcatcaaatcggtttccaatcaatgaagtgcctgtgggaggaaatctcaactctctttagaagtaaacaacaaagtcgattgcctcagctatgcggtatccgcagagtgagtcctaaatttaaaatctgactacatgtagaaaagcgtttcgtgtgacccatgaccaggaaataaatcgggtaatacaaacaggctcaggaatgagagaaatgattagaattgcgtgaaaatttgacatatcagtatgataactgatttcaaatatttaaaaaaacaacatgcaagaaagcagatatcatatcaagagaaattaacagtacagaatagccaaattaaattaaagagctagtataaaaaaagtatgtcttaattgaaaaaaattactgtatggccggctgatcaaattagacgtttcagaggaaaacattacccaacacacaattctagagaacctacagaatgagctacacacacacacacacacacacacacacacactgaaaacacacccatactcacacacacgcagaaactcacaagttctaacacacacagacacgcgcacccctgaagaaacagtgaaatataaaattaagcgagcctcacagacatgtaggaaaatatgaaaagatttcctgcatgtgggaagcaagtcacagtaaagagcaagggagtttggaatagaaacaaataccggaatcaaggatggctgataacttttcaattacgaagaacattaaaaaaaatcacagaatcgtgaaactcaagggatcatatagggaatttcggaaaaaaaacccaacctgtatgatgtacttttgtacatcacagttcgaaggtaacaaggcaaagatataataagaagaaacctgtcacgagaaactggaggaaaaagagctgtgtcttcctacaagtacactgatacaaattgccaatgtgttcacctcagaaacactggaagccagataccagggaatattgttaaaatgataatcaggaacaaaaagagatcaaccgggaatgctgaatccagcaataaaatgccttgaagatcatccatgtcggataaatgcatattgtgcactgccccaaagaaagaaaccggaaactgtcagaattggaaatcagcaggcttatgtaacaagagaggtgacccgaaggaattaggtagaagaagaattgaacaagaaaggaactttctgcagcccacgtaatgaagaatccagcaattggcaaatgtagatagatgtaaatgcaaaatattttcttgatcaaatttctatatctttgtaaatgagagttgactacttgaaacaaaatgatagcaagatatttaacttcagcatatgtagaggtaagaatttgaaatggtagcataaatcacgaagggattaattcgaagtgtaccgttgtaagtttctttacctcatgcacgatggtgtgtcatattaataaaagggtactgtgcgggttcgaagggatattgcaaatcctagagcaatcacaaaggtttgaactctgaggtttttggtataataagaatagtccatgcattcaaaagagggaagccaaggaagaactagaagtctttcaagagctcaggctcttatacatccagttgctcattgaaccagcttcctggaatggagggtctggggttgagactaggccacaagtctagagtctctagagagacagtgttggaaccccatggcccataatacatttcccattttctcaggcagccagaggtcatgaatgtgaggatactgggaggttggagcaacgttcttgggaggcataaggaagagcgaatgcttcaagatccccgcagcccaaactactcgcctgctttgccccctaatgcatttttctctgctgctccgtagctgtccgacctcttcagatctcttagtccaccctgccgtcttcctttatgccatgggtcccattgttctttcaactcatccccctttccctcagtcccggagtagctgcggccagcagagggtagactgagagcaggagagaaggacctgcctaggaaccccttctagagatactgcatcctgcctgggagcaagttttccagggcagctttgagaagtcttggagaaacaaacctactaaacctgacagacagtaatactatttgcacaatgcttttctgtgggaaaggtagagccttttcactacgtattgagtacatagagtgtgagggttgacctggaacggctatcctcctggatgacgtgcgttttctgaagaactacatgttcgttgcaactcccacattagaatatgaagtcctaccgagagagatacggagactagacagatacagatgcatttgcatgtgaatacacaatcccacaatacagacgtcaaaacccataccagttattccagagagatggattgggcagaaggcagaaggagaatactctgatcgtttttcggccacgtgtgtgtgttatctcagtgtttctaagaagcgtttgctactttagattttttatttaaaaaaaatagtaataatctattaagtatgagagatgtgcagagaggattagtgatcgagagccatttttgctggtggcaatcatatggtacttttaatgggaatattagaaaggcaccggtaatgaccttgttgcagcacaaaggagagagtgtggggtgcccctgcatgttgtcccacctcttgtgacgtgtatcgttttggaatttccagtggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaaggtaaggagtctgtggccagacatctacacgcttcgatgctgggatgaaaagccatggaaattcccactgatgcagccgccttcaatggtaaacggatgctcgagtgttgcctgagttctaccatgtaggaggaagcctccgtgcactctctgggggagccagcggagtgatttctggtgcaacgtggttgggctttgtctttaggatgggcacaaaccctccagggggatcgacttcaaaattcaccttgttgtaaaacgggctacctcagtgtcccagccaaaatttttattgtaacatgctgtcaggtgtgtcactctttccaagccagtaagcttttccggggatttcttcaagtagccagcattcagagcaatcttcagcattgcagattctgagaaatgtggctctggagcctgtcaccctcgagaaacctaagagggctgcattgattccatgtggccctgggtctatggagcagtacatgagctcccagtgctctaaggctcttcagccctaggctttgaagggagtgatttctcagtattcttaaacctctttctgatgacacttgtacctgtgaggggtctagagagaaagagtagtagactcctactttactacaattcaggatgcagggcatgagaggattccctctctcctccaagggaagaagcttttggcgtgcacacatccctgagaagcaaagtgtctttgtcttcagtcagatacataggaccgttttctgccccatggcccggaagccaaaggccttggctttcatgatcaacggtctagggaaacatgcaaaatttccatgtctgtcccaaactctgcccccgacagccaattaccacctgcagcccgcattgccaaatgcggtgccgtttgcatgaagattcagtagagtttcctagaaaggtgctacctcgtgagctcactttccaatgaggaatctgatctgttgtgtttctctaaggtgtcaggtgaaatatttccaagaacttactacagttctagaatgggaggaatctgttgctttggtgtttgtttgttggtcggttttctcacatccatctgcctatggataaggaaaagagaacggtcgtaattctcatagactcctttctggttgtgtcacaaatggcttcacatgtttctctatgctcagagatactcagcttgatttcccgtgttttcatttcagcaccgactgagcaaaggcctggggtgcaggagtgctaccatggtaatggacagagttatcgaggcacatactccaccactgtcacaggaagaacctgccaagcttggtcatctatgacaccacactcgcatagtcggaccccagaatactacccaaatgcgtatgtctttgttctttaccataagagaagaaagggccaagtgaagtttctgttacaagagatgtgtctcaagctgagttctccgaactcaacttgtgacagatgcagatggcgtagcaaaatgtctcaggatgattgccttggagctaagggtctgagagaagggaaatgttaagctccctctccttcctcctagttctattgagcagaagggaaatctggaggtgaggagatcacattatgaagaaagtcagaatgacaaaggaccagacacttagattacccttccacaacaccaactaaacgtcaatggagactttccagttggaattccgttattctggcttccacttcctgaagggaaggttgcgtttgccttttctctctgggttcaagaggaaagaataggtgcttatttatggacaggtgaattgatctgtttctatatctacgtatattccgattgtcagaaaaacactcgttcctaagtaccagtggcctgaagggatacaggttcccagcaagagaagatccaaggaaggaaggcagatgagagtcagcacagagagggatgctgaaaagtaaaagggatgggtggatggagagaagcccgggtctgaccacccaatggccaatattttggccacaagcgactaccagagacatggaaaaatggtttctacatgtgggacaacagatggtagaggacctagagaattgagagaggggcaatgatgggctccactccgcagatgccttggctttcttcctggatacccttcctgcactgaatagcaaggagatggagcccaagcagactgtagccatcttgctgaatggaggagagggattggagtttgggatgactgtggtagctgaaatttttctaggtctgctagaaataagaactggtttgtggaggaaaagagctctacaaatacgcatagaagtctcctccagtcgttggcctgacatgacgctgcctgtgcacaggaaatggttccacgagaaagtgtggcaaagaacatttactgagaaacagcaagtacaagagcacaggaagctcaataaagaagagagagatcacatagcactctgggatactggagttcttcccagctagaccagagagtcctcacggagcacattgccaattcagtggagaccccagaacagccgtaatttaaaggtacacttagtatattactagaataaagtcagctgcagacaaccccttgcacagctggaaagcaagtgtccaagcatcaaatcggtttccaatcaatgaagtgcctgtgagaggaaatctcaactctctttagaagtaaacaacaaagtcgattgcctcagctatgcggtatccgcagagtgagtcctaaatttaaaatctgactacatgtagaaaagcgtttcgtgtgacccatgaccaggaaataaatcgggtaatacaaacaggctcaggaatgagagaaatgattagaattgcgtgaaaatttgaaatatcagtatgataactgatttcaaatatttaaaaaaacaacatgcaagaaagcagatatcatatcaagagaaattaacagtacagaatagccaaattaaattaaagagctagtataaaaaaagtatgtcttaattgaaaaaaattactgtatggccggctgatcaatttagacgtttcagaggaaaacattacccaacacacaattctagagaacctacagaatgagctacacacacacacacacacacacacacaaactgaaaacacacccatactcacacacacgcagaaactcacaagttctaacacacacagacacgcgcacccctgaagaaacagtgaaatataaaattaagcgagcctcacagacatgtaggaaaatatgaaaagatttcctgcatgtgggaagcaagtcacagtaaagagcaagggagtttggaatagaaacaaataccagaatcaaggatggctgataacttttcaattacgaagaacattaaaaaaaatcacagaatcgtgaaactcaagggatcacatagggaatttcggaaaaaaaacccaacctgtatgatgtacttttgtacatcacagttcgaaggtaacaaggcaaagatataataagaagaaacctgtcacgagaaactggaggaaaaagagctgtgtcttcctacaagtacactgatacaaattgccaatgtgttcacctcagaaacactggaagccagataccagggaatattgttaaaatgataatcaggaacaaaaagagatcaaccgggaatgctgaatccagcaataaaatgccttgaagatcatccatgtcggataaatgcatattgtgcactgccccaaagaaagaaaccggaaactgtaagaattggaaatcagcaggcttatgtaacaagagaggtgacccgaaggaattaggtagaagaagaattgaacaagaaaggaactttctgcagcccacgtaatgaagaatccagcaattggcaaatgtagatagatgtaaatgcaaaatattttcttgatcaaatttctatatctttgtaaatgagagttgactacttgaaacaaaatgatagcaagatatttaacttcagcatatgtagaggtaagaatttgaaatggtagcataaatcacgaagggattaattcgaagtgtaccgttgtaagtttctttacctcatgcacgatggtgtgtcatattaataaaagggtactgtgcgggttcgaagggatattgcaaatcctagagcaatcacaaaggtttgaactctgaggtttttggtataataagaatagtccatgcattcaaaagagggaagccaaggaagaactagaagtctttcaagagctcaggctcttatacatccagttgctcattgaaccagcttcctggaatggagggtctggggttgagactaggccacaagtctagagtctctagagagacagtgttggaaccccatggcccataatacatttcccattttctcaggcagccagaggtcatgaatgtgaggatactgggaggttggagcaacgttcttgggaggcataaggaagagcgaatgcttcaagatccccgcagcccaaactactcgcctgctttgccccctaatgcatttttctctgctgctccgtagctgtccgacctcttcagatctcttagtccaccctgccgtcttcctttatgccatgggtcccactgttctttcaactcatccccctttccctcagtcccggagtagctgcggccagcagagggtagactgagagcaggagagaaggacctgcctaggaaccccttctagagatactgcatcctgcctgggagcaagttttccagggcagctttgagaagtcttggagaaacaaacctactaaacctgacagacagtaatactatttgcacaatgcttttctgtgggaaaggtagagccttttcactacgtattgagtacatagagtgtgagggttgacctggaacggctatcctcctggatgacgtgtgttttctgaagaactacatgttcgttgcaactcccacattagaatatgaagtcctaccgagagagatacggagactagacagatacagatgcatttgcatgtgaatacacaatcccacaatacagacgtcaaaacccataccagttattccagagagatggattgggcagaaggcagaaggagaatactctgatcgtttttcggccacgtgtgtgtgttatctcagtgtttctaagaagcgtttgctactttagattttttatttaaaaaaaatagtaataatctattaagtatgagagatgtgcagagaggattagtgatcgagagccatttttgctggtggcaatcatatggtacttttaatgggaatattagaaaggcaccggtaatgaccttgttgcagcacaaaggagagagtgtggggtgcccctgcatgttgtcccacctcttgtgacgtgtatcgttttggaatttccagtggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaaggtaaggagtctgtggccagacatctacacgcttcgatgctgggatgaaaagccatggaaattcccactgatgcagccgccttcaatggtaaacggatgctcgagtgttgcctgagttctaccatgtaggaggaagcctccgtgcactctctgggggagccagcggagtgatttctggtgcaacgtggttgggctttgtctttaggatgggcacaaaccctccagggggatcgacttcaaaattcaccttgttgtaaaacgggctacctcagtgtcccagccaaaatttttattgtaacatgctgtcaggtgtgtcactctttccaagccagtaagcttttccggggatttcttcaagtagccagcattcagagcaatcttcagcattgcagattctgagaaatgtggctctggagcctgtcaccctcgagaaacctaagagggctgcattgattccatgtggccctgggtctatggagcagtacatgagctcccagtgctctaaggctcttcagccctaggctttgaagggagtgatttctcagtattcttaaacctctttctgatgacacttgtacctgtgaggggtctagagagaaagagtagtagactcctactttactacaattcaggatgcagggcatgagaggattccctctctcctccaagggaagaagcttttggcgtgcacacatccctgagaagcaaagtgtctttgtcttcagtcagatacataggaccgttttctgccccatggcccggaagccaaaggccttggctttcatgatcaacggtctagggaaacatgcaaaatttccatgtctgtcccaaactcttcccccgacagccaattaccacctgcagcccgcattgccaaatgcggtgccgtttgcatgaagattcagtagagtttcctagaaaggtgctacctcgtgagctcactttccaatgaggaatctgatctgttgtgtttctctaaggtgtcaggtgaaatatttccaagaacttactacagttctagaatgggaggaatctgttgctttggtgtttgtttgttggtcggttttctcacatccatctgcctatggataaggaaaagagaacggtcgtaattctcatagactcctttctggttgtgtcacaaatggcttcacatgtttctctatgctcagagatactcagcttgatttcccgtgttttcatttcagcaccgactgagcaaaggcctggggtgcaggagtgctaccatggtaatggacagagttatcgaggcacatactccaccactgtcacaggaagaacctgccaagcttggtcatctatgacaccacactcgcatagtcggaccccagaatactacccaaatgcgtatgtctttgttctttaccataagagaagaaagggccaagtgaagtttctgttacaagagatgtgtctcaagctgagttctccgaactcaacttgtgacagatgcagatggcgtagcaaaatgtctcaggatgattgccttggagctaagggtctgagagaagggaaatgttaagctccctctccttcctcctagttctattgagcagaagggaaatctggaggtgagaagatcacattatgaagaaagtcagaatgacaaaggaccagacacttagattacccttccacaacaccaactaaacgtcaatggagactttccagttggaattccgttattctggcttccacttcctgaagggaaggttgcgtttgccttttctctctgggttcaagaggaaagaataggtgcttatttatggacaggtgaattgatctgtttctatatctacgtatattccgattgtcagaaaaacactcgttcctaagtaccagtggcctgaagggatacaggttcccagcaagagaagatccaaggaaggaaggcagatgagagtcagcacagagagggatgctgaaaagtaaaagggatgggtggatggagagaagcccgggtctgaccacccaatggccaatattttggccacaagcgactaccagagacatggaaaaatggtttctacatgtgggacaacagatggtagaggacctagagaattgagagaggggcaatgatgggctccactccgcagatgccttggctttcttcctggatacccttcctgcactgaatagcaaggagatggagcccaagcagactgtagccatcttgctgaatggaggagagggattggagtttgggatgactgtggtagctgaaatttttctaggtctgctagaaataagaactggtttgtgtggaggaaaagagctctacaaatacgcatagaagtctcctccagtcgttggcctgacatgacgctgcctgtgcacaggaaatggttccacgagaaagtgtggcaaagaacatttactgagaaacagcaagtacaagagcacaggaagctcaataaagaagagagagatcacatagcactctgggatactggagttcttcccagctagaccagagagtcctcacggagcacattgccaattcagtggagaccccagaacagccgtaatttaaaggtacacttagtatattactagaataaagtcagctgcagacaaccccttgcacagctggaaagcaagtgtccaagcatcaaatcggtttccaatcaatgaagtgcctgtgagaggaaatctcaactctctttagaagtaaacaacaaagtcgattgcctcagctatgcggtatccgcagagtgagtcctaaatttaaaatctgactacatgtagaaaagcgtttcgtgtgacccatgaccaggaaataaatcgggtaatacaaacaggctcaggaatgagagaaatgattagaattgcgtgaaaatttgacatatcagtatgataactgatttcaaatatttaaaaaaacaacatgcaagaaagcagatatcatatcaagagaaattaacagtacagaatagccaaattaaattaaagaggtagtataaaaaaagtatgtcttaattgaaaaaaattactgtatggccggctgatcaatttagacgtttcagaggaaaacattacccaacacacaattctagagaacctacagaatgagctacacacacacacacacacacacacacaaactgaaaacacacccatactcacacacacgcagaaactcacaagttctaacacacacagacacgcgcacccctgaagaaacagtgaaatataaaattaagcgagcctcacagacatgtaggaaaatatgaaaagatttcctgcatgtgggaagcaagtcacagtaaagagcaagggagtttggaatagaaacaaataccggaatcaaggatggctgataacttttcaattacgaagaacattaaaaaaaatcacagaatcgtgaaactcaagggatcacatagggaatttcggaaaaaaaacccaacctgtatgatgtacttttgtacatcacagttcgaaggtaacaaggcaaagatataataagaagaaacctgtcacgagaaactggaggaaaaagagctgtgtcttcctacaagtacactgatacaaattgccaatgtgttcacctcagaaacactggaagccagataccagggaatattgttaaaatgataatcaggaacaaaaagagatcaaccgggaatgctgaatccagcaataaaatgccttgaaggtcatccatgtcggataaatgcatattgtgcactgccccaaagaaagaaaccggaaactgtaagaattggaaatcagcaggcttatgtaacaagagaggtgacccgaaggaattaggtagaagaagaattgaacaagaaaggaactttctgcagcccacgtaatgaagaatccagcaattggcaaatgtagatagatgtaaatgcaaaatattttcttgatcaaatttctatatctttgtaaatgagagttgactacttgaaacaaaatgatagcaagatatttaacttcagcatatgtagaggtaagaatttgaaatggtagcataaatcacgaagggattaattcgaagtgtaccgttgtaagtttctttacctcatgcacgatggtgtgtcatattaataaaagggtactgtgcgggttcgaagggatattgcaaatcctagagcaatcacaaaggtttgaactctgaggtttttggtataataagaatagtccatgcattcaaaagagggaagccaaggaagaactagaagtctttcaagagctcaggctcttatacatccagttgctcattgaaccagcttcctggaatggagggtctggggttgagactaggccacaagtctagagtctctagagagacagtgttggaaccccatggcccataatacatttcccattttctcaggcagccagaggtcatgaatgtgaggatactgggaggttggagcaacgttcttgggaggcataaggaagagcgaatgcttcaagatccccgcagcccaaactactcgcctgctttgccccctaatgcatttttctctgctgctccgtagctgtccgacctcttcagatctcttagtccaccctgccgtcttcctttatgccatgggtcccactgttctttcaactcatccccctttccctcagtcccggagtagctgcggccagcagagggtagactgagagcaggagagaaggacctgcctaggaaccccttctagagatactgcatcctgcctgggagcaagttttccagggcagctttgagaagtcttggagaaacaaacctactaaacctgacagacagtaatactatttgcacaatgcttttctgtgggaaaggtagagccttttcactacgtattgagtacatagagtgtgagggttgacctggaacggctatcctcctggatgacgtgcgttttctgaagaactacatgttcgttgcaactcccacattagaatatgaagtcctaccgagagagatacggagactagacagatacagatgcatttgcatgtgaatacacaatcccacaatacagacgtcaaaacccataccagttattccagagagatggattgggcagaaggcagaaggagaatactctgatcgtttttcggccacgtgtgtgtgttatctcagtgtttctaagaagcgtttgctactttagattttttatttaaaaaaaatagtaataatctattaagtatgagagatgtgcagagaggattagtgatcgagagccatttttgctggtggcaatcatatggtacttttaatgggaatattagaaaggcaccggtaatgaccttgttgcagcacaaaggagagagtgtggggtgcccctgcatgttgtcccacctcttgtgacgtgtatcgttttggaatttccagtggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaaggtaaggagtctgtggccagacatctacacgcttcgatgctgggatgaaaagccatggaaattcccactgatgcagccgccttcaatggtaaacggatgctcgagtgttgcctgagttctaccatgtaggaggaagcctccgtgcactctctgggggagccagcggagtgatttctggtgcaacgtggttgggctttgtctttaggatgggcacaaaccctccagggggatcgacttcaaaattcaccttgttgtaaaacgggctacctcagtgtcccagccaaaatttttattgtaacatgctgtcaggtgtgtcactctttccaagccagtaagcttttccggggatttcttcaagtagccagcattcagagcaatcttcagcattgcagattctgagaaatgtggctctggagcctgtcaccctcgagaaacctaagagggctgcattgattccatgtggccctgggtctatggagcagtacatgagctcccagtgctctaaggctcttcagccctaggctttgaagggagtgatttctcagtattcttaaacctctttctgatgacacttgtacctgtgaggggtctagagagaaagagtagtagactcctactttactacaattcaggatgcagggcatgagaggattccctctctcctccaagggaagaagcttttggcgtgcacacatccctgagaagcaaagtgtctttgtcttcagtcagatacataggaccgttttctgccccatggcccggaagccaaaggccttggctttcatgatcaacggtctagggaaacatgcaaaatttccatgtctgtcccaaactctgcccccgacagccaattaccacctgcagcccgcattgccaaatgcggtgccgtttgcatgaagattcagtagagtttcctagaaaggtgctacctcgtgagctcactttccaatgaggaatctgatctgttgtgtttctctaaggtgtcaggtgaaatatttccaagaacttactacagttctagaatgggaggaatctgttgctttggtgtttgtttgttggtcggttttctcacatccatctgcctatggataaggaaaagagaacggtcgtaattctcatagactcctttctggttgtgtcacaaatggcttcacatgtttctctatgctcagagatactcagcttgatttcccgtgttttcatttcagcaccgactgagcaaaggcctggggtgcaggagtgctaccatggtaatggacagagttatcgaggcacatactccaccactgtcacaggaagaacctgccaagcttggtcatctatgacaccacactcgcatagtcggaccccagaatactacccaaatgcgtatgtctttgttctttaccataagagaagaaagggccaagtgaagtttctgttacaagagatgtgtctcaagctgagttctccgaactcaacttgtgacagatgcagatggcgtagcaaaatgtctcaggatgattgccttggagctaagggtctgagagaagggaaatgttaagctccctctccttcctcctagttctattgagcagaagggaaatctggaggtgaggagatcacattatgaagaaagtcagaatgacaaaggaccagacacttagattacccttccacaacaccaactaaacgtcaatggagactttccagttggaattccgttattctggcttccacttcctgaagggaaggttgcgtttgccttttctctctgggttcaagaggaaagaataggtgcttatttatggacaggtgaattgatctgtttctatatctacgtatattccgattgtcagaaaaacactcgttcctaagtaccagtggcctgaagggatacaggttcccagcaagagaagatccaaggaaggaaggcagatgagagtcagcacagagagggatgctgaaaagtaaaagggatgggtggatggagagaagcccgggtctgaccacccaatggccaatattttggccacaagcgactaccagagacatggaaaaatggtttctacatgtgggacaacagatggtagaggacctagagaattgagagaggggcaatgatgggctccactccgcagatgccttggctttcttcctggatacccttcctgcactgaatagcaaggagatggagcccaagcagactgtagccatcttgctgaatggaggagagggattggagtttgggatgactgtggtagctgaaatttttctaggtctgctagaaataagaactggtttgtgtggaggaaaagagctctacaaatacgcatagaagtctcctccagtcgttggcctgacatgacgctgcctgtgcacaggaaatggttccacgagaaagtgtggcaaagaacatttactgagaaacagcaagtacaagagcacaggaagctcaataaagaagagagagatcacatagcactctgggatactggagttcttcccagctagaccagagagtcctcacggagcacattgccaattcagtggagaccccagaacagccgtaatttaaaggtacacttagaatattactagaataaagtcagctgcagacaaccccttgcacagctggaaagcaagtgtccaagcatcaaatcggtttccaatcaatgaagtgcctgtgagaggaaatctcaactctctttagaagtaaacaacaaagtcgattgcctcagctatgcggtatccgcagagtgagtcctaaatttaaaatctgactacatgtagaaaagcgtttcgtgtgacccatgaccaggaaataaatcgggtaatacaaacaggctcaggaatgagagaaatgattagaattgcgtgaaaatttgacatatcagtatgataactgatttcaaatatttaaaaaaacaacatgcaagaaagcagatatcatatcaagagaaattaacagtacagaatagccaaattaaattaaagagctagtataaaaaaagtatgtcttaattgaaaaaaattactgtatggccggctgatcaaattagacgtttcagaggaaaacattacccaacacacaattttagagaacctacagaatgagctacacacacacacacacacacacacacacacacaaactgaaaacacacccatactcacacacacgcagaaactcacaagttctaacacacacagacacgcgcacccctgaagaaacagtgaaatataaaattaagcgagcctcacagacatgtaggaaaatatgaaaagatttcctgcatgtgggaagcaagtcacagtaaagagcaagggagtttataatagaaacaaataccagaatcaaggatggctgataacttttcaattacgaagaacattaaaaaaaatcacagaatcgtgaaactcaagggatcatatagggaatttcggaaaaaaaacccaacctgtatgatgtacttttgtacatcacagttcgaaggtaacaaggcaaagatgtaataagaagaaacctgtcacgagaaactggaggaaaaagagctgtgtcttcctacaagtacactgatacaaattgccaatgtgttcacctcagaaacactggaagccagataccagggaatattgttaaaatgataatcaggaacaaaaagagatcaaccgggaatgctgaatccagcaataaaatgccttgaaggtcatccatgtcggataaatgcatattgtgcactgccccaaagaaagaaaccggaaactgtaagaattggaaatcagcaggcttatgtaacaagagaggtgacccgaaggaattaggtagaagaagaattgaacaagaaaggaactttctgcagcccacgtaatgaagaatccagcaattggcaaatgtagatagatgtaaatgcaaaatattttcttgatcaaatttctatatctttgtaaatgagagttgactacttgaaacaaaatgatagcaagatatttaacttcagcatatgtagaggtaagaatttgaaatggtagcataaatcacgaagggattaattcgaagtgtaccgttgtaagtttctttacctcatgcacgatggtgtgtcatattaataaaagggtactgtgcgggttcgaagggatattgcaaatcctagagcaatcacaaaggtttgaactctgaggtttttggtataataagaatagtccatgcattcaaaagagggaagccaaggaagaactagaagtctttcaagagctcaggctcttatacatccagttgctcattgaaccagcttcctggaatggagggtctggggttgagactaggccacaagtctagagtctctagagagacagtgttggaaccccatggcccataatacatttcccattttctcaggcagccagaggtcatgaatgtgaggatactgggaggttggagcaacgttcttgggaggcataaggaagagcgaatgcttcaagatccccgcagcccaaactactcgcctgctttgccccctaatgcatttttctctgctgctccgtagctgtccgacctcttcagatctcttagtccaccctgccgtcttcctttatgccatgggtcccactgttctttcaactcatccccctttccctcagtcccggagtagctgcggccagcagagggtagactgagagcaggagagaaggacctgcctaggaaccccttctagagatactgcatcctgcctgggagcaagttttccagggcagctttgagaagtcttggagaaacaaacctactaaacctgacagacagtaatactatttgcacaatgcttttctgtgggaaaggtagagccttttcactacgtattgagtacatagagtgtgagggttgacctggaacggctatcctcctggatgacgtgcgttttctgaagaactacatgttcgttgcaactcccacattagaatatgaagtcctaccgagagagatacggagactagacagatacagatgcatttgcatgtgaatacacaatcccacaatacagacgtcaaaacccataccagttattccagagagatggattgggcagaaggcagaaggagaatactctgatcgtttttcggccacgtgtgtgtgttatctcagtgtttctaagaagcgtttgctactttagattttttatttaaaaaaaatagtaataatctattaagtatgagagatgtgcagagacgattagtgatcgagagccatttttgctggtggcaatcatatggtacttttaatgggaatattagaaaggcaccggtaatgaccttgttgcagcacaaaggagagagtgtggggtgcccctgcatgttgtcccacctcttgtgacgtgtatcgttttggaatttccagtggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaaggtaaggagtctgtggccagacatctacacgcttcgatgctgggatgaaaagccatggaaattcccactgatgcagccgccttcaatggtaaacggatgctcgagtgttgcctgagttctaccatgtaggaggaagcctccgtgcactctctgggggagccagcggagtgatttctggtgcaacgtggttgggctttgtctttaggatgggcacaaaccctccagggggatcgacttcaaaattcaccttgttgtaaaacgggctacctcagtgtcccagccaaaatttttattgtaacatgctgtcaggtgtgtcactctttccaagccagtaagcttttccggggatttcttcaagtagccagcattcagagcaatcttcagcattgcagattctgagaaatgtggctctggagcctgtcatcctcgagaaacctaacagggctgcattaattccatatggtcctgggtctatggagcagtatatgagctcccaatgctctaaggctcttcagtcctaggctttgaagggagtgatttctcagtgttcttaaacctctttctgatggcacttgtacctgtgaggggtctagagagaaaggttagtagacttctcctttactgcaattcaggatgcagggcatgagaagattccctccctcctccaagggaagaaggttttggcgtgcacacatccttgagaagcaaagtgtctttgccttcagtcagatatataggatcgttttctgccccatggcctggaagccagaggccttggctttcatgatcaacgatctagggaaacatgcaaaatttccatgtctttcccctcctctgccctcgacagccaattaccacctgcatcctgcattgccaaatgcagtgccctttgtatgaacattcagtagagtttcatagaaaggtgctacttcgtgagcgcactttgcagtgagaaggagtctgttctgttctgtttttctaaggatttcaggtgaaatatttcctagaacttactacagttctagattggtaggaatctgtaggtttgctgtatgttttttggttggttttctcccatccatctgcctacaggtaagggaaagataacgttcataattctcatagactcctttctggttgtgtcataaatggcttcacatatttcgttattctcagagatactcagtttatttcttgtgttttcatttcagcaccgactgagcagaggcctggggtgcaggagtgctaccacggtaatggacagagttatcgaggcacatactccaccactgtcactggaagaacctgccaagcttggtcatctatgacaccacactcgcatagtcggaccccagaatactacccaaatgcgtatgtctttgttctttaccataagagaataaagggccaactgaagtttctgtgacaagagacatgcttcaagctgagttctccgaactcaacttgtgtcagattcagatggtgtagcaaaatgtctcaggatgatttccttggagctaagggtctgagagaagagaaatgttaagctgcctcaccttcctcctagttttgtggagcagaagggaaatgaggaggcgaggagatcaccttatgaagaaagtcagaatgacgaaccaccaaacacttagattacccttgcccaacacccactaagcgtcaatgaagactttccagttggaattccgttattctgacttccaattcctgaagggaagattgtgtttgccttttctgtctgggctcatgaggaaagtttatgtgcttacttatggacaggtgaattgatctgtttctatttctacctgtattccaatagggagaaaatctcttggtcctaagtaccagtggcctgaaaggatagaggttcccagcaagagaagatccaaggaaggaaggcagatgagagtcagcacagagagggatgctgaaaagtaaaagggatgggtagatggatagaagccctggtctgaccaccccatggccaatcatttggccataatcaacaaccaaagacatggaaaaatggtttctacatgtgggacaacagatggtagaggacctagagaattgagagagggccaatgatgagctcaactccatagatgccttggctttcttcctggatacccttcctgcactgaatagcaaggagatggagctcaagcagcctgtagccatctagctgagcagaggagagggattggagtttgggatgactctggtattttctaggtccgctacaaataagaactggtttgtggaggaaaggagctctacaaatacgcatagaagtctcctccagtagttggcctcacatgacactgcatgtgcacagaaaatggttctacagaaagtgtggcaaagaacatttactgagaaacagcaactacaagagaacagcaagctcaattaagaagatagagatcacatagcactctgtgttattggagttcttaccagctagatgagagagtgctcacggaacacattgccaattcagtggagaccccagaacagccataatttcaaagtacaattagtatattactagaataaaggcagctgcagacaaccccttgcacagctgaaaagcaagtgtccaagcatcaaatgggtttccaatcaatgaagtgcctgtgagaggaaatctcaactctcttcagaagtaaacaacaaagtcaattgcctcagctatgcggtatccccagagtgagtcctaaattaaaaatttgactacgtgtagaaaagaatttcgtgtgatccatgaccagaaaataaatcaggcaatacaaacaggctcagaaatgacatcgataattagaattgcatgaaaatttgacatatcagtatgataactgatttcagatatttaaaaaaagtgcaacaaagcaggtatcatatcaagacaaattaatagtatagaatagccaaatcaaattaaagaactattatacaaaaagtatgtcttaaatgaagaaattactgtatgtccgcctgaaaaatttagatgtttcagaagaaaaaattaaccaaaaacaattctgcagaacctacagaatgagccacacacacacacattcaaaacacacccatacacacacacatgcaaaaactcacaagttctaacacacacacaaacacacacacacatgcacatccctaaagaaatagggaaatataaaattaaccgaccctcagagacatgcaggaaaatataagaagatttcctgcatgtgggaagcaagtcacagtaaagagcaagggagtttggagtagatacaaataccggaatcacggatggctgataacttttcaattatgaagaacgttagaaaaatcacagattcatgaaactaaagggatcaaataggaaatttcgagaaaaaaaactacatgatgcacttctctacatcacagttcaaaggtaacaaggcaaggatataagaagaagaaacatctcacgagaaactggagaaaaaagagctgtgtcttcctagagtacagtgatacaaattgctaatgcgttcacctcagaaacactggaagccagataccagggaatattattaaaatgataatgaggaacaagaagagatcaaccgagaatgctgaatccagcaataaaatgccttgaagatcatccatgttggataaatgcatattgtgcactgcccaaaacaaagaaactggaaagtgtaagactttggaatcagcaggcttatgtagcaacagaggtgacccgaaagaattaggtataagaagaatagaagaattgcatgaaaatttgacatatgactaagataactatttcaaatatttaaaaaaagatgaatatgtaataaaacagataaaatatcaaaagaaagtaacagtattgactagccaaatcaaattaaagacttagtgtaaaaagctatgtcttaaaagaaaaaattactggatggctgcctgatcaatttagacatttctgaataggaaactaaccaaaaatcaattctacagaaccaactacacacatatatacacatacaacacacccatacacacccacgcaaaaactcacaagttcacacacacacacacacacacacaaccctcaagaaatagtgaaatagaaaaccaaccgaacctcacagacatgttgcaaaataggaaaagatttcctgcatatgggaagcaagtcacagaaaagagaacgggagattggaaacagaaacaaataccggaatcaaggatggccgaaaacttttcattgatcaagaatattaacaaaatcgcaaaaacacgaaattcaatgcatcaaataggcgtttcgaaaaaaagaaaaaatctggtatgatgcacttttgtacttcacattttcacggtaagaagacaaagatataataacaagaaacttcttatgagaaactggggaaaaacaagctgtttcttgctagaagaacagtgatacaaattgctaatgcattctcgtcaaaaacactggaagccagataccgggaatgttattaatgtggtaaacaggaacaagaagagatcaaccaagaatgctaaatccagcaataaaatgccttgaagatcatccatgctgcataaatgtatgttgtgcactgccccaaacaaagaaaccggaaactgtaagaatttggaatcagcaggctgatgtaacaagagaggtgacccaaaggaattaggtagaagaagaatagtacaagaagggaactttctgcagcccatgtaatgaagaacccagcaattggcaaatgtagatgtaaatgcaaaatattttcttgaccaaatttctatatatttttaaatgagcgttgactactggaaacaaaatgatagcaatatatttaattttagcatatgtagaggtaagaatttgaacaagtagcgtaaatcatgtagggaataattagaagtgtaccattgtaagtttcttacctcatgcacaatggtatgtaatattaataaaatgttactgtgtgggttcaaggagatattgcaaatcctagagcaatcacaaagttttgaactctgaggtatattgtataataagaatattccatgtattcaaaagagagaagccaaggaagaaagaaatttgtcacgagtttgggctcttagtacatcctgtagctcattgaaccagcttcctggaatggagggtctgggattgacactaggccacatgtatagagtctctagagagacagtgtttcatccccatggcccgtaatacatttcccattttctcaggcagccacaggtcatgaatgtgaggatagagagaggttggagcaacgttcttgggaggcataaggaagagcaaatgcttcaagatccccgcagcccaaactcctacctgctttgccccctaatgcagtgttcctccgtagctgtccgacctcttcagatctcttagtctaccctgccatcttcctttatgccatgggtcccactgttctttcaactcatccccctttccctcagtgcagagtagctgcggccagcagagggtagactgagagcaggagagaaggtcctgcccaggaacccattctagagatgctgcattctgcctgggagcaagttttccagggcagctttgagaagtcttgcagaaacaaacctatttgacccacatgatatgggaatgacagaaagtaatacaatttgcacagtgcttttccatgggaaaagtagagccttttcgcgaggttttgagtacatagagagtgaaggttgacctggaaaggttatcctcctggatcccatgttttttctgaagaactacctgttagttgcaacttgcacattagaatatgaagtcctaccgagagagatacggagaactagataaatacagatacttttgtatgtgaataaacgattccacaatacacacatcaaaatccataccagttattccagagagatggattgggcagaaggcagaaggagaatactctgatcgttttttgcccacgtgtatgtattatctcagtgtttctaagaagcgtttgctactttagatttttttttataataataatcttttaagtatgagaaatgtgcagacaggattagtgattgagagccatttgtgcttgtggcaatcatatggtacttttatgggaatattagaaaggcactggtaatgaccttgttgcagcacaaaggagagggtgtggggtgcccctgcatattgtcccacctcttgtgacgtgtatcgttttggaatttccagtggcttgatcatgaactactgcaggaatccagatcctgtggcagccccttattgttatacgagggatcccagtgtcaggtgggagtactgcaacctgacacaatgctcagacgcagaagggactgccgtcgcgcctccaactattaccccgattccaagcctagaggctccttctgaacaaggtaaggagcctgtggccagaaacctacacgtttcgatgctgggatgaaaagccatggaaattcccactgatgcagcagcctccaatggtaaacggatgctcgagtgttgactgagttctgtcatgtaggaggaagcctccgtgcactctctgggggagccagcggattgatttctggtacaacgttgggtgggctgtgtctttagaattggcacaaaccctccagggtgatcgacttcacaactcacctcgttgaaaaatgggctatctcagtgtcttagccaaaatttttattgtaacatgctgtcagatgtgtgactctttccaagccagtaagcttttcctgggacttcttcaattagccagcattcagtgcaatcttcagcattgcagattcagagaaatgtggctctggagcctgtcacccttgagaaacagggctaacagggttgcattaattccaaatcaccctggttctatggagcagtacatgaactcccaatgatctatgtttcaggacttcctcagtcataggtgggctctgcagccctaggtttttaagtgagtgactgccccgtgttctggtggcagttgtacctgtgagcggtctggatagaaagagtcggagacttctgtattattgcaactcaggatgtgggtcatgagaggatttcatctctcctgcaggggagtaagctgttcgcctccacccatccctgataactgaagtgtctttgtctgcagtcctagacgaaggactgttgtctctcccatggcccagaagctgaagaccttgccttttgttatgaaacgttcattgttttcatgtctgtccgtttctctgcccctaacacccaatcaccatgtatggcctgtacccccaaatgcatcgtgctttgctgtttgctgccccatagtcctcatgaacattcagtagaaattcccataaatgtgcttgcacgtgagcacagtttccattgagaagccctctcatttgtcctttttttctaagcttttatgtgaaatatttctaagaacttactacagttctaaagtgttaggaatttgtttctttggtgtttttgtttgttggttggttgttgcttttctcaagtccatctgcctacaaataaagaaacaagaatgttacttgtcatattctcctgaggtcataattctcagagacttttttctggtttgtgccataagtggcttcacatgtttgtctcttcttggaaacactcagtttgatttcttttcttttcatttcagcaccaactgagcaaaggcctggggtgcaggagtgctaccacggaaatggacagagttatcaaggcacatacttcattactgtcacaggaagaacctgccaagcttggtcatctatgacaccacactcgcatagtcggaccccagcatactacccaaatgcgtatgtctattttctttaccataagtgaaggaagggtcagtggaaatttctgttagtagagtcatgcttcaagctgagtgttcaggactcaagttgtctcagatgaacagtgcatagcaaaatgtctcaggaacattgtctttgagcaaagagtctaagagaagacaaatgttaatctggctctccttcctcctagtttaatggagcagaaaggtatctggaggcaaggatatcacattaagaaacaagtcaagatgacaaatgatgaaactcttagagtacccttccacaacacccactaaggttcaatgcagccttttctccttggaattctattaaactaaactccaattcctgaagtgaaggttctgttggggttttctgttttggcttacaaggaaagtatatatgtatatctatggagaggcaaatctatctctttctatatctacgtctattccaatatgtagaaacacagtcggttctgaccaccagtggtctgaagggatactggttgttagagaataaaaatggcaggaaggcagatgagagtcagcaaagagagagatcctgtaaagtaaaagggtggatagatggacagaagcccaggtctgaccagcccatggccaggctttaggccataagtgacaccaaagacatggaaaaatggtttctacatgttggacaacagacagtagtggaccaaaagaatagtgacagggggaacaatgagatcaactccatagataccttggctttcttcctggaggcccttcttgcactgaagagcaaggtgatggagcccagatggactgtagccatcttcctgaatgcaggagagagattggaatttgggactactgtggtagctaggattttataggcctgctgagaatgagaatggatttgtggatgaaaggagctccaggggcacgcatagtagtctcctcgaatctttggctaaacatgacgttgcatgtgcccagaaaaaggttccacaagaaagtagagaaaagaatatatcctgaggaatagcaactgcgattgaacagtgagctcaataaagaggacagagccctcatagcattctgggatactggagttctgaccagctggaggagagacctcactgaacctcttgggaatacagtagagactccagaaaagtcatactttaggagtagaattagtaaatttctagaaaaaaaggcagctctagacaaaccctggcaaaactgaaaagcaagtctccaagcattaaaatcatttccaagtcaattaactgcctgggagaggaaaaccctctttagaggtaaacaacaaagtcaagtggctcagctatgtggtgttcacagtgtgagttctaaatttaaaacttgactacacatagagaagcttttagtatgaaccatgaccaggtgaaaaatcagtcaatacaaatagacctagaaatgacagaaatgattagaatggcaaaaaatttgacatatcaatatgtcaactgagttttaggttttaagaaaacatgaatacggaatgaagcagataccatatcaagagacagtaacagtatagaagagccaaattaaattaaagaactagtataagaaggtatgtcttaaatgaaaaaattactggatgtattcccaatggagtgagatgtttcagaagtaaaaactaactgaaaaacaattttataccacctacagaaccagctacacatacacaaatgacacacacatatacacacatactcacacatgcacaggcttagaaacatgcacgcacacacacacacacacacacacacacctccacaaatactaaaaaatgaaatccactgatcctcacagacaggcgggaaaatataaaaagatttcctgcatgtgggtaggaagtcacagaaggagaggaaggagagattgctacaggaacaaatactggaagcaaggatagctaaaaacttttcaaataagaagaatattaaaaaccacagattcaagaagctgaatgaatcagacagggaatttccaaaaaaaaaaaaaaaaaaactgtatgattcacttttgtacatcaccgttcaacagtcagaaggcaaagatataataacaagaaacatctcatgagaaactggaggaaaaagagctgtgtcttgctagaagaacagtgatacaaattgctaatgcattctcatcagaaacactggaacccagttaacaggggatatcattaaaatgataaactagaaaaaaaagagatcaaatgagaatgctacatccagcaataaaatgccttgaagatcatccatgttggataaatgcatattgtgcactgccccaaataaataaaccaaaaactaataatttggaatcagcaggcttgtgtaacaagagatgttgcccaaagaaaattagctagaagaagaatagttcaagaggagaactttctgcagcccacgtaatgaagaacccagcaaatggcaaatgtagatgtaaatgcaaaatattttcttgatcaaatttctatatctttttaaatgagagttgactacttgaagcaaaatgatagcaatatatttaactttagcatatgtagaggtaaaaatttgaacatatagactaaatcatgtggggaataattggaagtgtaccattgtaagtttcttaccttatccacgatggtatgtaatattaatgaaaggttgaatttgtgggtccaaagggatattgtaaatcctaaagcaatcataaaattttgaattctgagggatattatataataagaattttccatgtatccaaaagagggaagccaaggaagaaaaagaagtctttcaagtactcaagctctgagcacatccagttgctcattgaaccagcttcctggaatggagggtctgggcttgagactaggtcacatgtgtagagtctctagagagacagtgttggatccccatggcccataatacatttcccgttttcccaggcagccacaggtcacgaatgggaggattctgagaggttggagcaatgttcttaggaggcataaggaggagtgaatgctctgagatttccccagcctgaggtcctccatagctgcccgacctcttcagacctcatagtctgcccagctgtctccctttatgccatgagtgccactgttctttcaactcatcccccattccctcagtcccggaattgctgtggccagcagaggatggactgagagcaggagaggaagtcctgaccaggaacccatcctagagatactgcatcctgcctgaaagctaggtttccagggcagctttgagaagtcttgcagaaagaaacccacttgacccacctgatacggtatcgacagacaggaatactttttgtgcaatggttttacatgctgaacatagagccttttggctacattttgagtacattgaatgagactgctggcctgggaaggatatcatgctggatgccatttttttctctggagaactatgtgttagttccaactcgcacattactatatgaagtcctacacagagagatacggagagctagacagatagagatacttttgtatgtgcataaccaattccacaatacacacgtcaaaatccataccagttattccagagagatggattgggcagaaggcagaaggaggatattctgatccctttttggccacatgtatgtataatctcagtgtttctaggaagtgtgtgctgcattagattttttttctttaaaaaaagtgataatatattaagtatgagaaatgtgcagagaggattagagattgagagccatttgtcattgtggcaattgtatggtatctcttttgggaatatttcaaaggcaccagtaatgaccttgttgtagcaaaatatacagtgttcctgcatatgtacccattttttgtgatgtgtattcttttggaatttccagtggcttgatcaagaactactgccgaaatccagatcctgtggcagccccttggtgttatacaacagatcccagtgtcaggtgggagtactgcaacctgacacgatgctcagatgcagaatggactgccttcgtccctccgaatgttattctggctccaagcctagaggctttttttgaacaaggtaagaagttgtgccagacatttacctgcttggatgctgggatgaaaagccatggatacccccactgacgcacaacccttcagtgctacactggttctcgtgtgttggttctgggtctgccatgtgggaggaagccttagcgcactctctgggggagccagaggtgtgatttttggtgcaacctgtgcgagctgtgtctttaggatgggcggaaaccattctgggtgctcgacttcaccactcccctcattgtaaaaggggctatctcattgtcctagacaaaattcttattgtaatatgctgtcagatgtgtgtgtctttccaagccagtaaacttttccagggatttcttcaagtagacagcattcagtgcaatcttcagcattgcagattccgagaaatgtggctctagatcctgttatccttgagaaacctaactgggttgcattaattccatatctccctgggtctgtggagtagtacatgagctcccgaagctctatctctcaggtctttttcagtccgaggcaggttgtgcagttcttagctttgaagggagtgattttttcgtgtgcttttgcctctttctgatggaacttgtacctgcggggggtctggagaaaaagagtagtagacttttgctttattgcaatgcattatgctgggcacgagaggattccctatcttattgtaggtgataagcttttggcctccactcatccctgagaagtgaagtgttgttgcctacagttttagctgcaggactgttgtctgccccatcaccaggagtttaatgctttcttttttgagcaatcatctagggacacatgcaaggtttttatatgtccttgcctcctccccaaaaaaccattttaatgcttggagacttgcttttcagctttgccaaatgcatcaccctttcttctatgctgttccatgtcgtcatgaacactctgtagagattcctagaaatgagcttccatgttagtggagtttccgatgagaagcaatctgatatttcttttccactaagttttacatgaaatatttctaagaacttactacagttctagaatggtaggcatctcttactttcgtgtttgtttgtgtgttttctcatgtccatttgcctattaataaagaatagagaatggttgtaaatctcagtgactcttttttggtttatgtcataaatggcttcctgtatttttctgttctaggaaataataagcttgatgtcttctgttttaatttcagcactgactgaggaaacccccggggtacaggactgctactaccattatggacagagttaccgaggcacatactccaccactgtcacaggaagaacttgccaagcttggtcatctatgacaccacaccagcatagtcggaccccagaaaactacccaaatgcgtacgtctttgttctttaccataagcgaaggaagggccaatggaagtttctgttagaagagtcatgcttcaaggtgactgctcaggactcaacttggctcagatgcagaggaacatttcctgtgagcaaaagttcttagagaagactttgtttttttgagacagagtcttgctttgttgcccaggctggagtgcagtggcatgatctcggctcactgcaagctccgcctcccgggttcacaccattctcctgcttcagcctctctagcagctgggactacaggcacccaccaccacacccggctaattttttgtatttttagtagagacagggtttcactgttctagccaggatggtcttggtctcctgacctcgtgatccgcctgcctcagcctcccaaagtgctgggattacaggcgtgagccaccgtgcctggctgagaagacattttttaagctggctctccttcctcctagttttatggaagcagaaggatatatggagttgagaagatcttattaataaaacagccgggatgacaaatgaccaaagagttagagtatccttctacaacatcggctgagggttaatacaaccttttcaccttggaattctatcattctaagctctagtccctgaagtgaatgttgtgttggccttttgcatcttgggtcacagggaattgatacttgcacatctatggagaggcaaatctttttctatctacttctttttcaatgggtacaaacacacttggtcctgagcaccagtggtctgaagagatacggtctgcccagaggagaagaacaaaggcaggaaagcagatgagagtcagcaaaggggcgatgctgaaaagtaaaaggggcgggtagatggacagaagccatgatctggccattctatggccagtctttcggccataagtgactaccaaagacacggcaaaacggtttccacatgttgaacaacagatgctagaggaccaagagtattgcaagagggagaaaatgagatcaacccatcaatgccttggctttcttcaaggagacccttcctgcactgaagagcaaggagatggagcccaagctgactgtagccatgttgctgaacagaggagagtgattggactttgggattactcaggtagttaggattttctagccatgctaagagtaagaatggacttgtggaggataggagctccaggcatagaagtctcctcaagtgttagtctaaacataaagcagcacttgcatagaagattttccacaagaaaatatggcaaaaaaacaccatatattgaggaacaacaactacaagggaacagtgagcttaataaaggtgacagagctcacatagtgctctggaatattggagttttgaccagctagagagaagagacctcattgaaaatcttgggcattcagtagagacctcagaaaagtcagactttatgagtagactttgtatattcctagaataaaggcagctccagaaaaaacctagcaaagctgaaaagcaaatctccaagcattaaaatggtgtcctagtcaattaactgccttctagaagaaaactcaacactctttacaggtgaacaacaaagttaagttgctgagctatgcaatatccacagtgtgagtcctaaatttataactttactacacataaaaaagcatttagtgtgaaccataaccaggaaaataatcagtcaataaaaatagaaccaggaatgatagaaatgatttaaatggcatgagaatttgacatattagtatcataactgcattgctggatttaagaaaacataaacatggaacgtaacagatatcatatcaagggaaagtaaaaggataaaagagtcaaatcaaattaaaggactattaaaaggtatatcttaaatgaaaaattcactggatggtctcccaatcaggttagttgtttccagggaaaaaattaactgaaaaataattcaatagaatctacagaaatagctgcacatatatacacacaatggcacacgtgcacacacccacacccacacaggtgtgaatcctagagccacacgagcattgaaacatagagaagtaaaaattgttcattgaggaatatgtagcaatgctcaatgtgttttaccctaataagagcttttgtgatgtatgattgaaaaactgacacaactgaagagagaaatagataagcccacactctgagttagagatttccttgattctctcactatggttataaatctttcccaaacacaacaggctagaacaaatatgcagaaaattagacatagtatctttgttctcaataaaaacgtcgacctatttaacattataccgaactaccgagtacacattaaagtgtgcatggagcattcactgaggtgtactctacacatgaccttccagcaagtctccatagatttaaaagaattaaagtcatacagagtgtgtcactttattctcccagaataaagtgagatatgaataatgagaagtttgccagcttctcaaatatttgggagtcatacggtgcatttcaaaatactctttgggacaaagaaaacatcactaaggaatttagaaaagttttgaactgagtaagaatataacacaatttatccaaacttaggagatgcagtgaatgtctttaggcttttacataattttagatgctcttagggaaaaacagaagcatgtaataatcaagatttcaaactgcaattctcaaagtgtagtctagagaaacctgaggacctttgagtaccttcagagacagtccatgaggttaaaggactttgctacgtgaaaagtaagatgctattggccctttttactttcattttccaacaagagaagaggggagttttccagcagttacataatatgtaatggcatcatgtctctgatggctaagaaaatgggcaattgttgactttgtgtgttaaaaaaattctcagtgttggtttcttatactataaatattcatcttgtgttttgaaaaagaaaagctctttggaatcccctatgaacaaagactttgacagttgttgatctaagaccacagcttaaatatctacacaagaaaaaaaaaaaaagcaaataagagccaaggaaagcagatggaaggaagtagtccaaaccagtgacattcagtgaacaagaaaagagaccaacaagggagtaaactcttgaaacagaaagttgattctttgaaaagatccatatgattgaacacagtctggctaaacaaatgacagaccaatgagggtgcacaaccatcaccatctggagtaacagaggagaggtgccattactatagcatcttccagttctgaaagctgaaaagaagattttgagaacaattgtatgtgaataaattcaggaatgttaatcatgtgggccaattcctgaggaagacaacaaatcagcaaaccagatgctgaatagttagtgtagtcctgtagagagacatacagagaggctgacagagaaatatttgtatgtgcataaaacaatctacaagacacacttcaaaatcaatctcagttaatctggaggaacatatttcacagaaggtggaaggagggtattctgatcctcttgtacattgtacaacattgtacaatgtacagagtataattgtacaagtacaattgaagttgtacaagtacaagtgcaacttgcacaatgtacagagtaaacattgatgtttactctcaattttcttatggagcacagatgactttggatgtgttacaatatgaatgataatttgtctttgagatgttcgcagttgtttagaagttgaggaccatttgtgcatattatgggacctttagtgaaaatatttcaaagtctctttttacactttgttacagcaaaatgtagagggcgctaagtgcccttgaatcttctcccatctctggtgacctgtgttgttttgaaatttgcagtggcctgaccaggaactactgcaggaatccagatgctgagattcgcccttggtgttacaccatggatcccagtgtcaggtgggagtactgcaacctgacacaatgcctggtgacagaatcaagtgtccttgcaactctcacggtggtcccagatccaagcacagaggcttcttctgaagaaggtaggaagtctatggccagacaaccacaccctaggacgttgggatgaaaagagttgcaaaatcttagtgatatagaagccttccatgctcacacaattccaagtagaatgtggactcagggtcagccactgggaaggaacactcagcgccttctctgggagaaccagagctgtgatgtttggtaccctgtgaaagggtggtatctataggaagggtgcagaccctctagggcactggacttaccactcccctggttattcaaaggatcattttagtgtcttagccagaagaatattctaacattttgccaaatttgtgaagatttaccaagctcatgataagcctttcatggtatttcttcaagtagtcagtgttcattgcatctttggctttgcggtttcggaggaatgcggtttttgagtctgtcatccttgagaaacctaatatgacttttcttagttccatatacttctgggtccaggtagcagtacatagccaacaaatgctccatcgttctggcctatctccatcttaagccagtcctgcacaactaggctttgatgggagggatctctcagtgttcttgcccctccttctcatggaacatatatctgtgttggtctctgagaagaagagtagtggatatctactttgttgcaatgcagaatcctgggccaaagataccagccatccctccaagggaataaaattttggccagtagccctctctgagagacaatttgtctttgcctacgagtcctagatgcaggaccgcttcctgccccatcttcaagaagctgaaggctttggctttggaggatcagcagtctagggaaatgtgtgacggtttcatgtctgtccccactgacagtcaatcaccacctacaacctgcacagcctgatgcatagcagtctagtttcctgccttattctcaggaacacccagaagatgtctatattaaagagcatgcacatgagtgcaattttgactgataggcactctgatctttcctttggtgcctgtgttttaaaggaaatctttctaagaactcgttaaagttctagaatgctatgaatctttgggttttattattggtatgtccatctgcctgctagtacagaacagagcatggtagtctttctcagagacaatgatcctgtttcagtcacagatttcttctgatgcttctgtgttctagaaattactcagcttgatttctcctctttgaatttcagcaccaacggagcaaagccccggggtccaggattgctaccatggtgatggacagagttatcgaggctcattctctaccactgtcacaggaaggacatgtcagtcttggtcctctatgacaccacactggcatcagaggacaacagaatattatccaaatgggtacaaccttgagttttcttcaaagacagacagcagcccccttacatttctcttggaagggccatgcttccaactaacttcttatgacaaatttatctcagatctggaatgttgggtagaatgtctcaggcttctttcttcaggcacagtgtctgaaaggagagaaatgtcaggccagctctcttttctcatagttgacagaagcaggaggatatttgaaggtggtgagttctcatgaatagaaagctcaggacacatggccacgtgcttagaaatagcaccattccacaatgcccactaaagaccaatgcaatagttcaaccagggatttctgtcattctaatctccaagtcctgaagtgaaggttgtattagccatgttcatcttgggcaacaaataaaggatatctatgttgacatccagatcttccaatcactttctcctctaacctgtacctgggttctgagaacaaggtatctgaagagctatgtgttgccagcacatgaggggcaaaagtaggaaggcagctgagagtcaggaagtataaagattctgaagagttacacatgcaggaagatggacagaaacccagttcagaccacgtcagcgtttctgccatgaaggactatcaaatacataggaaaagtgttttcataggttggacaacagacatgacaggcctgagaaaattcagaaagggaatcaaaggagatcaaccttatcatgtccctggcatccttccttgagacccttgaagggcaagcagatggagcccagctgaccacagcagtcttgcttaactgaggagagagactggagtttgtgatgcctcaggcatctgacgtattctaggctggctaagaatgagaggggatttgtggaggaaaggagctccaagaatacacaccgaagtcttctcaaggctttggctaaatacaaagctgcgtatgcacaaggagagttttcacaaagaaagaacaataaagaaaagctactggggaaagaacaactgcaagggaacagtgagctcaatggagatgctagagctcacatagcactgggggatatttgagttctgaccactcagaggagagacacctcactgaacatcttgggcattcagtagaggtcaaagaaagccataatttgggagtaggatcttcggattcctagaaataaggtgactccagaaacactccagcaacccttcttccaagccagtctaaaaggatccaaatgatttccaagtaaattaactgccttccagaaaaaagtaaactcaaccctccttagaggtaaggaacgaatacaagtttctcagttatatgacatccccagagtgcaacttgcatttaaaaatttactagacacaaaagaagttttcactgtgatccataactgggagaaaaatcactcaacacaaataggcccagaaataatagaaattatggcattggcaagaacatttaaaatgcacctctgagaactgtgtttcaggaaaatgtcagcaaaagctgaccatgagagaaatgaatgcataatatcagaaaagaaaagaattgaagagccaaatggaaatttaaaaactgagaaaagttatatctgtaatgaggaattcactggatggccttataaccagtttagatattatggtaggaaaaggtgaacgagaaaatgattcaattaaagctagacaaaccacaagacagacagacagacacaaatacacatacacacaatgactgaaccaattaatcaacagagcctcaaggacatctaggaaaacatccacacatttaatatatgtgttaggcaagtcacagaaagagaggaaaaagataatgtgacagaagttatacttgaagccatgacggctgacaaatttccaaacatacagaaaatgagaaattcatagtcatgaagctcaatgactcaggtatagatttttaaagagcaaaactctgatttactggggtacatcatagttaaattgtctgatttcaaagctaagaagaaaaaaagggggttcctatgaacaaacattttgacagttgatctaagaccacagcttaaatatctaggcaaggaaaagcaaataagacacaaggaaaggggatggatggaaatagtccaaaccaatgacattcagtgaacaagaaaatagaccaacaaaggagtaaatccatgaaacagaaagttggttctttgaaaagattcatgtgattgaccacagtctggctgaacagatgacagaccaaggagggagtacaaccatcaccatttgaagtaacaggggagaggagccattgctataccatactccaggtctgaaagctgacaagaagatatcaagaaaaactgtatgtgaataaattcatgaatgtagatcatgtggatcaattccttaggtaaacaacaaatcagcaaaccagatactgaatagattgggtactcctatagaaagacatacagatagccagacagagaaacatttgtacgtgcataaaacaatctacaagactcacttcaaaatctctcagttaatccaaagtaacatatttggcagaaggtggaaggagggtattctgatcctttcttgtacacattgatgttttctctcggttttcttatggagtatagacgagtttggatgtgttacaataagaatgataatctgtctttgaaatgttcacagttgtttagaagttgaggacgatttgtgattgttacaggacctttagtgagaatatttcaaagtcactttttaccactttgttacaacaaaatgtagaggatgtctggtgcccttgtatcttctcccatctctggtgaactgtattgttttgtaatttgcagtggcctgaccaggaactactgcaggaatccagatgctgagattagtccttggtgttataccatggatcccaatgtcagatgggagtactgcaacctgacacaatgtccagtgacagaatcaagtgtccttgcgacgtccacggctgtttctgaacaaggtaagaagtctctggccagacaaccacacccttggacgttgggataaaaagagttgcaaaatcttagtgatacagaagccttccatgctgcacgggaatctgaatgtggactcagggtcagccaatgggaaggaagcctcagcgccttctctgggggaaccagggctgagatttttggcaccccgtgacagggtggtgtctttaggaagcgtgcagaccttctagggcactggatttaccactcccctggttattcaatagattatttcagtgtcctagtgaaaatggatattctaacatcctgccaaatttgtgatgatttaccaagctcatcatgagcctttcctggtatttcttcaagtagacagtactcattgcaaacttcagctttacagtttcagaggaatgtggtttttgagtctgtcatccttgagaaacctgatatgactttacttagttccatatcctcctgggtctaggtaacagtacatagccagcaaatgctctatctccctgtctaccttaatcttaggcaggtgctgcacacctaggctttgatggaagggatttcttagtgttcttgcccctccttctcatggaacacgtatctgtgttgctgtttgtgaagaagagtagtggatgtctactttgttgcaatgcaggatcctgggcccaagatttcccgccgtccctccaagggaataaaattttggccagtacccctctctgagagacaatgtgtctttgcctggaagtcctagatggaggaccacttcctgccccatcttccagaaacttaaggctttggctttggaggatcagtgctctggagaaatgtgtgacggtttcatgtctgcccccactgacaaccaccacctacagcctgcaccgcctgatgcatggcactctggtctcctgccttgttctcaggaacacccaaaagagatctttgccaaagaacaggcacatgagtgcaattttgactgataggcactctgatctgtcctttggtgcccaggttttaaagaaaatctttctaaaaactcattgaagttccagaatgctatgaatctttgagctttgttattggcatgtccatctgcctactaatgtagaacagagcatggtcgtcattttcagagatgatgtcctgtttctatcatggattttttttctcatgcttctgtgttctggaaattactcagtttgttttctcctctttgaatttcagcaccaacggagcaaagccccacagtccaggactgctaccatggtgatggacagagttatcgaggctcattctccaccactgttacaggaaggacatgtcagtcttggtcctctatgacaccacactggcatcagagaaccacagaatactacccaaatgggtatgtctttgagttttctcccaagagaaacagccacccacttaaatttctcctggaagagccatgcttccagctaacttcttatgacccaatttctctcagacccagaatgttggacagaatgtctcaggcttcttgctttgggcacagggtctgagaggagagaaatgtcaggccagctctcttttctcatagttgatagaagtaggaggatacttggaggtggtgaggtctcatgaatagaaagctcagaagaacatatgaccatgtgcttagaaatagcaccattccacaatgcccactaaagaccagtgaaatagttcaaccagggaattctgtcattctaatctccaagccctggagtgaaggttgtgtttgccatgtttgtcttgggtaacaagtgaaggatatctatattgacttcgagatcttccgatcactttctcctctaacctgtataaacacattgggttctgagaacaaggtgtctgaaaagctatgtgttgccagcccatgaggggcaaaaggaggaaggcagctgagagtcaggaagtatagagatgctgaagagttacacattcaggaagatggacagaaacccatgtctggctatgccagcctttctgccatgaaggactatcaaatacatgagaaaacagttttcacaggttggacaacagatatggtaggcttgagagaactgagaaagggaatcaaaggagatcaacttcatcattaacctgtcttccttcctggacacagtgttggattgaaggacaagcagatggagcccagctgaccacagcagtcttgcttaactgaggagagagactggagtctgcgatgcctcaggcagctgatgtgttctaggctggctaagaatgagaagggatttgtggaagaaaggagctccaggaatacacacagaagtctcctcaaggctttggctaaatacaaagctgcgtatgcacagggagagttttcataaagaaagaacaacaaagaaaagctacttgggaaagaacaactgcaggggaacagtaagctcaatggagatgccagagctcacatagcactgggggatatttgaattctgaccactcagaggagaaacacctcactacattttgggcattcagtagagaccaaagaaagctgtattttgggattgggatcatcttattcctagaatcaaggtgactccagaaaaactccaacaacccttcttccaagccagtctaaaaggatccaaatgatctccaagtaaattaactgcattccacaagaaaaaaaaaactcaaccccccttagaggcaagggacaaatacaagttgctcagttatatggcattcctattgcgttacttctatttaaaaatttaatagagacacaagaagctttcactgtgatacataactgggagaaaaaatcactcaacacaaacaggcccagaaattatagaattgatgacattggtgagaacatttaaaatgcacctctgagaactgtgtttcaggaaaatgtcagcaaaagctgaccatgagagaaacaaaagcagaatagcaagagaaaagaaaagaaccggagagccaaatgaaaattaaagaactgagaaaaggtacatctctaatgaagaactcactggatggccttatcatcactttagacattacggtaggaaaggtgacctagaaaataattcaataggagctacacaaatcacaggacagacagacagaccaacagacagaaacacacacacacacacacacacacacacacacacacacacacacacacaaagactgaacctattaatcaacagagcctcaagggcatctaggaaaaatccacacatttaatatatgtgttaggcaagtcacagaaggagaagaaaaagatatcatgacagacattatacttgaagcgatgatggctcgcaacacgccaaatatacagaaaacaagaaactcatagtcaagaagctaaatgactcaggtatagaattttaaagagcaaaactctatgatttactgggatatatcatagttaagttgcctcaattcaaagctaaaaagaaaaaaagggggttcctatgaacaacagctttgacagctgttgatctaagaccacagcttaaatatctaggcaaggaaaagcaaataaggcacaaggaaagaggatggaaggaaatagtccaaaccaatgacattcagtggaaaagaaaatagaccaacaaaggagtaaatccatgaaacagaaagttaggttctttgaaaagtctatatgattggccaaagtctggctaaacagatgacagaccaaggagggagcatatccatcaccatcatgagtaacaggagagagatgccattgctatagcatcctccaggtgtgaaagctgagaagtagatattgagatcaactgtatgtaaataaattcatgaatgtagatcatgtggatggattgcttaggtaaataacaaatcagcaaatcaaacactgaatagatcatgcagttttatagagacttacagacagcctgacagataaacatttgtatgtacgtgaaacaatctccaagacacacttcaaaatccctctcggttaatccaaaggaatgtatttggcagaaggtagaaggagggtattctgatcctttctggtacacattgatgttttctctcagttttcttataaagcatagattactttgaatgtgttacaataagaatcataagctgtctttgaaatgttgacagttgtttagaagttgaggaccatttgtgagtgttatgggactttagtgagaatatttcaaatttgcttgtttacactttgttacaagaaaacatagagggtgccaggtggtgctgtatcttctccaatctctggtgacctgtattgttttggaatttgcagtggcctgaccaggaactactgcaggaatccagatgctgagattcgcccttggtgttataccatggatcccagtgtcagatgggagtactgcaacctgacgcaatgtccagtgatggaatcaactctcctcacaactcccacggtggtcccagttccaagcacagagcttccttctgaagaaggtaagaagcctgcagtcagacaaccataccctcggacattgggataaaaagatttgcaaaatctttgtgatgcagaaaacttccatgctgcacaggaagtcgaaggtgaagtcatggacagccaatgggaaggaagcttcagtgccttctctggggggaccagagctgggatgttgagtgccttgtgagggatggtgtctttaaaaggggcacagaccctctaggacactggatttatcacttccctgttatcaaacgaatcatattagtgtcctagccaagatggatattctaacatcctgccaaacttgtgaagatataccaagctcctaagcctgtccagccctttcttcaagtaggcagtgtttattgcagtcttcagctttaccattttgaaggaatgccatttttgaggctgttgttcttgagaaacctaacatgtcttcattagatccgtattgtcctgagactttgaagcagtacatagccaccaaattgtttatctccccagcctaccttcatcttgggcatgccttccacacctaggatttgagggaagggatttctcagtgttctcatccctgcttctcatggaacatttatctccgttgttttttgagaagaagagtagtggatgtcagctttcttgtaatgagggatcctgggcccaagattccctgtctcccctcctaggctataaaattttggcctgtactccttctccctgagaggcaatgtgtctttacctacaagtcctagatgcaagatccttttctgccccacaccccagaatctgaaggcttttgctttggaggagcagtggtctagtgtgcaagggtttcatgtataccccccactaacagccaatcaccacctatagcctgaacagcttgatgcatggcaccctggtctcctgccttgttctcatgaacacccagaagaggtgtaagcaaaagaccattcacatgagtgtaattttgaagtataggcactctgatctgttttttgtttgtttctttgtttgtttgttttccagggttgaattaaaatatttatgactacttattaaatttctagaatcctataagtctatttgtatttttattctacatttcaatttgcatgctaatatagaagagtgtaaattgttaatcctcagattattccactttgtgtgtcataatttttttcacatttcccttttctaggcaatactgagcttgattttctcttttaatttcagcaccaactgaaaacagcactggggtccaggactgctaccgaggtgatggacagagttatcgaggcacactctccaccactatcacaggaagaacatgtcagtcttggtcgtctatgacaccacattggcatcggaggatcccattatactatccaaatgcgtatgtctatcatgttagccataaaaggaacaatagtcaactaaaatttctcttagctggcccatgctacaagctcacttcctaggtccaaatttctcatagactcagagtttgtagcaaaatgtctcaggaaacttacttttgagcaaaaggtctgaatgaagagaagttttaggattgctatctttcataacaatttgatggaagcagcaggatatatggaggtggtgaagtctcattaatgtaaagctaaggagatcaaatgaccaaatgctgagacaaagtatcattccacaatgcccactaaaggtccatgcagtctttcaaccatgcaattctatcattctatcctccattccctgaagtgaaatttgtgtttgccatttttgacacgaatcagaagtaacaaattcaggctgggtgcagtggctcaggcctgtgatcccaacactttgggaggacaagacgggcagatcaccagaggtcaggagttcaagaccagcctggctaacatggcaaaaccccatctctacgaaaaattaaaaaattagccggtcatggtggtgggtacctgtaattccaactacttgggaggctgaggcaggagaaacacttgagcctgggattcagagtttgctgtgagccgagaacatgccactgcactccagcctgggtgacagagcaagactcaatctcaaaaaaaaaaaaaaagaagaagaagaagaaaagaagaagaggaagaagaagaagaggaagaagaagaagaagaagaagaggaagaggaagaggaggaggaggaggaggaggaagaagaagaagaagaagaagaagaagaagaagaagaagaagaagaagaagaagaagaagaagaagaaaatagaaatgagtgcatatatttatatatgagtactagcctgtatgaacacactgggttctaagcaccagttttctgaagggatatgggttgtcaggcagagtaaaagcaggaatgcagatgagagtcaggaagtaaacagatgtggtgattaaaatgggcaggtacatggacaaaaaaatgcatgtctgacaaaaactggcctcttgccataagtgagtatgaataatatggaaaaactgtttgcacatgttgaacagcagacagtacaacctgagatagtttagaaagggaaacaaataagatcaaccccataattacccttcctagacttaagggcaaagagttttaaccaaagcattccacagcagtcttgctaaactggggagagagactggagttttgtttactaataaaaccgagattttctaggttaggtaataatgagaaagtatttgtggagaaaaggagctccaggaatacacacagaagtctcttcaagtctctggctgaacagaaagctgtgtatgcacagaaagagtttccagagagaaaggagaacaaagaacagctactggggaaagaacaactgctggggaacagtgagctcaatgaagatgccagagctcacatagcactgggaggtatttgagctctgaccagcctgaggagagacacttcattgaacatcttgggcattcagcaaagaccccaaaaaaccatacttcaggagtagaattaatgcattcctagaataaagtctactccagaaacaccctagaaaagcttagaaaccaagtctaaaaagatccaaatgatctccaagtaaattaattgcctgtcagaagaaaacaacctcttcagaggtaaacaacaaaattaaattgctcaattatatagtatgcacaatgtgtggcatacatttaaaaatttgctaaacatacaaaaagcatttagtgtgacccataaccaggagaaaaatcagtcaatacaaatagacccaaaaatgataaaaataacagaattggcaaggagatttaaaatgtatgtatcataattgtgttcaaggatttaaagaaagcgtggacaagaaataaataaatggataatatcaacagaaagaaaaattgtaaaaggaccaaatggagagtcaagaactgaaaaaaaagacatctctttaatgagaaaatcactacatggccttataatcatattagatagtacagatgataaagctaactagaaaatattagggtggtgcaaaccatagcacgcttatacaaagcctgagaagataaacagagcctcaaggacatctatgaaaatatcaaaatatttaatatttgtttaaagcaagtcacagaggaagggaaagagatattggaacagaaaaaatacttgaagcagtgatggctgatgactttctaaatatggaaaaaatgataaactcacatagtcaagaagctcaatggatcagatataggattttaaaaagtaaagctgtatgatttatttggacacatcataattaaattgtccataatcaaagatagaaagtaaaatcttatttgaagcccaagggaaaaaacatacctttacatagagtaacagtgacacaaatgactgatgccttctcatcagaaacaacacaaatcagaaacaatagaataacacctttagagtggtaagaagaaaaaaagatcaaatcagaaacaacaaaataacacgtttagagtggtaaggaggaaaacaagatcaaatcagaaacaatggaataacacctttagagtgtaagaaagaaaaaaagatcaaatcaggaacaacagaataacgccttcagagtggtaagaaggaaaacaagataaaatcagaaacaatgaaataacacctttagagtagtaagaagaagaaaagatcaggtcagaaaaaatggaataatatgctaagaagaaaaaaaaagatcaagtcagaaacaatggaataacacctttagagtgaaaagaaggaaaaaaacccagcaagcttaaacgctatgcacagcaaacaattccactgaaaatgaatgttacgtaagtacatattctgtcctcctaaaaacaaagaacaaataaaagaatgtttcatcagcaggattatgtaataaaagatgtgaaagaatgctatgtaagtagaagaaaaataataccatatgggaattggcatcaaaaccacaaaatactatcaaaacaaaaaaactttattgataaatttaacacaatatgcaaaagaactataccatgtatactacataacattggtgagaagaaaattagaagatctaaataaagacacatcatgcttatagattaaaaaatccaatgtcacttttcacaaaactgatctttagtttcaacccacacccaagcagaattcctgcagtcttttcttgaaaacctaacagaatgtatatgctagaatcaccaagacaatctttaaaaagaataaaaaacttggaataaaatcacaagtttgtgggatagatgcatatggtaatatggaaattctcataaagacacagtaatcaagacatgtggtattggctgggacgcttggctgtaatcctaacactttgggaggccaagatgagaggattgcctgagatgaggagttgcagacaagcctgggcaacatagcaagaccctcatctctacaaatatttaaaaaaattagccaggtttggtgccatgtgcctgtagtcccagctattcaggaagctgaggtgggaggatcactggagcccatgaggtggaggctgaaatgagccatgattgtgctactgaactttagcctgggagacagattaaaaccttccctctctctctcaaacaaacaaacaaaaaatacatagtattgggcaaaacatatgcaaacaaaaacagaaaagggtcagcataaatttacatatatggtcaatttattttcaatacaggtagcaaagcaatttaatgaggaaatttttttccaaaattggtctgaaacaactggatagccatagaaaaaaactataacaaatgtgacgcttgaatcctactgtatgactcaaattaaattaatttgagatagctcttagacctcaatgtaacagctaattctgaggctgaaatataagactgctatgaaaaagtatagtatcttataaccttggagaaggaaaaattttttgagggaagaaccagaaaacactaactgtaaaagaaaacaaatgataatgtggacattcattgaataaaaacttatgctcaccaaatatgactgttaagaaaataaataagtaagtaacacactggaagaaaaacactctcatccatatatctgacaaatggcctgtatccagagtatagaaacatttctcccactcactaatcagaggacaaacaacctaatcaaaatgggcaacaggcttgaatagtcatttcttaggagaagatgcacacagagccaacaatcacctgaaaaagtgcacaacatcttagccatcaaaaatcaagagttataaccctcataagatgacactgaacatccagtgtacatggatatcattaagaagacacaataataagtggtgtcaccgatttggagctagaatgtgccactctctcatatgctggtggaagttcaaaatcatacaacaaattaaaaaatcagtctgatgctttcttataaagttcgataaatatgcatctatcctacaaacctgtaattctattcttgaatatttaccccccaaaatgaaaacataagtccacaaaaatctatataaatattcatagcagctttatgttttataaactcaaaataaaaactatttcaatgttttcatcaaaagaaaatgaaaactatttaaatggtttcatcaaaagaaaatgaaaaaagaatttccagtatatttatacaaaggaatactattcatcaacaaggaacaagttactgatagtctcagaagcatgaacaaacctcaaaaatatattaaggaaagaagccagacgtcaaagtgtatagtctgtatgagtccattcatgtgagtttatagaaaacacaatttatggtgaaagaaaccaatagcatttgacactggccgtgggaagagggtagcagagattgattgagcagccacacaagggagtttctggggtggtgaaaatgttctgcattgtgagggcagtgtgggctacacaagtatatgtatttatcaaatctcatccagctacatttaagatctgtgcatctcactctatgtgaaaatatactcaactgaaaaacagagcaggtatctgtttcaggtgctacatcacttgatacgtccagttgtgttaaaaaccactgcctaacatcctcaaatgggggatctgggcttgagactaggtcacatgtgtagagtctctacagagaccgtgttggattcccatgctccataatacgttccaagttttctcagacagccacaggtcatgaatgtgaggattctgagaggttggagcaacgttcttgggaggcataatggggaaggcattctccaagattcctccagcctggggtcttcacctgctgtgcctcttactgcattgttttctgactcatccatagccacttgaccccttcagatcccatagtctacctagccgtctccctttatgccttgggtcccgctgttctttcaactcatcacccattccttcagtcccagagtggctgcagccagcagaggatggactgagagcaggagaggaggtcgtgcccatgaacccatcctagagaagcagcatcctgcctgggagctagttttccagggaagcttttataagtcctgtagacccaaacccacttgctctaccagatacagtatttatagtaatactattttcatgattattttatattgcaaatgtagagcatttatgctacactatgagtaaatagagtaagggggctggcatgggaattatataatcttggatgccacttcttccttggggaaatgtatttgagttccaacttacatattactatatagtcttatagagagagagacaaagagctagacagacagagatatctttgtatgtgcattaaaaaatctaagatacatatttcaaaatctgtgtcatttattctggaggaaagtatttggcagaaggtgaaaggaagatattctgatcctttcttgtacagacatgtattatctcagttttcatagagagcatatactacttttgatgttttaaaacaaaaattataatctgtgatgtgtccacagttgtttaaaagttgaagctgaagaccatttgtgcttgtggcaatattattgtggtataatgggaatatttcaaaggcacttgttaacactttgttacagcaaaatgtagagggcgctaagtgcccttgaatattctcccatctctggtgacctgtgttgttttgaaatttgcagtggcctgaccaggaactactgcaggaatccagatgctgagattcgcccttggtgttacaccatggatcccagtgtcaggtgggagtactgcaacctgacacgatgtccagtgacagaatcgagtgtcctcacaactcccacagtggccccggttccaagcacagaggctccttctgaacaaggtaagaaatttgtggttagacatctatatactgggatgaaaaaccatggaaaatcttactgatgcagaagccttcagtggtacactggagggttggttgagggtctgcaatgtggaggaaagcctcagcgccctctctgggggatccagaactgtgatttttggcacgctgtgaggaggcagtgtctttaggaagggcacggtgtctttaggaagggcacagacccgccagggcactggacttaccactcccctggttattaaatgggtcatttcagtgtcctagccaaaatggatattctaacagcctgccaaatatgtgaagatttccaagccaataagcctttccagtgatttaaagtagacttttttcattgcaatctacagtttgcagtttcttaagaacatggcctttgagtatgatatcctagagaaacctaaggagactgcattatttttctattgtcctggggctgcatagcaggaggtaaccaacgaatgctgtctctccctggcctatctcagtctttcacaggctctgttcacctcagctttgaagttagaaatttctaggtgttcttgcctcttcttctcatgaaacctgcattggcagtgagtctacagaagaagaggaagagaattctgctttgttacaattcaggactctgggcactagaagattccctatctctcctccaagggaataagttgtttgtctctaaccctccttgagaaacaatgagtctttgcctgcactcctaaatgtaggatgatttcctgcccaaattttcaaaagattaagccttttgccttggtatgagcaatggtctagggaaatgcgcaagggtcttgtgtcggcccctgactgaccaccagtcacctcctacagcctgcaccaaggaatgcattgcattctggtcttctgccctgtggttctcatgaaaaccagcagagattcatatgatggagctgcacatgaatgtaatttccaatgtccagcattctcctctgttctttatctttagatttaaaaataatgtttctatgaacttattaaaattctagaatactatgaatctactgggtcttttcacatccttttgctactagtagaaaaaagaatagtaataattttcagaggctactgtccagtatgtgacataaattgtctcccatgtttctctgctcatgcaattactgagtatgatttattttattttaatttcagcaccacctgagaaaagccctgtggtccaggattgctaccatggtgatggacggagttatcgaggcatatcctccaccactgtcacaggaaggacctgtcaatcttggtcatctatgataccacactggcatcagaggaccccagaaaactacccaaatgcgtatgtatttgattaaaaccataagaggagcaacagccaactcaaatattggttagaagacccatgctttaagctcacttcctagggacaaatttctcttagactcacattttggcaaaatgtctcaggacctttgcttttgagcaaagagtctaagagaagagaaattttaggcctgctatttttcctaatagttttatggaaggagtagaatatacggaagtggcgaagtcatattaatgtaaagctcagaagataaatgaccaaagcttaaacacagcaccattccacaatgcccactaaaaatcaatgtcatctttcactcgtgcaattctgtcattctaaatttcaattcccgaaggtttgtttgccatttttgtcatgggtaataagtaaaaaaaaaaaaattaagatgtgtatatatatatatatatatatatatatacacacacacacacacacacaaacatctgaatatttatatatatgtctgaatatttatatacttgtgtataaaacttatatttaaatttttgcataaatttatatatttttaatatttcattaaaaattatattgtttcactatgtatgtctgagtatttttatatattttaatataacattttaaatatttatatataaatattcaggtatgtaactgaatattcatttacacacacaaatatatgtgtgcatgtgtgtatatatatatatacccatatatatatatatatatatatatacatatatatatatatatatatatgtatatatatatatatatatatatatacacacacacacacacacacatacatacaggtataaacacactgggcctgaagcaccagtggtctgaaaggacatgtgttgccaggacttgaagagcaaaagcaggaaggcggatgagagtcaggaggtacacaaacgctgaaaagtaaaatggacaagtacatggacaaaaagcaggtataagcataacagccttttggaagtaaatgactataaaatatatgaaaatactgttttcacaagttgcacaacagatagtagtgtattgagataatttagaacagaaaacaaatgtgatcaaccccataagtgtgctgtatttcatcatggattgaaggaaaaagagatggagcccaagaagaccacagcagtcttgatgaactgagagacaccagagtttgggattacaaaggcagctgggattttctacacttggtaataatgagaaagaatttgtggagataaagagctacagtcatgtacctagaagtcacctcagtgtaatataaatctgcatatgcacagggagtgattccacaatgaaagtaggacaaagaacagctactggggaaagaataactacaagggaacaatgagttcaatggagatggcagagctcacaaagcactgggggatatttgagttcttaccagctagaaaagagacctcattgcaaatcttgggcattcagtagagaccccagaaaagccactctttggaaacagagttgatgtattttaagagcaaaatctactccacaaaaatcctagcaaaattgaaaagcaagtcagaaagaccaaaatcctctcaacataaattagttgcccatcagaagaaagcttaacctcttcataggtaaacaataaaatcaaattgctcagttatctggcatccacaatatgtgacataaatttaaaaatttactagacatacaagaagcatttagtgtgatccataaccaggagaaaaatcattcaatacaaatagacccagaaatgacagaaatgatagaattagcaaaaacatttaaaatatacatatgatcatttgatcttgtgatcagatatcacaagagaagaaagagatacttgaacagaaaaaatgcctgaagcaatgatggctgaaaactttccaaatatgaagaaaaaaaagctcacagattcaagaaaactaatcaatcagaaatatgattttgaaaagtaaaaatgtatgatttactttggcaaatcttcttggttaaattgtctaaaatcaaagaaagctaggaaaattttataagccagaggaaaaaagattgtttatataaaggaacagttacacaaatgactgatgccttctcatcagaaacaatgaaagtcagaaacaataaagtaacatctttaaagtaatagaagaaaaacccaagaggtgagggatcgtggcagacaggaggcaggactagattgcagctctggacagagcagcatgcagaggctcatattgtgaattttagccccatattgactgcaagaacagaccagcaatcctgagaggacccacagaccgtgtgaaggaagcagactgctcctgcaggataagggagacaccccaaatactgtgagttccccaactgcagaagtggaaaagggaggccttactccctcaaacacaccccacaactggagaagctgaaagtctgtttgcaggagaagttcccaactttacctgggcctcagtaaatttagagagctgagccaagcaaaatataggggtagaggaagcagcagagaagacctcagagcttgctggatccccaagcagctcattcctgcctggcaccacagagatccatcagaagtgtggccaaaggaacagagggtaaaactccacatggaggactgctctacctgaactttctaacaatttgaacagggggagaagcctcctggccagaacttgggggagggcatgaatctggtttgcagacttcacaggtgggggaaggactaaagcccttttctttcacagctgggaggtggaaagcctcaggcaagttttcaagcctgactttccccccacctggaaacagacttggagctgttgcggggttgggggcatggtgggagtaagaccagcccttcagtttgcatgggtgctgggtgaggcctgtgactgacagcttccctccacttccccgacaactcagatgactcagcagaggcagccataatcctcctaggtacacaactccagtgacctgggaacttcacccccacaccatacagaagcttcagtaagacgtgcccaaggaaagtctgagctcagacacgcctagtcccacccccaactgatggtccttccctacccaccctggtagcagaagacaaagagcatataatctttggagttctagggcccacccacctctagtccctctccacactagtatagctgatgcaggaggccaaccagcacaaaaatagagcattaaaccaccaaagctaggaacccctatggagtccattgcaccctcctccacctccaccagaacaggcactggtatccacagctgagagacccatagatggttcacatcacaggactctgtacagacagtccccagtaccagcccagagctgggtagacttgctaggtggcaagacccagaagacaggcaataatcactgcagttcagctcacaggaagccacatccataggaaaagagggagagtactacatcaagggaacaccccatgggataaaaacatctgaacaacagccttcagccctaccttccctctgacacagtctacccaaatgagaaggaaccagaaaaccaaccctggtaatatgacaaaacaaggctcatcacactcccagttcaccagcaatggatccaaaccaagaagaaatccctgatttacctgaaagagaattcaggaggttagttattaagctaatcagggagggaccagagaaaggcaaagcccaatgcaaggaaatccaaaaaaaaaaaggtataagaagtaaaaggtgaaatattcaacaaaatagatagcttaataaaaaaacaataaaaaattcagtagactttggacacacctttggaaatgtgacatgctctggaaagtctcagcaatagaactgaacaagtagaaaaaataaattcagagctcaaagacaaggacttcaaattaacccaatccaacaaagacaaagaataaaggataagaaaatatgaacaaagccttcaagatgtctgggattatgttaaatgaccaaatataagaataatcgtggctcctgaggaaaaagacaatactaaaagcttggaaaacatatttgggggaataactggggaaaacttacctggccttgctggacacctagacatgcaaatacaagaaacacaaagaacatgtaaatacaagcagcacaaagaacacctgggaaattcatcacaaaaagatcttagcctaggcacattctcatcaggttatgcaaagttaagacgaaggcaagaatcttaagagctgtgagacagaagcaccaggtaatgtataaaggaaaccctatcagattaacagccagtttttcagcaggaactgtacaagctataaaggattggagccctatcatagcctcctcaaacaaaacaattatcagtcaagaattttgtatccagcgaaagtaagcatcatatatgaaggaaagatacagtcgtttttggacaaacaaatgctaagagaattcaccattaccaagtcaccactagaagaactgctaaaaggagctctaaatcttgaaacaaatcctagaaacacatgaaaacagaatctctttaaagcataaatcacacaggacctataaaacaaaagtacaagttaaaaaacaaaaacaaaaaacaaaaccaaagtacggaggcaataaagaatatgatgaatgcagtggcacctcacatttcaatgctaaaattgaatctaaatggcctaaatgctccacttaaaggatacaaaaagagttggtggctggcaagatggctgaataggaacagctccagtctgccgctccccgtgagatcaacacatagggtgggtcatttctgcatttccaaccaaggtacccggctcatctcattgggactggttagacagtgggtgcagcccacagagggtgacctgaagcagggtggggtgtcacctcacctgggaagtggaaggggtcagggaactccctcccctagccaaaggaagccgtgagggactgtgccgtgaagaccagtgcattctggcacaaatactatgcttttcccacggtctttgcaacctgaagaccaggagattcccttgggtgcctacaccaccagggccctggatttcaagcccaaaactgggctggcatttgggcagacactaagctagctgcaggagttttttttcataccccagtggtccctggaatgccagcaagacagaaccattcacccccgtgaagaaagggctgaagccagggagctaagtggtctttctcagtggatcccacccccatggagcccagcaagctaagctccactggcttgaaattcttgctgccagcacagcagtctgaagttgacctgggacgctcaagcttggtgggaggaggggtatccacaaatactggggcttgagtaggaggttttcccctcacagtgtaagcaaaaccgctaggaagtttgaactgggcagggtgcactgcagcttggcaaagccattgtagcaagagtgcctctctagattcctcctctctgggcagggcatctctgaaagaaaggcagcagccccagtcagaagcttatagataaaactcccatctccctgggacagagcaactggaggaaggggtggctgtgagtgcagctccagcagacttagtttcctgcctgccagctctgaaaagagcaccagatcccccaacacagcactagagctctgataagggacagactgcctcctcaagtgggtcctggtttcagaagataataagaaactcctctgagctaaaggagcatgttctaacacaatgcaaggaagctaagaaccttgaaaaaggtcagaggaattgctaactacagtaagcagtttagagaagaacataaatgaccttagggagctgaaaaacacagcacgagaacttcatgacacatacacaagtatcaatagcaaaatcgatcaagtggaagaaaggatatcagagattgaaaatcaacttaatgaagtaaagcgtgaaaacaagattaaggaataaagaatgaaaaggaatgaacaaatcctccaagtatgggactatgtgaaaagattgaacctacgtttgattggtgtacctgaaagtgatgggagaatggaaccaagttggaaaacactcttcaggatattatccaggagaacttccccaacctagcaagacaggccaacattcaaattaaggaaatacagagaataccacattcaaattcaggaaatacagagaacaccacaaagatactcctcaagaagagcaacctgaagacacataatcgtcagattcaccaaggttgaaatgaaggaaaaaaatgttgagggcagccagagagaaagtttgggttacccacaaagggaaccccatcagactaacagtggatcttcctgcagaaactctacaagccagaagagagtgggaggccaatattcaacattcttttttactattattatactttaagttctagggtacatgtgcacaaggtgcaggtttgttacatatgtatacatgtgccatgttggtgtgctgcacccattaactcttcatttacattaggtatatctcctaatactatccctccccactccccccatcccatgacaggccccggtgtgtgatgttccccactctgtgtccatgtactctcattgttcaattcccacctatgagtgagaacattcggtgtttggatttctgtccttgtgatagtttgctgagaatgatggtttccagcttcatccacatccctacaaaggacatgaagtcatccttctttatggctgcatagtattccatggtgtatatgtgccacattttcttaatccagtctaccattgatggacgtttgtgttggttccaagtctttgctattgtgaatagtgccgcaataaacatatgtgtgcatgtgtctttatagcagcatgatttataatcctttagatatatatccagtaattgtatggctgtgtcaaatggtatttctagttctaaatccttgaggaatcaccgcactgtcttccacaatggttgaactagtttacagtcccaccaccagtgtaaaaatgttcctatttctccacatcctctctagcatctgttgtttcctgactttttaatgatcaccattctaactggtatgagatggtatctcattgtggttttgatttgcatttctctgatggccagtgatggtgagcactttttcatgtgtctcttgactgcataaaagttttcttttgagaattgtctgttaatatcctttgccaactttttgatggggttgtttgattttttttcttgtaaatttgtttatgttctttgtagattctggatattagccctttgtcagatgggtagattgtaaaaattttctcccattctgtagcttgcctgttcattctgagggtagtttcttttgctgtgcagaagctctttagtttaattagatcccattggtcaattttggcttttgttgctattgcttttggtgatttagtcatgaagtccttgcccatgcctatgtcctgaatggtattgcttaggttttcttctagggtttatatggttttaggtctaacatttaagtctttaatccatcttgaattaatttttatataaggtgtaaggaagggatccagtttcagctttctacatatggctaggcagttttcccagcaccatgtattaaatagggaaacctttccctatttcttgtttttgtcaggtttgtcatagatcagatggttgtagatgtgtggtattatttctgagggctctgttctgttccattggtctatatctctgttttggtaccagtaccatgctgttttggttactgtagccttgtaatgtagtttgaagtcaggcagagtgatgcctccagctttgcttttttggcttaggattgtcttggcaatgcatgctcttttttgttccatatgaactttaaagtagttttttccaattctgtgaagaaagtcattggtagcttgatggggatggcattgaatctataaattaccttaggcagtatggccattttcacaatattgattcttcctatccatgagcatggaatgttcttccatttgtttgtgtcctcttttatttcattaagcagtggtttgtagttctccttgaagaggtccttcccatcccttgtaagttggattcctaggtattttattctctttgaagcaattgtgaatgggagttcatccatgtccctacaaaggacatgaagtcatgtatgggaatgcttgtgatttttgcacattgattttgtatcttgagactttgctgaagttgcttatcagcttaaggagattttggtctgagaagatggggttttctaaatatacaatcatgtcatctgcaaacagggacaatttaacttcctcttttcctaactgaataccctttatttccttctcctgcctaattgccctggccagaacttccaacactatgttgaataggagtggtgagagagggcatccctgtcttgtgccagttttcaaagggaatgcttccagtttttgcccattcagtatgatattggctatgggtttgtcataaatagctcttattattttgagatatgtcccatcaatacatagtttattgagagttcagcatggagagctgttgaattttgtcaaaggccttttctgcatctattgagataatcatgtggtttttgtctttggttctgtttatatgatggattacatttattgatttgcatatgttgaaccagccttgcatcccagggataaagccaacttgatcatggtggataagctttttgatgtgctgctggattcggtttgccagtattttattgaggatttttgcatcaatgttcatcatggatgttggtctaaaattctcatttttgttgtgtctctgccaggatttggtatcaggatgatgctggcctcataaaatgagttagggaggattccctctttttctatgattggaatagtttcagaagaattggtaccagctcctctttgtatctgtggtagaattcggctatgaatctctcctggactttttttggttggtaggctcttaattattgcctcaatttcagagcctgttattggtctattcaaggattcaatttctttctggtttagtcttggtagggtgtatgtgtccaggaatttttccatttcttctagattttctagtttatttgcacagaggtgtttataatattctctgatggtagtttgtatttctgtgggattggtagtgatatcccctttatcattttttattgcatctatttgattcttctctcttttcttctttattagtcttgctagtggtctatcaattttgttgatcttttcaaaaaaccagctcctggattcattgatgttttgaaggtttttttgtgtctctatctccttcagttctgctctggtcttagttatttcttgccttctgctagctttttaatgtgtttgctcttgcttctctagttcttttaatggtgatgttagggtgtcaattttagatctttcctgctttctcttgtgggcatttagtgctgtaaatctccccctacacactgctttaaatgtgtcccagagattctggtatgttgtgtctttgttgtcattggtttcaaagaatatctttatttctgccttcatttcgttacatacccagtagtcactcaggtgcaggttgttcagtttccatatagttgagcagtttttaatgagtttcttaatcctgagtcctagtttgattgcactgtggtctgagagacagtttgttataatttctgttcttttacatttgctgaggaatgcctcacttccaactatctggtcaatttcagaataagtgcgatgtggtgctgagaagaatgtatattctgttgatttggggtggagagttctgtagatgtctattaggtctgcttggtgcagagctgagttcaattcctggatatccatgttaactttctgtctcattgatctgtctaatgttgacagtggggtgttaaagtctcccattattattgtgtgggagtctaagtctctttgtaggtctctaaggacttgctttatgaatctaggtgctcctgtattgggtgcatatatatttaggatagttagctcttcttgttaaattggtccctttaccattatgtaatggccttctttgtctcttttgatctttgttagtttaaagtctgttttatcagagactaggattgcaacccctgctttttttgttgttttccatttgcttggtagatcttcctccatccctttattttgagcctatgtgtgtctctgcacgtgagatgtgtcttcagaatacagcacactgatggatcttgactctttatccaattttccagtctgtgtcttttaattggagcatttagcccatttacatttaaggttaatatttttatgtgtgaatttgatcctgtcatcatgatgttcgctggttattttgctcattagttgatgcagtttcttcctagcatcgatggtttttacaatttggcatgtttgtgcagtggctgataccgattgtttctttccatgtttagtgcttccttcaggagctcttgtaaggcaggcctggtggtgacaaaatctctcagcatttgcttgtctgtaaaggattttatttctccttcacttatgaagcttagtttggctggatatgatattctcagttgaaaattcttttctttaagaatgttgaatattggctgccactctcttctggcttgtagagtttctgctgagagatctgctgttagtctgatgggcttccctttgtgggtaacccgacctttctggtgaatctgacaattatgtgtcttggagttactcttctcgaggagtatttttgtggcattctctgtatttcctgaatttgaatgttggcctgcctttgtaggttggggaagttctcctggataatatcctgaagagtgttttccaacttggttccattctcctcgtcactttcaggtacaccaagcagatgtagatttggtcttttcacatagtcccatatttattggaggctttgttcatttctttttactcctttttttctctaaacttctcttctcgcttcatttcattcatttgatctttaatcactgataccctttcttccacttgattgaatcaactactgaaacttgttcatgtgtcacgtagttctcgtgccatggttttcagctccattagatcatttaaggtcttctctatgctgtttattttagtctgccattcatctaaactttttcaaggtttttagcttctttgcaatgggttcgaacatccttctttagctcggagaaatttgttattacagatcgtctgaagccttcttctctcaactcatcaaagtcattctctgtccagctttgttctgttgctcgtgaggagctgcgttccttcggaggagaagaggcaccctgatttttagaattttcagctgttctgctctggtttctccccatctttgtggtttatctacctttggttcttgatgatggtgatgtacagatggggttttggtgtggatgtcttttctgtttgttagttttccttctaacagtcaggaccctcagctgcaggtctgttggagtttgctggaggtccactccagtccctgtttgcctgggtattaccagtggaggctgcagaacagcaaatattacagaacagcaaatgttgctgcctgattcttcctctggaagcttcatctcagaggggcacccagctgtatgaggtgtcagttggcccctactgggaggtgtcccccagttaggctactcgggggtcacggacccacttgaggaggcagtctgtccattctcagatctcaaactctctgctgggagaaccactactctcttcaaagctgtcagacagggatgtttaagtctgcagaagtttctgctgccttttgttcagctatgccctgcccccagaggtggagtctacagaggcaggcaggtctccttgagctgtggtgggctccacccagtttgagcttcctggtcgctttgtttacctactcaagtctcagcaatggcagacgcccctcccccagctttgctgccgccttgcagttcggtctcagactactgtgctagcagttcaatctcagactgctgtactagcagtgagcaaggctctgtgggcatgggaccctctgagccatgtgcaggatataatctcctggtgtgccgtttgctaagaccattggaaaagtgcaatattagggtgggagtgtcccgattttccgggtacatctgtcatggcttcccttggctaggaaagggaattccctgaccccttacacttcccgggtgaggcaatatcccgccttgcttcggctcactctccgtgggctgcacccactgtctgacaagccccggtgagatgaacccagtacctcagctggaaatgcagaaaccacccatcttctgctttgctcatgctgggaactgtggactggagctgttcctattcggccatcttgaaacctcccctctctcacgatcacaaggtcccacaataggccgtctgcaggctgaggagcaagaaaagccagtctgaattccaaaactgaagaaattggagtctgatgttcaagggcaggaaacatccagtgccaaagaaagatgtagaatattcaacattcttaaagaaaataattttcaacctagaatttcatatccagccaaactaagctttataacaaaggagaagtaaaatcctttacaaacaagcaaatgctgaggaattttgtcaacaccaggcctgccttacaagaggtcctgaagaaaacactaaatatggaaaggaaaaaccagtaacagctactgcaaaaacataccaaattgtaaacaccatcaacactataaagaaactgcatcaactaatgggcaaaatagccagctagcatcataatgacaggatcaaattcacacataacaatattaaccttaaatgtaaatgggctaaatgccccaattaaaagacacagactgggaaattgaataaagagtcaagacccattggtttgctgtgttcagaagacccatctcagggtgaaaagacatacatgggctcaaaataaagaaatgaaggaatatttaccaagcaaatggaaagaaaaaaaaagcagcggttgcaatcttagtctttgatgaaacagactttaaaccatcaaagatcaaaagagacaaaggagggcattacctaatggtaaaagtatcaatgcaacaagaagatctgactgtcctacttatatatgcacccaatacaggagcacccagattaataaagcaagttcttagagacctacaaagagacttagacttccacacaaaaatagtgggagactttaacaccccacagccaatattagatcgacgtgacagaaaattaacaaggatattcaggacgtgaattcagctctggaccaagctgacctaatagacatctacagaactcgacaccacaaatcaacagaatatacattcttctcagcaccacattgcacttattctaaaattgaccacataattggaagtaaaacacttctcagcaaatgccgtagaatggaaatcataacaaacagtctctcagaccaaagtgcaatcaaactagaactcaggattaataaactcactcaaaaccacacaactatatggaaactgaacaacctgctcctgaattactactgggtaaataacaaaattaaggcagaagtagataagttcttagaaaccaaagagaacaaagacacaatgtgccagaatctctggtacacagctaaagccatgtttagagggaaatttatagcactaaatgcccacaggagaaagcgggaaagatctaaaatcaacaccctaacatcacaattcaaagaaccagagaagcaagagcaaacaaatacaaaagctagcagaagacaagaaataactaagatcagagcagaactgaaggggataaagacacgaaaaccctttaaaaaattaataaatccaagagctggttttttgaaaagattaacaaaatacatagaagcctagccagactaataaagaagaaaatagagaagaatcaaatagacacaataaagaataataaaggggatatcaccaatgatgccacagaaatacaaactaccatcagagaatactttaaacacctctatgcaaataaaatagaaaatctaaaagaaatggataaattcctggacacatacaccctcccaagactaaaccaggaagaagtcaaatccctgaatagaccaataacaagttctgaaatcgaggcagtaattaatagcttaccaaccaaaaaaagcccagaccagagggattaacagtcaaatcctaacagaggtacaaagaagagctagtactattccttctgaaactattccacacaatagaaaaagagggactcctgcctaactcattttatgaggccagcatcattctgataccaaaacctggcagagacacaacaagaaaagaaaatttcaggccaacatccctgatgaacatcaatgtgaaaatcctcaataaaatactggcaaactgaatccagcagcacatcaaaaagcttatccaccatgatcaagttggcttcatccctgggatgcaaggctggttcaacatattcaaatcaataaacataatccatcacataaacagaaccaatgacaaaaaccgtatgattatcgcaatagacgcagaaaaggcctttgataaaattcaatacccaatcatgctaaaaactcttaataaactaggtattgatggagcatgtctcaaaataataagagctacttatgacaaatgcatagccaatatcatactgaatgagcagaagctggaagcattccctttgaaaaccagcacaagacaaggatgccctctctcaccactcctattcaacatagtattggaaattctgtccagggcaatcaggcaagagaaagaaataaaggtattcaagtgggaagagagggagtcaaattatttctctttgcagatgacatgattgtatatttagaaaactctatcatctcagcccaaaatctccttaagctgataagcaacttcagcaaagtctcaggatacaaaatcaatgtgcaaaaatcacaagcattcctatacaccaataagagacacagagccaaatcctgagtgaattcccattcacaattgctacaaagagaataaaatatacctaggaatccaacttacaagggatgtgaaggacctcttcaaggagaactacaaaccactgctcaaggaaataagataggacacaaacaaatggaaaaacattccatgctaatggattggaagaatcaatattgtgaaaattgccatactgcccaaagtgatttatagattcaatgttatccccatcaagctaccattgatttcttcacataattagaaaaaactactttcaatttcatatggaatagaaaaagggcctgtatatccaagacaacctaagcaaaaagaacaaagctggaggcatcatgctatctgacttcaaaatatactacaaggctacagtaacaaaaacagcatggtatggtactggtaccaaaacagatatatagaccaatagaacagaacagaggcctcagaaataacaccacacatctacaactattggatctttgacaaactggacaaaaataagcaatggggaaaggattccctatttaataaatggtgttgggaaaactggctagccatatgcagaaaactgaaactggatcccttccttacaccttatacacaaattaactcaagatagattaaagaattaaatgtaagacctaaaaccataaaaaccctagaagacactttgggaggccgaggtggatggatcacgaggtcaggagatcgagaccatcttggctaacacagtgaaagcccatctctactaaaaatacaaaaaattagctgggtgtggtcgtgggcacctgtagtcccagctacttgggaggctgaggcaggagaatggcatgagctgaggaggttgagcttgcagcaagccaagattgtgccactgcactccagcctgggcaacagagtgagactccatcaaaaaaacaaaaacaaaaacaaaaaatcaaaccctagaagaaaacataggcaataccattcaggacataggcatgggagaagacttcatgactaaaacagcaaaaccaatggcaacaaaagccaaaatttacaaatcagatctaattaaaataaagagcttctgcacagcaaaaaactctcatcagagtgaaaaagcaacctatggagaaaaattctgtggtctagccatctgacaaagggctaatgtttagaatgtacaagcaacttaaacaaatgtacaagaaaaaaaaaacaaccccatcaaaaagtgggcaaaggatatgaacagacacttctgacaggaagacctttatgtggctgacaaacatgaaaaaagctcatcatcactgttaattagagaaatgcaaatcgaaaccacaatgagataccatctcatgcccgttagaatggcgatcattaaaaagtcaggaaacaacagatgctgaagaggatgtgtggagaaagaggaacacatttacactgttggtgggagtgtaaattagttcaaccattgtggaagacagtgcggtgattcctcaaggatctagaaccagaagtaccatttgacccagcaatcccattactgggtatatacccaaaggattataaatcattctacaataaagacacatgcacacgtatgtttattgtagcactattcacaatagcaaagacttggaaccaactgaaatgcccatcaatgatagactggataaagaaaatgtggcacatatacactgtggaatactatgcagccataaaacaggatgagttcatgtcttttgcagggacatggatgaagctggaaaccatcattctcagcaaactaacacaagaacagaaaaccaaacaccatatgttctcactcataagtgtgagttgaacaatgagaacacatggacacaggaaggggaacatcacacacaggggcctgttggggagttgaggctaggggagggattggattaggagaaatacctaatgtagatgatgggttgctgggtgcagcaaaccaccatgacacgtgtatacctatgtaacaaacccacacattctacacatgtatctcagaacttaaagtataataataataagatacagaactgcagaatgaataagaactcaccaaccatctgctgccttcaggagactcatttaagacataaggactcacataaacttaaagtaaatgggtggaaataataataagtggtgtcactgatgtggaggtagattataaaactcttatcatatgctggtggaagatcaaaatgataaaacgaattaaaaaatcagtcagatggtttcttaaaaagttccatcaatatgcctctatcttacaaacctgcaattctattcctgaatctttatcccaaggaaatgaaaaagtaagtccacaaagagttctatatgaatatttataggagctttatttattataattcaaactgtaaaaataatttcaatgttcatcaataacaaaatgaaaaaataatttgcaacctactggtacacttgaatactattcagcactgagtatcttaaatagcatggatggagctcaaaaatatactcaggaaagaagccatgtatattctgtatgagttcatttacatgagatcatttacatttcctccaaaagaggaaaaactaatttctgttgaaagaaaccaatgtatttgcctctggcagtggtaagggggtagcacagattaattgggtagggactcaagagagtttctggggtcacagaaatgttccgtgtggtgatgggagtttgggctccacaggtataggtgttgatccaaaatcatcaaaaaaacaacattgcagatctgtgcatctcactctgtgggaaagtatatctcaactgtaaaaagggcagaaattgcttttaaacgctcagccttttagcacatccagttgcttggagaaccagcttactcaaatgggggtctaggctggagactaggtcacaggcatagagtctctaaactttcccatggcacataatacgtttcaggttttctcagagagctgcaggttagtaatctgaggattctgacaagttgggtcaacgttcctaggaggcatgaatgggagtgcattctctaagatccctccaccccagggtccttgctttctgtgcctcttactccattgttttctgactcctctgtagccactcgacctcttcagatcccattgtctacccagccatcgccctttatgacttgggtcccactgttctttcatctcatcctccattccctcagtttcggagtggctgccgctagcagaggatggactgagagcaggagaggtggtcctgcccaggaacccatcctagagaaatggcatcctgtctgggagctagttttttagggcaggttttataagtcttgtaaagccagacacacttgatctacctggtatgttatttacagtaatactattttcataattgcttttcactctaaaagtagagccttttagctacactgtgagtaaataaaggggctggcctgggaatggtatcatgttggatgttgtttcttccctgaagtaatatatatcagttacaatttacatgttactgcagagtcctagagagagacacagagaatgagacagataccaatacatttttatgtgcattaaaaaaatctaaggccaggcgcagtggctcacacctgtaatcccagcactttgggaggccgaggtgggtggatcacgaggtcaggagattgagaccatcctggctaacacggtgaaaccctgtctctactaaaaatacaaaaaattagccaggcgtggtggcgggcgcctgtagtcccagctactcaggagactgaggcaggagaatggcttgaacccaggaggcagaccttgcagtgagccgagattgcgccactgcactccagtctgggcgacagagcgagactccgtcacaaaaaaaaaaaaaaatctaaaatgcactcttcaaaatctatgtcatttattctggaggaatgcagttggcagaaggaggaagatattccgaatttttcttgtatacatttatgtatgatctcagtttttttatggatcatagaccaattttgatattttaaaataaaaattataatctatcttggaaatttacatggttctttagaacttgaggaccgtttttgcttttcggaatattattgtacctaaaatgggaatattacaacgtcactttttaacactttgttataacaaagtttagacagcgctgggtgcccctgaattttttcccgcctcttgtgacctgtgttgttttggaatttgcagtggcctgaccgagaactactgcaggaatccagattctgggaaacaaccctggtgttacacaaccgatccgtgtgtgaggtgggagtactgcaatctgacacaatgctcagaaacagaatcaggtgtcctagagactcccactgttgttccagttccaagcatggaggctcattctgaagcaggtaagaagtctgtggccagatatctacacatttgaacattgggatgaaaagagatggaaaatctgactgatgcagaagccttccatgctacacagaaacttgagggtatggcaggtggaaagaagcctcagcactctctctggtggagcaatttttggcgcaacgtgcgtgggcggtgacttcaggaatggtgcaaacccacctgggcacttgacttaccactcactttgttatgaaaggggttatctcggtgttccagacaaaattccaattctaacatcaggccaaatttgtgccaaatttcacactagtgagtgtttccaggcatttattaaaatggacagtgttcattgcaatcttcagcattgcagttgctgaggtatgtggccgctgagtttgtcatcctggggaaacctaatatgatgatatttattccatctaatcctggggctatttggcagtaaataccacagaatacactatttctctggcttatttcagtcttaggtaggctctgcacacctatgcttggaaggcaggaatttcttggtgttcttgtgccttcttctcatggaacgtgcatctttggtgtgtgttgagaggaagggtagtagacttctgctttgttgcaatgcaggatgctggaacaagaggattccctgtctctactgtaagggaataagattttagcctccatccttctctaagaagcaatgtgtctttgcctccaagtactagatgcaggaccatgaactgccccgtccaccagaagcttaaggctttggcttttcaggagcaatcatctagggaactgtgcagggttttcatgtctgtcccctactgacagccaatcaccatacagcctgcataacctaatccatcatcgtctggtttcctgcctcattgttttcatgaacaaccagtagagagccatacgaaagagcttgcacatgagtctttgttccaattgtaagagcactgataggtccttttcccaccaggttttgaatataaaatttctaagaacttattaaaatattagaatgttattaatctattgtttttgcttcagcatgtccttctgcttgtgagtatactaaagagaacagtcataattctgaaactactgtcctgtttgtgtcataaattgcttcacatgtttctgcatactagtagttactcagcttgattttgtctattttcagcaccaactgagcaaacccctgtggtccggcagtgctaccatggtaatggccagagttatcgaggcacattctccaccactgtcacaggaaggacatgtcaatcttggtcatccatgacaccacaccggcatcagaggaccccagaaaactacccaaatgagtatgtctttgatgttacttgtaagaggagcaacagccaacttaagttcctcctagaagagccttgcttcaagctaacttgttaggacaaatttcccttagacccagaaggtgtgtcaaaatgtccagacaactttgcttttgatcaaagagtctgagagaataggtattttaggcttgctatcttttctaatagtctgatggaagcagaaggctacatggagctgatgaggtctttttaatataaagctcaagagatcaaatgatcaaatacttagagtgccattctacaaggctcataaaagatcaatgcactctttcacccatgcaattctatcattctaacctcccttctctgaaatgaaggctttttgccatttttgtcatgggtcacaagtaaataattcacatgtatatgagtatatatataaccaggtgtgtttattcagactagtatgtatatatatacatatatatgttcatataagttagtattcatatatatgttcatatatatatgttcatacagactagtattcatatatatatacatatatatatacacacacatatatatatatatatatatgttctagggaaacatgcaaggtttttatgtctgtccctgactgatgaccaaataccctatagcctgcacagctgcaagctgtatagccatacaatttgcaggacacacacacatacacacacacacacacacacacacacactaacatataatataatataatataatataatataatataatataatataatataattaatatatataaacctgtgtgaacacactgggttctaagctccagttttctgaagggatatgggttgccaggagaggaagagcaaaagcaagaatgtagatgagaattaggaagtaaacagatatggagattaaaatgggcaggtacatggacaaaaaaccaggtctgacaaaaactggctttctgccataaatgactataaaagatattaaaaaacactttccacatgttggacaagagacagtacaggactgagataatttagaaaaggaaatgaatgagcgcaactccgtaactattatgactttcttcctggagaaccttcctggactgaagggcaaggaattggagccaaagccaaccacagcagtcttgctgaactgaggaaagagactggagtttgggatagctaagaaaatgtgtattttctatgctaggtaataatgagaaagaatttgtggtgaaaaggagctgaaggaatatgcatggaagtctaatataaactgcatatgcacagggagaaattctacaaagtgggacagagaaccactactggggaaaggacaaattcagggaaacagtgagctcaatggtgacgccagagctcacgtagcactgggggataccggggttctgatcagcccgaggagagacacctcattgaacatctcgggcattcagtagagaccccagaaaagtcatactttaggagtaggatttatgccttcttagaataaagactaccccagaaacaccctagtaaagcttaaaaaccaagtctaaaaggacccaaatgatctccaagtaaattaactgcctgacagaagaaaactcaaccatcactggaggtaaataacatgattacagtgctctgtaatgttgcattcacaaggagtgacatcatttaaaaatttatgaggcaggaaaaagcaattagtgtgatccataactaggagaaaaaccagtcaatacaaatagaccaagaaatagtagaaacgatggaattgacaaagaaattaaaactgtatatatgataattgtgttcaaagatttaaagaaaacatgaacatgagggaaacaaatgcagaatataaaaaaaagcaaatgcgtaaaacaaccaaatggaaattaaagaactacaaaaaagtataaccttaataaaatactcactggatggccttaatattagtttatacattacagaagaaaaagtgaaccagaagataactcaatgaaagccatacaatctgtaagacacacacacacgcacacgcgcgcgcgcgcacacacacacacacacagagagagagagagagagaaagagagagagagaaaggctgaaaaaaataaatagaaccttaaggatatcagtgaaaatagcaaaagatttaatatatgggtaaagcaagtcacagaaggacgggaaggagatattgggacagaaaaaaatactcaaagcaatgatggctgaagactttacacgtatgaagaaaatgataaactcacagtcaagaagctcaatgaatcagaaatagtatttttaaaagcaaaactctatgatttacttgggtacattatagataaatcgtccaacatcaaagataacaaggataatcttataagccagaggaaaacaatatcatttacatagagggacagtaatgaaagtgaccgatgccttctccttggaaacaatggcataacatctttaaagtgataaagagaaataaaaacagatcaacctaggacgacatgtccagccaaaacaaacaaataaacaaaaaaaccctttaaaataaacgtgatgtaaatacgtattctgccacctccagaggaaacaagcaaaaaaacaaaagaatgtttccaaggcaggcttctgtattaaaagattttaaggaaagttattcaggtagaagaaaaataataccagatgggaactttaatccatactaagtaatgaagagccctggaaatggcaaatggcaatgtcaatataaaatactcttatttatctaatttttaaatgtatttaaaggacaatttgtgatattaattaaaataataggaatatattgttgtttcaacgtatgtagtagtaaaattcataaaaacagtagcacaaataatgcagatgataactggaagtatactgttaatgagttttttgcattatccatgaagttatataatattaatagatggttgaatgtgatagtttaaggtgggatattataaatcctaggacaaccaaaaaaatttaaactgagaggaatggatagtaagaggaatagtccttttatgcaaaagaaggaagaaaaagaggaataaagaatataaaagatatggtgtaaacagaaaatacatagcattattgtagacacaaactgaactaccttatgagtatattaaatataaaaggattaagcattacaaataaaaggcagagattgtaaattgaataaaaaccacagctaagtgtgttctttttagaataaatactctttaagtgtaaagatctactttaaacaccaaaatatgaaaaaggatatataccatgaaaacctgaatcataaataagctggagtggtgattaatggatgcaggcactcctaaagactaataagtgaatgtggtcaaattgaagaaacaaaagtatatacgtgctcaatgtgcaaaaactttttctgtatacatgctatgatcctttggaaaattaaagttttaaagcaatatcactgacaatagtatcaaaaccaaaaaatatttagtgataaatttcacacactatgctcaaggactatacaccttgcactagaaaacaatgttgaggaaagaattaaaagatctaaatatacaccatgcttatagattaaaagactccatatcagttctcgtgaaattgatctttggatgaaacccacacccaagcactattgcaacagtccttttttggaaaaaaaaattggaggacttatataccttaatataaagacttataaaagtacaggaatcaagacatgtggtattggcctggccccttggctcatgcctgttaccccaacattttgggaggctgagtctggaggatggcttgagcccagatgttcaagaccagccttagcaacagagtgagaccctctctctacaaaaaataaacaattagatcgatgtgatgacttgcacatgtagtttcagctactcggaatgctgaggtgagaggattgcttgactcaggaggtctagccatgagtgagcattgatcatgcctctgcattccagcctggatgatggaatgagacactgtctcaaaaaaaaaaaaaaaaaaggatatgtgttattggccaaaaaagtatgcaaacctaaaaagggatggcccaccaccagacccacatacatatatggtaaatggattttccgtatagatggcaaagcaattcaatggagacaaaaatgttttacaaaatcattctgaaccatttggatatccatgatacaaaacaaaagcagaacttgacttttgcttttcatctcaaattattttgatatctcttccacctaagtgtcagagctaaaactgaacctgaaatatgaaagttccatgaaaaaatataaaatcttcacaaccttggagaaggcaaacttttttgaggcaggagtctgtaaacactcactataaaataaaacaaattataatgtgggctttcatgaaaactcatgcttaccaaaagtcattgttaagaaaataaataggcaagtaacacatgagaagaaaaatgctctctgtccatatatctgacaaatggcttgtgtccagaatataggaacatttctcccactcactaaacagaggacaaacaactaatgggcaacagattgaataggcatttcttggggatagatagatgtacacatagccaataagcacctgaaaaaatgtccagtatctcagccatgaaaaataaagagttataatcatcatgagatgtcaccaaacacccaatggacatggatattattaagaagacaccacagtaactgatgtcactgatgtagagcaaggatgtgaaactctctcatatgctggtgaaagtgcaaaatgatacaaccacttttgaaatcagtctgatagtttctccaaaagttcaataaatgcacttttaccctacaaacctgcaatcctgtttgtgaatatttaccccacagaaatggaaacataagtccacgaagacatctccaagaatattcatagcagctttattttttataaccccaaactgtagacaatttcaatgtcaatcaataagaaaatgaataaataatttgtgaactagtcatacaatggcatactgttcagcaataaaagggagcatgtttttgatactctcaaatagtatggaagatgctcaaaaatattacattaaagaaagatgccagataacaaaaatgaacattatgtatgagtctattgatgtaaggttccagaaaggtaaaactaatttctggtgaaagaaaccaatatcatttgcctctggccatgggaagagagtagcagagattgattgagcagtaaaacgaagtttttttctggggtgatgtaaatgtcctgtattgtgattgaagtgtgagttacacaagtgtacatgttcatcagaagtcatcaaactacatctaagatctgtgcatttgactatacatgaaaatatacctcagttgaaaatagatcaataacctccctcatatactatacttgctaacacagccagctgcttggagaaccagcttgctggaatggagaatctgggcttgagactgggtcacatgtatagagtctctacagagacaatgttgcattcccacggtacataatacatttcaaggtttctcagacagccacatgtcatgaatgtgaggattctgagaggttggagcaacattcctgggaggaacgaaggggagcacattctccaagatcccccaccaccggggtcctcaccggctgtgcttttttttttttttttcttgacagagtctcgctctgtcgccaggcaggagtgtaatggcccaatctcggctgattgcagcctccaactccagggttcaagagattctcctgcctcagcttcatgagtagctgggactacagatgtgcgccactgcgcccagctaatttttgtatttttagtagagacggggttttgccatgttggccaagatggtctcgctctgttgacctcgtgatccacccgccttggcttcccaaagtgctgggattacaggcgtgagccaaagcacccagcctgtgcctctcacttactcaattgtttttctgaaccctccatagctggtggaccttttcagatcccatagtctagccagccctctcactttatgccttgggtcccactgttccttcatctcatcccccttctgtcagtcccgcagtggctgtggccagtagaggatggactgagagtaggagaggaggttctgcccaggaacccatcctagagaaacagcatcctgcctgggacctagtcttccaggtcagcttttataagtcttttagactcaaactcacttgacccacctgaagtggtattgacaataatgctattttcatggttgtttttcactgtaaatgcagagccttttagctacacgactagtacagagagtaagggaggctggcctgggaatgatatcatcttggatggcatttcctccttggagaaatatatgttagttccaactcacatgttactatacagtcctgtagaaagagatacagagagttagacaggtatagacgcatttgtatatgcataacaatctataagacacacatcaaaatccgtataccggttcctctaggggtatgtgcttggcagaaggtagaaggagggtattctggttcctttcttttgcacatttatgtatgatctcagtttttatatggagcattgatagggtttggctatgtccccacccaaaatctcatcttgacttgtaatctctataatcctgataatccccatgtgtcaagggcaggaccaggtggaggtaactggatcatgggggcagtttctcccaggctgttctcatgacagtgagagagtctcctgagatctgatggttttgtaagtgtctggcatttcccctacttgcacttactctgtcctgccgcctgtgaagaaggtgcctgtttctcccttgccttctgccatgactgtaaatttccagaggcctccccagcaatgtggaactgtgagtcaattaaaactcttttctttgtaacttacccagtctgtctcgggtatttcctcatagcaatgtgagaacgggctaatacaagcatatactacttttgatattttaaaataaaaattatcatctatctttgaaaggcatgcacaaatgggaagttgaggaacatttgtgttgtggcaattgtatgatacctttaatgggaatatttcaaagacacttgttaagactttgttagaacaaaatgtagagggtgctggatgtccctgaatattcttccgcctcctgtaacttgtattgctttggaatttccagtggcctgacaatgaactactgcaggaatccagatgccgatacaggcccttggtgttttaccatggaccccagcatcaggtgggagtactgcaacctgacgcgatgctcagacacagaagggactgtggtcgctcctccgactgtcatccaggttccaagcctagggcctccttctgaacaaggtaagaagtctgtgtcttaccttgtctagcacatacctctctatgtgcttggacaacgggatgaaaagacatgaaaaaccacactgatgcagaagcctttagtgctacacgggagctcgagtgttggttgaggttctgccatgaccaaggaagtctcagtgccgtccctgggaaagccagagctgtgatttttggcacaacttgtgggagtagtgactttaggactggcgcaaaacctccagggtgctcaacttaaccactcaccttattctaaaatgggttatttcagtgtcccagtcaaattcctattctaacatgctgtcaactgtgtgattatttccaagccaataagcatttccagtaatttcttaaaatagtgttcattgcagtcttcagcgttgtggctcctgagggatgtggcccctgattctgtcgtcctagagaagcctgacatgactgcattgattctgtatcgtcctgggtctatgtggctgcctggctgtctgtaatcatctgttttatttttatttttttctacagactgtatgtttgggaatgggaaaggataccggggcaagaaggcaaccactgttactgggacgccatgccaggaatgggctgcccaggagccccatagacacagcacgttcattccagggacaaataaatgggcaggtctggaaaaaaatgtaagccactttgatttggactctttttccctttgctgacaaatcttttcaaacagaagaggggcagaggaaaatactggaaagacttcaggaggctaagcgtaattagccttagcatggaaagtgcaagcagcacaggccagcaaagccccacgcgtgtgggggttctcaggcctcttctcttttgacatttctttactgtttccattgttgggtgctgtttctcgtttctagtgcttgtcctctaagccaggggtccccactccagtactggtactggtactggtactggaactggtaattatctgtggcctgttaggaactgggctgcacagcaggaggtgagcttcgggggagcaaacaaagcttcatctgtattttctgctgcttcccatcactctcatagctgcctgagctctgccagctgtcagatcagaggcagcattagattatcatagcacaaaccctattgtgaactgcacatgtgaggaatctagattgcatgctccttatgagaatctaatgcctgatgatctgtcatgcttccatcacccccagatgggaccacctacttgcaggaaaattagctcagggctcccactgattttaccttatggtgagatgcacatttatttcattatatattacaatgtaataataattgaaataaagtgcacgataaatggaaggtacttgagtcatcctttaaccatcgccccctcaccccaggtgcacagaaaaattgccttttatgaaactggtctctggtgccaaaaaagttggggaaccacactgctctgggttctagtagtcagagatgccctctatgaggcttaagtcagatttttctagaaaagatttggatgggccatcaggtcaccatgagacttcccttagcctcatgcattctctgtgatggtttactttggggcctatgaatagggaagactgagatataggaaaaaccaaagtgtctgtgttcccccactctcacacccatgtaacataacacttctcacaccagatatggggggatttctcctcacaccccaagcgagtctccagcagataccagctgggtgtcctacaatgtaactcggtcctgacactctatctggagacagtgtcagatcccacaagttaaggctcagtcctacaagactgccccactgcagatgccaatcccaagttgcaggctgtgacctgtacttctgcccagctggataaagatctgtttttctatatgaccctccatgggtttgattactttgctagagtggctcacagaactcagggaaacacgttacttttatttacccatttattataaaagatattaaaaaggatcctggtgaacagccaggtggaagagatgcacagggcaaggcacgtgggaaggggctcagagcctctatgccctctccagtgcaccagtccccagtaccctaagtgttcagcaacccagaagctctccaagtgcagtcttgttgggtttttatggaggcttcattacagaggcacagttgattacatcattggccatcggtgatcggctcaccttcggcccctcttccctccctggaggttggagggtggggctgaacagttccaaccctcaagtcacatggttggttcccttggcaaccagcccctggggctatccaggaacccaccaagagttgcttcattgcagctcccttcacccaggaaactccaagggatttaggagctctgtgttaagaactggggggcagagacccaatatacatttcttattctatcacaatatcacaggaagctaaggatgatactgcctttgtgtgtcttggctgtggatggtgcataatgcatggaagtaagcatttctgaatcaacagcaaacaggctttatcaggtagaagacccctcagcgccccagggacaaagctcatcaatgatgtcccactgtcctctgaggctctagctctaagacctccagtgggtcaagctcctggagaagtggcacattctccaaagacccttcagggtcaccacaccctggttaagggtgtggcctcataactccttttgactatgactgatggcttacagcatagaaagaaataactttgtcaaaaaatataataatgatagaaaggaagaaggaacgctcccttttgtcttctaagaatagatgtgaaatgtgtgtgccttagaatatcttctccctctcctgctccacgtgagctggagcttacatgcctgcttgttttcagtactgccgtaaccctgatggtgacatcaatggtccctggtgctacacaatgaatccaagaaaactttttgactactgtgatatccctctctgtggtaagttgccttctgttttggtaaggaaactgcttccttaatatggatttggaaaaaaaaaagcaaaaaaaacagaaaatggcttttgagctgagtgcttctggggaggagatggctgccctctccaccagagcctgcttttcatcatggccaccttgaacctgccctactattggccccatttgttaggaaaacacccgcccctcccaccacacacacataaataaaataaatgtcaaattcccaaagggcaaacttagaggtgatctaatcagcccgggatagtcccaccgaacccttctttgtctagcgtgggatgcatgaaaaacaaatttagagtcattatgatgaaaaactgtcctcttctgcagctgagaagaaaaaaaaaatacgagcagcaggaaacagctaagcatgtaatgcacattgtaaacctcagatggccatcctaggaaatcaatgaagggtagtgcagctctttagccccagatggcctttctcgtaagattactactcatgagtcccattagcgacattgcttagagactgcttgttaggttccttcctcattgctctgagactcttattgggagtatgaggcttggatcaggggaaggggaattgacattagatcttaaatgattggggtaacaaatccatgggggaaaaaaagccacttgtacttgttccctattttcttcctgctgaccaatcaacttgtctgtccgagttacagaacaccaccctggacttttcttttgtgtaatttggttgcttgtggttgggtctgccatgtgaagggaccttgagctgggggaagaaggttggcctccaagtccactgaagaccagcatcctgagattgcctggggaggtggtacagggcagtgatgaagatcatgggagccacactgcccatcgtcacatttgggccactcctggggagagcaagagggaagaaggagaggttagggtgataggaaagattctacttggccaatattattataatgtggcattgtggtctctggatttagtgtgagttgatagctgacttttttctcgagtgggtgcttttgttctattttgtcggtgctattgcagaagcatcttggtggttcctctacctcaaagtctcttgatggggtcagttccagttctccgcttctggccccatctagtacacgccactgcctctcactgcctgggctctctatccttgacaggctgccttgaatttaagcccagtctgacttacctgcctcaaacacccacagtagtgcctgggactcatgcacctttgactcccatggaagggaagtgcagtagcttcccaggtgcaattctgctgtcctcacccacattgaggatgtatgagaatcaggttcttagagattggagaaagaaggaagaatgggaacaagatttcttccaatggactgtgaggttccccaccttactttgatgtaagacaagtgaggttaaccccaagcctggtgaggagggttcccatcagacacttggaaatcctgaggactgtttcctgcagaaggatgtggttggtgggatattcaggtttgactcatgattgagaaagttagagcctctggttggagaaagagtttaataactatttcatttccaccaacacattcagtacgaataataaataagtaaaaataaatagaaacattcagttttattttgaatagtaggagtagggtataatttctgtagttactcttttagtacaatgatgcatgtttactgtatgtaaggcatactagcagaaattgagctcagcactagaaaagatgattgcattccatgccatgcttcttttttacaaaagacttctatagatagattctcaaaacaacccacagcaaatgaaaagttatttggaaaactcaggttccagattcactggagtgtagaatctctggttggttggggaggaatttcctcttgcagttgttattaataattatatgaataattattaactatattaatatttatagttttgaagaccttgaagggctggagacaacagagaagcatttttgaacaccctctgtagcccctgcactgttgtaggcattgatgggtggtaccaaagatgggacactttccctacctccagagaccttgtgggcttgctgcagagagaaggcagggaggaggaaaagaagaatagaggcacatgtgtgtaaattacccccacagcagtcagttagtcatgggaggctccccagaagaactgtcctgaagctggctgagagaaggcaacatttcaacataggacagttatccttgctacataaaatcacatacacacatgcacatatgtccacacacagagactcacatgcaaaagaatcctttgtgcctttcagtaaactttacatggtttagaaagaacttatatttccttgaaaggagagtgtcctttgttgtttactaccactttttaaacttagaaagaaaaatctaaagagtgtttatgattttaccatttaatttcacctttgagatgtgaaaaactagtgcttggaattcgtcctgaattaaacgacacaattgctaacttggactcaaatgcgacttcttttcccaccttgtgccacagcatcctcttcatttgattgtgggaagcctcaagtggagccgaagaaatgtcctggaagcattgtaggggggtgtgtggcccacccacattcctggccctggcaagtcagtctcagaacaaggtaagaacaggcccagaaaccatctatactgtccttccatgtaagccccacaaaacccttctacatttacacagaacccacacagctgatgcatcaatacctgcctctctgttttctgaaggaggaaaaaatatagaaaaattaaaaaaagttatattattataggttctctacttggaaaatagccaaaatacaaatctttttcttgatctgggcagttccatcaaaatctgtaggcacagtgatttgcaccaagttccaatacttttggaaaatattgaagatgctctgagggtttctatggatatccattgtctcactgtcagatgaaaagaaagggaagtttttagaaatgtgacactttgcagtgagggaggacaagagcaaacttacctacagtctatcacaggcacagatttttttttacacttttgtgaatcattgaattcaatgccgaggctattcatctattcacaaacacatgaacaaattatgggttgtgatccccataaatgaagagtaatcagtccgaacccacagaacctggacattttgggtatcgtttcagtggaacatgcaattcgtaagttcagtttgcttgggtgtctcttaggaagaacacataggacacagacccatctgcctgcatgttttgcttcctcatctcctttctacaccagggcacctgtgctcaattgctgttctcctctaaagagacttccttctgtaagtttgtgaaatgccatcgacaaacctgatcgcatcgcatttcactctgctgttgagttgatttttctttactttatcgtttgtaacttcttgctctacagagctttcaccttccacatatttcagattcattctttcctaaactgtgtggtggtctatgtcctcactgactatcaacatactgccatcatgcacttcctatctctattcctcttcgttgcaatctggctccaagtggctcacaccattattctgatctatcaactgcctacacagtcctagaaagtaagtgagtcaagaaacatcccccaaaagtaaacttttcaggtaagatcagaagaccctcatgagtcactgctgctcaggatcgtatctggctccttgaagagtgaccttgcatagatcttgtcataaaaaatgaaagagaccttgggaaggtcttgggctggtcacttttgtcagagtccagggctgtggggtgaaagccacagctatagagcttcattctggagtcacttagctttgctctcctggggacaggctgtgcctattcttgcctcaggcatcaaaaaaagtggcacagatgggcccttctgaaaaatctcactactggagcacagctcgaagtttctactatcctgacgttgggcggtagtcctttgctttgggaatatgaacatgatcaaaactgagtgaacttgtcttcctggctttctgtacaatgaagtagaacaaaccatccaatttgaccaaagccttggcatgttttctttctaggtttggaaagcacttctgtggaggcaccttaatatccccagagtgggtgctgactgctgctcactgcttgaagaagtacgtttaagggaaaactgacatggggtcttatcttcaagacttttttcctccctctcttcctccatcccttctttcttcccaccctccccttccttcctccccacctctcttccttttctggaaggaacactaggaaccagggaatgcatgcagaatcctgaggcagaatttccagggcaattggatgagagaggagggaagtgtttctagagggaatctgcagagggaagacccagtgcaagtgattttttggacctgtataaaccgcaggacagagctgttcactaccagaggcatcaatctgtattgcattgctctagagcaatatctgaggctgaataatttataaagaaaagagtttaattggcacatgtttctgcaggctttacaggaagcaggatgctgtcatctcctctgcttctgtgtgggcctaaggaagattacaatcatggtggagggcaaagtgggagcaggcatgtcacatggccagagcaggagcaagagacagagagagatggggtgggggtgctgcacaataccaaatgaccagactttgcaagaactaagagtgagagctcactgatcaccatgaagatgtggcccaagccattcaagagggatgcacctctatgatccaaacccctttcacaggccatagctccatcactggggactacagttgaacacgagatttaggtggggacaaatatacaaactatatcacagtctctgatgaaacagattgagaacagaccttaactgtcagtttccagcaaattgtgaattttgtttcttgccactcataagtcactgattctgggtggccgagggtgtcagagggacagcgccaagttcatggcacagaggatacctgaaggggctggaccatatttttctcttgacatcctcatcttttctaggtcctcaaggccttcatcctacaaggtcatcctgggtgcacaccaagaagtgaacctcgaatctcatgttcaggaaatagaagtgtctaggctgttcttggagcccacacaagcagatattgccttgctaaagctaagcaggtactcgctcacctgtggtcttcaccccacgctggtgaagatatttgctttatgtctgggttttatgggccatggccactgcatggcagtggggaggaactgtctatcacatgaaaggctcaagggctttggggacagcatcaatcttcaaccccagccctgccacatgttagttgtgctctttaaaaaggcagaaggattcgtttcctcacgtggaaaaagagataccctgttacccgtaaaacttacttaatgttcaccagttcatccacattcatgatcagggaaaggttgttattccaggctaactattctcctttcataataatatgctggagagaatcaaatgagattgcatttcaaagcgcttgaaaaaccaccatatcgagccatgcttagtgtgggcgcctctaatcactgctattcaggaggctgacgaggaagaattgcttgagcccaggacttcaaggctgtaggcagctatgattgtgccactgcactccaggctgggtgacagatcaagaccctgtctcaacaaaagaaaagaaaacaaaacaaatgaacagaaatattccacaatgtcaaaaaaaaaaaaaacccacacaacatacaatttacaaatgcaaataataatattattgttgtcttctttgattttctctttcctggtgaaattttgttttattaagcctgacaaagtgatacctttgcttacatcacttaaagttagtctatttggacctaggtgacagtacaatcagctaagaaacagtatttgtaggagaggcaggtttgggacaggtgacaaggcatgtggggtgctcgctgtgctggtggctctggaaggcagggtgtcaatgcagacagggatgagcatggcctggttgggaaggcatggggcaggcaggagcctgagctgctctcctgggcctggtcacaagcccatggcagcttctctgggtctgtgaactgaggggtgatgtcctggaatcctctgacactctaggaaggagagaagggcctttctggctcagcctttataaacagtagctgatctccctcttgctccccagggtcctccccaccatcccagcaaatgtgcaaatacaagatctctgctcctcatggtcctcagagagctggggtgttctgatggcttgaacaagtcacttaggaaatgtggggttttggaggcattctctgataggctgatacgttttgagtttagagttcccaccgcacatccccacacccctagagtctagggcatttagtgctccatgagggaacctgtagagtgaggacatctgcatcacaggctgggccttctagtgtccagaagcagaaagtgtgtctgcttcaaagttggtgctaatgatgatttttggtcagaatacggcatttctcatttccattcctttatccccttgaacttactaaagtagaatcaggtctaaaaaccagagttctaatctttaagagtccctgggattctaaggtatatgaatgtccttggaaaacaataccatttagttcatgcaaggtgcttatttcccatcctctttcatttgatgtctagcattttactgcattcttaccaccacggtttagtaacattcacgaggaggaagtggaggatccagatggagcaacttgctctgggcacacaaggcatttgcaattttataccctcttgatgatgtctcagccagacattctgcccagtcatcaatgccctcttcaattaatatgaaaggacacacttggcatgagattccaatcgtgcacagaatatacatgagaagtgtgcctttgtcatccctactttcaaaggctaaggccaccctcagtttcttgcatgcaactgatgcctttcaaatgaaaccttacatctgtgtagtccataggcaaccacaggcaaatgtgagggtgaaacgctgtgttctacattgttctgtgtcagtgaagcaaggcagtgccagctcagagggctctggggcttcaaggcagggatgcctggttgtaggtactgccacttccagctgggcagtgaaacataactgctaatactttccttacaggcctgccgtcatcactgacaaagtaatgccagcttgtctgccatccccagactacatggtcaccgccaggactgaatgttacatcactggctggggagaaacccaaggtgagatcaattccattgcccacgtaacaaattgtttttgaccttcagtgcatgttacaaaatgagcattttggagatagttgtacaaattcctacccatgaatgtggtctacccactcctgactttgcctggacacctgtctatgtctccataatcagtcttcaagggacttgggcaaggggagcggtgccatttccttgagtctctctcttttttgttttcagaatcttttaattttttttgtaatgattgtatgtttcccttacaacaaaaacaaacaccagtagaggtctttgagtctcttaatcataatttcagcattcatattgcttccccaggtaagtggggttttgacccagccctcaagttaagggtgttagattatttttcatgtgaaattagacagactgcgtttctaaacatggtgcaaaacagtaacgacaaaagttgtaattaaactattcttcttcccaaatacccacatgtctaatgtgtgtgtgagggtgttaggcaggggacctgaagctgggggagaggcagacagttcccatggccccaagtctaggatggcatttggtattggttgatgggtgagagcaagagagggaatatttttgtgcatgatgtggtatcagcacctgtactacattttatggattccttcttctctttgcggtatgccctgacaataattatatccgtcagccttacccccttggcagtaggaaaactgaaactgtcttaaagtctcagctctactttctcagaggtgcaggcaagggcactgggagtctggggccctggaaaactgttctgactctgccacttgccagatagacctgaactagacacgttacctctttgtaccacttggctctaatcccttatctgtaaaaccagcattttcaaatggtgctttgcacatcagccttttgcataagctttgatttgataaaatgttttttgtgtttttaaaaagattaaaaaccacaggtttagataatttcaaagtaggcttccctttttctgtcattttcctattatttttaaaacctcacctccttgactccttgttccctttttctgcactgctgagtctgggagcactgaggccaggtaaaaggaaacttggcaaatgaggggcacctatgggtgtgggaggctgctcctggtgtttgcatattttaaaatttaaatgctacaaaccactgtgagttaggtattattgttcctattttaccattgaggaagctggggctcagagaaggtggagggtggtacagacaaacctgaattggaaccctggctcctgcctatgggctgtcaggacttagaaaagtcgtgagctctcgctgattgtttcctcagctgatgtgggctgcagggctgttatgggggaaataataagaaagtgcatcaagtgctgagcacatcctaagcactccatcatggcagctcctactactaataaagaatagaattatatctaacatgattctttcttgcaagtgacagaaaatccaactcaaattggattaagcaaaacaagggaaattcttagtgagctgcaaagttttcaggctcacatgatggccccaaatcccaggtcctcccaatcatggagtaggcactatttgggggcacaaaggtgacattcccatggctgcagatgctgtggtgctgtggctgtaccgggaaagaataagaaaggccactctcccaattatgtgaacaatagtctgcccactctgagaagtcaaacttgggtcacagtcctgcccctgaacccatcactgactggctctgacctgcaccaattgttccatgttggaggtgaaggcaagaccccactaatacccataaggggcaaaagttagatagatccttcaagaggattatgggaggtagggcaaaaagctgctgggcagccagaaagcaaacagagcctctatgatacctcaactgatgaaagcatgaagctaaaatcataaggatctgggtgtgagttctggctctcccatcttccatgtgacattgggcagttatttaatctcttttagcctccgctttctcatcttacatatgagataattgtgaggattaagattacacataatcatcatcatcaccgtccaccactaccaccatcatccccatcaacatcatcgccaccactatcatcattcttactggcactaccatcaccatcaccaccattccaccaccatcaccaatatcatcactgtcaacatcattaccaccatcaccatcaccaccaccatcatcattactaccactaccactactaccaccatcaccatcaccaccattccaccaccatcaccaatatcatcactctcaacatcatcaccatcaccatcaccaccaccatcatcatcattactaccactaccactactaccaccatcaccatcaccactgtcccactactatcagcatgacatcaccatcaccaccaccatcatcattaccaccgctactaccaacatcaccatcaccacaattctactgccatcaccattaacattaccaccaccatcatcactatcaccatcaccaccatcatcaccactgccattatcactgccaccatcatcactatcctctatatttcctcatctgtattatcattactaccaccatcactatcaccaccatcgtcaccatcataatcaccatcaacaccatctccaataccaccatcactgtaaccatcatcaccaccaccatgatcactatcaccatcatcacaatgatcactgtaaccatcattactacccaccaccatcaccactactccaccaccatcaccattatcattaccatcaccattatcaccaccatcatcatcaccagcaccaccatcatcaccagcaccaccatcaccatcaccatcattaacaccatcactatcaccattggtttaatcatcaccaccatcatcataaataaacatcacataaccagggtgtagctgggtgttgaccccagagcccactcactgtttcctctctcccacccccatccacacatttctaaccaccatcctgcactgggctcccagtctcctctggtctcacccacatgtccactgagaaaaggattttcagaacaccaactagaccaggaggagccacatacataactcaggcctgcttatcaactttctacatgttaataatgacatcagatcaatgggtgttctcagcttctcagaaggaggtcaaaattctccccctctccccttcatgtgtccagaccttcccggatttggatgtaccaagtgcagagtggtgttgaggccaaggggctcatccatgtaagtctcatctgcaatcactgggctgatcccgtggccctgtctccagggcgccatcagagagggcttcaatcctcaggttacctgtggcccaccctgccctcagaggtgccatctctacattggccacgagatggcagcacatactcatagactgcattaatttcccagcaactcctggtgggttttccctcttatcaggatgtttgccttgctcagagagcaaatctgagagcagtgacacctaacttaactttcagcaaaatattttgagaagggtgcccctttacacatctgtgcagtccaggtgatgcatcccatgcccaatgctcggtagtcaggaggagcttcctccatgcagctctgcggaagagactcttccacgctgctcatgtaaactccagattcggtgtcagttttctgacaccgaagacaatgatctaagtgcagtcaagggctttggggaaagcaggagagagtgcctcagttctagcctgtgccatgcttgcaaagttttgcaaaattctaatgagagctgggcttgcaacattggaaacttggattatttgtgagagcactgagaaatccctgggcatgtccatctggaaaaacagcatttcctctggcactttagcagaggttctgtttcaatttggcgaaggaaattaagcagtttttcacaaaagaagaactacaacgaggagaattgtccctagtatttcttctccctaattgtcaaggaagtgtaaattagaaaatgaatcaggacaatttccacctactatgttagctaatattttaaaaattgaatatcacaagggtgaggcaaagtaattgttttccagtgacattttccactgtcacacccttttagagaataatttggcaatgttactgtgagatagaaatatgtctatataattatgggaactgagacttcagaaagtaataaggaataagaatgaaatttatgaacaaacatgtggaaggttggaagcaagagtggggccaacacgcatggggaggaagcatttgggcagcgactccgcagacccagactcaagctgagctatacaacctccttacgcctcagtttcctcaactgaagaacaggaatgacaagtgcctgtttcataggaccgttgtgaggattaagtgagatataccacattatgagcttgtgcctggaaaggttgattcttagtaaatgatgactattcttttttattgcaataaaatttatacaacatagagttactattttaaccatttttgcaggtaccactgagtggcattcagtacattcacaatggtgtgcaaccgtcaccatatttccaggacatttttctcatccccaaaggaaacctcatgcccattaagcagtcactcctcattaaaatattagttatgaagactgtagcatttttttaaaaactcatgatataacattgattgaaaaaatcagtataggaaattgtgcattatgatgtaatagtaaaagaagcatataaaaatctgaaaaaagtatataaaaagaatagcaattgtatttctcagactctctttacattgtaaaaatcattttgatagcttcaaaagaaaagcaaaaagtacacaaacaacaaccaaccccaaagcagcatgacaaagcccagattgttgaatccaggtcttgggaacataaaatcttatatgacatttgcactttaatgggtcagagagtccagtggcattgggagctgccttgtgttctgcagcctcacggacagacaggaggtccagctccactgctctgttcttctggaatttcctcgtgaacaagctttggcctcagtaaccatttctttcatctttttaaacacaggtacctttgggactggccttctcaaggaagcccagctccttgttattgagaatgaagtgtgcaatcactataagtatatttgtgctgagcatttggccagaggcactgacagttgccaggtaagaaaagatcaatagatcaaagtcttgtgctctcccgtctcagtctcagtcccttagacgtcagtcccaaagtggcaaattcaggaaggttttgtcagtggaagaccccagtctaagtgttgctcagaaactccccagatctgtccctgaatgcatattcagatcatctaaggagacgtcttggggcttgagttccagatccatagcaagggagccgtaagtgccataactacctcaggccactcaccttcctggtgtgtgctggtcaccagtgactgaagtggtggcttttccagtagagaggaaggtagagggtacaggaccgagacaaattacacacacttaacaatgatgtccaggctagcccagtctaaaggaaacaccaagttaggaagcaatgcatgcaggattcacaagggattattttttttcccaggaaaaaactaagtgatgtggttttgttgaatagactttgctaagtacttaagcactgcagatgcttgagtaatatgctcataagttcctttctgatttgaattactgggaaaatgtacatatggataagagaaggatggcatcccatattaaaaggttggcagcttaaagctcacatgaattttcccctacctctgtttagggtgacagtggagggcctctggtttgcttcgagaaggacaaatacattttacaaggagtcacttcttggggtcttggctgtgcacgccccaataagcctggtgtctatgctcgtgtttcaaggtttgttacttggattgagggaatgatgagaaataattaattggacgggagacagagtgaagcatcaacctacttagaagctgaaacgtgggtaaggatttagcatgctggaaataatagacagcaatcaaacgaagacactgttcccagctaccagctatgccaaaccttggcatttttggtatttttgtgtataagcttttaaggtctgactgacaaattctgtattaaggtgtcatagctatgacatttgttaaaaataaactctgcacttattttgatttgaattaattttggttttggtcttcaaaattttcatgctcttttcatcccatctatttttatttttattttttagactttacgtcctggggtacatgtgcagaatgtgcaggtttgttacatagatgtacacgtgccatggtagtttgctgcacccatcaacctgtcatctaattcggtatttcttttagttctatccctcccctagccctccaccccttgacaggcccaggtgtgtgatgttgccctccctgtgtccatgtgttctcattgttcaactcacacttatgagtgagaacatgccgtgtttgtttttctgttcttgtgttagtttgctgagaatgatagtttccagcttcatccatgtccctgcaaaggacatgaactcatccttttttatggctgcatagaattccatggtgtatatgtgccacattttatccaatctaacattgatgggcaattgggttggttccaactctttgctattgtgaatagtgccacaataaacatacgtgtgcatgtgttttcatagcagaatgatttataatcctctgggtatatacccagtaatgggattgcagggtcaaatggtgtttctggtgctagatctttgaggaatcaccacactgtcttccacaatggttgaactaatttatgctcccaccaacaatatcaaggcattcctatttctccacatcctctccagcatctgttgtttcctgactttttaatgatcgccattctaactggcatgagatggtatctcattgtggttttgatttgcatttctctaatgatcagtgatgatgagcttttctcatatgtttgttggctgcataaatgccttttttggagaagcatctgttcatatcctttgcccactttttgatggtgttgtttttttctggtaaatttgtttaagttctttgtagattctggatattagccttttgtcagatggatagatggcaaaaattttatcctattatgtaggttgcctgttcactccgatgatagtttcttttgctgtgcagaagctctttggtttaattagatctcatttgtctattttggcttttgttaccattgcttttagtgttttagtcatgaagtcttctcccatgctatgtcctgaatggtattgcctaagttttcttccagggtttttatggttttaggttttgcatttaagtctttaatccatcttgagttaatttttgtataagtaatgcccttctttgtctcttttgatctttgttggcttaaagtatattttatcagagactagaattgcaatccctgcttttttttttctttttgctttccttttgcttggtaaatattcttccatccctttattttgagcctatgtatgtctgcacatgagataggtttcctgaatacagcacaccaatgggtcttgactctttattcaatttgccagtctgtgtcttttaattgggggcatttagtccatttacatttaaggttaatattgttatgtgtgaatttgatcctgtcattatgatgctagcgggttattttgcccattagttgatgcagtttcttcatagtgtggatggcctttacaatttggtagtttttgcagtggctggtaccaattgttcctttccatgtttagtgcttcgttcaggagctcttgtgaggcaggccttgtggtgacaaaatctttcagcatttgcttgtctgtaaaggattttatttctcctttgcttatgaagcttagtttcgctgggtatgaaattctgggttgaaaattattttcttttagaatgttgaatattggcccccactctcttcgggcttgttgggtttctgcagagagatccactgttagtctgattggcttccctttccgggtaacccaacctttctctctggctgcccttagaaatttttccttcatttcaaccttggtgaatctgacgattatgtcttgaggtggctcttctcgaggagtatctttgtggtgttctctgtatttcctgaatttgaatgttggtctgtcttgctaggttggggaagctctccttgataatatcctgaagagtgttttccaacttggttctattctccccatcactttcaggtacatcaatcaaatgtagatttggtcttttcacatagtcccatatttcttggagcctttgtttattccttttcattctttatcctctattcttgtcttcttgctttatttcattaagttgatcttcaatctctgatatcctttcttttgcttgatcgatttggctattgatacttgtatatgcttcacaaagttcttatgctgtgtttttcagtcagatcaggtcatttatgttcttctctaaactggttattctacttagcaattcatgtaaccttttttcaaggttcttagcttctttgcattgggttagaacatgctgctttagctcggaggattttgttattatacaccttatataatagcctgatataactataagatttttttgtaagcaccatcgtaaccacaaagcaaaaacctaaagtagatatacaaaagataaaaaggaatcaaagcataccactagagaaaatcacttaatcacaaataaagatacgaagagtggaataaaggaacgaagggtctacaaaacaaccagaaagcaattaacaaaatggtgatagcagatcttacctataaataattatcttgaatggaaatggattaaattttccaataaaaagacatacagtggccaaatagattaaaaaataagatccaactatatgatgcctataacacactcacttcacctgtaaggactcaaacagactgaaagtaaagggatggaaaaaatattctatgcaaatggaaacaagaagatagaggggtagttatacagattgagtatcactaatccaaacatctgaaatctgaaatactccaaaattaaaaatgtttaagtgccaacatgatgttcaaaggaaatgttcttcggagcattttggatttttgtgtttagggatgcaaaaacagtaaatatataatttgtattagtccattctcacactgctataaagaatactacaaagagactgagtaattataaaggaaagatgtttaattaactcagagttccacaggcttaacaggaagcatggctaaggaggccacaggaaacttataatcatggcggaagatgaaggagaagcaggcaccttcttcacaaggtggcaggacggagtgtgagtgtgtgaaggaggaactgtcaaacacttataaaaccatcagatcttgtgggaactcactcactctcacaagaacagcatagggaaaaccgcccccatgatccaatcccctcccactgggctcctcccttgacacatggggatcatgagggttacaattcacgatgagatttgggtgggacacagccaaaccatatcataatgcaaacattgcaaaaacaattcaaaattcaaaacatttctggtttcaggcattttggataagggaaactcaactcaacatgaggtaaagcagactttaagtcaaaaactgtaaaaagagacgaagaagaatgtaataataaggagatcagttcattacaaatatatagcaattataaatatatattaatatatatacccaaaattgtagtacctacatatagtaactaaaacaaacattaatagatctcacaggagagctacactgtaatataatcatagtagcacacttgaatagctccactttcactaatggacagatcatccagacagagaatcaatatggaaacacgagacttaaactacactttagccaagtagacctaacagaaatatatagaacattccatccaacagcagtagaatacacattattctcaagtgcacagggaatattctccagaatagatcatatgttaggtcacaaaactagtcaaaaaatgtaagaagattgaaatcatatcaggttttttttttagatcataatcgtatgaaactagaaatcaataatgggggaatattggaaaatccacaaatagatagaaattaatcaatatgctcctgaacaatcaatgagtcgaagaagatattaaaagaggaaattttaaaaaatcaagacatgagttcatgtcctttgcagggacatgaatgaagctggaaaccatcattctcagcaaactatcataaggacagaaatccaaacaccgcatgttctcactcataggtaggaattgaacaatgagaacacttggccacagggcggggaacatcacacaccagggcttgtcagggggtgggaagctggtgaagggatagcattaggagaaatatctaatgtaaatgacgagttgatgggtgcagcaaaccaacacggcacatgtatacctatgtaacaaacctgcacgttgtgcacatgtaccccagaacttaaagtataataataaaaaaagaaatatttgtttttgatttatatgccaatcagacaaaatgtgaaaagccctactgaaattaagtatcaccatgaaagataaattctggataattttttcaagttttaacaatgtagctttaattggagaaagctatcatttggaatgagttaatctatcctatactaaaataagtcacttgctttaaaacataataaatatgattttgaattgaaaacaaaaacaactcaagacaaaggaaaatggacacactaacataccaataatttatagtatgcagcaaaagtggttttaagagggaagcttttaccaataaacacttccattaaaaaagaagatctcaaataagcaacctaagattacacctcaacaaactagacaaagaactaactaacccaaaagttagtagaaggaaagaaataataaagatcacatcagaaatagtaaagactaaaaaactgataccaaaaagaaataaaactactagttggttttcaataaaataacaaaattgaccaacttttagctagattaagaaaaacagagaatactcaaataaaaccagaaagaggagacattacaatagatactacagaagtacaaacgatcataagagactactatgaataattacatgccaacaaattggataacttagaagaaatggatgaattcctagagcaaaaaacctacaaagactgactcagaaagaaatagaaaatctgaacagaccaataatgtgtacatgattgtatcagtaataacaagtctcccatcaatgaaaaggccaggacctaatggcttcactgctgaagcataccaaacattacaaagactaatatcaaccctcctcaaactcttcttaaaaactaaaaagaaggaatgctttcacattcattttatgaggatagcattacactgatactaaacacagaaaaataatacgctaataaaagaacattacaggcaatatccctgataaacatatgtgcaaaaatccgcaacaaaatactagaaaactgaatccagtagcactttaaaaagatcattcaccatgatcaagtgcgatttgtttcacgaatgcaagaatagttcaacttacacaaataaataaatgaaaggatggatgataaaaatgtgtatctatatatatatgttttatacacacacacacacacacacacacacacacacacagaggaatattattcagccttaatgaagaagaaaatcctgcctttgcatcaacctggaggacattataataagtgaaataagccagacacagaaaggcaaatactgtgtgatctcgcttacatatggaatctaagaaagtcaaattcctagaaatagagagtagcttagtgattgccagagccgtggaagggggaaatggagagatgttgatcaaaggatacaactgtatagctttgcaagataaataggttctggagatctaatgtgcagaatggtgactagagttaataatactgtattgcatacttgaaatttgctaaaagagttgatcttaagtgtcctcaccatatacacaaaagtattatgtgaggtggtgaatattttaattagcttatgataataatttcacagtgtacatctatattaaggcattacattgtacatcttaaatatatataatttttatttgtgaagtgtacctcaataaaactggaaaaaataattgaaaagtaatgaaaaaaattaaaagctattatgtgtcaaatgacattatcaagaaagtgaaaagcaacctactgatgaagcaaacctattgacaaaggcctggtgtccagaatatattaagatctctaggctgggagcagtggctcacacctgtaatcccagcacttggggaggccaaggtgggaggatcacttgagcctgggagttcgacactgcagtgagctatgattgggccactgccctccaggctgcgtgacagagtgagactgccatctcttaacccacttcttatttagaaaaagaaaatatgtagcttgctgcctgcatagtattcttggggcaaatgggaaatgagttaaaaaaaaaaaaaagaactcttacaactcaacaataaaaagaaaaacaagaacgtgaatagacattttttccaaaaaagatatacaaataggcaataagtacatgaaatgatggtcaacatcattagtcattaagaaaatgccaataaaatcacaatgaaataagacttcatatccattaaaatgtctataatttaaaaaatggaaaataacaagcatttgtgaggatgtggagaaattagaatcctgtatattgctggtgggaatgtacagggaaaatggtttggccactgtggaaaacaatttgacagttccttaaaatgctaaacatagaattaccatgtgatctaacaattttactcttaggtgtatatatacaagaattgaaaacaagtgcccaaacagataccttgcatgagaatgttcatagcagcactgttacaacagccacacccaaatgtcaatcaatagatgaggggataaacaaattgtggtttatacagctacaaaaaggaatgaagtactggtatccgctacatggctgaaacttgaaagcaagggctgggatggggtcatggaaagtaccagcttattgggtactgcattgtgctttggggtcatgaaaatgttttggaactggatggaggtggtggttgccaatgtgaacatactaaatacaacgcattgttcactataagactgctacttttcttatgagaatttcacttcaattaaaaaataccttccatgtatcctttctaaggatgatactagaatatttgctttggcaaaatgaggaagtaactttttttaaaaaggaagatgtgggatccatgaaacgggatcaaatatcagagaggaaagggggtcttctggatgacagtccatggagatcccacaactgcacagcaggccggctgtgcacccaggccacaccagagcagagccggtggttcccgaggagctctctggaagaaaaacgctagatggcctgattggtttgggggcatattgaaaaggtatataactgagaatttggagtggaattaggaaacagacataaaagcttacagaaaagaaaataatgaattctagggagaaatataaaaggatactacaggcctcagttacataaacactgaatatttacttaaccaaaattacaatataattacataattattttaggtacatatggcaaaaggatgtgtgggtgtatgtagtatgtacggtgtgtgaagtgtatgtgtgtggtatgtggacggtatgtgtatgctgtgtatgccaataaaatcacaatgaaataagacttcatatccattaaaatgtctataatttaaatgtctataattttaaaaatggaaaacacttctcatatggcaggagcaggagcaagggtgggggaggtaccacacacacttaaacaaccagatctcctgagaactcactatcaggagaacagcacctggagaaggtgctaaaccattcatgagttactgccctatgagccaatcacctcccatcagaccccgcctccacactaaggattacaatttgacttgaaatttgggcatgaacacagatcgaaaccatatcaataggtaatgactaaaactgaaaaaagaagtaccacagtcagaaagttatttagagagctgaaggtaaatgccaataggatcagttgaaagaattggaggtggccgggtgcggtggctcaggcctgtaatcccagcactttgggaggcggaggtgggtggatcgccctgaggtcaggagtttgagaccagcctggccaacatggtgaaacccagtctctactaaaaatacaaaaattagccaggcctggtggtggacgccgtagtcccagctactcaagaggctgaggcaggagaatcgcttgaaccagggaggtgaaggttgcagtgaaccgagatcgtgccactgcactccagcctgggtgacagagcaaaactccatctcaaaaataaatgaaataaagaattggaagtgtttgcctctggagagaaggaaacgcagtaattctgtaaaaacagaactttttactttttttctttttttttttttttttgagacagagtctccttctgtcacccaggctggagtgcagtggtgcagtcttggctcaccgcaacctctgcctcttgggttcaagcaattcccgtgcctcagcctcccaagtagctaggattacagatatgggctgctatatccagctaatttttttttatttttattagagatgaagtttcaccatgttggccaatctggtctcaagctcctggactcatgatcctcctgcctcggccttccaaattgctatgattacaggtgtgagccaccatgcctggacagaactttttgactctttaaactatgtgcatatataaagctgatttaaaaaaaaccaagtaaaataattttaaaatgttccaaaacagattggatgggtacacacttcatcatgagtggttgagggagactgggttagagatgaggaaattccagggactggggaaaagttaaaatgacaaactgttcacaattgttaactgcaggttgtgggaaagttggtaagttgctacagtgtttgttccctctgtaggtttgcatatatttaacatttcttaaattagcataataatgaactgtgtaatcagctgtagagttgagggtgtggagctggcacaggacagctgagctactggtttaaaataaatgacatttaaaaaaatggctatttgtagaattaacagatataagacaccctgatcaagggatgataagaaaggactccagggctctgtctcagctgtcttggcaacacctggaagacatgggcctctgcaaggtctcatactttcaggaggtgttgatgaaggatatggacagatctgaagctctgggcactgcatggtctgagaagagaagctccggaaacgcgggagctgagtgcagatgcagaagggctgtcatccagcagaggggtaggtgacaactggcctagcgagtgacccttatcatggctacatttgttgatcactttctttgtatgaggcactgctgtgattgcattaaatttccacttacctaaatccaacgttgtgcacttgtgaatttctactcttacaaaaaacacaacggcaacaacctcaaaccagtaatctagtcaaaaaagcaattcccaaggcatgacattcagattcatcagcactcacagagactacagtgattgctgataacgccaacttaatacctggccaacagcatggatcctgacctccacttttcttgtgtgtttacagaaccacaaaaaggtgcagtgttttca

SEQ ID NO: 3 - DNA, 138001 bp, Homo sapiens
  source 1..138001: mol_type = genomic DNA
ctctcccaaattgtcaaagaagtataaattagaaaatgaatcaggacaatttcaacctgttagattagctaatatttaaaaattgaacactcatacaagtgtggtgaagtgattgttttctagtgacattttacactgtcataaccttctagaaaataaattggcagtgttattgggagacagaaatatgtctatataatttatgggaacttaggctcagaaaatattaaggaataagaatgaactttatgaacaaagatgtggagggttggaagcaagaggggggccaacgcgcacggggaggaagcatttgggcagtgactccgcagacccaggctcaggttgaactagacaacctccttacacctcagtttccttaactgtagagcaggagtgatggaactgcctgtttcataggactgttgtgaggatgaagtgagatacaccacattataagcttgtgcctggaaaggataatgcttagtaaatgatgactattcttttttattgcaataaaatgtacacagcgtaagagttactattttaaccatttttgcagggtaccaccaagtggcatttagtacattcacagtggtgtgcaaccatcatcatatttccagaatattttcctcatccccaaaggaaacctcatgctcattaatcagtagctctcctttaaaatattagttatgaagatcatagcactatacaaaactcattatgtaatgttgagtgaaaaaatcagggtgtgaaattttgtgatatgatgtaattagtgaaagaagcatacaaaaagtctgaaaatataaaaacaatagcaattgcatttctcagactctacatttaaacattattctttatggttttaaaagcaaagaaaaaggtaaagaaacaacaaccaaccgcaaagcaccatgacaaagctcagattgttaaatccaggtttttggaacatagactcttatatgacgtttacactctccagggttcagagagtctggcagcattgggagctgccttgtgttctacagcctcacggacagacaggaggtccatcaccactgctctgttcttctggagtttccttgtgaacatgttgtggacgtagttaccatttctttcatctttttaaacacaggtacctttggggctggctttctcaaggaagcccagctccctgtgattgagaatgaagtgtgcaatcgctatgagtttctgaatggaagagtcaaatccactgagctctgtgctgggcatttggctggaggcattgacagttgcaaggtaagaaaagatcaagagaccaaagttagtcttgtgctctcctgtctcagtctcagtcccttagacttgagtcccaaagtagcgaattcaagtaggatttaatcaatggaagaccccagtctaagtgttgctcagaaactccctagatctgtcccaaatgtatattcagatcatccaaggggacttcttggggcttgagttccagatcagcagcaagggagccataagtgccataactacctcagaccactcaccctcctggggtgtcccggtggccagggactaaagtggtgatttttctggtagggaaggaggtagagggtacaggacagagactaactgcacacaatatctgagactggagctcagatattgctgatgatcagagttggcgtgtctccccaattgatttacaactggggcttggatactgttttaaacgggaggagcctcctaaccatcttgacacaaccactgacgtgactacactagagatagactctttccacttaattctaccactcttgctttacttcatgagaacgaaaatgtaagattgcaccatgaattcatttgcggaaagattgatactatgcttttattttattttattttattttattttattttattttattttattgagactctcaccccggttgaagtgcactgacgtgattttggctcactgcaacttccacctcctgggttcaagtgaatactccagcctccctagtagctgggattacaggtgcccaccaccacgcctggctaatttttgtatttttagtagagatggggtttcaccacattggcctggctggtctcaaactcctgaccttgtgatccacctgtcttggcctcccaaagtgctgggattacagagttgagccaccgcactcgaccctatgttttatttttaaaaatatttatttatttatttaagccacaactactagaataggaaggattgatattttattaattttatttggtatttattatttttttttctttcctgagacattcttgctctgtcacccaggctggagtgcagtggcacattcttggctcactgcaacctccatctcctgtgttcaagcaattctagtgcctcagcctacttagtagctgggatgactggcatgtgcctccacacccagctaatttttgtattttttgtagagacagggttttggcatgttgcccaggcttgtctcaaactcctggcctcaggtgatccatctgccgtggcctcccaaaatgctgggattataggcatgagccaccaccccctcctggaaggattgatatcttataacataatttataattacagaaaacatgtgagttcactaggaataaataaattttgaagataataaaagattttcacttatgttgtcatttcggcacagtttggtataggatgtggagatgttaacatttatacctagcttgctcgtaaactaagacctgaaagggttgtgtctatcagctgcacccctgggtagcgacacaacctcgggaaggcctcagccccctcctcgtacagcactgcctgttggaaagcttgagggaggctatggatgtgcagcacttggcagagggtctggtcatggaagttaccagcaaatatgagctacttttatgattttattttatccaaaagaaagagaatgaaagaagaggggaggaaacaagactaatcaggaaagatgaaggtctaggggtgagggaaggagtaaggagacataaaggcaatgtggagcagctgaggggggaaatggctttcaccacttcccagcatctattgacattgcactctcaaatattttataagactctatattcaaggtaatgtttgaaccctgctgagccagtggcatgggtctctgagagaatcattaacttaatttgactatctggtttgtgggtgcgtttactctcatgtaagtcaacaatgtcctgggattgggacacactttctgggcactgctggccagtcccaaaatggaacataaggaagtggttcttctacttcttttatttctgaaatcaggtaagacatagtttttttaaattataagaattattttttctcccacaatgtagtaaaaatacatatgccatggctttatgtgcaattcatttaatttttgattcatgaaattcccagttcaaaatcttgtatatgattgaaaaattcttaaaaaaataagtttaatttccccgtgaagactgtcacggtgctggaatgaatgggcagaaaaaataatggttgatttttctaatctaaaagagtgtgcctacatgatggccagtctggctgaaaaataaatagccattgtagctaactatgcaaaggatggctaagctcttcgcttggttctcagtttcattaatttatatcatctctgttcaggtgccatgctcccctcactagcaagttgaaacaatgaaataactctttgaatatgtttggttccttgacctgttcatggagtgggactcagcatttctctctttgttatggcctgagtaaggctttccatcggtatacatttgcttcttatccctggagaaattatacacatccatttgccagatgatatacgcatataatgattcaacaaatactcagggtatttgttgagtgggttaggtccccacatttttatacatacatacacacatacacaccgtgtgtgattgtgaatgtaagtgtgtgtcctttacaaatactagcttatttagctcatggtataggtagggtagcatagtcatccccattttataaacaaagaaatctagacttaggaaaatcatgttatttgtctcgtgaccaaattcccaaatcaaggaaataaagaaacctggatttaagccagatttccaagaaaaaatctagggctcttctcactttttcatctttgttccaacatttgaaaaaataaatctaaacacattccaatgtaactgaagagcaggttaattgtttgccacttgcagaatccaattaagaagagagaagtctggtataaagaaagtgatttgcttccaaagctagcttaggggaagaaatgcagcagtcctgccgtactacttcactttaggagcagaaagtggcacttttaaaaggcaacagaggaggcgagcaaggattcaggggtccatgctagcttgggcaccttatccaccaggtagttgagcagttgcctgctggtgcctttgtgagcagggtgttgtcccttgaggcaaatctctggagggtgagagttttgtagtgggcatgctttggtttataaatcacctgtgaactcaggagttccatcttgaagcacatacatagttagatgaacttgccctgcagggagagtctgatgaaagggaggtagatgcttgcaatttaatctataaattaccagataaaattttacaagttgactttaaagtcaaacacatttgaatttagtggaagccattcaagaaaatatcaaagaaaatacagagcaggagaagattaagcaaagagttttttggggaaattggtgtctatgtctgtgtgtgtagggagtgcaggggatatgaatattctatttcagcccatggaaactaggatgtagatcactgtgaacttattcagcaggctacacccaaaggctagaacaaacttctctgccacaggattaacatatgttttaatcgacctggggggcacattctctgataagctcttttggaaagccaggctttctgtggacgtgttatctttccaatgtgtgctggaatgcccggggagaggaaaaagtttcttttacagccatgctcagtgagaagcggagaaacatcttctattcacaaattgctaagtcttttacacatgcaaatatgcatacacattcacacaccacagtgaggaagaaattctcacaccattaataaaatacatttacttcagtagcaatatacatctacattttgcctataatataaaagtatttttcctattaaaagatttgtttaatgtttcttcaccaacaaataaaccctattaaatccccattgccatatgagccctggaggtgaatcagagaaacaaaaggattgtggaaaaatcatcaggttaaaaaaagaaaaattgattctgttttgggatatttcctagcaacatgagctggggaggggatctcagcagtgatgctctatgaagcataataaaatgacacagttacaggtaacttagttaaagggggaaataaatggaagtttcctctttttgaatatcaattgtagcctgctctgctacatttcaaaaacactcttcaaaatgtttaactgaactcactgtaggaagcaccttattaatttattgtgtgttttgaagtcacactgtgagctatagaatttacccaagcacaactcttcctggaaaagagagttcaaatgagaaacagtgcggggtgaagacatggatatgggcctaaaatatctatttctcaatgatattttgatatatctatcaagtgctttttagtggattaggttcagaatgcatcagccaatgcctgttcaataatccagttttccagcatagagcatattaaattgaggaaggacaaagtcacagaggtggggagcaggtggactgtggccaaggactttgcatgaaacagtgagcgtgcatcctcctccttgccctgccctcatggtctgtgtactctcaggaggtcaggacaggcctttctgagaatgagaatctgttcatctgcctttctactggatacttgtcatcggcatacaaacacatgttctctgcagtgtgtcatctttcagaacctcccctgaccctgtattccctagaagtctcgctgctttcagagccaggcttctctcctgctgccacccccactgctcttctagtcactctttaacccactccatctgcatgtggcccccaccacacccctcaaagtggtcaaggttgtcctgttgcttaattccatggaagcttggctatcttcattttattagcctcttttggcctctcaccctgtgaaaatcactacattttgtgccagagatggagctggcatctccaggcttggaagagggctgctgaagctcagccaggtgtcctaaggagcctcaggacaggggatgctcagtagccttgcaatgggaacacagctgagccccacttggccaccctttgccacaaccaggcagaaagcagcttttgaacagatttgttgcctcagatttgatctcaaagaaaaatcgtgggcagtattggtcccaggttctgcttttttacaatttcctctgaaatctggatgcctatcaacaccttggaaaaactgaattctccccaactaatagtggtgtgtcactgtagtaagcctagtacaaaaatggccttctttgtggaggagcttcatatcctccattttttttttgcttaatttttgcccaagatgagaacataatttagttcactttttatttattcccaacatcatccatgcaccaacatttttgtaactaaaggagggaccattcagaagatgcttatcaactgtcaaagtgacagtgttacaaccaatgcacatattgtaagaaatcaaacaatggcctccaaggttcatttctacacagggattagcagatcaacatcaatcttggcaacacagttgccactgatggtgtcttattttttttatcatgacatggcaatcaagagcaaacatgatttattcttatttaagattttatggttagactaggcagatagctagatatgagcaggaggtggaagcccctgagagaatggaggtctggagaatctgaaaccccagagattacccaagtcctgcatgctagacatgagtggaggagggggaatacctaggtagaaaagaatgccccttaagatgcccagcagtcgctcactgtgcagttaacttttcagaatgctgctagatacatgctgatagggagggaagagggcaaaggagaaattcctaagagatacacggttgcagttagtatacatctgagtgctatacaaccttctttgggtggtggcaagaagcaatgcagccattacgtagaattcatatcaaacacctgtatcacaggtgttaaagaaacaagaaacattgtacttcttgtattcttaataatgatttgcaatattgtctttagtatcactgcaaacctctataaatatgatttttaaaaagtatttctttaggttggaattacttctacgcattgacttatcttcctgggtttcattagccgtacccgttgtactttcttccttaccactgtttatctcaaactcttgagattaaagtatgggctcaggagggagcgaggagcttcaggactctcacggacctccagcacagtgtagctgccttatggaaaagtggccacactgttttctgcactggtccctgcccctactattcctcactgggcagagcacagccaccctggccctgcctgaacattttagtcagtgttggctctgtgcttctctggggaggaaatccaagagacaacccacagcccctctgccatttcagctgcagcagtaccaccgttaatgcccttgggcttgagaaagaagggacctggccacttccctgacacctccagcacacagcagggaaagaattccagtttctctttcttgtgagctttcacctgctactcttcaccaggcaaggctcctggcttgggcccacagtgcaggcacctcgaactcagttgaacatttccactggctgcactctgtgtttttgtggggtgaagctcccagaggtgactgaaagtccttctgccactaacactgcagtcatactgcccttgctgtacttggactagggaaggaaaaaagatcctgagtgctttactcacaccccagtgtgccccagccaccctatggaaaagaggccagtgtgtcatccctgcaagcaccctgaggcccctgcccctgctgcccccaagctgtagagccagaatataaagctggcagaaaaatgtaaaaaggctagactggcttagcctcccagcctacatctttctcctgtgctggatccttcctgctcttgaacatcggactccaagttcttcagctgtgggacttggactgtcttccttgctcctcagattgcaggtggcctattatgggaccttgtaatcttgtgagttaataccacttaataagctcccctttgtgtgagtatatctatatctatagatagatataggtatactcactatatatacacatatatacatatactctctctctctctctctcatatatatatatatataatctcctattagttctgtccctctagagaaccccgactaatacagattttcataccagaagtggttcttgaggaacagaatattaaggatggaattctttcattggttttgggacttctggtgttggctgattaatatgattagaccaaaaaatgctaaggactctacttctaatagtatggagaacactgatagtacttggcctgaattgtttagagagttatgcaaaataaatgcatttgacactactgattcatcacttatgagaggcaaggagtttagtgactctatacataatacctttgactatatgtggagaaccaaggaacataatgaagttggttgattgctcctaagttctctggagaaagagatgaaagaaaatgatgatctcaggggatctgtctcccaccttcagaagcagatactgagccacaaatctgctaagattgccctgaatgagagttttaactcctgtagagaaagagttgaaattgtgaaaaaacagagacaagctgttatcatgcgagtagctgatctgcaacaagaggtgcatgcacagccttgccaggtgtttactgttaaagtgagggcattgactggaaaaaaatgggaccctggaacttggagtggggatgtgtgggagaaccctgatgaagctgaggacactgagtttgtgaactctgatgaaacttttttgccagaagaaacagtttccccatccccagtagtggtaacatcccctccctgacccgtgctgccattagcctttccacctttgtctgaggatgtaaaccctgcactgcttgaggcaacagtgatggccttccctgaggcagctgccaggcaagataatgttgattctcctcaagaggcacccctaatgcccctgaatgcttctagacctataactaggctaaattccttgcgggccccagaggtgaggttcagagtgtgacccatgaggaggtgcattatactctaaaagaactgcttaagctttctaatttatattggcagaaatctggagaacaggcatgggaatggatattaagggtaagggataatggtggaagggacatagagttggatcaagctgaatttattggtttggccctactaagtagggattctgcatttaatgttgcagctcggggacttagaaaaggttctgatagggccgggagcagtggctcacgcctgtaatcccagcaccttgggaggcgggggcgggcagatcacgagatcaggagattgagacaattctggctaaaatggtgaaaccccatctctgctaaaaatacaaaaattagctgggcatggtgatgcgtaactgtaatctcatctacttgggaggctgaggcaagagaactgcttgaacctgtgaggcagagattgcagtgagccaagatcgccccactgcattccagcctggtaacagagcaagactccatttccaaaaaaaaaaaaaaaaaagttataatagtttatttgcttggttagctgaaatatggattaaaagatggtccaatgttagtgagctggaaatgccttggtttaatgtagaggaagtgatccaaaggcttagggagattaggatggtggagtggattagtcactttagacctactcatcccagctgggagggtccagaagatacacccttggccgaagctttgtgaaatagatttgtgagagcagcacctgtatttttgaagagcccgtaattgctcttctctgtatgtcagatctaacagtaggaaccacagtcactcaactacaaaatttaaatacaatgggaataattggatcctgaggtggcaggggccaagtgttggcactgaaccatcaaaggcaaggtgggcataactaccataatagacagcagaggcaaagcagccatcagaatagtctgactcatgtagagctctggcattggctaattaatcatggtgttcctagaagtgaaattgatgggaaacctactgtattcctacttgatttatataaacaaaaaactgccaggtagaatggactaaagactaatctgaattataaaaacagagaatcatgggccctcaatcaatttccagactcgaacctgttacagttccagaacccactgaatgaaggggaggctggatccccttgaggaaggacaccactaggctactgacaacttatgctgttactctttctcccatccttccctaaggagacctctggccttttaccagggtaactgtgtgtactggagaaagggaagtaatgagacatttcagaaagtactggacactggctctgagctgacgttgattccagggtacccaaaacgttattgtggttccccagttaaagtaggggcttatggaggttaggtaattaatggagttttagctcatttctgacttacagtggttccagtgggtccctggacttatcctctggtcattttcccagtgccaaaatgcataatttgtatagacatacttattagctggcagaaatgccacattggctccctgactggtaggatgagggctattatggtgggaaaggccaaacagaagccattagagctgtctctacctagaaaaataaaaaaatcaaaaacaatatcccatccctggagggactgaagtgattagtgtcaccatcaaggacttgaaagacgcaggggtggtgattcccaccacatccctgttcaactctcccatttgacctgtgcagaggacagatggatcttggaaaatgatggtggattattttaagcttaaccaagtggtgactccaattgcagctgctctaccagttgtggttttgttgcttgagcaaattaacacatctcctggtgcctggtatgcagccattggcttggcaagtggctttttctccattcctgtccataagacccaccagaagcaatttgccttcagctgacaaggccagcattatacctttaccaccctacctcaggggtgtatcaactctccagctttgtgtcataatcttatttggagagaccttgctcgcttttcacttccacgagatataacactggtccattacattcatgacattatgatgattggatacagtgagcaagaagtagcaaacacactgaacttattggtgagacatttgtatgccagaggatgggaaataaatccagctaaaatttagggactttctacctcggtaaaatttctagggttccagtggcatgagacctatggagatattccttctaaggtgaagcataacttgctgcgtttggcccctcttacaaccaagaaagaggcacaatgcctggtgggcctatttggattttggaggcaacacattcctcgtttgggtgtgttactctggcccatttatcgagtgacctgaaaggctgccagatttaagtgcagtctagaacaaaagaaggctctgaaacaggtccaggctgctgtgaaagctgctctgccatttgggccacatgaccccgcagatccaatggtgcttgaggtgtcagtggcagatagggatgctgtttggagcctttggcaggcccccataggtgaatcacagtggagacctctaggattttggagcaaggccctgccacttctgcagataactactctccttttgagagacagctattggtctgttattgggctttggtggtaactgaacgtttgactgtgggtcataaagtcaccatgctacctgaacctgcctatcatgaactggttgctttctgacccatctagccatgaagtgggtcagcacagcggcatttcatcatcaaattgaagtggtgtgtatgtgatcgggcttgagcaggtcctgaaggcacaagtaagttacataaggaagtggctcaaatgcccatgttctccactcatgccaccctgccttccctcccccagcctgcaccaatggcctcatggggagttccctatgatcagttgacagaggaagggaagactaaggactggttcatagatggttctgcacgatatgcaggcaccacccgaaagtggacagctgcagcactatatccactttctaaatgcatgtgtacacttgtgctaagaaaatatctttattttatttcctttatttttcctttatcatgtgaccttagatttatggacttcacatcagcatttaagcatttaagtgttgttcatatcagcatttaaatattgttaaccttatgtaataacttttggtttggggattggtgcgtttctggttgtatgaggatagttgtattatattaggcataattatgaccttattattgtctttatttgaagattatgtatgatttcaggatgtgtgtatgggttcaagttgacaaggagttggacttgtgatggttaatactgtcaacttgattggattgaaagatgcaaagtattaatctcggttatgtctgtgagggtgtggcaaaaggagattaacatttgagtcagtgggctgggaaggcagacccacccttaatctgggtacacaccatctaatcaagttccagtgtggccagattgtaaagcagggagaaaaatgtgaaaagactagactgaattagcttcccagcctacatctttctcctgtgccaaatgcttcctgctcttgaacatcggactccaagttcttcagcgttgggagttggactggctttcttgctcctcagcttgcagagggcctgttgtggaaccttgtgatccgctgagttaatactacttaataagatcccctttatatacatataatatattatattatatataatatatataatatatattatatataatatatataatatattatatattatatataatatatattatatattatatataatatatattatatataatatatattatatattatatattatatataatatatattatatataatatatataaaatatatatatatcctattagttctgtccctctagagaaccctgactaatacaatttatgtcattaatctcatttattgatttgtatacattgaaccaaccttatatcccaggaataaaacctacttgattgtggtggattagctttttgatgtactcttggattcaattgctggtattttattgagaatttttgcatctgtgttcatcaaggatattggcttgaagttttctttttttgttgttccatatcagaatgatgacgacctcatagaatgagttagtctgtcctcttttatcttttggaattgtttcaggaggcttgatatcagctcttctttatatgactggtatactttggctaggaatctctctggtccaggggtttttctggtgtaggtttttaattactgattcaacttcagaactcattactcattattgagttctaaaactcactttcatgtactcttcaaaagactgtcttcttctgttgttgagcggggtgttctctcaaggtcgtttaggtgaaggtggttgctggtgttcttctgtatccttactgcttgtctttctctttttttattgactactgaggattaatggtgatgtgtccaactttaactctagattagtctatttctcttttagattgtaactctgttttatatattttgaagctctgttgttaggcatgtgtatttggattgttaggtcttcttgatgatgacctttatcattatgtaatgtttcttcttatctctggaagtattcgttgttctgaagtctatttgtgctgatatgaatacagccttcacagctctattttcactagtatttgtatatctttttctcagcttttaaattgagatgttcagaccatttgcattaaagtagttgttaataggattaaatttaaatctaccattaagttggttatttctctttgtcccatttaaactttgttccttttttcatatttttctgccttcatttatattgagtttatctccacgacttacttattaaattaatttttaatggttttagtattttccacaatgtttataatatatactttgattttttcacattccaccttcaaatgacagaattatactggatatatagaaatcttacatcattgcacttctccttcctccctctcaaaatgttgtgctattgctctttgtaatagaggcttacttctattatgttatagctctcataatacattgacactatttttaccctgaataatcagttgttttttaaagtgattatgactacaaatattttgaataatttctttattttaccatttctggtgctccttatcttttacagtagatcccaatttccatctggagtcacattctttctgtgaaaaacaacctttagcatttcttatagcacgggactgctgttgctgttgtctttcagcttttctttgtctgaagaagtctttattttgccttcagtttttaaaagtgattttgctgagtatagatactgggttgagagtttcattccttgtatcattttaacaatgatgttccattatattccgttttgaatagtttctgactagaaatctgatctttgtttctttgtattcaatagttcctttttctctgactgcctttaagatattctcatctttgtttttcaacagtttgactataatttgtttattattaactttttgtatttattctgcttgaggtttcctgagctccttggatttgcagattgttgatttttattgtttttgtaaaattcatagccattatctattctactgttttgttttttttttcacttctctctctctgtattcttctttttggactgtaagtattcaaatgttagatcattcatattgcttcataaaccttatatgcttcttctgcttttttttttttgtcaggaactctttttttgtatctgtgttggtttggataagttctagtagactatgttcaagtttatggattattttgttagttgtgtctaattgactcctcagtgcattcagagaattcttcatctctgatattataaatctcttcctagcattttcatgttactcttttctatagtttccatctctttgctgaaattctccccctatccatggatattgtccacctttaccacaagattctttaacatattaacataggtatcatacaaacccaaactgatagtttccagatggtgtcttttctgagtctgtctgtcttgattgctttattatttaacagtgacttatcttccctcttcagcttttggtgtgtcttgtaattgtttaatcaaacactgggtatcataaatggaggaacagtagagattgcagtaaatattatttatgctttgaaatgggcacccatcttctgttgaaaatatgttttgtggtcaattgagtcaacctagtaactggttgaactgaatttggcatttgtgcttgttgcttttatcttaaatgcaccacaggtttaaattcctccagtgatgggttgctgctatcttttgcttagagtggggcctggggtgtggaagaattttctcagtgttcctatctattattagattttagcagtcactgcatgcctgcactacagaggggatatcttcatacacataatctaaccccattgaaactgctgtttcttcttaatgaatgctcaatctttggtggaaataaacaaatgctgtatctcctggagccacttcagtcttagtcaggttctgcagggctttgaagggaatgcattctcagtattcttgtgccttatttggatggaacttgaacctgtggtgggtttggagagaaagagtagcagacgtctgctatgttgcaatgcaggatgctgggcacaagaaaatttccagtctctcctccaaggaaataagatttgatcatctacctatccctgagaagtgaagggctttgcctgcggtgctagatgcaaaaccatttttctccccccattgcccagaaacttaaggctttggcttttctgagcagtggtctagggaattgtgcaaggttttcatatttgaccctgacagcccatcaccacctacagcttgcagtgccaaatgtatctccctctgatctctcctgtcctgtggtcctcatgaacattaagaagagatttctaaaaaagagcttgcacatgagcatagtttctggtgagaagaattctgatatgttaacttcctctaaacttttaaataaaatatttctaagaattaaataaagttctagaatgatatgaatctattcctttggttttttgcacgtctgtctgcctgctaatcaagagaagagaatggtcgtaattctcagagactttttcctgtttgtgtcataaatgacttcacatttttttctgttctaagaactattcagcttgatttcttctgttttaattttagcagcacctgagcaaagccatgtggtccaggattgctaccatggtgatggacagagttatcgaggcacgtactccaccactgtcacaggaaggacctgccaagcttggtcatctatgacaccacatcaacataataggaccacagaaaactacccaaatgcgtatgtcattaatcttacagtaagcaaaacaaggtccaagtaaaatttgtcttagaaaaggtgtgcgtcaagctaacttcttatgattaaatttttctcacacatagaatgcatggcaaaatgtctgagaaacattactttgagcaaagagtatgatagaagagaaatgttaagctggctctctttcctgagagtttgataaaatcaggagaatatctggcggtggtgaggccacaataatggaaaatcagaatgtttagacagagtcagcttcaacaacactcactaaaggtcaatgtgatctttaccccttgaaattctataattctaatctccaattcctgaagtgaaggttgtgttggccttttctgtcttggctcacaagtaaatgatatgtgcatatctatggaaaggcgaatctatctttttctatatctatgtctattccaacgggtagaaacaccctgggtcctgagcaccagtggtctgaaggaatacgggttgccaggaagagagaagcaaaggcaggaaggcagatgaaagtaagaaatgagacagatgctaaacaataaaaagtgcgggaagatagacagaagctggggtctgaccacaccatggccagtctttcacacataagtgactaccaaagacaagaaaaaatgatttccgcttgttggacaatagatggtagaggaccaagggaattgcgagagagagaacaatgagatcaactcaacagatgcactggttttcttcctggagacccttcctgcactgaagggcaggagatggagcccaaaaaaaactgtagccatcttgctgaacagaggagggacattggagtttgggattattcaggtggctaggattttctaggcctgctaacaatgagaacagatttgtggaggaaaggagttctagaaatatgcatagaaatctcctcgagtcattggctaaacatgaagctgcatgtacacagaaaatagatccacaagaaagtagggcaaagaacatctacggaagagcagcaactacaatggaacagtgagctcaataaacatgacagagctcaaatagcactaagggatattggagtttggaccacacagaggagagagacttcactgaacatcttgggcattcagtagagacccaggaaaagccatactttaggagtagaattagtatattcttagaataaaggcagctccacacaaacaatagcaaaactgaaaaggaagtctccaagcatcagaatgatgtccaagtcaatgaactgcctctgagaggaaaactcaaccatctttagaggtaaacatcaaagtcaagtggctcagctatgcagtatccacagtgtgaggcctaaatataaaacttgactacacatagaaaccttttagtgtgacccacaagcaggaggaaaatcagccaatacaaacagacccagaagagacagaaatgattagaatggcataaaaatttgacatatcactatataataattgagttctaggatttaagaaaacatgaatatagaatgcaacagacaccttatccagagacagtaagagtataaagagccaaatcgaagaactactaagagatatgtcttaaatgaaaaaattactagatggcctccccatctagttagacatttcagaagaaaataccaaatgaaaaataattgcatagaacctacagaaccagatacacacatacaaaacacacgcatgcatacacacacactcaaacatgtataagcttacaaacacacacacacatccacaaatgctgaaaaatgaaatcaaccgagccacacagacataaaggaaaacataaaaagatttcctacatgtgggaagcaagtcacagaaagggggaaggagattggaacagaaatatatactgaaagcaaggatggctgaaaattttccaaatataaagaagattaaaaaatcacggactcaagaagctcaatggatcagaaaaataatttctaaaatgacaattataggatgccactgggtacatagcagttcaactgtcagagggcaaagacataatacacagaaaaatctcgtaaggaacgggaaaaacaaaaagctgtgtcttgctagaggaacagtgatacaagtgactaatgtgttcccatcagaaacactgcaacctggacacaaaagaataacattaaagtaataaacgtaagaaagaagagctcaactgagaaggctacatccagcaataaaatgccttgaagttcatccatgttggaggaatgcacattgtgcactcccctaaacaaagaaaccggaaactgtaagactttggaatcagcaggcttatgtaacaaaagaggtgaccctaaggaattaaggagaagaagaatagaacaagaagggaactttctgcagcctatataatgaagaacctagcaattggcaaatgtagatgaaaatgctacatgttttcttgatcaaacgtttatatctttttaaatgagagttgacgagttgaagcaaaatgataccaatatatttaactttaccatatgtagaagtaaaaatttgaacatgtagcataaatcatgtagggattaattggaagtgtaccactgtaagtttcttacctcatgcacgatagtatgtaatactaataaaaggttaatgtgtgggttcaaagggatattgcaaatcctagagcaatcacaaagtttttaactctgaggtttgttgtataataacaatattttatgtattcaaaagagggaagccaaggaagaaaaaaaagtctttaaagagctctggctcttagtacatccagttgctcattgaatgagcttcctggaatggagggtctgggactgagactaggccacatgtgtagagccactagagacacaatgttggatccccatggcccataatacatttcccattttctcaggcagccacaggtcatgaatgtgaggatactgagaggttggagcaacgttcttgggaggcataaggaagagcgaatgcttcaagatccccgcagcccaaactcctcagctgctttgcctcctaattcattgttttttgctcctccatagctgtccgacctcttcagatctcttagtcttcctgccatcttcctttatgccatgggacccactgttctttcaactcatcccccagttctggagtggctgtggacagcagaggatagactgagagcaggagagaaggtcctgcccaggaacccattctagagatactgcattctgcctgggagcaagttttccagggcagctttgagaagtcttgcagaaacaaacctacttgaccgacatgatatgggaatgacagacagtaatactatttgcacaatgcttttccatgggaaaggtagagccttttcactaggttttgagtacatggagtgtgagagttgacctggaaaggttatcctccttgatgccatgttttctctgaagaactacatgttcgttgcaactcccacattagaatatgaagtcctaccgagagagatacggagactagacagatacagatgcatttgcatgtgaatacacaatcccacaatacagacgtcaaaacccataccagttattccagagagatggattgggcagaaggcagaaggagaatactctgatcgtttttcggccacgtgtgtgtgttatctcagtgtttctaagaagcgtttgctactttagattttttatttaaaaaaaatagtaataatctattaagtatgagagatgtgcagagaggattagtgatcgagagccatttttgctggtggcaatcatatggtacttttaatgggaatattagaaaggcaccggtaatgaccttgttgcagcacaaaggagagagtgtggggtgcccctgcatgttgtcccacctcttgtgacgtgtatcgttttggaatttccagtggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaaggtaaggagtctgtggccagacatctacacgcttcgatgctgggatgaaaagccatggaaattcccactgatgcagccgccttcaatggtaaacggatgctcgagtgttgcctgagttctaccatgtaggaggaagcctccgtgcactctctgggggagccagcggagtgatttctggtgcaacgtggttgggctttgtctttaggatgggcacaaaccctccagggggatcgacttcaaaattcaccttgttgtaaaacgggctacctcagtgtcccagccaaaatttttattgtaacatgctgtcaggtgtgtcactctttccaagccagtaagcttttccggggatttcttcaagtagccagcattcagagcaatcttcagcattgcagattctgagaaatgtggctctggagcctgtcaccctcgagaaacctaagagggctgcattgattccatgtggccctgggtctatggagcagtacatgagctcccagtgctctaaggctcttcagccctaggctttgaagggagtgatttctcagtattcttaaacctctttctgatgacacttgtacctgtgaggggtctagagagaaagagtagtagactcctactttactacaattcaggatgcagggcatgagaggattccctctctcctccaagggaagaagcttttggcgtgcacacatccctgagaagcaaagtgtctttgtcttcagtcagatacataggaccgttttctgccccatggcccggaagccaaaggccttggctttcatgatcaacggtctagggaaacatgcaaaatttccatgtctgtcccaaactctgcccccgacagccaattaccacctgcagcccgcattgccaaatgcggtgccgtttgcatgaagattcagtagagtttcctagaaaggtgctacctcgtgagctcactttccaatgaggaatctgatctgttgtgtttctctaaggtgtcaggtgaaatatttccaagaacttactacagttctagaatgggaggaatctgttgctttggtgtttgtttgttggtcggttttctcacatccatctgcctatggataaggaaaagagaacggtcgtaattctcatagactcctttctggttgtgtcacaaatggcttcacatgtttctctatgctcagagatactcagcttgatttcccgtgttttcatttcagcaccgactgagcaaaggcctggggtgcaggagtgctaccatggtaatggacagagttatcgaggcacatactccaccactgtcacaggaagaacctgccaagcttggtcatctatgacaccacactcgcatagtcggaccccagaatactacccaaatgcgtatgtctttgttctttaccataagagaagaaagggccaagtgaagtttctgttacaagagatgtgtctcaagctgagttctccgaactcaacttgtgacagatgcagatggcgtagcaaaatgtctcaggatgattgccttggagctaagggtctgagagaagggaaatgttaagctccctctccttcctcctagttctattgagcagaagggaaatctggaggtgaggagatcacattatgaagaaagtcagaatgacaaaggaccagacacttagattacccttccacaacaccaactaaacgtcaatggagactttccagttggaattccgttattctggcttccacttcctgaagggaaggttgcgtttgccttttctctctgggttcaagaggaaagaataggtgcttatttatggacaggtgaattgatctgtttctatatctacgtatattccgattgtcagaaaaacactcgttcctaagtaccagtggcctgaagggatacaggttcccagcaagagaagatccaaggaaggaaggcagatgagagtcagcacagagagggatgctgaaaagtaaaagggatgggtggatggagagaagcccgggtctgaccacccaatggccaatattttggccacaagcgactaccagagacatggaaaaatggtttctacatgtgggacaacagatggtagaggacctagagaattgagagaggggcaatgatgggctccactccgcagatgccttggctttcttcctggatacccttcctgcactgaatagcaaggagatggagcccaagcagactgtagccatcttgctgaatggaggagagggattggagtttgggatgactgtggtagctgaaatttttctaggtctgctagaaataagaactggtttgtgtggaggaaaagagctctacaaatacgcatagaagtctcctccagtcgttggcctgacatgacgctgcctgtgcacaggaaatggttccacgagaaagtgtggcaaagaacatttactgagaaacagcaagtacaagagcacaggaagctcaataaagaagagagagatcacatagcactctgggatactggagttcttcccagctagaccagagagtcctcacggagcacattgccaattcagtggagaccccagaacagccgtaatttaaaggtacacttagtatattactagaataaagtcagctgcagacaaccccttgcacagctggaaagcaagtgtccaagcatcaaatcggtttccaatcaatgaagtgcctgtgagaggaaatctcaactctctttagaagtaaacaacaaagtcgattgcctcagctatgcggtatccgcagagtgagtcctaaatttaaaatctgactacatgtagaaaagcgtttcgtgtgacccatgaccaggaaataaatcgggtaatacaaacaggctcaggaatgagagaaatgattagaattgcgtgaaaatttgacatatcagtatgataactgatttcaaatatttaaaaaaacaacatgcaagaaagcagatatcatatcaagagaaattaacagtacagaatagccaaattaaattaaagaggtagtataaaaaaagtatgtcttaattgaaaaaaattactgtatggccggctgatcaatttagacgtttcagaggaaaacattacccaacacacaattctagagaacctacagaatgagctacacacacacacacacacacacacacacacactgaaaacacacccatactcacacacacgcagaaactcacaagttctaacacacacagacacgcgcacccctgaagaaacagtgaaatataaaattaagcgagcctcacagacatgtaggaaaatatgaaaagatttcctgcatgtgggaagcaagtcacagtaaagagcaagggagtttataatagaaacaaataccagaatcaaggatggctgataacttttcaattacgaagaacattaaaaaaaatcacagaatcgtgaaactcaagggatcatatagggaatttcggaaaaaaaacccaacctgtatgatgtacttttgtacatcacagttcgaaggtaacaaggcaaagatgtaataagaagaaacctgtcacgagaaactggaggaaaaagagctgtgtcttcctacaagtacactgatacaaattgccaatgtgttcacctcagaaacactggaagccagataccagggaatattgttaaaatgataatcaggaacaaaaagagatcaaccgggaatgctgaatccagcaataaaatgccttgaaggtcatccatgtcggataaatgcatattgtgcactgccccaaagaaagaaaccggaaactgtaagaattggaaatcagcaggcttatgtaacaagagaggtgacccgaaggaattaggtagaagaagaattgaacaagaaaggaactttctgcagcccacgtaatgaagaatccagcaattggcaaatgtagatagatgtaaatgcaaaatattttcttgatcaaatttctatatctttgtaaatgagagttgactacttgaaacaaaatgatagcaagatatttaacttcagcatatgtagaggtaagaatttgaaatggtagcataaatcacgaagggattaattcgaagtgtaccgttgtaagtttctttacctcatgcacgatggtgtgtcatattaataaaagggtactgtgcgggttcgaagggatattgcaaatcctagagcaatcacaaaggtttgaactctgaggtttttggtataataagaatagtccatgcattcaaaagagggaagccaaggaagaactagaagtctttcaagagctcaggctcttatacatccagttgctcattgaaccagcttcctggaatggagggtctggggttgagactaggccacaagtctagagtctctagagagacagtgttggaaccccatggcccataatacatttcccattttctcaggcagccagaggtcatgaatgtgaggatactgggaggttggagcaacgttcttgggaggcataaggaagagcgaatgcttcaagatccccgcagcccaaactactcgcctgctttgccccctaatgcatttttctctgctgctccgtagctgtccgacctcttcagatctcttagtccaccctgccgtcttcctttatgccatgggtcccactgttctttcaactcatccccctttccctcagtcccggagtagctgcggccagcagagggtagactgagagcaggagagaaggacctgcctaggaaccccttctagagatactgcatcctgcctgggagcaagttttccagggcagctttgagaagtcttggagaaacaaacctactaaacctgacagacagtaatactatttgcacaatgcttttctgtgggaaaggtagagccttttcactacgtattgagtacatagagtgtgagggttgacctggaacggctatcctcctggatgacgtgtgttttctgaagaactacatgttcgttgcaactcccacattagaatatgaagtcctaccgagagagatacggagactagacagatacagatgcatttgcatgtgaatacacaatcccacaatacagacgtcaaaacccataccagttattccagagagatggattgggcagaaggcagaaggagaatactctgatcgtttttcggccacgtgtgtgtgttatctcagtgtttctaagaagcgtttgctactttagattttttatttaaaaaaatagtaataatctattaagtatgagagatgtgcagagaggattagtgatcgagagccatttttgctggtggcaatcatatggtacttttaatgggaatattagaaaggcaccggtaatgaccttgttgcagcacaaaggagagagtgtggggtgcccctgcatgttgtcccacctcttgtgacgtgtatcgttttggaatttccagtggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaaggtaaggagtctgtggccagacatctacacgcttcgatgctgggatgaaaagccatggaaattcccactgatgcagccgccttcaatggtaaacggatgctcgagtgttgcctgagttctaccatgtaggaggaagcctccgtgcactctctgggggagccagcggagtgatttctggtgcaacgtggttgggctttgtctttaggatgggcacaaaccctccagggggatcgacttcaaaattcaccttgttgtaaaacgggctacctcagtgtcccagccaaaatttttattgtaacatgctgtcaggtgtgtcactctttccaagccagtaagcttttccggggatttcttcaagtagccagcattcagagcaatcttcagcattgcagattctgagaaatgtggctctggagcctgtcaccctcgagaaacctaagagggctgcattgattccatgtggccctgggtctatggagcagtacatgagctcccagtgctctaaggctcttcagccctaggctttgaagggagtgatttctcagtattcttaaacctctttctgatgacacttgtacctgtgaggggtctagagagaaagagtagtagactcctactttactacaattcaggatgcagggcatgagaggattccctctctcctccaagggaagaagcttttggcgtgcacacatccctgagaagcaaagtgtctttgtcttcagtcagatacataggaccgttttctgccccatggcccggaagccaaaggccttggctttcatgatcaacggtctagggaaacatgcaaaatttccatgtctgtcccaaactctgcccccgacagccaattaccacctgcagcccgcattgccaaatgcggtgccgtttgcatgaagattcagtagagtttcctagaaaggtgctacctcgtgagctcactttccaatgaggaatctgatctgttgtgtttctctaaggtgtcaggtgaaatatttccaagaacttactacagttctagaatgggaggaatctgttgctttggtgtttgtttgttggtcggttttctcacatccatctgcctatggataaggaaaagagaacggtcgtaattctcatagactcctttctggttgtgtcacaaatggcttcacatgtttctctatgctcagagatactcagcttgatttcccgtgttttcatttcagcaccgactgagcaaaggcctggggtgcaggagtgctaccatggtaatggacagagttatcgaggcacatactccaccactgtcacaggaagaacctgccaagcttggtcatctatgacaccacactcgcatagtcggaccccagaatactacccaaatgcgtatgtctttgttctttaccataagagaagaaagggccaagtgaagtttctgttacaagagatgtgtctcaagctgagttctccgaactcaacttgtgacagatgcagatggcgtagcaaaatgtctcaggatgattgccttggagctaagggtctgagagaagggaaatgttaagctccctctccttcctcctagttctattgagcagaagggaaatctggaggtgaggagatcacattatgaagaaagtcagaatgacaaaggaccagacacttagattacccttccacaacaccaactaaacgtcaatggagactttccagttggaattccgttattctggcttccacttcctgaagggaaggttgcgtttgccttttctctctgggttcaagaggaaagaataggtgcttatttatggacaggtgaattgatctgtttctatatctacgtatattccgattgtcagaaaaacactcgttcctaagtaccagtggcctgaagggatacaggttcccagcaagagaagatccaaggaaggaaggcagatgagagtcagcacagagagggatgctgaaaagtaaaagggatgggtggatggagagaagcccgggtctgaccacccaatggccaatattttggccacaagcgactaccagagacatggaaaaatggtttctacatgtgggacaacagatggtagaggacctagagaattgagagaggggcaatgatgggctccactccgcagatgccttggctttcttcctggatacccttcctgcactgaatagcaaggagatggagcccaagcagactgtagccatcttgctgaatggaggagagggattggagtttgggatgactgtggtagctgaaatttttctaggtctgctagaaataagaactggtttgtggaggaaaagagctctacaaatacgcatagaagtctcctccagtcgttggcctgacatgacgctgcctgtgcacaggaaatggttccacgagaaagtgtggcaaagaacatttactgagaaacagcaagtacaagagcacaggaagctcaataaagaagagagagatcacatagcactctgggatactggagttcttcccagctagaccagagagtcctcacggagcacattgccaattcagtggagaccccagaacagccgtaatttaaaggtacacttagtatattactagaataaagtcagctgcagacaaccccttgcacagctggaaagcaagtgtccaagcatcaaatcggtttccaatcaatgaagtgcctgtgagaggaaatctcaactctctttagaagtaaacaacaaagtcgattgcctcagctatgcggtatccgcagagtgagtcctaaatttaaaatctgactacatgtagaaaagcgtttcgtgtgacccatgaccaggaaataaatcgggtaatacaaacaggctcaggaatgagagaaatgattagaattgcgtgaaaatttgacatatcagtatgataactgatttcaaatatttaaaaaaacaacatgcaagaaagcagatatcatatcaagagaaattaacagtacagaatagccaaattaaattaaagagctagtataaaaaaagtatgtcttaattgaaaaaaattactgtatggccggctgatcaatttagacgtttcagaggaaaacattacccaacacacaattctagagaacctacagaatgagctacacacacacacacacacacacacacaaactgaaaacacacccatactcacacacacgcagaaactcacaagttctaacacacacagacacgcgcacccctgaagaaacagtgaaatataaaattaagcgagcctcacagacatgtaggaaaatatgaaaagatttcctgcatgtgggaagcaagtcacagtaaagagcaagggagtttggaatagaaacaaataccggaatcaaggatggctgataacttttcaattacgaagaacattaaaaaaaatcacagaatcgtgaaactcaagggatcacatagggaatttcggaaaaaaaacccaacctgtatgatgtacttttgtacatcacagttcgaaggtaacaaggcaaagatataataagaagaaacctgtcacgagaaactggaggaaaaagagctgtgtcttcctacaagtacactgatacaaattgccaatgtgttcacctcagaaacactggaagccagataccagggaatattgttaaaatgataatcaggaacaaaaagagatcaaccgggaatgctgaatccagcaataaaatgccttgaagatcatccatgtcggataaatgcatattgtgcactgccccaaagaaagaaaccggaaactgtaagaattggaaatcagcaggcttatgtaacaagagaggtgacccgaaggaattaggtagaagaagaattgaacaagaaaggaactttctgcagcccacgtaatgaagaatccagcaattggcaaatgtagatagatgtaaatgcaaaatattttcttgatcaaatttctatatctttgtaaatgagagttgactacttgaaacaaaatgatagcaagatatttaacttcagcatatgtagaggtaagaatttgaaatggtagcataaatcacgaagggattaattcgaagtgtaccgttgtaagtttctttacctcatgcacgatggtgtgtcatattaataaaagggtactgtgcgggttcgaagggatattgcaaatcctagagcaatcacaaaggtttgaactctgaggtttttggtataataagaatagtccatgcattcaaaagagggaagccaaggaagaactagaagtctttcaagagctcaggctcttatacatccagttgctcattgaaccagcttcctggaatggagggtctggggttgagactaggccacaagtctagagtctctagagagacagtgttggaaccccatggcccataatacatttcccattttctcaggcagccagaggtcatgaatgtgaggatactgggaggttggagcaacgttcttgggaggcataaggaagagcgaatgcttcaagatccccgcagcccaaactactcgcctgctttgccccctaatgcatttttctctgctgctccgtagctgtccgacctcttcagatctcttagtccaccctgccgtcttcctttatgccatgggtcccactgttctttcaactcatccccctttccctcagtcccggagtagctgcggccagcagagggtagactgagagcaggagagaaggacctgcctaggaaccccttctagagatactgcatcctgcctgggagcaagttttccagggcagctttgagaagtcttggagaaacaaacctactaaacctgacagacagtaatactatttgcacaatgcttttctgtgggaaaggtagagccttttcactacgtattgagtacatagagtgtgagggttgacctggaacggctatcctcctggatgacgtgtgttttctgaagaactacatgttcgttgcaactcccacattagaatatgaagtcctaccgagagagatacggagactagacagatacagatgcatttgcatgtgaatacacaatcccacaatacagacgtcaaaacccataccagttattccagagagatggattgggtaggaggcagaaggagaatactctgatcgtttttcggccacgtgtgtgtgttatctcagtgtttctaagaagcgtttgctactttagattttttatttaaaaaaaatagtaataatctattaagtatgagagatgtgcagagaggattagtgatcgagagccatttttgctggtggcaatcatatggtacttttaatgggaatattagaaaggcaccggtaatgaccttgttgcagcacaaaggagagagtgtggggtgcccctgcatgttgtcccacctcttgtgacgtgtatcgttttggaatttccagtggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaaggtaaggagtctgtggccagacatctacacgcttcgatgctgggatgaaaagccatggaaattcccactgatgcagccgccttcaatggtaaacggatgctcgagtgttgccggagttctgccatgttgggggaagcctccgtgtactctctgggggagccagcggagtgatttctggtgcaacttgggtgggctttgtctttagaatgggcacaaaccttccagggtgatgggcttcacaactcacctccttctaaaatgggctatctcagtgtcttagccaaaatttttattgtaacgtgctgtcaggtgtgtgattctttctgtcgcagtaagcttttctggggatttcttcaagtagccagcagtcagtgcaatcttcagcattgcagatttcaaaaaatgtggctctggagcctgtcatcctcgagaaacctaacagggctgcattaattccatatggtcctgggtctatggagcagtatatgagctcccaatgctctaaggctcttcagtcctaggctttgaagggagtgatttctcagtgttcttaaacctctttctgatggcacttgtacctgtgaggggtctagagagaaaggttagtagacttctcctttactgcaattcaggatgcagggcatgagaagattccctccctcctccaagggaagaaggttttggcgtgcacacatccttgagaagcaaagtgtctttgccttcagtcagatatataggatcgttttctgccccatggcctggaagccagaggccttggctttcatgatcaacgatctagggaaacatgcaaaatttccatgtctttcccctcctctgccctcgacagccaattaccacctgcatcctgcattgccaaatgcagtgccctttgtatgaacattcagtagagtttcatagaaaggtgctacttcgtgagcgcactttgcagtgagaaggagtctgttctgttctgtttttctaaggatttcaggtgaaatatttcctagaacttactacagttctagattggtaggaatctgtaggtttgctgtatgttttttggttggttttctcccatccatctgcctacaggtaagggaaagataacgttcgtaattctcatagactcctttctggttgtgtcataaatggcttcacatatttcgttattctcagagatactcagtttatttcttgtgttttcatttcagcaccgactgagcagaggcctggggtgcaggagtgctaccacggtaatggacagagttatcgaggcacatactccaccactgtcactggaagaacctgccaagcttggtcatctatgacaccacactcgcatagtcggaccccagaatactacccaaatgcgtatgtctttgttctttaccataagagaagaaagggccaagtgaagtttctgttacaagagatgtgtctcaagctgagttctccgaactcaacttgtgacagatgcagatggcgtagcaaaatgtctcaggatgattgccttggagctaagggtctgagagaagggaaatgttaagctccctctccttcctcctagttctattgagcagaagggaaatctggaggtgaggagatcacattatgaagaaagtcagaatgacaaaggaccagacacttagattacccttccacaacaccaactaaacgtcaatggagactttccagttggaattccgttattctggcttccacttcctgaagggaaggttgcgtttgccttttctctctgggttcaagaggaaagaataggtgcttatttatggacaggtgaattgatctgtttctatatctacgtatattccgattgtcagaaaaacactcgttcctaagtaccagtggcctgaagggatacaggttcccagcaagagaagatccaaggaaggaaggcagatgagagccagcacagagagggatgctgaaaagtaaaagggatgggtggatggagagaagcccgggtctgaccacccaatggccaatattttggccacaagcgactaccagagacatggaaaaatggtttctacatgtgggacaacagatggtagaggacctagagaattgagagaggggcaatgatgggctccactccgcagatgccttggctttcttcctggatacccttcctgcactgaatagcaaggagatggagcccaagcagactgtagccatcttgctgaatggaggagagggattggagtttgggatgactgtggtagctgaaatttttctaggtctgctagaaataagaactggtttgtggaggaaaagagctctacaaatacgcatagaagtctcctccagtcgttggcctgacatgacgctgcctgtgcacaggaaatggttccacgagaaagtgtggcaaagaacatttactgagaaacagcaagtacaagagcacaggaagctcaataaagaagagagagatcacatagcactctgggatactggagttcttcccagctagaccagagagtcctcacggagcacattgccaattcagtggagaccccagaacagccgtaatttaaaggtacacttagtatattactagaataaagtcagctgcagacaaccccttgcacagctggaaagcaagtgtccaagcatcaaatcggtttccaatcaatgaagtgcctgtgggaggaaatctcaactctctttagaagtaaacaacaaagtcgattgcctcagctatgcggtatccgcagagtgagtcctaaatttaaaatctgactacatgtagaaaagcgtttcgtgtgacccatgaccaggaaataaatcgggtaatacaaacaggctcaggaatgagagaaatgattagaattgcgtgaaaatttgacatatcagtatgataactgatttcaaatatttaaaaaaacaacatgcaagaaagcagatatcatatcaagagaaattaacagtacagaatagccaaattaaattaaagagctagtataaaaaaagtatgtcttaattgaaaaaaattactgtatggccggctgatcaaattagacgtttcagaggaaaacattacccaacacacaattctagagaacctacagaatgagctacacacacacacacacacacacacacacacactgaaaacacacccatactcacacacacgcagaaactcacaagttctaacacacacagacacgcgcacccctgaagaaacagtgaaatataaaattaagcgagcctcacagacatgtaggaaaatatgaaaagatttcctgcatgtgggaagcaagtcacagtaaagagcaagggagtttggaatagaaacaaataccggaatcaaggatggctgataacttttcaattacgaagaacattaaaaaaaatcacagaatcgtgaaactcaagggatcatatagggaatttcggaaaaaaaacccaacctgtatgatgtacttttgtacatcacagttcgaaggtaacaaggcaaagatataataagaagaaacctgtcacgagaaactggaggaaaaagagctgtgtcttcctacaagtacactgatacaaattgccaatgtgttcacctcagaaacactggaagccagataccagggaatattgttaaaatgataatcaggaacaaaaagagatcaaccgggaatgctgaatccagcaataaaatgccttgaagatcatccatgtcggataaatgcatattgtgcactgccccaaagaaagaaaccggaaactgtcagaattggaaatcagcaggcttatgtaacaagagaggtgacccgaaggaattaggtagaagaagaattgaacaagaaaggaactttctgcagcccacgtaatgaagaatccagcaattggcaaatgtagatagatgtaaatgcaaaatattttcttgatcaaatttctatatctttgtaaatgagagttgactacttgaaacaaaatgatagcaagatatttaacttcagcatatgtagaggtaagaatttgaaatggtagcataaatcacgaagggattaattcgaagtgtaccgttgtaagtttctttacctcatgcacgatggtgtgtcatattaataaaagggtactgtgcgggttcgaagggatattgcaaatcctagagcaatcacaaaggtttgaactctgaggtttttggtataataagaatagtccatgcattcaaaagagggaagccaaggaagaactagaagtctttcaagagctcaggctcttatacatccagttgctcattgaaccagcttcctggaatggagggtctggggttgagactaggccacaagtctagagtctctagagagacagtgttggaaccccatggcccataatacatttcccattttctcaggcagccagaggtcatgaatgtgaggatactgggaggttggagcaacgttcttgggaggcataaggaagagcgaatgcttcaagatccccgcagcccaaactactcgcctgctttgccccctaatgcatttttctctgctgctccgtagctgtccgacctcttcagatctcttagtccaccctgccgtcttcctttatgccatgggtcccattgttctttcaactcatccccctttccctcagtcccggagtagctgcggccagcagagggtagactgagagcaggagagaaggacctgcctaggaaccccttctagagatactgcatcctgcctgggagcaagttttccagggcagctttgagaagtcttggagaaacaaacctactaaacctgacagacagtaatactatttgcacaatgcttttctgtgggaaaggtagagccttttcactacgtattgagtacatagagtgtgagggttgacctggaacggctatcctcctggatgacgtgcgttttctgaagaactacatgttcgttgcaactcccacattagaatatgaagtcctaccgagagagatacggagactagacagatacagatgcatttgcatgtgaatacacaatcccacaatacagacgtcaaaacccataccagttattccagagagatggattgggcagaaggcagaaggagaatactctgatcgtttttcggccacgtgtgtgtgttatctcagtgtttctaagaagcgtttgctactttagattttttatttaaaaaaaatagtaataatctattaagtatgagagatgtgcagagaggattagtgatcgagagccatttttgctggtggcaatcatatggtacttttaatgggaatattagaaaggcaccggtaatgaccttgttgcagcacaaaggagagagtgtggggtgcccctgcatgttgtcccacctcttgtgacgtgtatcgttttggaatttccagtggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaaggtaaggagtctgtggccagacatctacacgcttcgatgctgggatgaaaagccatggaaattcccactgatgcagccgccttcaatggtaaacggatgctcgagtgttgcctgagttctaccatgtaggaggaagcctccgtgcactctctgggggagccagcggagtgatttctggtgcaacgtggttgggctttgtctttaggatgggcacaaaccctccagggggatcgacttcaaaattcaccttgttgtaaaacgggctacctcagtgtcccagccaaaatttttattgtaacatgctgtcaggtgtgtcactctttccaagccagtaagcttttccggggatttcttcaagtagccagcattcagagcaatcttcagcattgcagattctgagaaatgtggctctggagcctgtcaccctcgagaaacctaagagggctgcattgattccatgtggccctgggtctatggagcagtacatgagctcccagtgctctaaggctcttcagccctaggctttgaagggagtgatttctcagtattcttaaacctctttctgatgacacttgtacctgtgaggggtctagagagaaagagtagtagactcctactttactacaattcaggatgcagggcatgagaggattccctctctcctccaagggaagaagcttttggcgtgcacacatccctgagaagcaaagtgtctttgtcttcagtcagatacataggaccgttttctgccccatggcccggaagccaaaggccttggctttcatgatcaacggtctagggaaacatgcaaaatttccatgtctgtcccaaactctgcccccgacagccaattaccacctgcagcccgcattgccaaatgcggtgccgtttgcatgaagattcagtagagtttcctagaaaggtgctacctcgtgagctcactttccaatgaggaatctgatctgttgtgtttctctaaggtgtcaggtgaaatatttccaagaacttactacagttctagaatgggaggaatctgttgctttggtgtttgtttgttggtcggttttctcacatccatctgcctatggataaggaaaagagaacggtcgtaattctcatagactcctttctggttgtgtcacaaatggcttcacatgtttctctatgctcagagatactcagcttgatttcccgtgttttcatttcagcaccgactgagcaaaggcctggggtgcaggagtgctaccatggtaatggacagagttatcgaggcacatactccaccactgtcacaggaagaacctgccaagcttggtcatctatgacaccacactcgcatagtcggaccccagaatactacccaaatgcgtatgtctttgttctttaccataagagaagaaagggccaagtgaagtttctgttacaagagatgtgtctcaagctgagttctccgaactcaacttgtgacagatgcagatggcgtagcaaaatgtctcaggatgattgccttggagctaagggtctgagagaagggaaatgttaagctccctctccttcctcctagttctattgagcagaagggaaatctggaggtgaggagatcacattatgaagaaagtcagaatgacaaaggaccagacacttagattacccttccacaacaccaactaaacgtcaatggagactttccagttggaattccgttattctggcttccacttcctgaagggaaggttgcgtttgccttttctctctgggttcaagaggaaagaataggtgcttatttatggacaggtgaattgatctgtttctatatctacgtatattccgattgtcagaaaaacactcgttcctaagtaccagtggcctgaagggatacaggttcccagcaagagaagatccaaggaaggaaggcagatgagagtcagcacagagagggatgctgaaaagtaaaagggatgggtggatggagagaagcccgggtctgaccacccaatggccaatattttggccacaagcgactaccagagacatggaaaaatggtttctacatgtgggacaacagatggtagaggacctagagaattgagagaggggcaatgatgggctccactccgcagatgccttggctttcttcctggatacccttcctgcactgaatagcaaggagatggagcccaagcagactgtagccatcttgctgaatggaggagagggattggagtttgggatgactgtggtagctgaaatttttctaggtctgctagaaataagaactggtttgtggaggaaaagagctctacaaatacgcatagaagtctcctccagtcgttggcctgacatgacgctgcctgtgcacaggaaatggttccacgagaaagtgtggcaaagaacatttactgagaaacagcaagtacaagagcacaggaagctcaataaagaagagagagatcacatagcactctgggatactggagttcttcccagctagaccagagagtcctcacggagcacattgccaattcagtggagaccccagaacagccgtaatttaaaggtacacttagtatattactagaataaagtcagctgcagacaaccccttgcacagctggaaagcaagtgtccaagcatcaaatcggtttccaatcaatgaagtgcctgtgagaggaaatctcaactctctttagaagtaaacaacaaagtcgattgcctcagctatgcggtatccgcagagtgagtcctaaatttaaaatctgactacatgtagaaaagcgtttcgtgtgacccatgaccaggaaataaatcgggtaatacaaacaggctcaggaatgagagaaatgattagaattgcgtgaaaatttgaaatatcagtatgataactgatttcaaatatttaaaaaaacaacatgcaagaaagcagatatcatatcaagagaaattaacagtacagaatagccaaattaaattaaagagctagtataaaaaaagtatgtcttaattgaaaaaaattactgtatggccggctgatcaatttagacgtttcagaggaaaacattacccaacacacaattctagagaacctacagaatgagctacacacacacacacacacacacacacaaactgaaaacacacccatactcacacacacgcagaaactcacaagttctaacacacacagacacgcgcacccctgaagaaacagtgaaatataaaattaagcgagcctcacagacatgtaggaaaatatgaaaagatttcctgcatgtgggaagcaagtcacagtaaagagcaagggagtttggaatagaaacaaataccagaatcaaggatggctgataacttttcaattacgaagaacattaaaaaaaatcacagaatcgtgaaactcaagggatcacatagggaatttcggaaaaaaaacccaacctgtatgatgtacttttgtacatcacagttcgaaggtaacaaggcaaagatataataagaagaaacctgtcacgagaaactggaggaaaaagagctgtgtcttcctacaagtacactgatacaaattgccaatgtgttcacctcagaaacactggaagccagataccagggaatattgttaaaatgataatcaggaacaaaaagagatcaaccgggaatgctgaatccagcaataaaatgccttgaagatcatccatgtcggataaatgcatattgtgcactgccccaaagaaagaaaccggaaactgtaagaattggaaatcagcaggcttatgtaacaagagaggtgacccgaaggaattaggtagaagaagaattgaacaagaaaggaactttctgcagcccacgtaatgaagaatccagcaattggcaaatgtagatagatgtaaatgcaaaatattttcttgatcaaatttctatatctttgtaaatgagagttgactacttgaaacaaaatgatagcaagatatttaacttcagcatatgtagaggtaagaatttgaaatggtagcataaatcacgaagggattaattcgaagtgtaccgttgtaagtttctttacctcatgcacgatggtgtgtcatattaataaaagggtactgtgcgggttcgaagggatattgcaaatcctagagcaatcacaaaggtttgaactctgaggtttttggtataataagaatagtccatgcattcaaaagagggaagccaaggaagaactagaagtctttcaagagctcaggctcttatacatccagttgctcattgaaccagcttcctggaatggagggtctggggttgagactaggccacaagtctagagtctctagagagacagtgttggaaccccatggcccataatacatttcccattttctcaggcagccagaggtcatgaatgtgaggatactgggaggttggagcaacgttcttgggaggcataaggaagagcgaatgcttcaagatccccgcagcccaaactactcgcctgctttgccccctaatgcatttttctctgctgctccgtagctgtccgacctcttcagatctcttagtccaccctgccgtcttcctttatgccatgggtcccactgttctttcaactcatccccctttccctcagtcccggagtagctgcggccagcagagggtagactgagagcaggagagaaggacctgcctaggaaccccttctagagatactgcatcctgcctgggagcaagttttccagggcagctttgagaagtcttggagaaacaaacctactaaacctgacagacagtaatactatttgcacaatgcttttctgtgggaaaggtagagccttttcactacgtattgagtacatagagtgtgagggttgacctggaacggctatcctcctggatgacgtgtgttttctgaagaactacatgttcgttgcaactcccacattagaatatgaagtcctaccgagagagatacggagactagacagatacagatgcatttgcatgtgaatacacaatcccacaatacagacgtcaaaacccataccagttattccagagagatggattgggcagaaggcagaaggagaatactctgatcgtttttcggccacgtgtgtgtgttatctcagtgtttctaagaagcgtttgctactttagattttttatttaaaaaaaatagtaataatctattaagtatgagagatgtgcagagaggattagtgatcgagagccatttttgctggtggcaatcatatggtacttttaatgggaatattagaaaggcaccggtaatgaccttgttgcagcacaaaggagagagtgtggggtgcccctgcatgttgtcccacctcttgtgacgtgtatcgttttggaatttccagtggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaaggtaaggagtctgtggccagacatctacacgcttcgatgctgggatgaaaagccatggaaattcccactgatgcagccgccttcaatggtaaacggatgctcgagtgttgcctgagttctaccatgtaggaggaagcctccgtgcactctctgggggagccagcggagtgatttctggtgcaacgtggttgggctttgtctttaggatgggcacaaaccctccagggggatcgacttcaaaattcaccttgttgtaaaacgggctacctcagtgtcccagccaaaatttttattgtaacatgctgtcaggtgtgtcactctttccaagccagtaagcttttccggggatttcttcaagtagccagcattcagagcaatcttcagcattgcagattctgagaaatgtggctctggagcctgtcaccctcgagaaacctaagagggctgcattgattccatgtggccctgggtctatggagcagtacatgagctcccagtgctctaaggctcttcagccctaggctttgaagggagtgatttctcagtattcttaaacctctttctgatgacacttgtacctgtgaggggtctagagagaaagagtagtagactcctactttactacaattcaggatgcagggcatgagaggattccctctctcctccaagggaagaagcttttggcgtgcacacatccctgagaagcaaagtgtctttgtcttcagtcagatacataggaccgttttctgccccatggcccggaagccaaaggccttggctttcatgatcaacggtctagggaaacatgcaaaatttccatgtctgtcccaaactcttcccccgacagccaattaccacctgcagcccgcattgccaaatgcggtgccgtttgcatgaagattcagtagagtttcctagaaaggtgctacctcgtgagctcactttccaatgaggaatctgatctgttgtgtttctctaaggtgtcaggtgaaatatttccaagaacttactacagttctagaatgggaggaatctgttgctttggtgtttgtttgttggtcggttttctcacatccatctgcctatggataaggaaaagagaacggtcgtaattctcatagactcctttctggttgtgtcacaaatggcttcacatgtttctctatgctcagagatactcagcttgatttcccgtgttttcatttcagcaccgactgagcaaaggcctggggtgcaggagtgctaccatggtaatggacagagttatcgaggcacatactccaccactgtcacaggaagaacctgccaagcttggtcatctatgacaccacactcgcatagtcggaccccagaatactacccaaatgcgtatgtctttgttctttaccataagagaagaaagggccaagtgaagtttctgttacaagagatgtgtctcaagctgagttctccgaactcaacttgtgacagatgcagatggcgtagcaaaatgtctcaggatgattgccttggagctaagggtctgagagaagggaaatgttaagctccctctccttcctcctagttctattgagcagaagggaaatctggaggtgagaagatcacattatgaagaaagtcagaatgacaaaggaccagacacttagattacccttccacaacaccaactaaacgtcaatggagactttccagttggaattccgttattctggcttccacttcctgaagggaaggttgcgtttgccttttctctctgggttcaagaggaaagaataggtgcttatttatggacaggtgaattgatctgtttctatatctacgtatattccgattgtcagaaaaacactcgttcctaagtaccagtggcctgaagggatacaggttcccagcaagagaagatccaaggaaggaaggcagatgagagtcagcacagagagggatgctgaaaagtaaaagggatgggtggatggagagaagcccgggtctgaccacccaatggccaatattttggccacaagcgactaccagagacatggaaaaatggtttctacatgtgggacaacagatggtagaggacctagagaattgagagaggggcaatgatgggctccactccgcagatgccttggctttcttcctggatacccttcctgcactgaatagcaaggagatggagcccaagcagactgtagccatcttgctgaatggaggagagggattggagtttgggatgactgtggtagctgaaatttttctaggtctgctagaaataagaactggtttgtgtggaggaaaagagctctacaaatacgcatagaagtctcctccagtcgttggcctgacatgacgctgcctgtgcacaggaaatggttccacgagaaagtgtggcaaagaacatttactgagaaacagcaagtacaagagcacaggaagctcaataaagaagagagagatcacatagcactctgggatactggagttcttcccagctagaccagagagtcctcacggagcacattgccaattcagtggagaccccagaacagccgtaatttaaaggtacacttagtatattactagaataaagtcagctgcagacaaccccttgcacagctggaaagcaagtgtccaagcatcaaatcggtttccaatcaatgaagtgcctgtgagaggaaatctcaactctctttagaagtaaacaacaaagtcgattgcctcagctatgcggtatccgcagagtgagtcctaaatttaaaatctgactacatgtagaaaagcgtttcgtgtgacccatgaccaggaaataaatcgggtaatacaaacaggctcaggaatgagagaaatgattagaattgcgtgaaaatttgacatatcagtatgataactgatttcaaatatttaaaaaaacaacatgcaagaaagcagatatcatatcaagagaaattaacagtacagaatagccaaattaaattaaagaggtagtataaaaaaagtatgtcttaattgaaaaaaattactgtatggccggctgatcaatttagacgtttcagaggaaaacattacccaacacacaattctagagaacctacagaatgagctacacacacacacacacacacacacacaaactgaaaacacacccatactcacacacacgcagaaactcacaagttctaacacacacagacacgcgcacccctgaagaaacagtgaaatataaaattaagcgagcctcacagacatgtaggaaaatatgaaaagatttcctgcatgtgggaagcaagtcacagtaaagagcaagggagtttggaatagaaacaaataccggaatcaaggatggctgataacttttcaattacgaagaacattaaaaaaaatcacagaatcgtgaaactcaagggatcacatagggaatttcggaaaaaaaacccaacctgtatgatgtacttttgtacatcacagttcgaaggtaacaaggcaaagatataataagaagaaacctgtcacgagaaactggaggaaaaagagctgtgtcttcctacaagtacactgatacaaattgccaatgtgttcacctcagaaacactggaagccagataccagggaatattgttaaaatgataatcaggaacaaaaagagatcaaccgggaatgctgaatccagcaataaaatgccttgaaggtcatccatgtcggataaatgcatattgtgcactgccccaaagaaagaaaccggaaactgtaagaattggaaatcagcaggcttatgtaacaagagaggtgacccgaaggaattaggtagaagaagaattgaacaagaaaggaactttctgcagcccacgtaatgaagaatccagcaattggcaaatgtagatagatgtaaatgcaaaatattttcttgatcaaatttctatatctttgtaaatgagagttgactacttgaaacaaaatgatagcaagatatttaacttcagcatatgtagaggtaagaatttgaaatggtagcataaatcacgaagggattaattcgaagtgtaccgttgtaagtttctttacctcatgcacgatggtgtgtcatattaataaaagggtactgtgcgggttcgaagggatattgcaaatcctagagcaatcacaaaggtttgaactctgaggtttttggtataataagaatagtccatgcattcaaaagagggaagccaaggaagaactagaagtctttcaagagctcaggctcttatacatccagttgctcattgaaccagcttcctggaatggagggtctggggttgagactaggccacaagtctagagtctctagagagacagtgttggaaccccatggcccataatacatttcccattttctcaggcagccagaggtcatgaatgtgaggatactgggaggttggagcaacgttcttgggaggcataaggaagagcgaatgcttcaagatccccgcagcccaaactactcgcctgctttgccccctaatgcatttttctctgctgctccgtagctgtccgacctcttcagatctcttagtccaccctgccgtcttcctttatgccatgggtcccactgttctttcaactcatccccctttccctcagtcccggagtagctgcggccagcagagggtagactgagagcaggagagaaggacctgcctaggaaccccttctagagatactgcatcctgcctgggagcaagttttccagggcagctttgagaagtcttggagaaacaaacctactaaacctgacagacagtaatactatttgcacaatgcttttctgtgggaaaggtagagccttttcactacgtattgagtacatagagtgtgagggttgacctggaacggctatcctcctggatgacgtgcgttttctgaagaactacatgttcgttgcaactcccacattagaatatgaagtcctaccgagagagatacggagactagacagatacagatgcatttgcatgtgaatacacaatcccacaatacagacgtcaaaacccataccagttattccagagagatggattgggcagaaggcagaaggagaatactctgatcgtttttcggccacgtgtgtgtgttatctcagtgtttctaagaagcgtttgctactttagattttttatttaaaaaaaatagtaataatctattaagtatgagagatgtgcagagaggattagtgatcgagagccatttttgctggtggcaatcatatggtacttttaatgggaatattagaaaggcaccggtaatgaccttgttgcagcacaaaggagagagtgtggggtgcccctgcatgttgtcccacctcttgtgacgtgtatcgttttggaatttccagtggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaaggtaaggagtctgtggccagacatctacacgcttcgatgctgggatgaaaagccatggaaattcccactgatgcagccgccttcaatggtaaacggatgctcgagtgttgcctgagttctaccatgtaggaggaagcctccgtgcactctctgggggagccagcggagtgatttctggtgcaacgtggttgggctttgtctttaggatgggcacaaaccctccagggggatcgacttcaaaattcaccttgttgtaaaacgggctacctcagtgtcccagccaaaatttttattgtaacatgctgtcaggtgtgtcactctttccaagccagtaagcttttccggggatttcttcaagtagccagcattcagagcaatcttcagcattgcagattctgagaaatgtggctctggagcctgtcaccctcgagaaacctaagagggctgcattgattccatgtggccctgggtctatggagcagtacatgagctcccagtgctctaaggctcttcagccctaggctttgaagggagtgatttctcagtattcttaaacctctttctgatgacacttgtacctgtgaggggtctagagagaaagagtagtagactcctactttactacaattcaggatgcagggcatgagaggattccctctctcctccaagggaagaagcttttggcgtgcacacatccctgagaagcaaagtgtctttgtcttcagtcagatacataggaccgttttctgccccatggcccggaagccaaaggccttggctttcatgatcaacggtctagggaaacatgcaaaatttccatgtctgtcccaaactctgcccccgacagccaattaccacctgcagcccgcattgccaaatgcggtgccgtttgcatgaagattcagtagagtttcctagaaaggtgctacctcgtgagctcactttccaatgaggaatctgatctgttgtgtttctctaaggtgtcaggtgaaatatttccaagaacttactacagttctagaatgggaggaatctgttgctttggtgtttgtttgttggtcggttttctcacatccatctgcctatggataaggaaaagagaacggtcgtaattctcatagactcctttctggttgtgtcacaaatggcttcacatgtttctctatgctcagagatactcagcttgatttcccgtgttttcatttcagcaccgactgagcaaaggcctggggtgcaggagtgctaccatggtaatggacagagttatcgaggcacatactccaccactgtcacaggaagaacctgccaagcttggtcatctatgacaccacactcgcatagtcggaccccagaatactacccaaatgcgtatgtctttgttctttaccataagagaagaaagggccaagtgaagtttctgttacaagagatgtgtctcaagctgagttctccgaactcaacttgtgacagatgcagatggcgtagcaaaatgtctcaggatgattgccttggagctaagggtctgagagaagggaaatgttaagctccctctccttcctcctagttctattgagcagaagggaaatctggaggtgaggagatcacattatgaagaaagtcagaatgacaaaggaccagacacttagattacccttccacaacaccaactaaacgtcaatggagactttccagttggaattccgttattctggcttccacttcctgaagggaaggttgcgtttgccttttctctctgggttcaagaggaaagaataggtgcttatttatggacaggtgaattgatctgtttctatatctacgtatattccgattgtcagaaaaacactcgttcctaagtaccagtggcctgaagggatacaggttcccagcaagagaagatccaaggaaggaaggcagatgagagtcagcacagagagggatgctgaaaagtaaaagggatgggtggatggagagaagcccgggtctgaccacccaatggccaatattttggccacaagcgactaccagagacatggaaaaatggtttctacatgtgggacaacagatggtagaggacctagagaattgagagaggggcaatgatgggctccactccgcagatgccttggctttcttcctggatacccttcctgcactgaatagcaaggagatggagcccaagcagactgtagccatcttgctgaatggaggagagggattggagtttgggatgactgtggtagctgaaatttttctaggtctgctagaaataagaactggtttgtgtggaggaaaagagctctacaaatacgcatagaagtctcctccagtcgttggcctgacatgacgctgcctgtgcacaggaaatggttccacgagaaagtgtggcaaagaacatttactgagaaacagcaagtacaagagcacaggaagctcaataaagaagagagagatcacatagcactctgggatactggagttcttcccagctagaccagagagtcctcacggagcacattgccaattcagtggagaccccagaacagccgtaatttaaaggtacacttagaatattactagaataaagtcagctgcagacaaccccttgcacagctggaaagcaagtgtccaagcatcaaatcggtttccaatcaatgaagtgcctgtgagaggaaatctcaactctctttagaagtaaacaacaaagtcgattgcctcagctatgcggtatccgcagagtgagtcctaaatttaaaatctgactacatgtagaaaagcgtttcgtgtgacccatgaccaggaaataaatcgggtaatacaaacaggctcaggaatgagagaaatgattagaattgcgtgaaaatttgacatatcagtatgataactgatttcaaatatttaaaaaaacaacatgcaagaaagcagatatcatatcaagagaaattaacagtacagaatagccaaattaaattaaagagctagtataaaaaaagtatgtcttaattgaaaaaaattactgtatggccggctgatcaaattagacgtttcagaggaaaacattacccaacacacaattttagagaacctacagaatgagctacacacacacacacacacacacacacacacacaaactgaaaacacacccatactcacacacacgcagaaactcacaagttctaacacacacagacacgcgcacccctgaagaaacagtgaaatataaaattaagcgagcctcacagacatgtaggaaaatatgaaaagatttcctgcatgtgggaagcaagtcacagtaaagagcaagggagtttataatagaaacaaataccagaatcaaggatggctgataacttttcaattacgaagaacattaaaaaaaatcacagaatcgtgaaactcaagggatcatatagggaatttcggaaaaaaaacccaacctgtatgatgtacttttgtacatcacagttcgaaggtaacaaggcaaagatgtaataagaagaaacctgtcacgagaaactggaggaaaaagagctgtgtcttcctacaagtacactgatacaaattgccaatgtgttcacctcagaaacactggaagccagataccagggaatattgttaaaatgataatcaggaacaaaaagagatcaaccgggaatgctgaatccagcaataaaatgccttgaaggtcatccatgtcggataaatgcatattgtgcactgccccaaagaaagaaaccggaaactgtaagaattggaaatcagcaggcttatgtaacaagagaggtgacccgaaggaattaggtagaagaagaattgaacaagaaaggaactttctgcagcccacgtaatgaagaatccagcaattggcaaatgtagatagatgtaaatgcaaaatattttcttgatcaaatttctatatctttgtaaatgagagttgactacttgaaacaaaatgatagcaagatatttaacttcagcatatgtagaggtaagaatttgaaatggtagcataaatcacgaagggattaattcgaagtgtaccgttgtaagtttctttacctcatgcacgatggtgtgtcatattaataaaagggtactgtgcgggttcgaagggatattgcaaatcctagagcaatcacaaaggtttgaactctgaggtttttggtataataagaatagtccatgcattcaaaagagggaagccaaggaagaactagaagtctttcaagagctcaggctcttatacatccagttgctcattgaaccagcttcctggaatggagggtctggggttgagactaggccacaagtctagagtctctagagagacagtgttggaaccccatggcccataatacatttcccattttctcaggcagccagaggtcatgaatgtgaggatactgggaggttggagcaacgttcttgggaggcataaggaagagcgaatgcttcaagatccccgcagcccaaactactcgcctgctttgccccctaatgcatttttctctgctgctccgtagctgtccgacctcttcagatctcttagtccaccctgccgtcttcctttatgccatgggtcccactgttctttcaactcatccccctttccctcagtcccggagtagctgcggccagcagagggtagactgagagcaggagagaaggacctgcctaggaaccccttctagagatactgcatcctgcctgggagcaagttttccagggcagctttgagaagtcttggagaaacaaacctactaaacctgacagacagtaatactatttgcacaatgcttttctgtgggaaaggtagagccttttcactacgtattgagtacatagagtgtgagggttgacctggaacggctatcctcctggatgacgtgcgttttctgaagaactacatgttcgttgcaactcccacattagaatatgaagtcctaccgagagagatacggagactagacagatacagatgcatttgcatgtgaatacacaatcccacaatacagacgtcaaaacccataccagttattccagagagatggattgggcagaaggcagaaggagaatactctgatcgtttttcggccacgtgtgtgtgttatctcagtgtttctaagaagcgtttgctactttagattttttatttaaaaaaaatagtaataatctattaagtatgagagatgtgcagagacgattagtgatcgagagccatttttgctggtggcaatcatatggtacttttaatgggaatattagaaaggcaccggtaatgaccttgttgcagcacaaaggagagagtgtggggtgcccctgcatgttgtcccacctcttgtgacgtgtatcgttttggaatttccagtggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaaggtaaggagtctgtggccagacatctacacgcttcgatgctgggatgaaaagccatggaaattcccactgatgcagccgccttcaatggtaaacggatgctcgagtgttgcctgagttctaccatgtaggaggaagcctccgtgcactctctgggggagccagcggagtgatttctggtgcaacgtggttgggctttgtctttaggatgggcacaaaccctccagggggatcgacttcaaaattcaccttgttgtaaaacgggctacctcagtgtcccagccaaaatttttattgtaacatgctgtcaggtgtgtcactctttccaagccagtaagcttttccggggatttcttcaagtagccagcattcagagcaatcttcagcattgcagattctgagaaatgtggctctggagcctgtcatcctcgagaaacctaacagggctgcattaattccatatggtcctgggtctatggagcagtatatgagctcccaatgctctaaggctcttcagtcctaggctttgaagggagtgatttctcagtgttcttaaacctctttctgatggcacttgtacctgtgaggggtctagagagaaaggttagtagacttctcctttactgcaattcaggatgcagggcatgagaagattccctccctcctccaagggaagaaggttttggcgtgcacacatccttgagaagcaaagtgtctttgccttcagtcagatatataggatcgttttctgccccatggcctggaagccagaggccttggctttcatgatcaacgatctagggaaacatgcaaaatttccatgtctttcccctcctctgccctcgacagccaattaccacctgcatcctgcattgccaaatgcagtgccctttgtatgaacattcagtagagtttcatagaaaggtgctacttcgtgagcgcactttgcagtgagaaggagtctgttctgttctgtttttctaaggatttcaggtgaaatatttcctagaacttactacagttctagattggtaggaatctgtaggtttgctgtatgttttttggttggttttctcccatccatctgcctacaggtaagggaaagataacgttcataattctcatagactcctttctggttgtgtcataaatggcttcacatatttcgttattctcagagatactcagtttatttcttgtgttttcatttcagcaccgactgagcagaggcctggggtgcaggagtgctaccacggtaatggacagagttatcgaggcacatactccaccactgtcactggaagaacctgccaagcttggtcatctatgacaccacactcgcatagtcggaccccagaatactacccaaatgcgtatgtctttgttctttaccataagagaataaagggccaactgaagtttctgtgacaagagacatgcttcaagctgagttctccgaactcaacttgtgtcagattcagatggtgtagcaaaatgtctcaggatgatttccttggagctaagggtctgagagaagagaaatgttaagctgcctcaccttcctcctagttttgtggagcagaagggaaatgaggaggcgaggagatcaccttatgaagaaagtcagaatgacgaaccaccaaacacttagattacccttgcccaacacccactaagcgtcaatgaagactttccagttggaattccgttattctgacttccaattcctgaagggaagattgtgtttgccttttctgtctgggctcatgaggaaagtttatgtgcttacttatggacaggtgaattgatctgtttctatttctacctgtattccaatagggagaaaatctcttggtcctaagtaccagtggcctgaaaggatagaggttcccagcaagagaagatccaaggaaggaaggcagatgagagtcagcacagagagggatgctgaaaagtaaaagggatgggtagatggatagaagccctggtctgaccaccccatggccaatcatttggccataatcaacaaccaaagacatggaaaaatggtttctacatgtgggacaacagatggtagaggacctagagaattgagagagggccaatgatgagctcaactccatagatgccttggctttcttcctggatacccttcctgcactgaatagcaaggagatggagctcaagcagcctgtagccatctagctgagcagaggagagggattggagtttgggatgactctggtattttctaggtccgctacaaataagaactggtttgtggaggaaaggagctctacaaatacgcatagaagtctcctccagtagttggcctcacatgacactgcatgtgcacagaaaatggttctacagaaagtgtggcaaagaacatttactgagaaacagcaactacaagagaacagcaagctcaattaagaagatagagatcacatagcactctgtgttattggagttcttaccagctagatgagagagtgctcacggaacacattgccaattcagtggagaccccagaacagccataatttcaaagtacaattagtatattactagaataaaggcagctgcagacaaccccttgcacagctgaaaagcaagtgtccaagcatcaaatgggtttccaatcaatgaagtgcctgtgagaggaaatctcaactctcttcagaagtaaacaacaaagtcaattgcctcagctatgcggtatccccagagtgagtcctaaattaaaaatttgactacgtgtagaaaagaatttcgtgtgatccatgaccagaaaataaatcaggcaatacaaacaggctcagaaatgacatcgataattagaattgcatgaaaatttgacatatcagtatgataactgatttcagatatttaaaaaaagtgcaacaaagcaggtatcatatcaagacaaattaatagtatagaatagccaaatcaaattaaagaactattatacaaaaagtatgtcttaaatgaagaaattactgtatgtccgcctgaaaaatttagatgtttcagaagaaaaaattaaccaaaaacaattctgcagaacctacagaatgagccacacacacacacattcaaaacacacccatacacacacacatgcaaaaactcacaagttctaacacacacacaaacacacacacacatgcacatccctaaagaaatagggaaatataaaattaaccgaccctcagagacatgcaggaaaatataagaagatttcctgcatgtgggaagcaagtcacagtaaagagcaagggagtttggagtagatacaaataccggaatcacggatggctgataacttttcaattatgaagaacgttagaaaaatcacagattcatgaaactaaagggatcaaataggaaatttcgagaaaaaaaactacatgatgcacttctctacatcacagttcaaaggtaacaaggcaaggatataagaagaagaaacatctcacgagaaactggagaaaaaagagctgtgtcttcctagagtacagtgatacaaattgctaatgcgttcacctcagaaacactggaagccagataccagggaatattattaaaatgataatgaggaacaagaagagatcaaccgagaatgctgaatccagcaataaaatgccttgaagatcatccatgttggataaatgcatattgtgcactgcccaaaacaaagaaactggaaagtgtaagactttggaatcagcaggcttatgtagcaacagaggtgacccgaaagaattaggtataagaagaatagaagaattgcatgaaaatttgacatatgactaagataactatttcaaatatttaaaaaaagatgaatatgtaataaaacagataaaatatcaaaagaaagtaacagtattgactagccaaatcaaattaaagacttagtgtaaaaagctatgtcttaaaagaaaaaattactggatggctgcctgatcaatttagacatttctgaataggaaactaaccaaaaatcaattctacagaaccaactacacacatatatacacatacaacacacccatacacacccacgcaaaaactcacaagttcacacacacacacacacacacacaaccctcaagaaatagtgaaatagaaaaccaaccgaacctcacagacatgttgcaaaataggaaaagatttcctgcatatgggaagcaagtcacagaaaagagaacgggagattggaaacagaaacaaataccggaatcaaggatggccgaaaacttttcattgatcaagaatattaacaaaatcgcaaaaacacgaaattcaatgcatcaaataggcgtttcgaaaaaaagaaaaaatctggtatgatgcacttttgtacttcacattttcacggtaagaagacaaagatataataacaagaaacttcttatgagaaactggggaaaaacaagctgtttcttgctagaagaacagtgatacaaattgctaatgcattctcgtcaaaaacactggaagccagataccgggaatgttattaatgtggtaaacaggaacaagaagagatcaaccaagaatgctaaatccagcaataaaatgccttgaagatcatccatgctgcataaatgtatgttgtgcactgccccaaacaaagaaaccggaaactgtaagaatttggaatcagcaggctgatgtaacaagagaggtgacccaaaggaattaggtagaagaagaatagtacaagaagggaactttctgcagcccatgtaatgaagaacccagcaattggcaaatgtagatgtaaatgcaaaatattttcttgaccaaatttctatatatttttaaatgagcgttgactactggaaacaaaatgatagcaatatatttaattttagcatatgtagaggtaagaatttgaacaagtagcgtaaatcatgtagggaataattagaagtgtaccattgtaagtttcttacctcatgcacaatggtatgtaatattaataaaatgttactgtgtgggttcaaggagatattgcaaatcctagagcaatcacaaagttttgaactctgaggtatattgtataataagaatattccatgtattcaaaagagagaagccaaggaagaaagaaatttgtcacgagtttgggctcttagtacatcctgtagctcattgaaccagcttcctggaatggagggtctgggattgacactaggccacatgtatagagtctctagagagacagtgtttcatccccatggcccgtaatacatttcccattttctcaggcagccacaggtcatgaatgtgaggatagagagaggttggagcaacgttcttgggaggcataaggaagagcaaatgcttcaagatccccgcagcccaaactcctacctgctttgccccctaatgcagtgttcctccgtagctgtccgacctcttcagatctcttagtctaccctgccatcttcctttatgccatgggtcccactgttctttcaactcatccccctttccctcagtgcagagtagctgcggccagcagagggtagactgagagcaggagagaaggtcctgcccaggaacccattctagagatgctgcattctgcctgggagcaagttttccagggcagctttgagaagtcttgcagaaacaaacctatttgacccacatgatatgggaatgacagaaagtaatacaatttgcacagtgcttttccatgggaaaagtagagccttttcgcgaggttttgagtacatagagagtgaaggttgacctggaaaggttatcctcctggatcccatgttttttctgaagaactacctgttagttgcaacttgcacattagaatatgaagtcctaccgagagagatacggagaactagataaatacagatacttttgtatgtgaataaacgattccacaatacacacatcaaaatccataccagttattccagagagatggattgggcagaaggcagaaggagaatactctgatcgttttttgcccacgtgtatgtattatctcagtgtttctaagaagcgtttgctactttagatttttttttataataataatcttttaagtatgagaaatgtgcagacaggattagtgattgagagccatttgtgcttgtggcaatcatatggtacttttatgggaatattagaaaggcactggtaatgaccttgttgcagcacaaaggagagggtgtggggtgcccctgcatattgtcccacctcttgtgacgtgtatcgttttggaatttccagtggcttgatcatgaactactgcaggaatccagatcctgtggcagccccttattgttatacgagggatcccagtgtcaggtgggagtactgcaacctgacacaatgctcagacgcagaagggactgccgtcgcgcctccaactattaccccgattccaagcctagaggctccttctgaacaaggtaaggagcctgtggccagaaacctacacgtttcgatgctgggatgaaaagccatggaaattcccactgatgcagcagcctccaatggtaaacggatgctcgagtgttgactgagttctgtcatgtaggaggaagcctccgtgcactctctgggggagccagcggattgatttctggtacaacgttgggtgggctgtgtctttagaattggcacaaaccctccagggtgatcgacttcacaactcacctcgttgaaaaatgggctatctcagtgtcttagccaaaatttttattgtaacatgctgtcagatgtgtgactctttccaagccagtaagcttttcctgggacttcttcaattagccagcattcagtgcaatcttcagcattgcagattcagagaaatgtggctctggagcctgtcacccttgagaaacagggctaacagggttgcattaattccaaatcaccctggttctatggagcagtacatgaactcccaatgatctatgtttcaggacttcctcagtcataggtgggctctgcagccctaggtttttaagtgagtgactgccccgtgttctggtggcagttgtacctgtgagcggtctggatagaaagagtcggagacttctgtattattgcaactcaggatgtgggtcatgagaggatttcatctctcctgcaggggagtaagctgttcgcctccacccatccctgataactgaagtgtctttgtctgcagtcctagacgaaggactgttgtctctcccatggcccagaagctgaagaccttgccttttgttatgaaacgttcattgttttcatgtctgtccgtttctctgcccctaacacccaatcaccatgtatggcctgtacccccaaatgcatcgtgctttgctgtttgctgccccatagtcctcatgaacattcagtagaaattcccataaatgtgcttgcacgtgagcacagtttccattgagaagccctctcatttgtcctttttttctaagcttttatgtgaaatatttctaagaacttactacagttctaaagtgttaggaatttgtttctttggtgtttttgtttgttggttggttgttgcttttctcaagtccatctgcctacaaataaagaaacaagaatgttacttgtcatattctcctgaggtcataattctcagagacttttttctggtttgtgccataagtggcttcacatgtttgtctcttcttggaaacactcagtttgatttcttttcttttcatttcagcaccaactgagcaaaggcctggggtgcaggagtgctaccacggaaatggacagagttatcaaggcacatacttcattactgtcacaggaagaacctgccaagcttggtcatctatgacaccacactcgcatagtcggaccccagcatactacccaaatgcgtatgtctattttctttaccataagtgaaggaagggtcagtggaaatttctgttagtagagtcatgcttcaagctgagtgttcaggactcaagttgtctcagatgaacagtgcatagcaaaatgtctcaggaacattgtctttgagcaaagagtctaagagaagacaaatgttaatctggctctccttcctcctagtttaatggagcagaaaggtatctggaggcaaggatatcacattaagaaacaagtcaagatgacaaatgatgaaactcttagagtacccttccacaacacccactaaggttcaatgcagccttttctccttggaattctattaaactaaactccaattcctgaagtgaaggttctgttggggttttctgttttggcttacaaggaaagtatatatgtatatctatggagaggcaaatctatctctttctatatctacgtctattccaatatgtagaaacacagtcggttctgaccaccagtggtctgaagggatactggttgttagagaataaaaatggcaggaaggcagatgagagtcagcaaagagagagatcctgtaaagtaaaagggtggatagatggacagaagcccaggtctgaccagcccatggccaggctttaggccataagtgacaccaaagacatggaaaaatggtttctacatgttggacaacagacagtagtggaccaaaagaatagtgacagggggaacaatgagatcaactccatagataccttggctttcttcctggaggcccttcttgcactgaagagcaaggtgatggagcccagatggactgtagccatcttcctgaatgcaggagagagattggaatttgggactactgtggtagctaggattttataggcctgctgagaatgagaatggatttgtggatgaaaggagctccaggggcacgcatagtagtctcctcgaatctttggctaaacatgacgttgcatgtgcccagaaaaaggttccacaagaaagtagagaaaagaatatatcctgaggaatagcaactgcgattgaacagtgagctcaataaagaggacagagccctcatagcattctgggatactggagttctgaccagctggaggagagacctcactgaacctcttgggaatacagtagagactccagaaaagtcatactttaggagtagaattagtaaatttctagaaaaaaaggcagctctagacaaaccctggcaaaactgaaaagcaagtctccaagcattaaaatcatttccaagtcaattaactgcctgggagaggaaaaccctctttagaggtaaacaacaaagtcaagtggctcagctatgtggtgttcacagtgtgagttctaaatttaaaacttgactacacatagagaagcttttagtatgaaccatgaccaggtgaaaaatcagtcaatacaaatagacctagaaatgacagaaatgattagaatggcaaaaaatttgacatatcaatatgtcaactgagttttaggttttaagaaaacatgaatacggaatgaagcagataccatatcaagagacagtaacagtatagaagagccaaattaaattaaagaactagtataagaaggtatgtcttaaatgaaaaaattactggatgtattcccaatggagtgagatgtttcagaagtaaaaactaactgaaaaacaattttataccacctacagaaccagctacacatacacaaatgacacacacatatacacacatactcacacatgcacaggcttagaaacatgcacgcacacacacacacacacacacacacacctccacaaatactaaaaaatgaaatccactgatcctcacagacaggcgggaaaatataaaaagatttcctgcatgtgggtaggaagtcacagaaggagaggaaggagagattgctacaggaacaaatactggaagcaaggatagctaaaaacttttcaaataagaagaatattaaaaaccacagattcaagaagctgaatgaatcagacagggaatttccaaaaaaaaaaaaaaaaaaactgtatgattcacttttgtacatcaccgttcaacagtcagaaggcaaagatataataacaagaaacatctcatgagaaactggaggaaaaagagctgtgtcttgctagaagaacagtgatacaaattgctaatgcattctcatcagaaacactggaacccagttaacaggggatatcattaaaatgataaactagaaaaaaaagagatcaaatgagaatgctacatccagcaataaaatgccttgaagatcatccatgttggataaatgcatattgtgcactgccccaaataaataaaccaaaaactaataatttggaatcagcaggcttgtgtaacaagagatgttgcccaaagaaaattagctagaagaagaatagttcaagaggagaactttctgcagcccacgtaatgaagaacccagcaaatggcaaatgtagatgtaaatgcaaaatattttcttgatcaaatttctatatctttttaaatgagagttgactacttgaagcaaaatgatagcaatatatttaactttagcatatgtagaggtaaaaatttgaacatatagactaaatcatgtggggaataattggaagtgtaccattgtaagtttcttaccttatccacgatggtatgtaatattaatgaaaggttgaatttgtgggtccaaagggatattgtaaatcctaaagcaatcataaaattttgaattctgagggatattatataataagaattttccatgtatccaaaagagggaagccaaggaagaaaaagaagtctttcaagtactcaagctctgagcacatccagttgctcattgaaccagcttcctggaatggagggtctgggcttgagactaggtcacatgtgtagagtctctagagagacagtgttggatccccatggcccataatacatttcccgttttcccaggcagccacaggtcacgaatgggaggattctgagaggttggagcaatgttcttaggaggcataaggaggagtgaatgctctgagatttccccagcctgaggtcctccatagctgcccgacctcttcagacctcatagtctgcccagctgtctccctttatgccatgagtgccactgttctttcaactcatcccccattccctcagtcccggaattgctgtggccagcagaggatggactgagagcaggagaggaagtcctgaccaggaacccatcctagagatactgcatcctgcctgaaagctaggtttccagggcagctttgagaagtcttgcagaaagaaacccacttgacccacctgatacggtatcgacagacaggaatactttttgtgcaatggttttacatgctgaacatagagccttttggctacattttgagtacattgaatgagactgctggcctgggaaggatatcatgctggatgccatttttttctctggagaactatgtgttagttccaactcgcacattactatatgaagtcctacacagagagatacggagagctagacagatagagatacttttgtatgtgcataaccaattccacaatacacacgtcaaaatccataccagttattccagagagatggattgggcagaaggcagaaggaggatattctgatccctttttggccacatgtatgtataatctcagtgtttctaggaagtgtgtgctgcattagattttttttctttaaaaaaagtgataatatattaagtatgagaaatgtgcagagaggattagagattgagagccatttgtcattgtggcaattgtatggtatctcttttgggaatatttcaaaggcaccagtaatgaccttgttgtagcaaaatatacagtgttcctgcatatgtacccattttttgtgatgtgtattcttttggaatttccagtggcttgatcaagaactactgccgaaatccagatcctgtggcagccccttggtgttatacaacagatcccagtgtcaggtgggagtactgcaacctgacacgatgctcagatgcagaatggactgccttcgtccctccgaatgttattctggctccaagcctagaggctttttttgaacaaggtaagaagttgtgccagacatttacctgcttggatgctgggatgaaaagccatggatacccccactgacgcacaacccttcagtgctacactggttctcgtgtgttggttctgggtctgccatgtgggaggaagccttagcgcactctctgggggagccagaggtgtgatttttggtgcaacctgtgcgagctgtgtctttaggatgggcggaaaccattctgggtgctcgacttcaccactcccctcattgtaaaaggggctatctcattgtcctagacaaaattcttattgtaatatgctgtcagatgtgtgtgtctttccaagccagtaaacttttccagggatttcttcaagtagacagcattcagtgcaatcttcagcattgcagattccgagaaatgtggctctagatcctgttatccttgagaaacctaactgggttgcattaattccatatctccctgggtctgtggagtagtacatgagctcccgaagctctatctctcaggtctttttcagtccgaggcaggttgtgcagttcttagctttgaagggagtgattttttcgtgtgcttttgcctctttctgatggaacttgtacctgcggggggtctggagaaaaagagtagtagacttttgctttattgcaatgcattatgctgggcacgagaggattccctatcttattgtaggtgataagcttttggcctccactcatccctgagaagtgaagtgttgttgcctacagttttagctgcaggactgttgtctgccccatcaccaggagtttaatgctttcttttttgagcaatcatctagggacacatgcaaggtttttatatgtccttgcctcctccccaaaaaaccattttaatgcttggagacttgcttttcagctttgccaaatgcatcaccctttcttctatgctgttccatgtcgtcatgaacactctgtagagattcctagaaatgagcttccatgttagtggagtttccgatgagaagcaatctgatatttcttttccactaagttttacatgaaatatttctaagaacttactacagttctagaatggtaggcatctcttactttcgtgtttgtttgtgtgttttctcatgtccatttgcctattaataaagaatagagaatggttgtaaatctcagtgactcttttttggtttatgtcataaatggcttcctgtatttttctgttctaggaaataataagcttgatgtcttctgttttaatttcagcactgactgaggaaacccccggggtacaggactgctactaccattatggacagagttaccgaggcacatactccaccactgtcacaggaagaacttgccaagcttggtcatctatgacaccacaccagcatagtcggaccccagaaaactacccaaatgcgtacgtctttgttctttaccataagcgaaggaagggccaatggaagtttctgttagaagagtcatgcttcaaggtgactgctcaggactcaacttggctcagatgcagaggaacatttcctgtgagcaaaagttcttagagaagactttgtttttttgagacagagtcttgctttgttgcccaggctggagtgcagtggcatgatctcggctcactgcaagctccgcctcccgggttcacaccattctcctgcttcagcctctctagcagctgggactacaggcacccaccaccacacccggctaattttttgtatttttagtagagacagggtttcactgttctagccaggatggtcttggtctcctgacctcgtgatccgcctgcctcagcctcccaaagtgctgggattacaggcgtgagccaccgtgcctggctgagaagacattttttaagctggctctccttcctcctagttttatggaagcagaaggatatatggagttgagaagatcttattaataaaacagccgggatgacaaatgaccaaagagttagagtatccttctacaacatcggctgagggttaatacaaccttttcaccttggaattctatcattctaagctctagtccctgaagtgaatgttgtgttggccttttgcatcttgggtcacagggaattgatacttgcacatctatggagaggcaaatctttttctatctacttctttttcaatgggtacaaacacacttggtcctgagcaccagtggtctgaagagatacggtctgcccagaggagaagaacaaaggcaggaaagcagatgagagtcagcaaaggggcgatgctgaaaagtaaaaggggcgggtagatggacagaagccatgatctggccattctatggccagtctttcggccataagtgactaccaaagacacggcaaaacggtttccacatgttgaacaacagatgctagaggaccaagagtattgcaagagggagaaaatgagatcaacccatcaatgccttggctttcttcaaggagacccttcctgcactgaagagcaaggagatggagcccaagctgactgtagccatgttgctgaacagaggagagtgattggactttgggattactcaggtagttaggattttctagccatgctaagagtaagaatggacttgtggaggataggagctccaggcatagaagtctcctcaagtgttagtctaaacataaagcagcacttgcatagaagattttccacaagaaaatatggcaaaaaaacaccatatattgaggaacaacaactacaagggaacagtgagcttaataaaggtgacagagctcacatagtgctctggaatattggagttttgaccagctagagagaagagacctcattgaaaatcttgggcattcagtagagacctcagaaaagtcagactttatgagtagactttgtatattcctagaataaaggcagctccagaaaaaacctagcaaagctgaaaagcaaatctccaagcattaaaatggtgtcctagtcaattaactgccttctagaagaaaactcaacactctttacaggtgaacaacaaagttaagttgctgagctatgcaatatccacagtgtgagtcctaaatttataactttactacacataaaaaagcatttagtgtgaaccataaccaggaaaataatcagtcaataaaaatagaaccaggaatgatagaaatgatttaaatggcatgagaatttgacatattagtatcataactgcattgctggatttaagaaaacataaacatggaacgtaacagatatcatatcaagggaaagtaaaaggataaaagagtcaaatcaaattaaaggactattaaaaggtatatcttaaatgaaaaattcactggatggtctcccaatcaggttagttgtttccagggaaaaaattaactgaaaaataattcaatagaatctacagaaatagctgcacatatatacacacaatggcacacgtgcacacacccacacccacacaggtgtgaatcctagagccacacgagcattgaaacatagagaagtaaaaattgttcattgaggaatatgtagcaatgctcaatgtgttttaccctaataagagcttttgtgatgtatgattgaaaaactgacacaactgaagagagaaatagataagcccacactctgagttagagatttccttgattctctcactatggttataaatctttcccaaacacaacaggctagaacaaatatgcagaaaattagacatagtatctttgttctcaataaaaacgtcgacctatttaacattataccgaactaccgagtacacattaaagtgtgcatggagcattcactgaggtgtactctacacatgaccttccagcaagtctccatagatttaaaagaattaaagtcatacagagtgtgtcactttattctcccagaataaagtgagatatgaataatgagaagtttgccagcttctcaaatatttgggagtcatacggtgcatttcaaaatactctttgggacaaagaaaacatcactaaggaatttagaaaagttttgaactgagtaagaatataacacaatttatccaaacttaggagatgcagtgaatgtctttaggcttttacataattttagatgctcttagggaaaaacagaagcatgtaataatcaagatttcaaactgcaattctcaaagtgtagtctagagaaacctgaggacctttgagtaccttcagagacagtccatgaggttaaaggactttgctacgtgaaaagtaagatgctattggccctttttactttcattttccaacaagagaagaggggagttttccagcagttacataatatgtaatggcatcatgtctctgatggctaagaaaatgggcaattgttgactttgtgtgttaaaaaaattctcagtgttggtttcttatactataaatattcatcttgtgttttgaaaaagaaaagctctttggaatcccctatgaacaaagactttgacagttgttgatctaagaccacagcttaaatatctacacaagaaaaaaaaaaaaagcaaataagagccaaggaaagcagatggaaggaagtagtccaaaccagtgacattcagtgaacaagaaaagagaccaacaagggagtaaactcttgaaacagaaagttgattctttgaaaagatccatatgattgaacacagtctggctaaacaaatgacagaccaatgagggtgcacaaccatcaccatctggagtaacagaggagaggtgccattactatagcatcttccagttctgaaagctgaaaagaagattttgagaacaattgtatgtgaataaattcaggaatgttaatcatgtgggccaattcctgaggaagacaacaaatcagcaaaccagatgctgaatagttagtgtagtcctgtagagagacatacagagaggctgacagagaaatatttgtatgtgcataaaacaatctacaagacacacttcaaaatcaatctcagttaatctggaggaacatatttcacagaaggtggaaggagggtattctgatcctcttgtacattgtacaacattgtacaatgtacagagtataattgtacaagtacaattgaagttgtacaagtacaagtgcaacttgcacaatgtacagagtaaacattgatgtttactctcaattttcttatggagcacagatgactttggatgtgttacaatatgaatgataatttgtctttgagatgttcgcagttgtttagaagttgaggaccatttgtgcatattatgggacctttagtgaaaatatttcaaagtctctttttacactttgttacagcaaaatgtagagggcgctaagtgcccttgaatcttctcccatctctggtgacctgtgttgttttgaaatttgcagtggcctgaccaggaactactgcaggaatccagatgctgagattcgcccttggtgttacaccatggatcccagtgtcaggtgggagtactgcaacctgacacaatgcctggtgacagaatcaagtgtccttgcaactctcacggtggtcccagatccaagcacagaggcttcttctgaagaaggtaggaagtctatggccagacaaccacaccctaggacgttgggatgaaaagagttgcaaaatcttagtgatatagaagccttccatgctcacacaattccaagtagaatgtggactcagggtcagccactgggaaggaacactcagcgccttctctgggagaaccagagctgtgatgtttggtaccctgtgaaagggtggtatctataggaagggtgcagaccctctagggcactggacttaccactcccctggttattcaaaggatcattttagtgtcttagccagaagaatattctaacattttgccaaatttgtgaagatttaccaagctcatgataagcctttcatggtatttcttcaagtagtcagtgttcattgcatctttggctttgcggtttcggaggaatgcggtttttgagtctgtcatccttgagaaacctaatatgacttttcttagttccatatacttctgggtccaggtagcagtacatagccaacaaatgctccatcgttctggcctatctccatcttaagccagtcctgcacaactaggctttgatgggagggatctctcagtgttcttgcccctccttctcatggaacatatatctgtgttggtctctgagaagaagagtagtggatatctactttgttgcaatgcagaatcctgggccaaagataccagccatccctccaagggaataaaattttggccagtagccctctctgagagacaatttgtctttgcctacgagtcctagatgcaggaccgcttcctgccccatcttcaagaagctgaaggctttggctttggaggatcagcagtctagggaaatgtgtgacggtttcatgtctgtccccactgacagtcaatcaccacctacaacctgcacagcctgatgcatagcagtctagtttcctgccttattctcaggaacacccagaagatgtctatattaaagagcatgcacatgagtgcaattttgactgataggcactctgatctttcctttggtgcctgtgttttaaaggaaatctttctaagaactcgttaaagttctagaatgctatgaatctttgggttttattattggtatgtccatctgcctgctagtacagaacagagcatggtagtctttctcagagacaatgatcctgtttcagtcacagatttcttctgatgcttctgtgttctagaaattactcagcttgatttctcctctttgaatttcagcaccaacggagcaaagccccggggtccaggattgctaccatggtgatggacagagttatcgaggctcattctctaccactgtcacaggaaggacatgtcagtcttggtcctctatgacaccacactggcatcagaggacaacagaatattatccaaatgggtacaaccttgagttttcttcaaagacagacagcagcccccttacatttctcttggaagggccatgcttccaactaacttcttatgacaaatttatctcagatctggaatgttgggtagaatgtctcaggcttctttcttcaggcacagtgtctgaaaggagagaaatgtcaggccagctctcttttctcatagttgacagaagcaggaggatatttgaaggtggtgagttctcatgaatagaaagctcaggacacatggccacgtgcttagaaatagcaccattccacaatgcccactaaagaccaatgcaatagttcaaccagggatttctgtcattctaatctccaagtcctgaagtgaaggttgtattagccatgttcatcttgggcaacaaataaaggatatctatgttgacatccagatcttccaatcactttctcctctaacctgtacctgggttctgagaacaaggtatctgaagagctatgtgttgccagcacatgaggggcaaaagtaggaaggcagctgagagtcaggaagtataaagattctgaagagttacacatgcaggaagatggacagaaacccagttcagaccacgtcagcgtttctgccatgaaggactatcaaatacataggaaaagtgttttcataggttggacaacagacatgacaggcctgagaaaattcagaaagggaatcaaaggagatcaaccttatcatgtccctggcatccttccttgagacccttgaagggcaagcagatggagcccagctgaccacagcagtcttgcttaactgaggagagagactggagtttgtgatgcctcaggcatctgacgtattctaggctggctaagaatgagaggggatttgtggaggaaaggagctccaagaatacacaccgaagtcttctcaaggctttggctaaatacaaagctgcgtatgcacaaggagagttttcacaaagaaagaacaataaagaaaagctactggggaaagaacaactgcaagggaacagtgagctcaatggagatgctagagctcacatagcactgggggatatttgagttctgaccactcagaggagagacacctcactgaacatcttgggcattcagtagaggtcaaagaaagccataatttgggagtaggatcttcggattcctagaaataaggtgactccagaaacactccagcaacccttcttccaagccagtctaaaaggatccaaatgatttccaagtaaattaactgccttccagaaaaaagtaaactcaaccctccttagaggtaaggaacgaatacaagtttctcagttatatgacatccccagagtgcaacttgcatttaaaaatttactagacacaaaagaagttttcactgtgatccataactgggagaaaaatcactcaacacaaataggcccagaaataatagaaattatggcattggcaagaacatttaaaatgcacctctgagaactgtgtttcaggaaaatgtcagcaaaagctgaccatgagagaaatgaatgcataatatcagaaaagaaaagaattgaagagccaaatggaaatttaaaaactgagaaaagttatatctgtaatgaggaattcactggatggccttataaccagtttagatattatggtaggaaaaggtgaacgagaaaatgattcaattaaagctagacaaaccacaagacagacagacagacacaaatacacatacacacaatgactgaaccaattaatcaacagagcctcaaggacatctaggaaaacatccacacatttaatatatgtgttaggcaagtcacagaaagagaggaaaaagataatgtgacagaagttatacttgaagccatgacggctgacaaatttccaaacatacagaaaatgagaaattcatagtcatgaagctcaatgactcaggtatagatttttaaagagcaaaactctgatttactggggtacatcatagttaaattgtctgatttcaaagctaagaagaaaaaaagggggttcctatgaacaaacattttgacagttgatctaagaccacagcttaaatatctaggcaaggaaaagcaaataagacacaaggaaaggggatggatggaaatagtccaaaccaatgacattcagtgaacaagaaaatagaccaacaaaggagtaaatccatgaaacagaaagttggttctttgaaaagattcatgtgattgaccacagtctggctgaacagatgacagaccaaggagggagtacaaccatcaccatttgaagtaacaggggagaggagccattgctataccatactccaggtctgaaagctgacaagaagatatcaagaaaaactgtatgtgaataaattcatgaatgtagatcatgtggatcaattccttaggtaaacaacaaatcagcaaaccagatactgaatagattgggtactcctatagaaagacatacagatagccagacagagaaacatttgtacgtgcataaaacaatctacaagactcacttcaaaatctctcagttaatccaaagtaacatatttggcagaaggtggaaggagggtattctgatcctttcttgtacacattgatgttttctctcggttttcttatggagtatagacgagtttggatgtgttacaataagaatgataatctgtctttgaaatgttcacagttgtttagaagttgaggacgatttgtgattgttacaggacctttagtgagaatatttcaaagtcactttttaccactttgttacaacaaaatgtagaggatgtctggtgcccttgtatcttctcccatctctggtgaactgtattgttttgtaatttgcagtggcctgaccaggaactactgcaggaatccagatgctgagattagtccttggtgttataccatggatcccaatgtcagatgggagtactgcaacctgacacaatgtccagtgacagaatcaagtgtccttgcgacgtccacggctgtttctgaacaaggtaagaagtctctggccagacaaccacacccttggacgttgggataaaaagagttgcaaaatcttagtgatacagaagccttccatgctgcacgggaatctgaatgtggactcagggtcagccaatgggaaggaagcctcagcgccttctctgggggaaccagggctgagatttttggcaccccgtgacagggtggtgtctttaggaagcgtgcagaccttctagggcactggatttaccactcccctggttattcaatagattatttcagtgtcctagtgaaaatggatattctaacatcctgccaaatttgtgatgatttaccaagctcatcatgagcctttcctggtatttcttcaagtagacagtactcattgcaaacttcagctttacagtttcagaggaatgtggtttttgagtctgtcatccttgagaaacctgatatgactttacttagttccatatcctcctgggtctaggtaacagtacatagccagcaaatgctctatctccctgtctaccttaatcttaggcaggtgctgcacacctaggctttgatggaagggatttcttagtgttcttgcccctccttctcatggaacacgtatctgtgttgctgtttgtgaagaagagtagtggatgtctactttgttgcaatgcaggatcctgggcccaagatttcccgccgtccctccaagggaataaaattttggccagtacccctctctgagagacaatgtgtctttgcctggaagtcctagatggaggaccacttcctgccccatcttccagaaacttaaggctttggctttggaggatcagtgctctggagaaatgtgtgacggtttcatgtctgcccccactgacaaccaccacctacagcctgcaccgcctgatgcatggcactctggtctcctgccttgttctcaggaacacccaaaagagatctttgccaaagaacaggcacatgagtgcaattttgactgataggcactctgatctgtcctttggtgcccaggttttaaagaaaatctttctaaaaactcattgaagttccagaatgctatgaatctttgagctttgttattggcatgtccatctgcctactaatgtagaacagagcatggtcgtcattttcagagatgatgtcctgtttctatcatggattttttttctcatgcttctgtgttctggaaattactcagtttgttttctcctctttgaatttcagcaccaacggagcaaagccccacagtccaggactgctaccatggtgatggacagagttatcgaggctcattctccaccactgttacaggaaggacatgtcagtcttggtcctctatgacaccacactggcatcagagaaccacagaatactacccaaatgggtatgtctttgagttttctcccaagagaaacagccacccacttaaatttctcctggaagagccatgcttccagctaacttcttatgacccaatttctctcagacccagaatgttggacagaatgtctcaggcttcttgctttgggcacagggtctgagaggagagaaatgtcaggccagctctcttttctcatagttgatagaagtaggaggatacttggaggtggtgaggtctcatgaatagaaagctcagaagaacatatgaccatgtgcttagaaatagcaccattccacaatgcccactaaagaccagtgaaatagttcaaccagggaattctgtcattctaatctccaagccctggagtgaaggttgtgtttgccatgtttgtcttgggtaacaagtgaaggatatctatattgacttcgagatcttccgatcactttctcctctaacctgtataaacacattgggttctgagaacaaggtgtctgaaaagctatgtgttgccagcccatgaggggcaaaaggaggaaggcagctgagagtcaggaagtatagagatgctgaagagttacacattcaggaagatggacagaaacccatgtctggctatgccagcctttctgccatgaaggactatcaaatacatgagaaaacagttttcacaggttggacaacagatatggtaggcttgagagaactgagaaagggaatcaaaggagatcaacttcatcattaacctgtcttccttcctggacacagtgttggattgaaggacaagcagatggagcccagctgaccacagcagtcttgcttaactgaggagagagactggagtctgcgatgcctcaggcagctgatgtgttctaggctggctaagaatgagaagggatttgtggaagaaaggagctccaggaatacacacagaagtctcctcaaggctttggctaaatacaaagctgcgtatgcacagggagagttttcataaagaaagaacaacaaagaaaagctacttgggaaagaacaactgcaggggaacagtaagctcaatggagatgccagagctcacatagcactgggggatatttgaattctgaccactcagaggagaaacacctcactacattttgggcattcagtagagaccaaagaaagctgtattttgggattgggatcatcttattcctagaatcaaggtgactccagaaaaactccaacaacccttcttccaagccagtctaaaaggatccaaatgatctccaagtaaattaactgcattccacaagaaaaaaaaaactcaaccccccttagaggcaagggacaaatacaagttgctcagttatatggcattcctattgcgttacttctatttaaaaatttaatagagacacaagaagctttcactgtgatacataactgggagaaaaaatcactcaacacaaacaggcccagaaattatagaattgatgacattggtgagaacatttaaaatgcacctctgagaactgtgtttcaggaaaatgtcagcaaaagctgaccatgagagaaacaaaagcagaatagcaagagaaaagaaaagaaccggagagccaaatgaaaattaaagaactgagaaaaggtacatctctaatgaagaactcactggatggccttatcatcactttagacattacggtaggaaaggtgacctagaaaataattcaataggagctacacaaatcacaggacagacagacagaccaacagacagaaacacacacacacacacacacacacacacacacacacacacacacacacaaagactgaacctattaatcaacagagcctcaagggcatctaggaaaaatccacacatttaatatatgtgttaggcaagtcacagaaggagaagaaaaagatatcatgacagacattatacttgaagcgatgatggctcgcaacacgccaaatatacagaaaacaagaaactcatagtcaagaagctaaatgactcaggtatagaattttaaagagcaaaactctatgatttactgggatatatcatagttaagttgcctcaattcaaagctaaaaagaaaaaaagggggttcctatgaacaacagctttgacagctgttgatctaagaccacagcttaaatatctaggcaaggaaaagcaaataaggcacaaggaaagaggatggaaggaaatagtccaaaccaatgacattcagtggaaaagaaaatagaccaacaaaggagtaaatccatgaaacagaaagttaggttctttgaaaagtctatatgattggccaaagtctggctaaacagatgacagaccaaggagggagcatatccatcaccatcatgagtaacaggagagagatgccattgctatagcatcctccaggtgtgaaagctgagaagtagatattgagatcaactgtatgtaaataaattcatgaatgtagatcatgtggatggattgcttaggtaaataacaaatcagcaaatcaaacactgaatagatcatgcagttttatagagacttacagacagcctgacagataaacatttgtatgtacgtgaaacaatctccaagacacacttcaaaatccctctcggttaatccaaaggaatgtatttggcagaaggtagaaggagggtattctgatcctttctggtacacattgatgttttctctcagttttcttataaagcatagattactttgaatgtgttacaataagaatcataagctgtctttgaaatgttgacagttgtttagaagttgaggaccatttgtgagtgttatgggactttagtgagaatatttcaaatttgcttgtttacactttgttacaagaaaacatagagggtgccaggtggtgctgtatcttctccaatctctggtgacctgtattgttttggaatttgcagtggcctgaccaggaactactgcaggaatccagatgctgagattcgcccttggtgttataccatggatcccagtgtcagatgggagtactgcaacctgacgcaatgtccagtgatggaatcaactctcctcacaactcccacggtggtcccagttccaagcacagagcttccttctgaagaaggtaagaagcctgcagtcagacaaccataccctcggacattgggataaaaagatttgcaaaatctttgtgatgcagaaaacttccatgctgcacaggaagtcgaaggtgaagtcatggacagccaatgggaaggaagcttcagtgccttctctggggggaccagagctgggatgttgagtgccttgtgagggatggtgtctttaaaaggggcacagaccctctaggacactggatttatcacttccctgttatcaaacgaatcatattagtgtcctagccaagatggatattctaacatcctgccaaacttgtgaagatataccaagctcctaagcctgtccagccctttcttcaagtaggcagtgtttattgcagtcttcagctttaccattttgaaggaatgccatttttgaggctgttgttcttgagaaacctaacatgtcttcattagatccgtattgtcctgagactttgaagcagtacatagccaccaaattgtttatctccccagcctaccttcatcttgggcatgccttccacacctaggatttgagggaagggatttctcagtgttctcatccctgcttctcatggaacatttatctccgttgttttttgagaagaagagtagtggatgtcagctttcttgtaatgagggatcctgggcccaagattccctgtctcccctcctaggctataaaattttggcctgtactccttctccctgagaggcaatgtgtctttacctacaagtcctagatgcaagatccttttctgccccacaccccagaatctgaaggcttttgctttggaggagcagtggtctagtgtgcaagggtttcatgtataccccccactaacagccaatcaccacctatagcctgaacagcttgatgcatggcaccctggtctcctgccttgttctcatgaacacccagaagaggtgtaagcaaaagaccattcacatgagtgtaattttgaagtataggcactctgatctgttttttgtttgtttctttgtttgtttgttttccagggttgaattaaaatatttatgactacttattaaatttctagaatcctataagtctatttgtatttttattctacatttcaatttgcatgctaatatagaagagtgtaaattgttaatcctcagattattccactttgtgtgtcataatttttttcacatttcccttttctaggcaatactgagcttgattttctcttttaatttcagcaccaactgaaaacagcactggggtccaggactgctaccgaggtgatggacagagttatcgaggcacactctccaccactatcacaggaagaacatgtcagtcttggtcgtctatgacaccacattggcatcggaggatcccattatactatccaaatgcgtatgtctatcatgttagccataaaaggaacaatagtcaactaaaatttctcttagctggcccatgctacaagctcacttcctaggtccaaatttctcatagactcagagtttgtagcaaaatgtctcaggaaacttacttttgagcaaaaggtctgaatgaagagaagttttaggattgctatctttcataacaatttgatggaagcagcaggatatatggaggtggtgaagtctcattaatgtaaagctaaggagatcaaatgaccaaatgctgagacaaagtatcattccacaatgcccactaaaggtccatgcagtctttcaaccatgcaattctatcattctatcctccattccctgaagtgaaatttgtgtttgccatttttgacacgaatcagaagtaacaaattcaggctgggtgcagtggctcaggcctgtgatcccaacactttgggaggacaagacgggcagatcaccagaggtcaggagttcaagaccagcctggctaacatggcaaaaccccatctctacgaaaaattaaaaaattagccggtcatggtggtgggtacctgtaattccaactacttgggaggctgaggcaggagaaacacttgagcctgggattcagagtttgctgtgagccgagaacatgccactgcactccagcctgggtgacagagcaagactcaatctcaaaaaaaaaaaaaaagaagaagaagaagaaaagaagaagaggaagaagaagaagaggaagaagaagaagaagaagaagaggaagaggaagaggaggaggaggaggaggaggaagaagaagaagaagaagaagaagaagaagaagaagaagaagaagaagaagaagaagaagaagaagaaaatagaaatgagtgcatatatttatatatgagtactagcctgtatgaacacactgggttctaagcaccagttttctgaagggatatgggttgtcaggcagagtaaaagcaggaatgcagatgagagtcaggaagtaaacagatgtggtgattaaaatgggcaggtacatggacaaaaaaatgcatgtctgacaaaaactggcctcttgccataagtgagtatgaataatatggaaaaactgtttgcacatgttgaacagcagacagtacaacctgagatagtttagaaagggaaacaaataagatcaaccccataattacccttcctagacttaagggcaaagagttttaaccaaagcattccacagcagtcttgctaaactggggagagagactggagttttgtttactaataaaaccgagattttctaggttaggtaataatgagaaagtatttgtggagaaaaggagctccaggaatacacacagaagtctcttcaagtctctggctgaacagaaagctgtgtatgcacagaaagagtttccagagagaaaggagaacaaagaacagctactggggaaagaacaactgctggggaacagtgagctcaatgaagatgccagagctcacatagcactgggaggtatttgagctctgaccagcctgaggagagacacttcattgaacatcttgggcattcagcaaagaccccaaaaaaccatacttcaggagtagaattaatgcattcctagaataaagtctactccagaaacaccctagaaaagcttagaaaccaagtctaaaaagatccaaatgatctccaagtaaattaattgcctgtcagaagaaaacaacctcttcagaggtaaacaacaaaattaaattgctcaattatatagtatgcacaatgtgtggcatacatttaaaaatttgctaaacatacaaaaagcatttagtgtgacccataaccaggagaaaaatcagtcaatacaaatagacccaaaaatgataaaaataacagaattggcaaggagatttaaaatgtatgtatcataattgtgttcaaggatttaaagaaagcgtggacaagaaataaataaatggataatatcaacagaaagaaaaattgtaaaaggaccaaatggagagtcaagaactgaaaaaaaagacatctctttaatgagaaaatcactacatggccttataatcatattagatagtacagatgataaagctaactagaaaatattagggtggtgcaaaccatagcacgcttatacaaagcctgagaagataaacagagcctcaaggacatctatgaaaatatcaaaatatttaatatttgtttaaagcaagtcacagaggaagggaaagagatattggaacagaaaaaatacttgaagcagtgatggctgatgactttctaaatatggaaaaaatgataaactcacatagtcaagaagctcaatggatcagatataggattttaaaaagtaaagctgtatgatttatttggacacatcataattaaattgtccataatcaaagatagaaagtaaaatcttatttgaagcccaagggaaaaaacatacctttacatagagtaacagtgacacaaatgactgatgccttctcatcagaaacaacacaaatcagaaacaatagaataacacctttagagtggtaagaagaaaaaaagatcaaatcagaaacaacaaaataacacgtttagagtggtaaggaggaaaacaagatcaaatcagaaacaatggaataacacctttagagtgtaagaaagaaaaaaagatcaaatcaggaacaacagaataacgccttcagagtggtaagaaggaaaacaagataaaatcagaaacaatgaaataacacctttagagtagtaagaagaagaaaagatcaggtcagaaaaaatggaataatatgctaagaagaaaaaaaaagatcaagtcagaaacaatggaataacacctttagagtgaaaagaaggaaaaaaacccagcaagcttaaacgctatgcacagcaaacaattccactgaaaatgaatgttacgtaagtacatattctgtcctcctaaaaacaaagaacaaataaaagaatgtttcatcagcaggattatgtaataaaagatgtgaaagaatgctatgtaagtagaagaaaaataataccatatgggaattggcatcaaaaccacaaaatactatcaaaacaaaaaaactttattgataaatttaacacaatatgcaaaagaactataccatgtatactacataacattggtgagaagaaaattagaagatctaaataaagacacatcatgcttatagattaaaaaatccaatgtcacttttcacaaaactgatctttagtttcaacccacacccaagcagaattcctgcagtcttttcttgaaaacctaacagaatgtatatgctagaatcaccaagacaatctttaaaaagaataaaaaacttggaataaaatcacaagtttgtgggatagatgcatatggtaatatggaaattctcataaagacacagtaatcaagacatgtggtattggctgggacgcttggctgtaatcctaacactttgggaggccaagatgagaggattgcctgagatgaggagttgcagacaagcctgggcaacatagcaagaccctcatctctacaaatatttaaaaaaattagccaggtttggtgccatgtgcctgtagtcccagctattcaggaagctgaggtgggaggatcactggagcccatgaggtggaggctgaaatgagccatgattgtgctactgaactttagcctgggagacagattaaaaccttccctctctctctcaaacaaacaaacaaaaaatacatagtattgggcaaaacatatgcaaacaaaaacagaaaagggtcagcataaatttacatatatggtcaatttattttcaatacaggtagcaaagcaatttaatgaggaaatttttttccaaaattggtctgaaacaactggatagccatagaaaaaaactataacaaatgtgacgcttgaatcctactgtatgactcaaattaaattaatttgagatagctcttagacctcaatgtaacagctaattctgaggctgaaatataagactgctatgaaaaagtatagtatcttataaccttggagaaggaaaaattttttgagggaagaaccagaaaacactaactgtaaaagaaaacaaatgataatgtggacattcattgaataaaaacttatgctcaccaaatatgactgttaagaaaataaataagtaagtaacacactggaagaaaaacactctcatccatatatctgacaaatggcctgtatccagagtatagaaacatttctcccactcactaatcagaggacaaacaacctaatcaaaatgggcaacaggcttgaatagtcatttcttaggagaagatgcacacagagccaacaatcacctgaaaaagtgcacaacatcttagccatcaaaaatcaagagttataaccctcataagatgacactgaacatccagtgtacatggatatcattaagaagacacaataataagtggtgtcaccgatttggagctagaatgtgccactctctcatatgctggtggaagttcaaaatcatacaacaaattaaaaaatcagtctgatgctttcttataaagttcgataaatatgcatctatcctacaaacctgtaattctattcttgaatatttaccccccaaaatgaaaacataagtccacaaaaatctatataaatattcatagcagctttatgttttataaactcaaaataaaaactatttcaatgttttcatcaaaagaaaatgaaaactatttaaatggtttcatcaaaagaaaatgaaaaaagaatttccagtatatttatacaaaggaatactattcatcaacaaggaacaagttactgatagtctcagaagcatgaacaaacctcaaaaatatattaaggaaagaagccagacgtcaaagtgtatagtctgtatgagtccattcatgtgagtttatagaaaacacaatttatggtgaaagaaaccaatagcatttgacactggccgtgggaagagggtagcagagattgattgagcagccacacaagggagtttctggggtggtgaaaatgttctgcattgtgagggcagtgtgggctacacaagtatatgtatttatcaaatctcatccagctacatttaagatctgtgcatctcactctatgtgaaaatatactcaactgaaaaacagagcaggtatctgtttcaggtgctacatcacttgatacgtccagttgtgttaaaaaccactgcctaacatcctcaaatgggggatctgggcttgagactaggtcacatgtgtagagtctctacagagaccgtgttggattcccatgctccataatacgttccaagttttctcagacagccacaggtcatgaatgtgaggattctgagaggttggagcaacgttcttgggaggcataatggggaaggcattctccaagattcctccagcctggggtcttcacctgctgtgcctcttactgcattgttttctgactcatccatagccacttgaccccttcagatcccatagtctacctagccgtctccctttatgccttgggtcccgctgttctttcaactcatcacccattccttcagtcccagagtggctgcagccagcagaggatggactgagagcaggagaggaggtcgtgcccatgaacccatcctagagaagcagcatcctgcctgggagctagttttccagggaagcttttataagtcctgtagacccaaacccacttgctctaccagatacagtatttatagtaatactattttcatgattattttatattgcaaatgtagagcatttatgctacactatgagtaaatagagtaagggggctggcatgggaattatataatcttggatgccacttcttccttggggaaatgtatttgagttccaacttacatattactatatagtcttatagagagagagacaaagagctagacagacagagatatctttgtatgtgcattaaaaaatctaagatacatatttcaaaatctgtgtcatttattctggaggaaagtatttggcagaaggtgaaaggaagatattctgatcctttcttgtacagacatgtattatctcagttttcatagagagcatatactacttttgatgttttaaaacaaaaattataatctgtgatgtgtccacagttgtttaaaagttgaagctgaagaccatttgtgcttgtggcaatattattgtggtataatgggaatatttcaaaggcacttgttaacactttgttacagcaaaatgtagagggcgctaagtgcccttgaatattctcccatctctggtgacctgtgttgttttgaaatttgcagtggcctgaccaggaactactgcaggaatccagatgctgagattcgcccttggtgttacaccatggatcccagtgtcaggtgggagtactgcaacctgacacgatgtccagtgacagaatcgagtgtcctcacaactcccacagtggccccggttccaagcacagaggctccttctgaacaaggtaagaaatttgtggttagacatctatatactgggatgaaaaaccatggaaaatcttactgatgcagaagccttcagtggtacactggagggttggttgagggtctgcaatgtggaggaaagcctcagcgccctctctgggggatccagaactgtgatttttggcacgctgtgaggaggcagtgtctttaggaagggcacggtgtctttaggaagggcacagacccgccagggcactggacttaccactcccctggttattaaatgggtcatttcagtgtcctagccaaaatggatattctaacagcctgccaaatatgtgaagatttccaagccaataagcctttccagtgatttaaagtagacttttttcattgcaatctacagtttgcagtttcttaagaacatggcctttgagtatgatatcctagagaaacctaaggagactgcattatttttctattgtcctggggctgcatagcaggaggtaaccaacgaatgctgtctctccctggcctatctcagtctttcacaggctctgttcacctcagctttgaagttagaaatttctaggtgttcttgcctcttcttctcatgaaacctgcattggcagtgagtctacagaagaagaggaagagaattctgctttgttacaattcaggactctgggcactagaagattccctatctctcctccaagggaataagttgtttgtctctaaccctccttgagaaacaatgagtctttgcctgcactcctaaatgtaggatgatttcctgcccaaattttcaaaagattaagccttttgccttggtatgagcaatggtctagggaaatgcgcaagggtcttgtgtcggcccctgactgaccaccagtcacctcctacagcctgcaccaaggaatgcattgcattctggtcttctgccctgtggttctcatgaaaaccagcagagattcatatgatggagctgcacatgaatgtaatttccaatgtccagcattctcctctgttctttatctttagatttaaaaataatgtttctatgaacttattaaaattctagaatactatgaatctactgggtcttttcacatccttttgctactagtagaaaaaagaatagtaataattttcagaggctactgtccagtatgtgacataaattgtctcccatgtttctctgctcatgcaattactgagtatgatttattttattttaatttcagcaccacctgagaaaagccctgtggtccaggattgctaccatggtgatggacggagttatcgaggcatatcctccaccactgtcacaggaaggacctgtcaatcttggtcatctatgataccacactggcatcagaggaccccagaaaactacccaaatgcgtatgtatttgattaaaaccataagaggagcaacagccaactcaaatattggttagaagacccatgctttaagctcacttcctagggacaaatttctcttagactcacattttggcaaaatgtctcaggacctttgcttttgagcaaagagtctaagagaagagaaattttaggcctgctatttttcctaatagttttatggaaggagtagaatatacggaagtggcgaagtcatattaatgtaaagctcagaagataaatgaccaaagcttaaacacagcaccattccacaatgcccactaaaaatcaatgtcatctttcactcgtgcaattctgtcattctaaatttcaattcccgaaggtttgtttgccatttttgtcatgggtaataagtaaaaaaaaaaaaattaagatgtgtatatatatatatatatatatatatatacacacacacacacacacacaaacatctgaatatttatatatatgtctgaatatttatatacttgtgtataaaacttatatttaaatttttgcataaatttatatatttttaatatttcattaaaaattatattgtttcactatgtatgtctgagtatttttatatattttaatataacattttaaatatttatatataaatattcaggtatgtaactgaatattcatttacacacacaaatatatgtgtgcatgtgtgtatatatatatatacccatatatatatatatatatatatatacatatatatatatatatatatatgtatatatatatatatatatatatatacacacacacacacacacacatacatacaggtataaacacactgggcctgaagcaccagtggtctgaaaggacatgtgttgccaggacttgaagagcaaaagcaggaaggcggatgagagtcaggaggtacacaaacgctgaaaagtaaaatggacaagtacatggacaaaaagcaggtataagcataacagccttttggaagtaaatgactataaaatatatgaaaatactgttttcacaagttgcacaacagatagtagtgtattgagataatttagaacagaaaacaaatgtgatcaaccccataagtgtgctgtatttcatcatggattgaaggaaaaagagatggagcccaagaagaccacagcagtcttgatgaactgagagacaccagagtttgggattacaaaggcagctgggattttctacacttggtaataatgagaaagaatttgtggagataaagagctacagtcatgtacctagaagtcacctcagtgtaatataaatctgcatatgcacagggagtgattccacaatgaaagtaggacaaagaacagctactggggaaagaataactacaagggaacaatgagttcaatggagatggcagagctcacaaagcactgggggatatttgagttcttaccagctagaaaagagacctcattgcaaatcttgggcattcagtagagaccccagaaaagccactctttggaaacagagttgatgtattttaagagcaaaatctactccacaaaaatcctagcaaaattgaaaagcaagtcagaaagaccaaaatcctctcaacataaattagttgcccatcagaagaaagcttaacctcttcataggtaaacaataaaatcaaattgctcagttatctggcatccacaatatgtgacataaatttaaaaatttactagacatacaagaagcatttagtgtgatccataaccaggagaaaaatcattcaatacaaatagacccagaaatgacagaaatgatagaattagcaaaaacatttaaaatatacatatgatcatttgatcttgtgatcagatatcacaagagaagaaagagatacttgaacagaaaaaatgcctgaagcaatgatggctgaaaactttccaaatatgaagaaaaaaaagctcacagattcaagaaaactaatcaatcagaaatatgattttgaaaagtaaaaatgtatgatttactttggcaaatcttcttggttaaattgtctaaaatcaaagaaagctaggaaaattttataagccagaggaaaaaagattgtttatataaaggaacagttacacaaatgactgatgccttctcatcagaaacaatgaaagtcagaaacaataaagtaacatctttaaagtaatagaagaaaaacccaagaggtgagggatcgtggcagacaggaggcaggactagattgcagctctggacagagcagcatgcagaggctcatattgtgaattttagccccatattgactgcaagaacagaccagcaatcctgagaggacccacagaccgtgtgaaggaagcagactgctcctgcaggataagggagacaccccaaatactgtgagttccccaactgcagaagtggaaaagggaggccttactccctcaaacacaccccacaactggagaagctgaaagtctgtttgcaggagaagttcccaactttacctgggcctcagtaaatttagagagctgagccaagcaaaatataggggtagaggaagcagcagagaagacctcagagcttgctggatccccaagcagctcattcctgcctggcaccacagagatccatcagaagtgtggccaaaggaacagagggtaaaactccacatggaggactgctctacctgaactttctaacaatttgaacagggggagaagcctcctggccagaacttgggggagggcatgaatctggtttgcagacttcacaggtgggggaaggactaaagcccttttctttcacagctgggaggtggaaagcctcaggcaagttttcaagcctgactttccccccacctggaaacagacttggagctgttgcggggttgggggcatggtgggagtaagaccagcccttcagtttgcatgggtgctgggtgaggcctgtgactgacagcttccctccacttccccgacaactcagatgactcagcagaggcagccataatcctcctaggtacacaactccagtgacctgggaacttcacccccacaccatacagaagcttcagtaagacgtgcccaaggaaagtctgagctcagacacgcctagtcccacccccaactgatggtccttccctacccaccctggtagcagaagacaaagagcatataatctttggagttctagggcccacccacctctagtccctctccacactagtatagctgatgcaggaggccaaccagcacaaaaatagagcattaaaccaccaaagctaggaacccctatggagtccattgcaccctcctccacctccaccagaacaggcactggtatccacagctgagagacccatagatggttcacatcacaggactctgtacagacagtccccagtaccagcccagagctgggtagacttgctaggtggcaagacccagaagacaggcaataatcactgcagttcagctcacaggaagccacatccataggaaaagagggagagtactacatcaagggaacaccccatgggataaaaacatctgaacaacagccttcagccctaccttccctctgacacagtctacccaaatgagaaggaaccagaaaaccaaccctggtaatatgacaaaacaaggctcatcacactcccagttcaccagcaatggatccaaaccaagaagaaatccctgatttacctgaaagagaattcaggaggttagttattaagctaatcagggagggaccagagaaaggcaaagcccaatgcaaggaaatccaaaaaaaaaaaggtataagaagtaaaaggtgaaatattcaacaaaatagatagcttaataaaaaaacaataaaaaattcagtagactttggacacacctttggaaatgtgacatgctctggaaagtctcagcaatagaactgaacaagtagaaaaaataaattcagagctcaaagacaaggacttcaaattaacccaatccaacaaagacaaagaataaaggataagaaaatatgaacaaagccttcaagatgtctgggattatgttaaatgaccaaatataagaataatcgtggctcctgaggaaaaagacaatactaaaagcttggaaaacatatttgggggaataactggggaaaacttacctggccttgctggacacctagacatgcaaatacaagaaacacaaagaacatgtaaatacaagcagcacaaagaacacctgggaaattcatcacaaaaagatcttagcctaggcacattctcatcaggttatgcaaagttaagacgaaggcaagaatcttaagagctgtgagacagaagcaccaggtaatgtataaaggaaaccctatcagattaacagccagtttttcagcaggaactgtacaagctataaaggattggagccctatcatagcctcctcaaacaaaacaattatcagtcaagaattttgtatccagcgaaagtaagcatcatatatgaaggaaagatacagtcgtttttggacaaacaaatgctaagagaattcaccattaccaagtcaccactagaagaactgctaaaaggagctctaaatcttgaaacaaatcctagaaacacatgaaaacagaatctctttaaagcataaatcacacaggacctataaaacaaaagtacaagttaaaaaacaaaaacaaaaaacaaaaccaaagtacggaggcaataaagaatatgatgaatgcagtggcacctcacatttcaatgctaaaattgaatctaaatggcctaaatgctccacttaaaggatacaaaaagagttggtggctggcaagatggctgaataggaacagctccagtctgccgctccccgtgagatcaacacatagggtgggtcatttctgcatttccaaccaaggtacccggctcatctcattgggactggttagacagtgggtgcagcccacagagggtgacctgaagcagggtggggtgtcacctcacctgggaagtggaaggggtcagggaactccctcccctagccaaaggaagccgtgagggactgtgccgtgaagaccagtgcattctggcacaaatactatgcttttcccacggtctttgcaacctgaagaccaggagattcccttgggtgcctacaccaccagggccctggatttcaagcccaaaactgggctggcatttgggcagacactaagctagctgcaggagttttttttcataccccagtggtccctggaatgccagcaagacagaaccattcacccccgtgaagaaagggctgaagccagggagctaagtggtctttctcagtggatcccacccccatggagcccagcaagctaagctccactggcttgaaattcttgctgccagcacagcagtctgaagttgacctgggacgctcaagcttggtgggaggaggggtatccacaaatactggggcttgagtaggaggttttcccctcacagtgtaagcaaaaccgctaggaagtttgaactgggcagggtgcactgcagcttggcaaagccattgtagcaagagtgcctctctagattcctcctctctgggcagggcatctctgaaagaaaggcagcagccccagtcagaagcttatagataaaactcccatctccctgggacagagcaactggaggaaggggtggctgtgagtgcagctccagcagacttagtttcctgcctgccagctctgaaaagagcaccagatcccccaacacagcactagagctctgataagggacagactgcctcctcaagtgggtcctggtttcagaagataataagaaactcctctgagctaaaggagcatgttctaacacaatgcaaggaagctaagaaccttgaaaaaggtcagaggaattgctaactacagtaagcagtttagagaagaacataaatgaccttagggagctgaaaaacacagcacgagaacttcatgacacatacacaagtatcaatagcaaaatcgatcaagtggaagaaaggatatcagagattgaaaatcaacttaatgaagtaaagcgtgaaaacaagattaaggaataaagaatgaaaaggaatgaacaaatcctccaagtatgggactatgtgaaaagattgaacctacgtttgattggtgtacctgaaagtgatgggagaatggaaccaagttggaaaacactcttcaggatattatccaggagaacttccccaacctagcaagacaggccaacattcaaattaaggaaatacagagaataccacattcaaattcaggaaatacagagaacaccacaaagatactcctcaagaagagcaacctgaagacacataatcgtcagattcaccaaggttgaaatgaaggaaaaaaatgttgagggcagccagagagaaagtttgggttacccacaaagggaaccccatcagactaacagtggatcttcctgcagaaactctacaagccagaagagagtgggaggccaatattcaacattcttttttactattattatactttaagttctagggtacatgtgcacaaggtgcaggtttgttacatatgtatacatgtgccatgttggtgtgctgcacccattaactcttcatttacattaggtatatctcctaatactatccctccccactccccccatcccatgacaggccccggtgtgtgatgttccccactctgtgtccatgtactctcattgttcaattcccacctatgagtgagaacattcggtgtttggatttctgtccttgtgatagtttgctgagaatgatggtttccagcttcatccacatccctacaaaggacatgaagtcatccttctttatggctgcatagtattccatggtgtatatgtgccacattttcttaatccagtctaccattgatggacgtttgtgttggttccaagtctttgctattgtgaatagtgccgcaataaacatatgtgtgcatgtgtctttatagcagcatgatttataatcctttagatatatatccagtaattgtatggctgtgtcaaatggtatttctagttctaaatccttgaggaatcaccgcactgtcttccacaatggttgaactagtttacagtcccaccaccagtgtaaaaatgttcctatttctccacatcctctctagcatctgttgtttcctgactttttaatgatcaccattctaactggtatgagatggtatctcattgtggttttgatttgcatttctctgatggccagtgatggtgagcactttttcatgtgtctcttgactgcataaaagttttcttttgagaattgtctgttaatatcctttgccaactttttgatggggttgtttgattttttttcttgtaaatttgtttatgttctttgtagattctggatattagccctttgtcagatgggtagattgtaaaaattttctcccattctgtagcttgcctgttcattctgagggtagtttcttttgctgtgcagaagctctttagtttaattagatcccattggtcaattttggcttttgttgctattgcttttggtgatttagtcatgaagtccttgcccatgcctatgtcctgaatggtattgcttaggttttcttctagggtttatatggttttaggtctaacatttaagtctttaatccatcttgaattaatttttatataaggtgtaaggaagggatccagtttcagctttctacatatggctaggcagttttcccagcaccatgtattaaatagggaaacctttccctatttcttgtttttgtcaggtttgtcatagatcagatggttgtagatgtgtggtattatttctgagggctctgttctgttccattggtctatatctctgttttggtaccagtaccatgctgttttggttactgtagccttgtaatgtagtttgaagtcaggcagagtgatgcctccagctttgcttttttggcttaggattgtcttggcaatgcatgctcttttttgttccatatgaactttaaagtagttttttccaattctgtgaagaaagtcattggtagcttgatggggatggcattgaatctataaattaccttaggcagtatggccattttcacaatattgattcttcctatccatgagcatggaatgttcttccatttgtttgtgtcctcttttatttcattaagcagtggtttgtagttctccttgaagaggtccttcccatcccttgtaagttggattcctaggtattttattctctttgaagcaattgtgaatgggagttcatccatgtccctacaaaggacatgaagtcatgtatgggaatgcttgtgatttttgcacattgattttgtatcttgagactttgctgaagttgcttatcagcttaaggagattttggtctgagaagatggggttttctaaatatacaatcatgtcatctgcaaacagggacaatttaacttcctcttttcctaactgaataccctttatttccttctcctgcctaattgccctggccagaacttccaacactatgttgaataggagtggtgagagagggcatccctgtcttgtgccagttttcaaagggaatgcttccagtttttgcccattcagtatgatattggctatgggtttgtcataaatagctcttattattttgagatatgtcccatcaatacatagtttattgagagttcagcatggagagctgttgaattttgtcaaaggccttttctgcatctattgagataatcatgtggtttttgtctttggttctgtttatatgatggattacatttattgatttgcatatgttgaaccagccttgcatcccagggataaagccaacttgatcatggtggataagctttttgatgtgctgctggattcggtttgccagtattttattgaggatttttgcatcaatgttcatcatggatgttggtctaaaattctcatttttgttgtgtctctgccaggatttggtatcaggatgatgctggcctcataaaatgagttagggaggattccctctttttctatgattggaatagtttcagaagaattggtaccagctcctctttgtatctgtggtagaattcggctatgaatctctcctggactttttttggttggtaggctcttaattattgcctcaatttcagagcctgttattggtctattcaaggattcaatttctttctggtttagtcttggtagggtgtatgtgtccaggaatttttccatttcttctagattttctagtttatttgcacagaggtgtttataatattctctgatggtagtttgtatttctgtgggattggtagtgatatcccctttatcattttttattgcatctatttgattcttctctcttttcttctttattagtcttgctagtggtctatcaattttgttgatcttttcaaaaaaccagctcctggattcattgatgttttgaaggtttttttgtgtctctatctccttcagttctgctctggtcttagttatttcttgccttctgctagctttttaatgtgtttgctcttgcttctctagttcttttaatggtgatgttagggtgtcaattttagatctttcctgctttctcttgtgggcatttagtgctgtaaatctccccctacacactgctttaaatgtgtcccagagattctggtatgttgtgtctttgttgtcattggtttcaaagaatatctttatttctgccttcatttcgttacatacccagtagtcactcaggtgcaggttgttcagtttccatatagttgagcagtttttaatgagtttcttaatcctgagtcctagtttgattgcactgtggtctgagagacagtttgttataatttctgttcttttacatttgctgaggaatgcctcacttccaactatctggtcaatttcagaataagtgcgatgtggtgctgagaagaatgtatattctgttgatttggggtggagagttctgtagatgtctattaggtctgcttggtgcagagctgagttcaattcctggatatccatgttaactttctgtctcattgatctgtctaatgttgacagtggggtgttaaagtctcccattattattgtgtgggagtctaagtctctttgtaggtctctaaggacttgctttatgaatctaggtgctcctgtattgggtgcatatatatttaggatagttagctcttcttgttaaattggtccctttaccattatgtaatggccttctttgtctcttttgatctttgttagtttaaagtctgttttatcagagactaggattgcaacccctgctttttttgttgttttccatttgcttggtagatcttcctccatccctttattttgagcctatgtgtgtctctgcacgtgagatgtgtcttcagaatacagcacactgatggatcttgactctttatccaattttccagtctgtgtcttttaattggagcatttagcccatttacatttaaggttaatatttttatgtgtgaatttgatcctgtcatcatgatgttcgctggttattttgctcattagttgatgcagtttcttcctagcatcgatggtttttacaatttggcatgtttgtgcagtggctgataccgattgtttctttccatgtttagtgcttccttcaggagctcttgtaaggcaggcctggtggtgacaaaatctctcagcatttgcttgtctgtaaaggattttatttctccttcacttatgaagcttagtttggctggatatgatattctcagttgaaaattcttttctttaagaatgttgaatattggctgccactctcttctggcttgtagagtttctgctgagagatctgctgttagtctgatgggcttccctttgtgggtaacccgacctttctggtgaatctgacaattatgtgtcttggagttactcttctcgaggagtatttttgtggcattctctgtatttcctgaatttgaatgttggcctgcctttgtaggttggggaagttctcctggataatatcctgaagagtgttttccaacttggttccattctcctcgtcactttcaggtacaccaagcagatgtagatttggtcttttcacatagtcccatatttattggaggctttgttcatttctttttactcctttttttctctaaacttctcttctcgcttcatttcattcatttgatctttaatcactgataccctttcttccacttgattgaatcaactactgaaacttgttcatgtgtcacgtagttctcgtgccatggttttcagctccattagatcatttaaggtcttctctatgctgtttattttagtctgccattcatctaaactttttcaaggtttttagcttctttgcaatgggttcgaacatccttctttagctcggagaaatttgttattacagatcgtctgaagccttcttctctcaactcatcaaagtcattctctgtccagctttgttctgttgctcgtgaggagctgcgttccttcggaggagaagaggcaccctgatttttagaattttcagctgttctgctctggtttctccccatctttgtggtttatctacctttggttcttgatgatggtgatgtacagatggggttttggtgtggatgtcttttctgtttgttagttttccttctaacagtcaggaccctcagctgcaggtctgttggagtttgctggaggtccactccagtccctgtttgcctgggtattaccagtggaggctgcagaacagcaaatattacagaacagcaaatgttgctgcctgattcttcctctggaagcttcatctcagaggggcacccagctgtatgaggtgtcagttggcccctactgggaggtgtcccccagttaggctactcgggggtcacggacccacttgaggaggcagtctgtccattctcagatctcaaactctctgctgggagaaccactactctcttcaaagctgtcagacagggatgtttaagtctgcagaagtttctgctgccttttgttcagctatgccctgcccccagaggtggagtctacagaggcaggcaggtctccttgagctgtggtgggctccacccagtttgagcttcctggtcgctttgtttacctactcaagtctcagcaatggcagacgcccctcccccagctttgctgccgccttgcagttcggtctcagactactgtgctagcagttcaatctcagactgctgtactagcagtgagcaaggctctgtgggcatgggaccctctgagccatgtgcaggatataatctcctggtgtgccgtttgctaagaccattggaaaagtgcaatattagggtgggagtgtcccgattttccgggtacatctgtcatggcttcccttggctaggaaagggaattccctgaccccttacacttcccgggtgaggcaatatcccgccttgcttcggctcactctccgtgggctgcacccactgtctgacaagccccggtgagatgaacccagtacctcagctggaaatgcagaaaccacccatcttctgctttgctcatgctgggaactgtggactggagctgttcctattcggccatcttgaaacctcccctctctcacgatcacaaggtcccacaataggccgtctgcaggctgaggagcaagaaaagccagtctgaattccaaaactgaagaaattggagtctgatgttcaagggcaggaaacatccagtgccaaagaaagatgtagaatattcaacattcttaaagaaaataattttcaacctagaatttcatatccagccaaactaagctttataacaaaggagaagtaaaatcctttacaaacaagcaaatgctgaggaattttgtcaacaccaggcctgccttacaagaggtcctgaagaaaacactaaatatggaaaggaaaaaccagtaacagctactgcaaaaacataccaaattgtaaacaccatcaacactataaagaaactgcatcaactaatgggcaaaatagccagctagcatcataatgacaggatcaaattcacacataacaatattaaccttaaatgtaaatgggctaaatgccccaattaaaagacacagactgggaaattgaataaagagtcaagacccattggtttgctgtgttcagaagacccatctcagggtgaaaagacatacatgggctcaaaataaagaaatgaaggaatatttaccaagcaaatggaaagaaaaaaaaagcagcggttgcaatcttagtctttgatgaaacagactttaaaccatcaaagatcaaaagagacaaaggagggcattacctaatggtaaaagtatcaatgcaacaagaagatctgactgtcctacttatatatgcacccaatacaggagcacccagattaataaagcaagttcttagagacctacaaagagacttagacttccacacaaaaatagtgggagactttaacaccccacagccaatattagatcgacgtgacagaaaattaacaaggatattcaggacgtgaattcagctctggaccaagctgacctaatagacatctacagaactcgacaccacaaatcaacagaatatacattcttctcagcaccacattgcacttattctaaaattgaccacataattggaagtaaaacacttctcagcaaatgccgtagaatggaaatcataacaaacagtctctcagaccaaagtgcaatcaaactagaactcaggattaataaactcactcaaaaccacacaactatatggaaactgaacaacctgctcctgaattactactgggtaaataacaaaattaaggcagaagtagataagttcttagaaaccaaagagaacaaagacacaatgtgccagaatctctggtacacagctaaagccatgtttagagggaaatttatagcactaaatgcccacaggagaaagcgggaaagatctaaaatcaacaccctaacatcacaattcaaagaaccagagaagcaagagcaaacaaatacaaaagctagcagaagacaagaaataactaagatcagagcagaactgaaggggataaagacacgaaaaccctttaaaaaattaataaatccaagagctggttttttgaaaagattaacaaaatacatagaagcctagccagactaataaagaagaaaatagagaagaatcaaatagacacaataaagaataataaaggggatatcaccaatgatgccacagaaatacaaactaccatcagagaatactttaaacacctctatgcaaataaaatagaaaatctaaaagaaatggataaattcctggacacatacaccctcccaagactaaaccaggaagaagtcaaatccctgaatagaccaataacaagttctgaaatcgaggcagtaattaatagcttaccaaccaaaaaaagcccagaccagagggattaacagtcaaatcctaacagaggtacaaagaagagctagtactattccttctgaaactattccacacaatagaaaaagagggactcctgcctaactcattttatgaggccagcatcattctgataccaaaacctggcagagacacaacaagaaaagaaaatttcaggccaacatccctgatgaacatcaatgtgaaaatcctcaataaaatactggcaaactgaatccagcagcacatcaaaaagcttatccaccatgatcaagttggcttcatccctgggatgcaaggctggttcaacatattcaaatcaataaacataatccatcacataaacagaaccaatgacaaaaaccgtatgattatcgcaatagacgcagaaaaggcctttgataaaattcaatacccaatcatgctaaaaactcttaataaactaggtattgatggagcatgtctcaaaataataagagctacttatgacaaatgcatagccaatatcatactgaatgagcagaagctggaagcattccctttgaaaaccagcacaagacaaggatgccctctctcaccactcctattcaacatagtattggaaattctgtccagggcaatcaggcaagagaaagaaataaaggtattcaagtgggaagagagggagtcaaattatttctctttgcagatgacatgattgtatatttagaaaactctatcatctcagcccaaaatctccttaagctgataagcaacttcagcaaagtctcaggatacaaaatcaatgtgcaaaaatcacaagcattcctatacaccaataagagacacagagccaaatcctgagtgaattcccattcacaattgctacaaagagaataaaatatacctaggaatccaacttacaagggatgtgaaggacctcttcaaggagaactacaaaccactgctcaaggaaataagataggacacaaacaaatggaaaaacattccatgctaatggattggaagaatcaatattgtgaaaattgccatactgcccaaagtgatttatagattcaatgttatccccatcaagctaccattgatttcttcacataattagaaaaaactactttcaatttcatatggaatagaaaaagggcctgtatatccaagacaacctaagcaaaaagaacaaagctggaggcatcatgctatctgacttcaaaatatactacaaggctacagtaacaaaaacagcatggtatggtactggtaccaaaacagatatatagaccaatagaacagaacagaggcctcagaaataacaccacacatctacaactattggatctttgacaaactggacaaaaataagcaatggggaaaggattccctatttaataaatggtgttgggaaaactggctagccatatgcagaaaactgaaactggatcccttccttacaccttatacacaaattaactcaagatagattaaagaattaaatgtaagacctaaaaccataaaaaccctagaagacactttgggaggccgaggtggatggatcacgaggtcaggagatcgagaccatcttggctaacacagtgaaagcccatctctactaaaaatacaaaaaattagctgggtgtggtcgtgggcacctgtagtcccagctacttgggaggctgaggcaggagaatggcatgagctgaggaggttgagcttgcagcaagccaagattgtgccactgcactccagcctgggcaacagagtgagactccatcaaaaaaacaaaaacaaaaacaaaaaatcaaaccctagaagaaaacataggcaataccattcaggacataggcatgggagaagacttcatgactaaaacagcaaaaccaatggcaacaaaagccaaaatttacaaatcagatctaattaaaataaagagcttctgcacagcaaaaaactctcatcagagtgaaaaagcaacctatggagaaaaattctgtggtctagccatctgacaaagggctaatgtttagaatgtacaagcaacttaaacaaatgtacaagaaaaaaaaaacaaccccatcaaaaagtgggcaaaggatatgaacagacacttctgacaggaagacctttatgtggctgacaaacatgaaaaaagctcatcatcactgttaattagagaaatgcaaatcgaaaccacaatgagataccatctcatgcccgttagaatggcgatcattaaaaagtcaggaaacaacagatgctgaagaggatgtgtggagaaagaggaacacatttacactgttggtgggagtgtaaattagttcaaccattgtggaagacagtgcggtgattcctcaaggatctagaaccagaagtaccatttgacccagcaatcccattactgggtatatacccaaaggattataaatcattctacaataaagacacatgcacacgtatgtttattgtagcactattcacaatagcaaagacttggaaccaactgaaatgcccatcaatgatagactggataaagaaaatgtggcacatatacactgtggaatactatgcagccataaaacaggatgagttcatgtcttttgcagggacatggatgaagctggaaaccatcattctcagcaaactaacacaagaacagaaaaccaaacaccatatgttctcactcataagtgtgagttgaacaatgagaacacatggacacaggaaggggaacatcacacacaggggcctgttggggagttgaggctaggggagggattggattaggagaaatacctaatgtagatgatgggttgctgggtgcagcaaaccaccatgacacgtgtatacctatgtaacaaacccacacattctacacatgtatctcagaacttaaagtataataataataagatacagaactgcagaatgaataagaactcaccaaccatctgctgccttcaggagactcatttaagacataaggactcacataaacttaaagtaaatgggtggaaataataataagtggtgtcactgatgtggaggtagattataaaactcttatcatatgctggtggaagatcaaaatgataaaacgaattaaaaaatcagtcagatggtttcttaaaaagttccatcaatatgcctctatcttacaaacctgcaattctattcctgaatctttatcccaaggaaatgaaaaagtaagtccacaaagagttctatatgaatatttataggagctttatttattataattcaaactgtaaaaataatttcaatgttcatcaataacaaaatgaaaaaataatttgcaacctactggtacacttgaatactattcagcactgagtatcttaaatagcatggatggagctcaaaaatatactcaggaaagaagccatgtatattctgtatgagttcatttacatgagatcatttacatttcctccaaaagaggaaaaactaatttctgttgaaagaaaccaatgtatttgcctctggcagtggtaagggggtagcacagattaattgggtagggactcaagagagtttctggggtcacagaaatgttccgtgtggtgatgggagtttgggctccacaggtataggtgttgatccaaaatcatcaaaaaaacaacattgcagatctgtgcatctcactctgtgggaaagtatatctcaactgtaaaaagggcagaaattgcttttaaacgctcagccttttagcacatccagttgcttggagaaccagcttactcaaatgggggtctaggctggagactaggtcacaggcatagagtctctaaactttcccatggcacataatacgtttcaggttttctcagagagctgcaggttagtaatctgaggattctgacaagttgggtcaacgttcctaggaggcatgaatgggagtgcattctctaagatccctccaccccagggtccttgctttctgtgcctcttactccattgttttctgactcctctgtagccactcgacctcttcagatcccattgtctacccagccatcgccctttatgacttgggtcccactgttctttcatctcatcctccattccctcagtttcggagtggctgccgctagcagaggatggactgagagcaggagaggtggtcctgcccaggaacccatcctagagaaatggcatcctgtctgggagctagttttttagggcaggttttataagtcttgtaaagccagacacacttgatctacctggtatgttatttacagtaatactattttcataattgcttttcactctaaaagtagagccttttagctacactgtgagtaaataaaggggctggcctgggaatggtatcatgttggatgttgtttcttccctgaagtaatatatatcagttacaatttacatgttactgcagagtcctagagagagacacagagaatgagacagataccaatacatttttatgtgcattaaaaaaatctaaggccaggcgcagtggctcacacctgtaatcccagcactttgggaggccgaggtgggtggatcacgaggtcaggagattgagaccatcctggctaacacggtgaaaccctgtctctactaaaaatacaaaaaattagccaggcgtggtggcgggcgcctgtagtcccagctactcaggagactgaggcaggagaatggcttgaacccaggaggcagaccttgcagtgagccgagattgcgccactgcactccagtctgggcgacagagcgagactccgtcacaaaaaaaaaaaaaaatctaaaatgcactcttcaaaatctatgtcatttattctggaggaatgcagttggcagaaggaggaagatattccgaatttttcttgtatacatttatgtatgatctcagtttttttatggatcatagaccaattttgatattttaaaataaaaattataatctatcttggaaatttacatggttctttagaacttgaggaccgtttttgcttttcggaatattattgtacctaaaatgggaatattacaacgtcactttttaacactttgttataacaaagtttagacagcgctgggtgcccctgaattttttcccgcctcttgtgacctgtgttgttttggaatttgcagtggcctgaccgagaactactgcaggaatccagattctgggaaacaaccctggtgttacacaaccgatccgtgtgtgaggtgggagtactgcaatctgacacaatgctcagaaacagaatcaggtgtcctagagactcccactgttgttccagttccaagcatggaggctcattctgaagcaggtaagaagtctgtggccagatatctacacatttgaacattgggatgaaaagagatggaaaatctgactgatgcagaagccttccatgctacacagaaacttgagggtatggcaggtggaaagaagcctcagcactctctctggtggagcaatttttggcgcaacgtgcgtgggcggtgacttcaggaatggtgcaaacccacctgggcacttgacttaccactcactttgttatgaaaggggttatctcggtgttccagacaaaattccaattctaacatcaggccaaatttgtgccaaatttcacactagtgagtgtttccaggcatttattaaaatggacagtgttcattgcaatcttcagcattgcagttgctgaggtatgtggccgctgagtttgtcatcctggggaaacctaatatgatgatatttattccatctaatcctggggctatttggcagtaaataccacagaatacactatttctctggcttatttcagtcttaggtaggctctgcacacctatgcttggaaggcaggaatttcttggtgttcttgtgccttcttctcatggaacgtgcatctttggtgtgtgttgagaggaagggtagtagacttctgctttgttgcaatgcaggatgctggaacaagaggattccctgtctctactgtaagggaataagattttagcctccatccttctctaagaagcaatgtgtctttgcctccaagtactagatgcaggaccatgaactgccccgtccaccagaagcttaaggctttggcttttcaggagcaatcatctagggaactgtgcagggttttcatgtctgtcccctactgacagccaatcaccatacagcctgcataacctaatccatcatcgtctggtttcctgcctcattgttttcatgaacaaccagtagagagccatacgaaagagcttgcacatgagtctttgttccaattgtaagagcactgataggtccttttcccaccaggttttgaatataaaatttctaagaacttattaaaatattagaatgttattaatctattgtttttgcttcagcatgtccttctgcttgtgagtatactaaagagaacagtcataattctgaaactactgtcctgtttgtgtcataaattgcttcacatgtttctgcatactagtagttactcagcttgattttgtctattttcagcaccaactgagcaaacccctgtggtccggcagtgctaccatggtaatggccagagttatcgaggcacattctccaccactgtcacaggaaggacatgtcaatcttggtcatccatgacaccacaccggcatcagaggaccccagaaaactacccaaatgagtatgtctttgatgttacttgtaagaggagcaacagccaacttaagttcctcctagaagagccttgcttcaagctaacttgttaggacaaatttcccttagacccagaaggtgtgtcaaaatgtccagacaactttgcttttgatcaaagagtctgagagaataggtattttaggcttgctatcttttctaatagtctgatggaagcagaaggctacatggagctgatgaggtctttttaatataaagctcaagagatcaaatgatcaaatacttagagtgccattctacaaggctcataaaagatcaatgcactctttcacccatgcaattctatcattctaacctcccttctctgaaatgaaggctttttgccatttttgtcatgggtcacaagtaaataattcacatgtatatgagtatatatataaccaggtgtgtttattcagactagtatgtatatatatacatatatatgttcatataagttagtattcatatatatgttcatatatatatgttcatacagactagtattcatatatatatacatatatatatacacacacatatatatatatatatatatgttctagggaaacatgcaaggtttttatgtctgtccctgactgatgaccaaataccctatagcctgcacagctgcaagctgtatagccatacaatttgcaggacacacacacatacacacacacacacacacacacacacactaacatataatataatataatataatataatataatataatataatataatataattaatatatataaacctgtgtgaacacactgggttctaagctccagttttctgaagggatatgggttgccaggagaggaagagcaaaagcaagaatgtagatgagaattaggaagtaaacagatatggagattaaaatgggcaggtacatggacaaaaaaccaggtctgacaaaaactggctttctgccataaatgactataaaagatattaaaaaacactttccacatgttggacaagagacagtacaggactgagataatttagaaaaggaaatgaatgagcgcaactccgtaactattatgactttcttcctggagaaccttcctggactgaagggcaaggaattggagccaaagccaaccacagcagtcttgctgaactgaggaaagagactggagtttgggatagctaagaaaatgtgtattttctatgctaggtaataatgagaaagaatttgtggtgaaaaggagctgaaggaatatgcatggaagtctaatataaactgcatatgcacagggagaaattctacaaagtgggacagagaaccactactggggaaaggacaaattcagggaaacagtgagctcaatggtgacgccagagctcacgtagcactgggggataccggggttctgatcagcccgaggagagacacctcattgaacatctcgggcattcagtagagaccccagaaaagtcatactttaggagtaggatttatgccttcttagaataaagactaccccagaaacaccctagtaaagcttaaaaaccaagtctaaaaggacccaaatgatctccaagtaaattaactgcctgacagaagaaaactcaaccatcactggaggtaaataacatgattacagtgctctgtaatgttgcattcacaaggagtgacatcatttaaaaatttatgaggcaggaaaaagcaattagtgtgatccataactaggagaaaaaccagtcaatacaaatagaccaagaaatagtagaaacgatggaattgacaaagaaattaaaactgtatatatgataattgtgttcaaagatttaaagaaaacatgaacatgagggaaacaaatgcagaatataaaaaaaagcaaatgcgtaaaacaaccaaatggaaattaaagaactacaaaaaagtataaccttaataaaatactcactggatggccttaatattagtttatacattacagaagaaaaagtgaaccagaagataactcaatgaaagccatacaatctgtaagacacacacacacgcacacgcgcgcgcgcgcacacacacacacacacagagagagagagagagagaaagagagagagagaaaggctgaaaaaaataaatagaaccttaaggatatcagtgaaaatagcaaaagatttaatatatgggtaaagcaagtcacagaaggacgggaaggagatattgggacagaaaaaaatactcaaagcaatgatggctgaagactttacacgtatgaagaaaatgataaactcacagtcaagaagctcaatgaatcagaaatagtatttttaaaagcaaaactctatgatttacttgggtacattatagataaatcgtccaacatcaaagataacaaggataatcttataagccagaggaaaacaatatcatttacatagagggacagtaatgaaagtgaccgatgccttctccttggaaacaatggcataacatctttaaagtgataaagagaaataaaaacagatcaacctaggacgacatgtccagccaaaacaaacaaataaacaaaaaaaccctttaaaataaacgtgatgtaaatacgtattctgccacctccagaggaaacaagcaaaaaaacaaaagaatgtttccaaggcaggcttctgtattaaaagattttaaggaaagttattcaggtagaagaaaaataataccagatgggaactttaatccatactaagtaatgaagagccctggaaatggcaaatggcaatgtcaatataaaatactcttatttatctaatttttaaatgtatttaaaggacaatttgtgatattaattaaaataataggaatatattgttgtttcaacgtatgtagtagtaaaattcataaaaacagtagcacaaataatgcagatgataactggaagtatactgttaatgagttttttgcattatccatgaagttatataatattaatagatggttgaatgtgatagtttaaggtgggatattataaatcctaggacaaccaaaaaaatttaaactgagaggaatggatagtaagaggaatagtccttttatgcaaaagaaggaagaaaaagaggaataaagaatataaaagatatggtgtaaacagaaaatacatagcattattgtagacacaaactgaactaccttatgagtatattaaatataaaaggattaagcattacaaataaaaggcagagattgtaaattgaataaaaaccacagctaagtgtgttctttttagaataaatactctttaagtgtaaagatctactttaaacaccaaaatatgaaaaaggatatataccatgaaaacctgaatcataaataagctggagtggtgattaatggatgcaggcactcctaaagactaataagtgaatgtggtcaaattgaagaaacaaaagtatatacgtgctcaatgtgcaaaaactttttctgtatacatgctatgatcctttggaaaattaaagttttaaagcaatatcactgacaatagtatcaaaaccaaaaaatatttagtgataaatttcacacactatgctcaaggactatacaccttgcactagaaaacaatgttgaggaaagaattaaaagatctaaatatacaccatgcttatagattaaaagactccatatcagttctcgtgaaattgatctttggatgaaacccacacccaagcactattgcaacagtccttttttggaaaaaaaaattggaggacttatataccttaatataaagacttataaaagtacaggaatcaagacatgtggtattggcctggccccttggctcatgcctgttaccccaacattttgggaggctgagtctggaggatggcttgagcccagatgttcaagaccagccttagcaacagagtgagaccctctctctacaaaaaataaacaattagatcgatgtgatgacttgcacatgtagtttcagctactcggaatgctgaggtgagaggattgcttgactcaggaggtctagccatgagtgagcattgatcatgcctctgcattccagcctggatgatggaatgagacactgtctcaaaaaaaaaaaaaaaaaaggatatgtgttattggccaaaaaagtatgcaaacctaaaaagggatggcccaccaccagacccacatacatatatggtaaatggattttccgtatagatggcaaagcaattcaatggagacaaaaatgttttacaaaatcattctgaaccatttggatatccatgatacaaaacaaaagcagaacttgacttttgcttttcatctcaaattattttgatatctcttccacctaagtgtcagagctaaaactgaacctgaaatatgaaagttccatgaaaaaatataaaatcttcacaaccttggagaaggcaaacttttttgaggcaggagtctgtaaacactcactataaaataaaacaaattataatgtgggctttcatgaaaactcatgcttaccaaaagtcattgttaagaaaataaataggcaagtaacacatgagaagaaaaatgctctctgtccatatatctgacaaatggcttgtgtccagaatataggaacatttctcccactcactaaacagaggacaaacaactaatgggcaacagattgaataggcatttcttggggatagatagatgtacacatagccaataagcacctgaaaaaatgtccagtatctcagccatgaaaaataaagagttataatcatcatgagatgtcaccaaacacccaatggacatggatattattaagaagacaccacagtaactgatgtcactgatgtagagcaaggatgtgaaactctctcatatgctggtgaaagtgcaaaatgatacaaccacttttgaaatcagtctgatagtttctccaaaagttcaataaatgcacttttaccctacaaacctgcaatcctgtttgtgaatatttaccccacagaaatggaaacataagtccacgaagacatctccaagaatattcatagcagctttattttttataaccccaaactgtagacaatttcaatgtcaatcaataagaaaatgaataaataatttgtgaactagtcatacaatggcatactgttcagcaataaaagggagcatgtttttgatactctcaaatagtatggaagatgctcaaaaatattacattaaagaaagatgccagataacaaaaatgaacattatgtatgagtctattgatgtaaggttccagaaaggtaaaactaatttctggtgaaagaaaccaatatcatttgcctctggccatgggaagagagtagcagagattgattgagcagtaaaacgaagtttttttctggggtgatgtaaatgtcctgtattgtgattgaagtgtgagttacacaagtgtacatgttcatcagaagtcatcaaactacatctaagatctgtgcatttgactatacatgaaaatatacctcagttgaaaatagatcaataacctccctcatatactatacttgctaacacagccagctgcttggagaaccagcttgctggaatggagaatctgggcttgagactgggtcacatgtatagagtctctacagagacaatgttgcattcccacggtacataatacatttcaaggtttctcagacagccacatgtcatgaatgtgaggattctgagaggttggagcaacattcctgggaggaacgaaggggagcacattctccaagatcccccaccaccggggtcctcaccggctgtgcttttttttttttttttcttgacagagtctcgctctgtcgccaggcaggagtgtaatggcccaatctcggctgattgcagcctccaactccagggttcaagagattctcctgcctcagcttcatgagtagctgggactacagatgtgcgccactgcgcccagctaatttttgtatttttagtagagacggggttttgccatgttggccaagatggtctcgctctgttgacctcgtgatccacccgccttggcttcccaaagtgctgggattacaggcgtgagccaaagcacccagcctgtgcctctcacttactcaattgtttttctgaaccctccatagctggtggaccttttcagatcccatagtctagccagccctctcactttatgccttgggtcccactgttccttcatctcatcccccttctgtcagtcccgcagtggctgtggccagtagaggatggactgagagtaggagaggaggttctgcccaggaacccatcctagagaaacagcatcctgcctgggacctagtcttccaggtcagcttttataagtcttttagactcaaactcacttgacccacctgaagtggtattgacaataatgctattttcatggttgtttttcactgtaaatgcagagccttttagctacacgactagtacagagagtaagggaggctggcctgggaatgatatcatcttggatggcatttcctccttggagaaatatatgttagttccaactcacatgttactatacagtcctgtagaaagagatacagagagttagacaggtatagacgcatttgtatatgcataacaatctataagacacacatcaaaatccgtataccggttcctctaggggtatgtgcttggcagaaggtagaaggagggtattctggttcctttcttttgcacatttatgtatgatctcagtttttatatggagcattgatagggtttggctatgtccccacccaaaatctcatcttgacttgtaatctctataatcctgataatccccatgtgtcaagggcaggaccaggtggaggtaactggatcatgggggcagtttctcccaggctgttctcatgacagtgagagagtctcctgagatctgatggttttgtaagtgtctggcatttcccctacttgcacttactctgtcctgccgcctgtgaagaaggtgcctgtttctcccttgccttctgccatgactgtaaatttccagaggcctccccagcaatgtggaactgtgagtcaattaaaactcttttctttgtaacttacccagtctgtctcgggtatttcctcatagcaatgtgagaacgggctaatacaagcatatactacttttgatattttaaaataaaaattatcatctatctttgaaaggcatgcacaaatgggaagttgaggaacatttgtgttgtggcaattgtatgatacctttaatgggaatatttcaaagacacttgttaagactttgttagaacaaaatgtagagggtgctggatgtccctgaatattcttccgcctcctgtaacttgtattgctttggaatttccagtggcctgacaatgaactactgcaggaatccagatgccgatacaggcccttggtgttttaccatggaccccagcatcaggtgggagtactgcaacctgacgcgatgctcagacacagaagggactgtggtcgctcctccgactgtcatccaggttccaagcctagggcctccttctgaacaaggtaagaagtctgtgtcttaccttgtctagcacatacctctctatgtgcttggacaacgggatgaaaagacatgaaaaaccacactgatgcagaagcctttagtgctacacgggagctcgagtgttggttgaggttctgccatgaccaaggaagtctcagtgccgtccctgggaaagccagagctgtgatttttggcacaacttgtgggagtagtgactttaggactggcgcaaaacctccagggtgctcaacttaaccactcaccttattctaaaatgggttatttcagtgtcccagtcaaattcctattctaacatgctgtcaactgtgtgattatttccaagccaataagcatttccagtaatttcttaaaatagtgttcattgcagtcttcagcgttgtggctcctgagggatgtggcccctgattctgtcgtcctagagaagcctgacatgactgcattgattctgtatcgtcctgggtctatgtggctgcctggctgtctgtaatcatctgttttatttttatttttttctacagactgtatgtttgggaatgggaaaggataccggggcaagaaggcaaccactgttactgggacgccatgccaggaatgggctgcccaggagccccatagacacagcacgttcattccagggacaaataaatgggcaggtctggaaaaaaatgtaagccactttgatttggactctttttccctttgctgacaaatcttttcaaacagaagaggggcagaggaaaatactggaaagacttcaggaggctaagcgtaattagccttagcatggaaagtgcaagcagcacaggccagcaaagccccacgcgtgtgggggttctcaggcctcttctcttttgacatttctttactgtttccattgttgggtgctgtttctcgtttctagtgcttgtcctctaagccaggggtccccactccagtactggtactggtactggtactggaactggtaattatctgtggcctgttaggaactgggctgcacagcaggaggtgagcttcgggggagcaaacaaagcttcatctgtattttctgctgcttcccatcactctcatagctgcctgagctctgccagctgtcagatcagaggcagcattagattatcatagcacaaaccctattgtgaactgcacatgtgaggaatctagattgcatgctccttatgagaatctaatgcctgatgatctgtcatgcttccatcacccccagatgggaccacctacttgcaggaaaattagctcagggctcccactgattttaccttatggtgagatgcacatttatttcattatatattacaatgtaataataattgaaataaagtgcacgataaatggaaggtacttgagtcatcctttaaccatcgccccctcaccccaggtgcacagaaaaattgccttttatgaaactggtctctggtgccaaaaaagttggggaaccacactgctctgggttctagtagtcagagatgccctctatgaggcttaagtcagatttttctagaaaagatttggatgggccatcaggtcaccatgagacttcccttagcctcatgcattctctgtgatggtttactttggggcctatgaatagggaagactgagatataggaaaaaccaaagtgtctgtgttcccccactctcacacccatgtaacataacacttctcacaccagatatggggggatttctcctcacaccccaagcgagtctccagcagataccagctgggtgtcctacaatgtaactcggtcctgacactctatctggagacagtgtcagatcccacaagttaaggctcagtcctacaagactgccccactgcagatgccaatcccaagttgcaggctgtgacctgtacttctgcccagctggataaagatctgtttttctatatgaccctccatgggtttgattactttgctagagtggctcacagaactcagggaaacacgttacttttatttacccatttattataaaagatattaaaaaggatcctggtgaacagccaggtggaagagatgcacagggcaaggcacgtgggaaggggctcagagcctctatgccctctccagtgcaccagtccccagtaccctaagtgttcagcaacccagaagctctccaagtgcagtcttgttgggtttttatggaggcttcattacagaggcacagttgattacatcattggccatcggtgatcggctcaccttcggcccctcttccctccctggaggttggagggtggggctgaacagttccaaccctcaagtcacatggttggttcccttggcaaccagcccctggggctatccaggaacccaccaagagttgcttcattgcagctcccttcacccaggaaactccaagggatttaggagctctgtgttaagaactggggggcagagacccaatatacatttcttattctatcacaatatcacaggaagctaaggatgatactgcctttgtgtgtcttggctgtggatggtgcataatgcatggaagtaagcatttctgaatcaacagcaaacaggctttatcaggtagaagacccctcagcgccccagggacaaagctcatcaatgatgtcccactgtcctctgaggctctagctctaagacctccagtgggtcaagctcctggagaagtggcacattctccaaagacccttcagggtcaccacaccctggttaagggtgtggcctcataactccttttgactatgactgatggcttacagcatagaaagaaataactttgtcaaaaaatataataatgatagaaaggaagaaggaacgctcccttttgtcttctaagaatagatgtgaaatgtgtgtgccttagaatatcttctccctctcctgctccacgtgagctggagcttacatgcctgcttgttttcagtactgccgtaaccctgatggtgacatcaatggtccctggtgctacacaatgaatccaagaaaactttttgactactgtgatatccctctctgtggtaagttgccttctgttttggtaaggaaactgcttccttaatatggatttggaaaaaaaaaagcaaaaaaaacagaaaatggcttttgagctgagtgcttctggggaggagatggctgccctctccaccagagcctgcttttcatcatggccaccttgaacctgccctactattggccccatttgttaggaaaacacccgcccctcccaccacacacacataaataaaataaatgtcaaattcccaaagggcaaacttagaggtgatctaatcagcccgggatagtcccaccgaacccttctttgtctagcgtgggatgcatgaaaaacaaatttagagtcattatgatgaaaaactgtcctcttctgcagctgagaagaaaaaaaaaatacgagcagcaggaaacagctaagcatgtaatgcacattgtaaacctcagatggccatcctaggaaatcaatgaagggtagtgcagctctttagccccagatggcctttctcgtaagattactactcatgagtcccattagcgacattgcttagagactgcttgttaggttccttcctcattgctctgagactcttattgggagtatgaggcttggatcaggggaaggggaattgacattagatcttaaatgattggggtaacaaatccatgggggaaaaaaagccacttgtacttgttccctattttcttcctgctgaccaatcaacttgtctgtccgagttacagaacaccaccctggacttttcttttgtgtaatttggttgcttgtggttgggtctgccatgtgaagggaccttgagctgggggaagaaggttggcctccaagtccactgaagaccagcatcctgagattgcctggggaggtggtacagggcagtgatgaagatcatgggagccacactgcccatcgtcacatttgggccactcctggggagagcaagagggaagaaggagaggttagggtgataggaaagattctacttggccaatattattataatgtggcattgtggtctctggatttagtgtgagttgatagctgacttttttctcgagtgggtgcttttgttctattttgtcggtgctattgcagaagcatcttggtggttcctctacctcaaagtctcttgatggggtcagttccagttctccgcttctggccccatctagtacacgccactgcctctcactgcctgggctctctatccttgacaggctgccttgaatttaagcccagtctgacttacctgcctcaaacacccacagtagtgcctgggactcatgcacctttgactcccatggaagggaagtgcagtagcttcccaggtgcaattctgctgtcctcacccacattgaggatgtatgagaatcaggttcttagagattggagaaagaaggaagaatgggaacaagatttcttccaatggactgtgaggttccccaccttactttgatgtaagacaagtgaggttaaccccaagcctggtgaggagggttcccatcagacacttggaaatcctgaggactgtttcctgcagaaggatgtggttggtgggatattcaggtttgactcatgattgagaaagttagagcctctggttggagaaagagtttaataactatttcatttccaccaacacattcagtacgaataataaataagtaaaaataaatagaaacattcagttttattttgaatagtaggagtagggtataatttctgtagttactcttttagtacaatgatgcatgtttactgtatgtaaggcatactagcagaaattgagctcagcactagaaaagatgattgcattccatgccatgcttcttttttacaaaagacttctatagatagattctcaaaacaacccacagcaaatgaaaagttatttggaaaactcaggttccagattcactggagtgtagaatctctggttggttggggaggaatttcctcttgcagttgttattaataattatatgaataattattaactatattaatatttatagttttgaagaccttgaagggctggagacaacagagaagcatttttgaacaccctctgtagcccctgcactgttgtaggcattgatgggtggtaccaaagatgggacactttccctacctccagagaccttgtgggcttgctgcagagagaaggcagggaggaggaaaagaagaatagaggcacatgtgtgtaaattacccccacagcagtcagttagtcatgggaggctccccagaagaactgtcctgaagctggctgagagaaggcaacatttcaacataggacagttatccttgctacataaaatcacatacacacatgcacatatgtccacacacagagactcacatgcaaaagaatcctttgtgcctttcagtaaactttacatggtttagaaagaacttatatttccttgaaaggagagtgtcctttgttgtttactaccactttttaaacttagaaagaaaaatctaaagagtgtttatgattttaccatttaatttcacctttgagatgtgaaaaactagtgcttggaattcgtcctgaattaaacgacacaattgctaacttggactcaaatgcgacttcttttcccaccttgtgccacagcatcctcttcatttgattgtgggaagcctcaagtggagccgaagaaatgtcctggaagcattgtaggggggtgtgtggcccacccacattcctggccctggcaagtcagtctcagaacaaggtaagaacaggcccagaaaccatctatactgtccttccatgtaagccccacaaaacccttctacatttacacagaacccacacagctgatgcatcaatacctgcctctctgttttctgaaggaggaaaaaatatagaaaaattaaaaaaagttatattattataggttctctacttggaaaatagccaaaatacaaatctttttcttgatctgggcagttccatcaaaatctgtaggcacagtgatttgcaccaagttccaatacttttggaaaatattgaagatgctctgagggtttctatggatatccattgtctcactgtcagatgaaaagaaagggaagtttttagaaatgtgacactttgcagtgagggaggacaagagcaaacttacctacagtctatcacaggcacagatttttttttacacttttgtgaatcattgaattcaatgccgaggctattcatctattcacaaacacatgaacaaattatgggttgtgatccccataaatgaagagtaatcagtccgaacccacagaacctggacattttgggtatcgtttcagtggaacatgcaattcgtaagttcagtttgcttgggtgtctcttaggaagaacacataggacacagacccatctgcctgcatgttttgcttcctcatctcctttctacaccagggcacctgtgctcaattgctgttctcctctaaagagacttccttctgtaagtttgtgaaatgccatcgacaaacctgatcgcatcgcatttcactctgctgttgagttgatttttctttactttatcgtttgtaacttcttgctctacagagctttcaccttccacatatttcagattcattctttcctaaactgtgtggtggtctatgtcctcactgactatcaacatactgccatcatgcacttcctatctctattcctcttcgttgcaatctggctccaagtggctcacaccattattctgatctatcaactgcctacacagtcctagaaagtaagtgagtcaagaaacatcccccaaaagtaaacttttcaggtaagatcagaagaccctcatgagtcactgctgctcaggatcgtatctggctccttgaagagtgaccttgcatagatcttgtcataaaaaatgaaagagaccttgggaaggtcttgggctggtcacttttgtcagagtccagggctgtggggtgaaagccacagctatagagcttcattctggagtcacttagctttgctctcctggggacaggctgtgcctattcttgcctcaggcatcaaaaaaagtggcacagatgggcccttctgaaaaatctcactactggagcacagctcgaagtttctactatcctgacgttgggcggtagtcctttgctttgggaatatgaacatgatcaaaactgagtgaacttgtcttcctggctttctgtacaatgaagtagaacaaaccatccaatttgaccaaagccttggcatgttttctttctaggtttggaaagcacttctgtggaggcaccttaatatccccagagtgggtgctgactgctgctcactgcttgaagaagtacgtttaagggaaaactgacatggggtcttatcttcaagacttttttcctccctctcttcctccatcccttctttcttcccaccctccccttccttcctccccacctctcttccttttctggaaggaacactaggaaccagggaatgcatgcagaatcctgaggcagaatttccagggcaattggatgagagaggagggaagtgtttctagagggaatctgcagagggaagacccagtgcaagtgattttttggacctgtataaaccgcaggacagagctgttcactaccagaggcatcaatctgtattgcattgctctagagcaatatctgaggctgaataatttataaagaaaagagtttaattggcacatgtttctgcaggctttacaggaagcaggatgctgtcatctcctctgcttctgtgtgggcctaaggaagattacaatcatggtggagggcaaagtgggagcaggcatgtcacatggccagagcaggagcaagagacagagagagatggggtgggggtgctgcacaataccaaatgaccagactttgcaagaactaagagtgagagctcactgatcaccatgaagatgtggcccaagccattcaagagggatgcacctctatgatccaaacccctttcacaggccatagctccatcactggggactacagttgaacacgagatttaggtggggacaaatatacaaactatatcacagtctctgatgaaacagattgagaacagaccttaactgtcagtttccagcaaattgtgaattttgtttcttgccactcataagtcactgattctgggtggccgagggtgtcagagggacagcgccaagttcatggcacagaggatacctgaaggggctggaccatatttttctcttgacatcctcatcttttctaggtcctcaaggccttcatcctacaaggtcatcctgggtgcacaccaagaagtgaacctcgaatctcatgttcaggaaatagaagtgtctaggctgttcttggagcccacacaagcagatattgccttgctaaagctaagcaggtactcgctcacctgtggtcttcaccccacgctggtgaagatatttgctttatgtctgggttttatgggccatggccactgcatggcagtggggaggaactgtctatcacatgaaaggctcaagggctttggggacagcatcaatcttcaaccccagccctgccacatgttagttgtgctctttaaaaaggcagaaggattcgtttcctcacgtggaaaaagagataccctgttacccgtaaaacttacttaatgttcaccagttcatccacattcatgatcagggaaaggttgttattccaggctaactattctcctttcataataatatgctggagagaatcaaatgagattgcatttcaaagcgcttgaaaaaccaccatatcgagccatgcttagtgtgggcgcctctaatcactgctattcaggaggctgacgaggaagaattgcttgagcccaggacttcaaggctgtaggcagctatgattgtgccactgcactccaggctgggtgacagatcaagaccctgtctcaacaaaagaaaagaaaacaaaacaaatgaacagaaatattccacaatgtcaaaaaaaaaaaaaacccacacaacatacaatttacaaatgcaaataataatattattgttgtcttctttgattttctctttcctggtgaaattttgttttattaagcctgacaaagtgatacctttgcttacatcacttaaagttagtctatttggacctaggtgacagtacaatcagctaagaaacagtatttgtaggagaggcaggtttgggacaggtgacaaggcatgtggggtgctcgctgtgctggtggctctggaaggcagggtgtcaatgcagacagggatgagcatggcctggttgggaaggcatggggcaggcaggagcctgagctgctctcctgggcctggtcacaagcccatggcagcttctctgggtctgtgaactgaggggtgatgtcctggaatcctctgacactctaggaaggagagaagggcctttctggctcagcctttataaacagtagctgatctccctcttgctccccagggtcctccccaccatcccagcaaatgtgcaaatacaagatctctgctcctcatggtcctcagagagctggggtgttctgatggcttgaacaagtcacttaggaaatgtggggttttggaggcattctctgataggctgatacgttttgagtttagagttcccaccgcacatccccacacccctagagtctagggcatttagtgctccatgagggaacctgtagagtgaggacatctgcatcacaggctgggccttctagtgtccagaagcagaaagtgtgtctgcttcaaagttggtgctaatgatgatttttggtcagaatacggcatttctcatttccattcctttatccccttgaacttactaaagtagaatcaggtctaaaaaccagagttctaatctttaagagtccctgggattctaaggtatatgaatgtccttggaaaacaataccatttagttcatgcaaggtgcttatttcccatcctctttcatttgatgtctagcattttactgcattcttaccaccacggtttagtaacattcacgaggaggaagtggaggatccagatggagcaacttgctctgggcacacaaggcatttgcaattttataccctcttgatgatgtctcagccagacattctgcccagtcatcaatgccctcttcaattaatatgaaaggacacacttggcatgagattccaatcgtgcacagaatatacatgagaagtgtgcctttgtcatccctactttcaaaggctaaggccaccctcagtttcttgcatgcaactgatgcctttcaaatgaaaccttacatctgtgtagtccataggcaaccacaggcaaatgtgagggtgaaacgctgtgttctacattgttctgtgtcagtgaagcaaggcagtgccagctcagagggctctggggcttcaaggcagggatgcctggttgtaggtactgccacttccagctgggcagtgaaacataactgctaatactttccttacaggcctgccgtcatcactgacaaagtaatgccagcttgtctgccatccccagactacatggtcaccgccaggactgaatgttacatcactggctggggagaaacccaaggtgagatcaattccattgcccacgtaacaaattgtttttgaccttcagtgcatgttacaaaatgagcattttggagatagttgtacaaattcctacccatgaatgtggtctacccactcctgactttgcctggacacctgtctatgtctccataatcagtcttcaagggacttgggcaaggggagcggtgccatttccttgagtctctctcttttttgttttcagaatcttttaattttttttgtaatgattgtatgtttcccttacaacaaaaacaaacaccagtagaggtctttgagtctcttaatcataatttcagcattcatattgcttccccaggtaagtggggttttgacccagccctcaagttaagggtgttagattatttttcatgtgaaattagacagactgcgtttctaaacatggtgcaaaacagtaacgacaaaagttgtaattaaactattcttcttcccaaatacccacatgtctaatgtgtgtgtgagggtgttaggcaggggacctgaagctgggggagaggcagacagttcccatggccccaagtctaggatggcatttggtattggttgatgggtgagagcaagagagggaatatttttgtgcatgatgtggtatcagcacctgtactacattttatggattccttcttctctttgcggtatgccctgacaataattatatccgtcagccttacccccttggcagtaggaaaactgaaactgtcttaaagtctcagctctactttctcagaggtgcaggcaagggcactgggagtctggggccctggaaaactgttctgactctgccacttgccagatagacctgaactagacacgttacctctttgtaccacttggctctaatcccttatctgtaaaaccagcattttcaaatggtgctttgcacatcagccttttgcataagctttgatttgataaaatgttttttgtgtttttaaaaagattaaaaaccacaggtttagataatttcaaagtaggcttccctttttctgtcattttcctattatttttaaaacctcacctccttgactccttgttccctttttctgcactgctgagtctgggagcactgaggccaggtaaaaggaaacttggcaaatgaggggcacctatgggtgtgggaggctgctcctggtgtttgcatattttaaaatttaaatgctacaaaccactgtgagttaggtattattgttcctattttaccattgaggaagctggggctcagagaaggtggagggtggtacagacaaacctgaattggaaccctggctcctgcctatgggctgtcaggacttagaaaagtcgtgagctctcgctgattgtttcctcagctgatgtgggctgcagggctgttatgggggaaataataagaaagtgcatcaagtgctgagcacatcctaagcactccatcatggcagctcctactactaataaagaatagaattatatctaacatgattctttcttgcaagtgacagaaaatccaactcaaattggattaagcaaaacaagggaaattcttagtgagctgcaaagttttcaggctcacatgatggccccaaatcccaggtcctcccaatcatggagtaggcactatttgggggcacaaaggtgacattcccatggctgcagatgctgtggtgctgtggctgtaccgggaaagaataagaaaggccactctcccaattatgtgaacaatagtctgcccactctgagaagtcaaacttgggtcacagtcctgcccctgaacccatcactgactggctctgacctgcaccaattgttccatgttggaggtgaaggcaagaccccactaatacccataaggggcaaaagttagatagatccttcaagaggattatgggaggtagggcaaaaagctgctgggcagccagaaagcaaacagagcctctatgatacctcaactgatgaaagcatgaagctaaaatcataaggatctgggtgtgagttctggctctcccatcttccatgtgacattgggcagttatttaatctcttttagcctccgctttctcatcttacatatgagataattgtgaggattaagattacacataatcatcatcatcaccgtccaccactaccaccatcatccccatcaacatcatcgccaccactatcatcattcttactggcactaccatcaccatcaccaccattccaccaccatcaccaatatcatcactgtcaacatcattaccaccatcaccatcaccaccaccatcatcattactaccactaccactactaccaccatcaccatcaccaccattccaccaccatcaccaatatcatcactctcaacatcatcaccatcaccatcaccaccaccatcatcatcattactaccactaccactactaccaccatcaccatcaccactgtcccactactatcagcatgacatcaccatcaccaccaccatcatcattaccaccgctactaccaacatcaccatcaccacaattctactgccatcaccattaacattaccaccaccatcatcactatcaccatcaccaccatcatcaccactgccattatcactgccaccatcatcactatcctctatatttcctcatctgtattatcattactaccaccatcactatcaccaccatcgtcaccatcataatcaccatcaacaccatctccaataccaccatcactgtaaccatcatcaccaccaccatgatcactatcaccatcatcacaatgatcactgtaaccatcattactacccaccaccatcaccactactccaccaccatcaccattatcattaccatcaccattatcaccaccatcatcatcaccagcaccaccatcatcaccagcaccaccatcaccatcaccatcattaacaccatcactatcaccattggtttaatcatcaccaccatcatcataaataaacatcacataaccagggtgtagctgggtgttgaccccagagcccactcactgtttcctctctcccacccccatccacacatttctaaccaccatcctgcactgggctcccagtctcctctggtctcacccacatgtccactgagaaaaggattttcagaacaccaactagaccaggaggagccacatacataactcaggcctgcttatcaactttctacatgttaataatgacatcagatcaatgggtgttctcagcttctcagaaggaggtcaaaattctccccctctccccttcatgtgtccagaccttcccggatttggatgtaccaagtgcagagtggtgttgaggccaaggggctcatccatgtaagtctcatctgcaatcactgggctgatcccgtggccctgtctccagggcgccatcagagagggcttcaatcctcaggttacctgtggcccaccctgccctcagaggtgccatctctacattggccacgagatggcagcacatactcatagactgcattaatttcccagcaactcctggtgggttttccctcttatcaggatgtttgccttgctcagagagcaaatctgagagcagtgacacctaacttaactttcagcaaaatattttgagaagggtgcccctttacacatctgtgcagtccaggtgatgcatcccatgcccaatgctcggtagtcaggaggagcttcctccatgcagctctgcggaagagactcttccacgctgctcatgtaaactccagattcggtgtcagttttctgacaccgaagacaatgatctaagtgcagtcaagggctttggggaaagcaggagagagtgcctcagttctagcctgtgccatgcttgcaaagttttgcaaaattctaatgagagctgggcttgcaacattggaaacttggattatttgtgagagcactgagaaatccctgggcatgtccatctggaaaaacagcatttcctctggcactttagcagaggttctgtttcaatttggcgaaggaaattaagcagtttttcacaaaagaagaactacaacgaggagaattgtccctagtatttcttctccctaattgtcaaggaagtgtaaattagaaaatgaatcaggacaatttccacctactatgttagctaatattttaaaaattgaatatcacaagggtgaggcaaagtaattgttttccagtgacattttccactgtcacacccttttagagaataatttggcaatgttactgtgagatagaaatatgtctatataattatgggaactgagacttcagaaagtaataaggaataagaatgaaatttatgaacaaacatgtggaaggttggaagcaagagtggggccaacacgcatggggaggaagcatttgggcagcgactccgcagacccagactcaagctgagctatacaacctccttacgcctcagtttcctcaactgaagaacaggaatgacaagtgcctgtttcataggaccgttgtgaggattaagtgagatataccacattatgagcttgtgcctggaaaggttgattcttagtaaatgatgactattcttttttattgcaataaaatttatacaacatagagttactattttaaccatttttgcaggtaccactgagtggcattcagtacattcacaatggtgtgcaaccgtcaccatatttccaggacatttttctcatccccaaaggaaacctcatgcccattaagcagtcactcctcattaaaatattagttatgaagactgtagcatttttttaaaaactcatgatataacattgattgaaaaaatcagtataggaaattgtgcattatgatgtaatagtaaaagaagcatataaaaatctgaaaaaagtatataaaaagaatagcaattgtatttctcagactctctttacattgtaaaaatcattttgatagcttcaaaagaaaagcaaaaagtacacaaacaacaaccaaccccaaagcagcatgacaaagcccagattgttgaatccaggtcttgggaacataaaatcttatatgacatttgcactttaatgggtcagagagtccagtggcattgggagctgccttgtgttctgcagcctcacggacagacaggaggtccagctccactgctctgttcttctggaatttcctcgtgaacaagctttggcctcagtaaccatttctttcatctttttaaacacaggtacctttgggactggccttctcaaggaagcccagctccttgttattgagaatgaagtgtgcaatcactataagtatatttgtgctgagcatttggccagaggcactgacagttgccaggtaagaaaagatcaatagatcaaagtcttgtgctctcccgtctcagtctcagtcccttagacgtcagtcccaaagtggcaaattcaggaaggttttgtcagtggaagaccccagtctaagtgttgctcagaaactccccagatctgtccctgaatgcatattcagatcatctaaggagacgtcttggggcttgagttccagatccatagcaagggagccgtaagtgccataactacctcaggccactcaccttcctggtgtgtgctggtcaccagtgactgaagtggtggcttttccagtagagaggaaggtagagggtacaggaccgagacaaattacacacacttaacaatgatgtccaggctagcccagtctaaaggaaacaccaagttaggaagcaatgcatgcaggattcacaagggattattttttttcccaggaaaaaactaagtgatgtggttttgttgaatagactttgctaagtacttaagcactgcagatgcttgagtaatatgctcataagttcctttctgatttgaattactgggaaaatgtacatatggataagagaaggatggcatcccatattaaaaggttggcagcttaaagctcacatgaattttcccctacctctgtttagggtgacagtggagggcctctggtttgcttcgagaaggacaaatacattttacaaggagtcacttcttggggtcttggctgtgcacgccccaataagcctggtgtctatgctcgtgtttcaaggtttgttacttggattgagggaatgatgagaaataattaattggacgggagacagagtgaagcatcaacctacttagaagctgaaacgtgggtaaggatttagcatgctggaaataatagacagcaatcaaacgaagacactgttcccagctaccagctatgccaaaccttggcatttttggtatttttgtgtataagcttttaaggtctgactgacaaattctgtattaaggtgtcatagctatgacatttgttaaaaataaactctgcacttattttgatttgaattaattttggttttggtcttcaaaattttcatgctcttttcatcccatctatttttatttttattttttagactttacgtcctggggtacatgtgcagaatgtgcaggtttgttacatagatgtacacgtgccatggtagtttgctgcacccatcaacctgtcatctaattcggtatttcttttagttctatccctcccctagccctccaccccttgacaggcccaggtgtgtgatgttgccctccctgtgtccatgtgttctcattgttcaactcacacttatgagtgagaacatgccgtgtttgtttttctgttcttgtgttagtttgctgagaatgatagtttccagcttcatccatgtccctgcaaaggacatgaactcatccttttttatggctgcatagaattccatggtgtatatgtgccacattttatccaatctaacattgatgggcaattgggttggttccaactctttgctattgtgaatagtgccacaataaacatacgtgtgcatgtgttttcatagcagaatgatttataatcctctgggtatatacccagtaatgggattgcagggtcaaatggtgtttctggtgctagatctttgaggaatcaccacactgtcttccacaatggttgaactaatttatgctcccaccaacaatatcaaggcattcctatttctccacatcctctccagcatctgttgtttcctgactttttaatgatcgccattctaactggcatgagatggtatctcattgtggttttgatttgcatttctctaatgatcagtgatgatgagcttttctcatatgtttgttggctgcataaatgccttttttggagaagcatctgttcatatcctttgcccactttttgatggtgttgtttttttctggtaaatttgtttaagttctttgtagattctggatattagccttttgtcagatggatagatggcaaaaattttatcctattatgtaggttgcctgttcactccgatgatagtttcttttgctgtgcagaagctctttggtttaattagatctcatttgtctattttggcttttgttaccattgcttttagtgttttagtcatgaagtcttctcccatgctatgtcctgaatggtattgcctaagttttcttccagggtttttatggttttaggttttgcatttaagtctttaatccatcttgagttaatttttgtataagtaatgcccttctttgtctcttttgatctttgttggcttaaagtatattttatcagagactagaattgcaatccctgcttttttttttctttttgctttccttttgcttggtaaatattcttccatccctttattttgagcctatgtatgtctgcacatgagataggtttcctgaatacagcacaccaatgggtcttgactctttattcaatttgccagtctgtgtcttttaattgggggcatttagtccatttacatttaaggttaatattgttatgtgtgaatttgatcctgtcattatgatgctagcgggttattttgcccattagttgatgcagtttcttcatagtgtggatggcctttacaatttggtagtttttgcagtggctggtaccaattgttcctttccatgtttagtgcttcgttcaggagctcttgtgaggcaggccttgtggtgacaaaatctttcagcatttgcttgtctgtaaaggattttatttctcctttgcttatgaagcttagtttcgctgggtatgaaattctgggttgaaaattattttcttttagaatgttgaatattggcccccactctcttcgggcttgttgggtttctgcagagagatccactgttagtctgattggcttccctttccgggtaacccaacctttctctctggctgcccttagaaatttttccttcatttcaaccttggtgaatctgacgattatgtcttgaggtggctcttct

SEQ ID NO: 4 - RNA, 13938 nt, Homo sapiens
  source 1..13938: mol_type = mRNA
ctgggattgggacacactttctggacactgctggccagtcccaaaatggaacataaggaagtggttcttctacttcttttatttctgaaatcagcagcacctgagcaaagccatgtggtccaggattgctaccatggtgatggacagagttatcgaggcacgtactccaccactgtcacaggaaggacctgccaagcttggtcatctatgacaccacatcaacataataggaccacagaaaactacccaaatgctggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaagcaccgactgagcaaaggcctggggtgcaggagtgctaccatggtaatggacagagttatcgaggcacatactccaccactgtcacaggaagaacctgccaagcttggtcatctatgacaccacactcgcatagtcggaccccagaatactacccaaatgctggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaagcaccgactgagcaaaggcctggggtgcaggagtgctaccatggtaatggacagagttatcgaggcacatactccaccactgtcacaggaagaacctgccaagcttggtcatctatgacaccacactcgcatagtcggaccccagaatactacccaaatgctggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaagcaccgactgagcaaaggcctggggtgcaggagtgctaccatggtaatggacagagttatcgaggcacatactccaccactgtcacaggaagaacctgccaagcttggtcatctatgacaccacactcgcatagtcggaccccagaatactacccaaatgctggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaagcaccgactgagcaaaggcctggggtgcaggagtgctaccatggtaatggacagagttatcgaggcacatactccaccactgtcacaggaagaacctgccaagcttggtcatctatgacaccacactcgcatagtcggaccccagaatactacccaaatgctggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaagcaccgactgagcaaaggcctggggtgcaggagtgctaccatggtaatggacagagttatcgaggcacatactccaccactgtcacaggaagaacctgccaagcttggtcatctatgacaccacactcgcatagtcggaccccagaatactacccaaatgctggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaagcaccgactgagcaaaggcctggggtgcaggagtgctaccatggtaatggacagagttatcgaggcacatactccaccactgtcacaggaagaacctgccaagcttggtcatctatgacaccacactcgcatagtcggaccccagaatactacccaaatgctggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaagcaccgactgagcaaaggcctggggtgcaggagtgctaccatggtaatggacagagttatcgaggcacatactccaccactgtcacaggaagaacctgccaagcttggtcatctatgacaccacactcgcatagtcggaccccagaatactacccaaatgctggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaagcaccgactgagcaaaggcctggggtgcaggagtgctaccatggtaatggacagagttatcgaggcacatactccaccactgtcacaggaagaacctgccaagcttggtcatctatgacaccacactcgcatagtcggaccccagaatactacccaaatgctggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaagcaccgactgagcaaaggcctggggtgcaggagtgctaccatggtaatggacagagttatcgaggcacatactccaccactgtcacaggaagaacctgccaagcttggtcatctatgacaccacactcgcatagtcggaccccagaatactacccaaatgctggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaagcaccgactgagcaaaggcctggggtgcaggagtgctaccatggtaatggacagagttatcgaggcacatactccaccactgtcacaggaagaacctgccaagcttggtcatctatgacaccacactcgcatagtcggaccccagaatactacccaaatgctggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaagcaccgactgagcaaaggcctggggtgcaggagtgctaccatggtaatggacagagttatcgaggcacatactccaccactgtcacaggaagaacctgccaagcttggtcatctatgacaccacactcgcatagtcggaccccagaatactacccaaatgctggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaagcaccgactgagcaaaggcctggggtgcaggagtgctaccatggtaatggacagagttatcgaggcacatactccaccactgtcacaggaagaacctgccaagcttggtcatctatgacaccacactcgcatagtcggaccccagaatactacccaaatgctggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaagcaccgactgagcaaaggcctggggtgcaggagtgctaccatggtaatggacagagttatcgaggcacatactccaccactgtcacaggaagaacctgccaagcttggtcatctatgacaccacactcgcatagtcggaccccagaatactacccaaatgctggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaagcaccgactgagcaaaggcctggggtgcaggagtgctaccatggtaatggacagagttatcgaggcacatactccaccactgtcacaggaagaacctgccaagcttggtcatctatgacaccacactcgcatagtcggaccccagaatactacccaaatgctggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaagcaccgactgagcaaaggcctggggtgcaggagtgctaccatggtaatggacagagttatcgaggcacatactccaccactgtcacaggaagaacctgccaagcttggtcatctatgacaccacactcgcatagtcggaccccagaatactacccaaatgctggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaagcaccgactgagcaaaggcctggggtgcaggagtgctaccatggtaatggacagagttatcgaggcacatactccaccactgtcacaggaagaacctgccaagcttggtcatctatgacaccacactcgcatagtcggaccccagaatactacccaaatgctggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaagcaccgactgagcaaaggcctggggtgcaggagtgctaccatggtaatggacagagttatcgaggcacatactccaccactgtcacaggaagaacctgccaagcttggtcatctatgacaccacactcgcatagtcggaccccagaatactacccaaatgctggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaagcaccgactgagcaaaggcctggggtgcaggagtgctaccatggtaatggacagagttatcgaggcacatactccaccactgtcacaggaagaacctgccaagcttggtcatctatgacaccacactcgcatagtcggaccccagaatactacccaaatgctggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaagcaccgactgagcaaaggcctggggtgcaggagtgctaccatggtaatggacagagttatcgaggcacatactccaccactgtcacaggaagaacctgccaagcttggtcatctatgacaccacactcgcatagtcggaccccagaatactacccaaatgctggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaagcaccgactgagcaaaggcctggggtgcaggagtgctaccatggtaatggacagagttatcgaggcacatactccaccactgtcacaggaagaacctgccaagcttggtcatctatgacaccacactcgcatagtcggaccccagaatactacccaaatgctggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaagcaccgactgagcaaaggcctggggtgcaggagtgctaccatggtaatggacagagttatcgaggcacatactccaccactgtcacaggaagaacctgccaagcttggtcatctatgacaccacactcgcatagtcggaccccagaatactacccaaatgctggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaagcaccgactgagcaaaggcctggggtgcaggagtgctaccatggtaatggacagagttatcgaggcacatactccaccactgtcacaggaagaacctgccaagcttggtcatctatgacaccacactcgcatagtcggaccccagaatactacccaaatgctggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaagcaccgactgagcagaggcctggggtgcaggagtgctaccacggtaatggacagagttatcgaggcacatactccaccactgtcactggaagaacctgccaagcttggtcatctatgacaccacactcgcatagtcggaccccagaatactacccaaatgctggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaagcaccgactgagcaaaggcctggggtgcaggagtgctaccatggtaatggacagagttatcgaggcacatactccaccactgtcacaggaagaacctgccaagcttggtcatctatgacaccacactcgcatagtcggaccccagaatactacccaaatgctggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaagcaccgactgagcaaaggcctggggtgcaggagtgctaccatggtaatggacagagttatcgaggcacatactccaccactgtcacaggaagaacctgccaagcttggtcatctatgacaccacactcgcatagtcggaccccagaatactacccaaatgctggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaagcaccgactgagcagaggcctggggtgcaggagtgctaccacggtaatggacagagttatcgaggcacatactccaccactgtcactggaagaacctgccaagcttggtcatctatgacaccacactcgcatagtcggaccccagaatactacccaaatgctggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaagcaccgactgagcagaggcctggggtgcaggagtgctaccacggtaatggacagagttatcgaggcacatactccaccactgtcactggaagaacctgccaagcttggtcatctatgacaccacactcgcatagtcggaccccagaatactacccaaatgctggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaagcaccgactgagcagaggcctggggtgcaggagtgctaccacggtaatggacagagttatcgaggcacatactccaccactgtcactggaagaacctgccaagcttggtcatctatgacaccacactcgcatagtcggaccccagaatactacccaaatgctggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaagcaccgactgagcagaggcctggggtgcaggagtgctaccacggtaatggacagagttatcgaggcacatactccaccactgtcactggaagaacctgccaagcttggtcatctatgacaccacactcgcatagtcggaccccagaatactacccaaatgctggcttgatcatgaactactgcaggaatccagatcctgtggcagccccttattgttatacgagggatcccagtgtcaggtgggagtactgcaacctgacacaatgctcagacgcagaagggactgccgtcgcgcctccaactattaccccgattccaagcctagaggctccttctgaacaagcaccaactgagcaaaggcctggggtgcaggagtgctaccacggaaatggacagagttatcaaggcacatacttcattactgtcacaggaagaacctgccaagcttggtcatctatgacaccacactcgcatagtcggaccccagcatactacccaaatgctggcttgatcaagaactactgccgaaatccagatcctgtggcagccccttggtgttatacaacagatcccagtgtcaggtgggagtactgcaacctgacacgatgctcagatgcagaatggactgccttcgtccctccgaatgttattctggctccaagcctagaggctttttttgaacaagcactgactgaggaaacccccggggtacaggactgctactaccattatggacagagttaccgaggcacatactccaccactgtcacaggaagaacttgccaagcttggtcatctatgacaccacaccagcatagtcggaccccagaaaactacccaaatgctggcctgaccaggaactactgcaggaatccagatgctgagattcgcccttggtgttacaccatggatcccagtgtcaggtgggagtactgcaacctgacacaatgcctggtgacagaatcaagtgtccttgcaactctcacggtggtcccagatccaagcacagaggcttcttctgaagaagcaccaacggagcaaagccccggggtccaggattgctaccatggtgatggacagagttatcgaggctcattctctaccactgtcacaggaaggacatgtcagtcttggtcctctatgacaccacactggcatcagaggacaacagaatattatccaaatggtggcctgaccaggaactactgcaggaatccagatgctgagattagtccttggtgttataccatggatcccaatgtcagatgggagtactgcaacctgacacaatgtccagtgacagaatcaagtgtccttgcgacgtccacggctgtttctgaacaagcaccaacggagcaaagccccacagtccaggactgctaccatggtgatggacagagttatcgaggctcattctccaccactgttacaggaaggacatgtcagtcttggtcctctatgacaccacactggcatcagagaaccacagaatactacccaaatggtggcctgaccaggaactactgcaggaatccagatgctgagattcgcccttggtgttataccatggatcccagtgtcagatgggagtactgcaacctgacgcaatgtccagtgatggaatcaactctcctcacaactcccacggtggtcccagttccaagcacagagcttccttctgaagaagcaccaactgaaaacagcactggggtccaggactgctaccgaggtgatggacagagttatcgaggcacactctccaccactatcacaggaagaacatgtcagtcttggtcgtctatgacaccacattggcatcggaggatcccattatactatccaaatgctggcctgaccaggaactactgcaggaatccagatgctgagattcgcccttggtgttacaccatggatcccagtgtcaggtgggagtactgcaacctgacacgatgtccagtgacagaatcgagtgtcctcacaactcccacagtggccccggttccaagcacagaggctccttctgaacaagcaccacctgagaaaagccctgtggtccaggattgctaccatggtgatggacggagttatcgaggcatatcctccaccactgtcacaggaaggacctgtcaatcttggtcatctatgataccacactggcatcagaggaccccagaaaactacccaaatgctggcctgaccgagaactactgcaggaatccagattctgggaaacaaccctggtgttacacaaccgatccgtgtgtgaggtgggagtactgcaatctgacacaatgctcagaaacagaatcaggtgtcctagagactcccactgttgttccagttccaagcatggaggctcattctgaagcagcaccaactgagcaaacccctgtggtccggcagtgctaccatggtaatggccagagttatcgaggcacattctccaccactgtcacaggaaggacatgtcaatcttggtcatccatgacaccacaccggcatcagaggaccccagaaaactacccaaatgatggcctgacaatgaactactgcaggaatccagatgccgatacaggcccttggtgttttaccatggaccccagcatcaggtgggagtactgcaacctgacgcgatgctcagacacagaagggactgtggtcgctcctccgactgtcatccaggttccaagcctagggcctccttctgaacaagactgtatgtttgggaatgggaaaggataccggggcaagaaggcaaccactgttactgggacgccatgccaggaatgggctgcccaggagccccatagacacagcacgttcattccagggacaaataaatgggcaggtctggaaaaaaattactgccgtaaccctgatggtgacatcaatggtccctggtgctacacaatgaatccaagaaaactttttgactactgtgatatccctctctgtgcatcctcttcatttgattgtgggaagcctcaagtggagccgaagaaatgtcctggaagcattgtaggggggtgtgtggcccacccacattcctggccctggcaagtcagtctcagaacaaggtttggaaagcacttctgtggaggcaccttaatatccccagagtgggtgctgactgctgctcactgcttgaagaagtcctcaaggccttcatcctacaaggtcatcctgggtgcacaccaagaagtgaacctcgaatctcatgttcaggaaatagaagtgtctaggctgttcttggagcccacacaagcagatattgccttgctaaagctaagcaggcctgccgtcatcactgacaaagtaatgccagcttgtctgccatccccagactacatggtcaccgccaggactgaatgttacatcactggctggggagaaacccaaggtacctttgggactggccttctcaaggaagcccagctccttgttattgagaatgaagtgtgcaatcactataagtatatttgtgctgagcatttggccagaggcactgacagttgccagggtgacagtggagggcctctggtttgcttcgagaaggacaaatacattttacaaggagtcacttcttggggtcttggctgtgcacgccccaataagcctggtgtctatgctcgtgtttcaaggtttgttacttggattgagggaatgatgagaaataattaattggacgggagacagagtgaagcatcaacctacttagaagctgaaacgtgggtaaggatttagcatgctggaaataatagacagcaatcaaacgaagacactgttcccagctaccagctatgccaaaccttggcatttttggtatttttgtgtataagcttttaaggtctgactgacaaattctgtattaaggtgtcatagctatgacatttgttaaaaataaactctgcacttattttgatttga

SEQ ID NO: 5 - RNA, 533 nt, Homo sapiens
  source 1..533: mol_type = mRNA
tgctcagttcatccctagaggcagctgctccaggaacagaggtgccatgcagccccgggtactccttgttgttgccctcctggcgctcctggcctctgcccgagcttcagaggccgaggatgcctcccttctcagcttcatgcagggttacatgaagcacgccaccaagaccgccaaggatgcactgagcagcgtgcaggagtcccaggtggcccagcaggccaggggctgggtgaccgatggcttcagttccctgaaagactactggagcaccgttaaggacaagttctctgagttctgggatttggaccctgaggtcagaccaacttcagccgtggctgcctgagacctcaataccccaagtccacctgcctatccatcctgcgagctccttgggtcctgcaatctccagggctgcccctgtaggttgcttaaaagggacagtattctcagtgctctcctaccccacctcatgcctggcccccctccaggcatgctggcctcccaataaagctggacaagaagctgctatg

SEQ ID NO: 6 - DNA, 3964 bp, Homo sapiens
  source 1..3964: mol_type = genomic DNA
ctactccaggctgtgttcagggcttggggctggtggagggaggggcctgaaattccagtgtgaaaggctgagatgggcccgaggcccctggcctatgtccaagccatttcccctctcaccagcctctccctggggagccagtcagctaggaaggaatgagggctccccaggcccacccccagttcctgagctcatctgggctgcagggctggcgggacagcagcgtggactcagtctcctagggatttcccaactctcccgcccgcttgctgcatctggacaccctgcctcaggccctcatctccactggtcagcaggtgacctttgcccagcgccctgggtcctcagtgcctgctgccctggagatgatataaaacaggtcagaaccctcctgcctgtctgctcagttcatccctagaggcagctgctccaggtaatgccctctggggaggggaaagaggaggggaggaggatgaagaggggcaagaggagctccctgcccagcccagccagcaagcctggagaagcacttgctagagctaaggaagcctcggagctggacgggtgccccccacccctcatcataacctgaagaacatggaggcccgggaggggtgtcacttgcccaaagctacacagggggtggggctggaagtggctccaagtgcaggttcccccctcattcttcaggcttagggctggaggaagccttagacagcccagtcctaccccagacagggaaactgaggcctggagagggccagaaatcacccaaagacacacagcatgttggctggactggacggagatcagtccagaccgcaggtgccttgatgttcagtctggtgggttttctgctccatcccacccacctccctttgggcctcgatccctcgcccctcaccagtcccccttctgagagcccgtattagcagggagccggcccctactccttctggcagacccagctaaggttctaccttaggggccacgccacctccccagggaggggtccagaggcatggggacctggggtgcccctcacaggacacttccttgcaggaacagaggtgccatgcagccccgggtactccttgttgttgccctcctggcgctcctggcctctgcccgtaagcacttggtgggactgggctgggggcagggtggaggcaacttggggatcccagtcccaatgggtggtcaagcaggagcccagggctcgtccagaggccgatccaccccactcagccctgctctttcctcaggagcttcagaggccgaggatgcctcccttctcagcttcatgcagggttacatgaagcacgccaccaagaccgccaaggatgcactgagcagcgtgcaggagtcccaggtggcccagcaggccaggtacacccgctggcctccctccccatcccccctgccagctgcctccattcccacccgcccctgccctggtgagatcccaacaatggaatggaggtgctccagcctcccctgggcctgtgcctcttcagcctcctctttcctcacagggcctttgtcaggctgctgcgggagagatgacagagttgagactgcattcctcccaggtccctcctttctccccggagcagtcctagggcgtgccgttttagccctcatttccattttcctttcctttccctttctttctctttctatttctttctttctttctttctttctttctttctttctttctttctttctttctttctttctttctttcctttctttctttcctttctttctttcctttctttctttctttcctttctttctctttctttctttctttcctttttctttctttccctctcttcctttctctctttctttcttcttcttttttttttaatggagtctccctctgtcacctaggctggagtgcagtggtgccatctcggctcactgcaacctccgtctcccgggttcaacccattctcctgcctcagcctcccaagtagctgggattacaggcacgcgccaccacacccagctaatttttgtatttttagcagagatggggtttcaccatgttggccaggttggtcttgaattcctgacctcaggggatcctcctgcctcggcctcccaaagtgctgggattacaggcatgagccactgcgcctggccccattttccttttctgaaggtctggctagagcagtggtcctcagcctttttggcaccagggaccagttttgtggtggacaatttttccatgggccagcggggatggttttgggatgaagctgttccacctcagatcatcaggcattagattctcataaggagccctccacctagatccctggcatgtgcagttcacaatagggttcacactcctatgagaatgtaaggccacttgatctgacaggaggcggagctcaggcggtattgctcactcacccaccactcacttcgtgctgtgcagcccggctcctaacagtccatggaccagtacctatctatgacttgggggttggggacccctgggctaggggtttgccttgggaggccccacctgacccaattcaagcccgtgagtgcttctgctttgttctaagacctggggccagtgtgagcagaagtgtgtccttcctctcccatcctgcccctgcccatcagtactctcctctcccctactcccttctccacctcaccctgactggcattagctggcatagcagaggtgttcataaacattcttagtccccagaaccggctttggggtaggtgttattttctcactttgcagatgagaaaattgaggctcagagcgattaggtgacctgccccagatcacacaactaatcaatcctccaatgactttccaaatgagaggctgcctccctctgtcctaccctgctcagagccaccaggttgtgcaactccaggcggtgctgtttgcacagaaaacaatgacagccttgacctttcacatctccccaccctgtcactttgtgcctcaggcccaggggcataaacatctgaggtgacctggagatggcagggtttgacttgtgctggggttcctgcaaggatatctcttctcccagggtggcagctgtgggggattcctgcctgaggtctcagggctgtcgtccagtgaagttgagagggtggtgtggtcctgactggtgtcgtccagtggggacatgggtgtgggtcccatggttgcctacagaggagttctcatgccctgctctgttgcttcccctgactgatttaggggctgggtgaccgatggcttcagttccctgaaagactactggagcaccgttaaggacaagttctctgagttctgggatttggaccctgaggtcagaccaacttcagccgtggctgcctgagacctcaataccccaagtccacctgcctatccatcctgcgagctccttgggtcctgcaatctccagggctgcccctgtaggttgcttaaaagggacagtattctcagtgctctcctaccccacctcatgcctggcccccctccaggcatgctggcctcccaataaagctggacaagaagctgctatgagtgggccgtcgcaagtgtgccatctgtgtctgggcatgggaaagggccgaggctgttctgtgggtgggcactggacagactccaggtcaggcaggcatggaggccagcgctctatccaccttctggtagctgggcagtctctgggcctcagtttcttcatctctaaggtaggaatcaccctccgtaccctgccttccttgacagctttgtgcggaaggtcaaacaggacaataagtttgctgatactttgataaactgttaggtgctgcacaacatgacttgagtgtgtgccccatgccagccactatgcctggcacttaagttgtcatcagagttgagactgtgtgtgtttactcaaaactgtggagctgacctcccctatccaggccccctagccct

SEQ ID NO: 7 - DNA, 20 bp, synthetic construct
  source 1..20: mol_type = other DNA
tgctccgttggtgcttgttc

SEQ ID NO: 8 - DNA, 20 bp, synthetic construct
  source 1..20: mol_type = other DNA
tcttcctgtgacagtggtgg

SEQ ID NO: 9 - DNA, 20 bp, synthetic construct
  source 1..20: mol_type = other DNA
ttcttcctgtgacagtggtg

SEQ ID NO: 10 - DNA, 20 bp, synthetic construct
  source 1..20: mol_type = other DNA
ggttcttcctgtgacagtgg

SEQ ID NO: 11 - DNA, 20 bp, synthetic construct
  source 1..20: mol_type = other DNA
cgactatgcgagtgtggtgt

SEQ ID NO: 12 - DNA, 20 bp, synthetic construct
  source 1..20: mol_type = other DNA
ccgactatgcgagtgtggtg

SEQ ID NO: 13 - DNA, 20 bp, synthetic construct
  source 1..20: mol_type = other DNA
agcttcttgtccagctttat